(19) United States
(12) Patent Application Publication
(10) Pub. No.: US 2019/0185559 A1
(43) Pub. Date: Jun. 20, 2019

(54) COMPOSITIONS AND METHODS FOR TREATING NEOPLASIAS

(71) Applicants: Russell J.H RYAN, Boston, MA (US); Bradley E. BERNSTEIN, Boston, MA (US); Jon ASTER, Boston, MA (US); Warren PEAR, Philadelphia, PA (US); THE BRIGHAM AND WOMEN'S HOSPITAL, INC., BOSTON, MA (US); THE GENERAL HOSPITAL CORPORATION, BOSTON, MA (US); THE TRUSTEES OF THE UNIVERSITY OF PENNSYLVANIA, PHILADELPHIA, PA (US)

(72) Inventors: RYAN J.H. RUSSELL, BOSTON, MA (US); BRADLEY E. BERNSTEIN, BOSTON, MA (US); JON ASTER, BOSTON, MA (US); WARREN S. PEAR, PHILADELPHIA, PA (US)

(73) Assignees: THE BRIGHAM AND WOMEN'S HOSPITAL, INC., BOSTON, MA (US); THE GENERAL HOSPITAL CORPORATION, BOSTON, MA (US); THE TRUSTEES OF THE UNIVERSITY OF PENNSYLVANIA, PHILADELPHIA, PA (US)

(21) Appl. No.: 16/328,080

(22) PCT Filed: Sep. 1, 2017
(86) PCT No.: PCT/US2017/049829

§ 371 Date: Feb. 25, 2019

Related U.S. Application Data
(60) Provisional application No. 62/383,111, filed on Sep. 2, 2016.

(51) Int. Cl. C07K 16/28; A61P 35/00; A61K 39/395; A61K 31/506; A61K 31/52; A61K 31/675; A61K 31/553; A61K 31/454; A61K 31/517

(52) U.S. Cl. CPC C07K 16/28; A61P 35/00; A61K 39/3955; A61K 31/506; A61K 31/517; A61K 31/675; A61K 31/553; A61K 31/454; A61K 31/52

(57) ABSTRACT

The invention provides therapeutic combinations comprising an agent that inhibits Notch signaling and an agent that inhibits B cell receptor signaling, and methods of using such agents to inhibit the survival or proliferation of a neoplastic cell.

### CROSS-REFERENCE TO RELATED APPLICATION

This application claims priority to U.S. Provisional Patent Application Ser. No. 62/383,111, filed on Sep. 2, 2016. The entire content of this application is hereby incorporated by reference herein.

### BACKGROUND OF THE INVENTION

Chronic lymphocytic leukemia (CLL) and mantle cell lymphoma (MCL) are two prevalent lymphoid malignancies that share the phenotype of small, mature, non-germinal center B-cells, but demonstrate distinctive clinical and biological features. Somatic mutations of the NOTCH1 gene are seen in 8-15% of CLL and MCL patients, while recurrent NOTCH2 mutations have also been reported in MCL. Notch gene mutations are associated with decreased overall survival and reduced time to treatment in both CLL and MCL, while in CLL, NOTCH1 mutations also appear to increase the risk of high-grade transformation, and reduce responsiveness to anti-CD20 monoclonal antibody therapy. In recent years, the clinical development of drugs targeting B-cell receptor (BCR) signaling and anti-apoptotic pathways have provided new options for patients with small B-cell lymphomas, but new approaches are still needed to improve response rate and prevent development of secondary drug resistance.

### SUMMARY OF THE INVENTION

The invention provides therapeutic combinations comprising an agent that inhibits Notch signaling and an agent that inhibits B cell receptor signaling, and methods of using such agents to inhibit the survival or proliferation of a neoplastic cell.

In one aspect, the invention provides a pharmaceutical composition containing an effective amount of an agent that inhibits the expression or activity of a Notch polynucleotide or polypeptide and an effective amount of an agent that inhibits the expression or activity of a functional component of a B cell receptor polypeptide or polynucleotide.

In another aspect, the invention provides a method of inhibiting the survival or proliferation of a neoplastic cell, the method involving contacting the cell with an agent that inhibits expression or activity of a Notch polynucleotide or polypeptide and an effective amount of an agent that inhibits expression or activity of a functional component of a B cell receptor polypeptide or polynucleotide

In yet another aspect, the invention provides a method of inhibiting the survival or proliferation of a neoplastic cell, the method involving contacting the cell with a gamma secretase inhibitor and ibrutinib, thereby inhibiting the survival or proliferation of the neoplastic cell.

In still another aspect, the invention provides a method of treating a neoplasia in a subject, the method involving administering to the subject an agent that inhibits the expression or activity of a Notch polynucleotide or polypeptide and an effective amount of an agent that inhibits the expression or activity of a functional component of a B cell receptor polypeptide or polynucleotide, thereby treating cancer in the subject.

In still another aspect, the invention provides a method of treating a subject having a leukemia or lymphoma, the method involving administering to the subject a gamma secretase inhibitor and ibrutinib.

In still another aspect, the invention provides a method of treating a subject having a leukemia or lymphoma that has developed resistance to a B cell receptor signaling inhibitor, the method involving administering a gamma secretase inhibitor and an agent that inhibits expression or activity of a functional component of the B cell receptor.

In various embodiments of any of the above aspects or any other aspect of the invention delineated herein, the agent is a small compound, polypeptide, or polynucleotide. In various embodiments of any of the above aspects or any other aspect of the invention delineated herein, the agent that inhibits Notch expression or activity is a gamma secretase inhibitor (e.g., Compound E, MK-0752, PF03084014, RO-4929097, DAPT, N-[N-(3,5-difluorophenacetyl)-L-alanyl]-S-phenylglycine t-butyl ester, tetralin imidazole PF-03084014, LY3039478, and BMS906-024), a Notch signaling pathway inhibitory antibody (e.g., anti-Delta-like-4 antibody), or an anti-Notch1 antibody (e.g., OMP-52M521). In various embodiments of any of the above aspects, the agent that inhibits Notch expression or activity is an inhibitory nucleic acid molecule. In various embodiments of any of the above aspects, the agent that inhibits B cell receptor signaling is a PI3 kinase inhibitor (e.g., idelalisib), BTK inhibitor (e.g., ibrutinib, ACP-196, ONO/GS-4059, BGB-3111, and CC-292), SRC family kinase inhibitor (e.g., Dasatinib), SYK inhibitor (e.g., Fostamatinib), or a protein kinase C inhibitor (e.g., Midostaurin, Enzastuarin, or Sotrasturin). In embodiments of any of the above aspects, the agents are formulated together or are formulated separately for simultaneous, separate or sequential co-administration. In embodiments of any of the above aspects or any other aspect of the invention delineated herein, a composition of the invention contains an agent that inhibits Notch expression or activity, an agent that inhibits B cell receptor expression or activity, and one or more additional therapeutic agents. In embodiments of any of the above aspects, the Notch activity is signaling. In embodiments of any of the above aspects, B cell receptor activity is signaling. The method further involves administration of one or more additional therapeutic agents. In embodiments of any of the above aspects, the neoplastic cell is derived from a leukemia or lymphoma. In embodiments of any of the above aspects, the leukemia is any one or more of a chronic lymphocytic leukemia, B cell acute lymphoblastic leukemia, T-cell acute lymphoblastic leukemia, and early T cell acute lymphoblastic leukemia. In embodiments of any of the above aspects, the lymphoma is any one or more of small B-cell lymphomas, mantle cell lymphoma, small lymphocytic lymphoma, diffuse large B cell lymphoma, splenic marginal zone lymphoma, follicular lymphoma, splenic red pulp lymphoma, and MALT lymphoma. In embodiments of any of the above aspects, the neoplastic cell is a murine, rat, or human cell. In embodiments of any of the above aspects, the cell is in vitro or in vivo.

### DEFINITIONS

Unless defined otherwise, all technical and scientific terms used herein have the meaning commonly understood by a person of ordinary skill in the art to which this invention belongs. The following references provide a person of ordinary skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “B cell receptor activity” is meant activation of proteins within the B-cell receptor (BCR) pathway that result in B cell activation. Such activation can take the form of tyrosine kinase phosphorylation (e.g., phosphorylation by a Src family kinase, Lyn, spleen tyrosine kinase (Syk), Bruton tyrosine kinase (Btk), Phospholipase C gamma 2 (PLCG2)), as well as activation or modulation of proteins in downstream pathways as a result of BCR signaling (e.g. phosphoinositol-3-kinase (PI3K)/AKT pathway protein phosphorylation, mitogen-activated protein kinase (MAPK) pathway protein phosphorylation, or protein kinase C/nuclear factor kappa B (NF-κB) phosphorylation, altered proteolysis, altered ubiquitination, or altered subcellular localization). In one embodiment, B cell receptor activity is B cell receptor signaling.

By “Notch activity” is meant activation of proteins within the Notch pathway that results in modifications in cell growth or proliferation. Such protein activation can take the form of proteolytic cleavage of Notch receptor proteins (or chimaeric proteins incorporating a portion of a Notch receptor protein), altered subcellular localization of Notch receptor proteins or a portion thereof from cellular membranes to the nucleus, cytoplasm, or other organelles, binding of Notch receptor proteins or a portion thereof to DNA (either directly or via binding of Notch proteins to other DNA-bound proteins), or binding of Notch proteins to transcriptional regulatory proteins independendent of association with DNA. In one embodiment, Notch activity is Notch signaling.

By “B cell receptor” is meant a transmembrane receptor protein complex present on B cells comprising a membrane bound immunoglobulin, CD79A and CD79B as functional components.

By “CD79A protein” is meant a polypeptide having at least about 85% amino acid identity to the sequence provided at NCBI Reference Sequence: P11912, or a fragment thereof, and having signal transduction activity.

>sp|P11912|CD79A_HUMAN B-cell antigen receptor
complex-associated protein alpha chain OS = Homo
sapiens GN = CD79A PE = 1 SV = 2
MPGGPGVLQALPATIFLLFLLSAVYLGPGCQALWMHKVPASLMVSLGEDA
HFQCPHNSSNNANVTWWRVLHGNYTWPPEFLGPGEDPNGTLIIQNVNKSH
GGIYVCRVQEGNESYQQSCGTYLRVRQPPPRPFLDMGEGTKNRIITAEGI
ILLFCAVVPGILLLFRKRWQNEKLGLDAGDEYEDENLYEGLNLDDCSMYE
DISRGLQGTYQDVGSLNIGDVQLEKP

By “CD79A polynucleotide” is meant a nucleic acid molecule encoding the CD79A protein.

By “CD79B protein” is meant a polypeptide having at least about 85% amino acid identity to the sequence provided at NCBI Reference Sequence: P40259, or a fragment thereof, and having signal transduction activity.

>sp|P40259|CD79B_HUMAN B-cell antigen receptor
complex-associated protein beta chain OS = Homo
sapiens GN = CD79B PE = 1 SV = 1
MARLALSPVPSHWMVALLLLLSAEPVPAARSEDRYRNPKGSACSRIWQSP
RFIARKRGFTVKMHCYMNSASGNVSWLWKQEMDENPQQLKLEKGRMEESQ
NESLATLTIQGIRFEDNGIYFCQQKCNNTSEVYQGCGTELRVMGFSTLAQ
LKQRNTLKDGIIMIQTLLIILFIIVPIFLLLDKDDSKAGMEEDHTYEGLD
IDQTATYEDIVTLRTGEVKWSVGEHPGQE

By “CD79B polynucleotide” is meant a nucleic acid molecule encoding the CD79B protein.

By “Bruton's tyrosine kinase (BTK) polypeptide” is meant a protein having at least about 85% amino acid identity to the sequence provided at NCBI Reference Sequence: Q06187.3, or a fragment thereof, and having tyrosine kinase activity. An exemplary BTK amino acid sequence is provided below:

| | |
|---|---|
| 1 | maavilesif lkrsqqkkkt splnfkkrlf lltvhklsyy eydfergrrg skkgsidvek |
| 61 | itcvetvvpe knppperqip rrgeesseme qisiierfpy pfqvvydegp lyvfspteel |
| 121 | rkrwihqlkn virynsdlvq kyhpcfwidg qylccsqtak namgcqilen rngslkpgss |
| 181 | hrktkkplpp tpeedqilkk plppepaaap vstselkkvv alydympmna ndlqlrkgde |
| 241 | yfileesnlp wwrardkngq egyipsnyvt eaedsiemye wyskhmtrsq aeqllkqegk |
| 301 | eggfivrdss kagkytvsvf akstgdpqgv irhyvvcstp qsqyylaekh lfstipelin |
| 361 | yhqhnsagli srlkypvsqq nknapstagl gygsweidpk dltflkelgt gqfgvvkygk |
| 421 | wrgqydvaik mikegsmsed efieeakvmm nlsheklvql ygvctkqrpi fiiteymang |
| 481 | cllnylremr hrfqtqqlle mckdvceame yleskqflhr dlaarnclvn dqgvvkvsdf |
| 541 | glsryvldde ytssvgskfp vrwsppevlm yskfssksdi wafgvlmwei yslgkmpyer |
| 601 | ftnsetaehi aqglrlyrph lasekvytim yscwhekade rptfkillsn ildvmdees |

By “BTK polynucleotide” is meant a nucleic acid molecule encoding a BTK polypeptide. An exemplary BTK polynucleotide sequence is provided at NCBI Reference Sequence: NM 000061.2, and reproduced herein below.

| | |
|---|---|
| 1 | aactgagtgg ctgtgaaagg gtggggtttg ctcagactgt ccttcctctc tggactgtaa |
| 61 | gaatatgtct ccagggccag tgtctgctgc gatcgagtcc caccttccaa gtcctggcat |
| 121 | ctcaatgcat ctgggaagct acctgcatta agtcaggact gagcacacag gtgaactcca |
| 181 | gaaagaagaa gctatggccg cagtgattct ggagagcatc tttctgaagc gatcccaaca |
| 241 | gaaaaagaaa acatcacctc taaacttcaa gaagcgcctg tttctcttga ccgtgcacaa |
| 301 | actctcctac tatgagtatg actttgaacg tgggagaaga ggcagtaaga agggttcaat |
| 361 | agatgttgag aagatcactt gtgttgaaac agtggttcct gaaaaaaatc ctcctccaga |
| 421 | aagacagatt ccgagaagag gtgaagagtc cagtgaaatg gagcaaattt caatcattga |
| 481 | aaggttccct tatcccttcc aggttgtata tgatgaaggg cctctctacg tcttctcccc |
| 541 | aactgaagaa ctaaggaagc ggtggattca ccagctcaaa aacgtaatcc ggtacaacag |
| 601 | tgatctggtt cagaaatatc acccttgctt ctggatcgat gggcagtatc tctgctgctc |
| 661 | tcagacagcc aaaaatgcta tgggctgcca aattttggag aacaggaatg gaagcttaaa |
| 721 | acctgggagt tctcaccgga agacaaaaaa gcctcttccc ccaacgcctg aggaggacca |
| 781 | gatcttgaaa aagccactac cgcctgagcc agcagcagca ccagtctcca caagtgagct |
| 841 | gaaaaaggtt gtggcccttt atgattacat gccaatgaat gcaaatgatc tacagctgcg |
| 901 | gaagggtgat gaatatttta tcttggagga aagcaactta ccatggtgga gagcacgaga |
| 961 | taaaaatggg caggaaggct acattcctag taactatgtc actgaagcag aagactccat |
| 1021 | agaaatgtat gagtggtatt ccaaacacat gactcggagt caggctgagc aactgctaaa |
| 1081 | gcaagagggg aaagaaggag gtttcattgt cagagactcc agcaaagctg gcaaatatac |
| 1141 | agtgtctgtg tttgctaaat ccacagggga ccctcaaggg gtgatacgtc attatgttgt |
| 1201 | gtgttccaca cctcagagcc agtattacct ggctgagaag caccttttca gcaccatccc |
| 1261 | tgagctcatt aactaccatc agcacaactc tgcaggactc atatccaggc tcaaatatcc |
| 1321 | agtgtctcaa caaaacaaga atgcaccttc cactgcaggc ctgggatacg gatcatggga |
| 1381 | aattgatcca aaggacctga ccttcttgaa ggagctgggg actggacaat ttggggtagt |
| 1441 | gaagtatggg aaatggagag gccagtacga cgtggccatc aagatgatca aagaaggctc |
| 1501 | catgtctgaa gatgaattca ttgaagaagc caaagtcatg atgaatcttt cccatgagaa |
| 1561 | gctggtgcag ttgtatggcg tctgcaccaa gcagcgcccc atcttcatca tcactgagta |
| 1621 | catggccaat ggctgcctcc tgaactacct gagggagatg cgccaccgct tccagactca |
| 1681 | gcagctgcta gagatgtgca aggatgtctg tgaagccatg gaatacctgg agtcaaagca |
| 1741 | gttccttcac cgagacctgg cagctcgaaa ctgtttggta aacgatcaag gagttgttaa |
| 1801 | agtatctgat ttcggcctgt ccaggtatgt cctggatgat gaatacacaa gctcagtagg |
| 1861 | ctccaaattt ccagtccggt ggtccccacc ggaagtcctg atgtatagca agttcagcag |
| 1921 | caaatctgac atttgggctt ttggggtttt gatgtgggaa atttactccc tggggaagat |
| 1981 | gccatatgag agatttacta acagtgagac tgctgaacac attgcccaag gcctacgtct |
| 2041 | ctacaggcct catctggctt cagagaaggt atataccatc atgtacagtt gctggcatga |
| 2101 | gaaagcagat gagcgtccca ctttcaaaat tcttctgagc aatattctag atgtcatgga |
| 2161 | tgaagaatcc tgagctcgcc aataagcttc ttggttctac ttctcttctc cacaagcccc |
| 2221 | aatttcactt tctcagagga aatcccaagc ttaggagccc tggagccttt gtgctcccac |
| 2281 | tcaatacaaa aaggcccctc tctacatctg ggaatgcacc tcttctttga ttccctggga |
| 2341 | tagtggcttc tgagcaaagg ccaagaaatt attgtgcctg aaatttcccg agagaattaa |
| 2401 | gacagactga atttgcgatg aaaatatttt ttaggaggga ggatgtaaat agccgcacaa |
| 2461 | aggggtccaa cagctctttg agtaggcatt tggtagagct tgggggtgtg tgtgtggggg |
| 2521 | tggaccgaat ttggcaagaa tgaaatggtg tcataaagat gggaggggag ggtgttttga |
| 2581 | taaaataaaa ttactagaaa gcttgaaagt c |

By “myc proto-oncogene protein (MYC of c-MYC) polypeptide” is meant a protein having at least about 85% amino acid identity to the sequence provided at NCBI Reference

Sequence: NP_002458.2, or a fragment thereof, and having growth regulatory activity. Growth regulatory activity includes, but is not limited to, cell division or increase in cell size. An exemplary MYC amino acid sequence is provided below:

| | |
|---|---|
| 1 | mdffrvvenq qppatmplnv sftnrnydld ydsvqpyfyc deeenfyqqq qqselqppap |
| 61 | sediwkkfel lptpplspsr rsglcspsyv avtpfslrgd ndggggsfst adqlemvtel |
| 121 | lggdmvnqsf icdpddetfi kniiiqdcmw sgfsaaaklv seklasyqaa rkdsgspnpa |
| 181 | rghsvcstss lylqdlsaaa secidpsvvf pyplndsssp kscasqdssa fspssdslls |
| 241 | stesspqgsp eplvlheetp pttssdseee qedeeeidvv svekrqapgk rsesgspsag |
| 301 | ghskpphspl vlkrchvsth qhnyaappst rkdypaakrv kldsvrvlrq isnnrkctsp |
| 361 | rssdteenvk rrthnvlerq rrnelkrsff alrdqipele nnekapkvvi lkkatayils |
| 421 | vqaeeqklis eedllrkrre qlkhkleqlr nsca |

By “MYC polynucleotide” is meant a nucleic acid molecule encoding a MYC polypeptide. An exemplary MYC polynucleotide sequence is provided at NCBI Reference Sequence: V00568.1, and reproduced herein below.

| | |
|---|---|
| 1 | ctgctcgcgg ccgccaccgc cgggccccgg ccgtccctgg ctcccctcct gcctcgagaa |
| 61 | gggcagggct tctcagaggc ttggcgggaa aaaagaacgg agggagggat cgcgctgagt |
| 121 | ataaaagccg gttttcgggg ctttatctaa ctcgctgtag taattccagc gagaggcaga |
| 181 | gggagcgagc gggcggccgg ctagggtgga agagccgggc gagcagagct gcgctgcggg |
| 241 | cgtcctggga agggagatcc ggagcgaata gggggcttcg cctctggccc agccctcccg |
| 301 | cttgatcccc caggccagcg gtccgcaacc cttgccgcat ccacgaaact ttgcccatag |
| 361 | cagcgggcgg gcactttgca ctggaactta caacacccga gcaaggacgc gactctcccg |
| 421 | acgcggggag gctattctgc ccatttgggg acacttcccc gccgctgcca ggacccgctt |
| 481 | ctctgaaagg ctctccttgc agctgcttag acgctggatt tttttcgggt agtggaaaac |
| 541 | cagcagcctc ccgcgacgat gcccctcaac gttagcttca ccaacaggaa ctatgacctc |
| 601 | gactacgact cggtgcagcc gtatttctac tgcgacgagg aggagaactt ctaccagcag |
| 661 | cagcagcaga gcgagctgca gcccccggcg cccagcgagg atatctggaa gaaattcgag |
| 721 | ctgctgccca ccccgcccct gtcccctagc cgccgctccg ggctctgctc gccctcctac |
| 781 | gttgcggtca cacccttctc ccttcgggga gacaacgacg gcggtggcgg gagcttctcc |
| 841 | acggccgacc agctggagat ggtgaccgag ctgctgggag gagacatggt gaaccagagt |
| 901 | ttcatctgcg acccggacga cgagaccttc atcaaaaaca tcatcatcca ggactgtatg |
| 961 | tggagcggct tctcggccgc cgccaagctc gtctcagaga agctggcctc ctaccaggct |
| 1021 | gcgcgcaaag acagcggcag cccgaacccc gcccgcggcc acagcgtctg ctccacctcc |
| 1081 | agcttgtacc tgcaggatct gagcgccgcc gcctcagagt gcatcgaccc ctcggtggtc |
| 1141 | ttcccctacc ctctcaacga cagcagctcg cccaagtcct gcgcctcgca agactccagc |
| 1201 | gccttctctc cgtcctcgga ttctctgctc tcctcgacgg agtcctcccc gcagggcagc |
| 1261 | cccgagcccc tggtgctcca tgaggagaca ccgcccacca ccagcagcga ctctgaggag |
| 1321 | gaacaagaag atgaggaaga aatcgatgtt gtttctgtgg aaaagaggca ggctcctggc |
| 1381 | aaaaggtcag agtctggatc accttctgct ggaggccaca gcaaacctcc tcacagccca |
| 1441 | ctggtcctca agaggtgcca cgtctccaca catcagcaca actacgcagc gcctccctcc |
| 1501 | actcggaagg actatcctgc tgccaagagg gtcaagttgg acagtgtcag agtcctgaga |
| 1561 | cagatcagca acaaccgaaa atgcaccagc cccaggtcct cggacaccga ggagaatgtc |
| 1621 | aagaggcgaa cacacaacgt cttggagcgc cagaggagga acgagctaaa acggagcttt |
| 1681 | tttgccctgc gtgaccagat cccggagttg gaaaacaatg aaaaggcccc caaggtagtt |
| 1741 | atccttaaaa aagccacagc atacatcctg tccgtccaag cagaggagca aaagctcatt |
| 1801 | tctgaagagg acttgttgcg gaaacgacga gaacagttga aacacaaact tgaacagcta |
| 1861 | cggaactctt gtgcgtaagg aaaagtaagg aaaacgattc cttctaacag aaatgtcctg |
| 1921 | agcaatcacc tatgaacttg tttcaaatgc atgatcaaat gcaacctcac aaccttggct |
| 1981 | gagtcttgag actgaaagat ttagccataa tgtaaactgc ctcaaattgg actttgggca |
| 2041 | taaaagaact tttttatgct taccatcttt tttttttctt taacagattt gtatttaaga |
| 2101 | attgttttta aaaaatttta a |

By “Notch protein” or “Notch receptor” is meant any one of Notch 1, 2, 3, or 4.

By “Neurogenic locus notch homolog protein 1 (Notch1) polypeptide” is meant a protein having at least about 85% amino acid identity to the sequence provided at NCBI Reference Sequence: P46531.4, or a fragment thereof, and having Notch receptor activity. Examples of Notch receptor activity include interaction with Notch ligands at the cell surface, proteolytic cleavage of the Notch protein by ADAM family metalloproteases and/or gamma secretase (either following interaction with Notch ligands, or through ligand-independent mechanisms), altered sub-cellular localization of an intracellular portion of the Notch protein following a proteolytic cleavage event, binding of a Notch protein (or portion thereof) to other transcriptional regulatory proteins in the nucleus or cytoplasm, or binding of a Notch protein (or portion thereof) to DNA-bound chromatin complexes. An exemplary Notch1 amino acid sequence is provided below:

| | |
|---|---|
| 1 | mppllapllc lallpalaar gprcsqpget clnggkceaa ngteacvcgg afvgprcqdp |
| 61 | npclstpckn agtchvvdrr gvadyacsca lgfsgplclt pldnacltnp crnggtcdll |
| 121 | tlteykcrcp pgwsgkscqq adpcasnpca nggqclpfea syichcppsf hgptcrqdvn |
| 181 | ecgqkpglcr hggtchnevg syrcvcrath tgpncerpyv pcspspcqng gtcrptgdvt |
| 241 | hecaclpgft gqnceenidd cpgnnckngg acvdgvntyn crcppewtgq yctedvdecq |
| 301 | lmpnacqngg tchnthggyn cvcvngwtge dcseniddca saacfhgatc hdrvasfyce |
| 361 | cphgrtgllc hlndacisnp cnegsncdtn pvngkaictc psgytgpacs qdvdecslga |
| 421 | npcehagkci ntlgsfecqc lqgytgprce idvnecvsnp cqndatcldq igefqcicmp |
| 481 | gyegvhcevn tdecasspcl hngrcldkin efqcecptgf tghlcqydvd ecastpckng |
| 541 | akcldgpnty tcvctegytg thcevdidec dpdpchygsc kdgvatftcl crpgytghhc |
| 601 | etninecssq perhggtcqd rdnaylcfcl kgttgpncei nlddcasspc dsgtcldkid |
| 661 | gyecacepgy tgsmcninid ecagnpchng gtcedgingf tcrcpegyhd ptclsevnec |
| 721 | nsnpcvhgac rdslngykcd cdpgwsgtnc dinnnecesn pcvnggtckd mtsgyvctcr |
| 781 | egfsgpncqt ninecasnpc lnqgtciddv agykcncllp ytgatcevvl apcapspcrn |
| 841 | ggecrqsedy esfscvcptg wqgqtcevdi necvlspcrh gascqnthgg yrchcqagys |
| 901 | grncetdidd crpnpchngg sctdgintaf cdclpgfrgt fceedineca sdpcrnganc |
| 961 | tdcvdsytct cpagfsgihc enntpdctes scfnggtcvd ginsftclcp pgftgsycqh |
| 1021 | dvnecdsqpc lhggtcqdgc gsyrctcpqg ytgpncqnlv hwcdsspckn ggkcwqthtq |
| 1081 | yrcecpsgwt glycdvpsvs cevaaqrqgv dvarlcqhgg lcvdagnthh crcqagytgs |
| 1141 | ycedlvdecs pspcqngatc tdylggysck cvagyhgvnc seeideclsh pcqnggtcld |
| 1201 | lpntykcscp rgtqgvhcei nvddcnppvd pvsrspkcfn ngtcvdqvgg ysctcppgfv |
| 1261 | gercegdvne clsnpcdarg tqncvqrvnd fhcecraght grrcesving ckgkpckngg |
| 1321 | tcavasntar gfickcpagf egatcendar tcgslrclng gtcisgprsp tclclgpftg |
| 1381 | pecqfpassp clggnpcynq gtceptsesp fyrclcpakf ngllchildy sfgggagrdi |
| 1441 | ppplieeace lpecqedagn kvcslqcnnh acgwdggdcs lnfndpwknc tqslqcwkyf |
| 1501 | sdghcdsqcn sagclfdgfd cgraegqcnp lydqyckdhf sdghcdqgcn saecewdgld |
| 1561 | caehvperla agtlvvvvlm ppeqlrnssf hflrelsrvl htnvvfkrda hgqqmifpyy |
| 1621 | greeelrkhp ikraaegwaa pdallgqvka sllpggsegg rrrreldpmd vrgsivylei |
| 1681 | dnrqcvqass qcfqsatdva aflgalaslg slnipykiea vqsetveppp paqlhfmyva |
| 1741 | aaafvllffv gcgvllsrkr rrqhgqlwfp egfkvseask kkrreplged svglkplkna |
| 1801 | sdgalmddnq newgdedlet kkfrfeepvv lpdlddqtdh rqwtqqhlda adlrmsamap |
| 1861 | tppqgevdad cmdvnvrgpd gftplmiasc sgggletgns eeeedapavi sdfiyqgasl |
| 1921 | hnqtdrtget alhlaarysr sdaakrllea sadaniqdnm grtplhaavs adaqgvfqil |
| 1981 | irnratdlda rmhdgttpli laarlavegm ledlinshad vnavddlgks alhwaaavnn |
| 2041 | vdaavvllkn gankdmqnnr eetplflaar egsyetakvl ldhfanrdit dhmdrlprdi |
| 2101 | aqermhhdiv rlldeynlvr spqlhgaplg gtptlspplc spngylgslk pgvqgkkvrk |
| 2161 | psskglacgs keakdlkarr kksqdgkgcl ldssgmlspv dslesphgyl sdvasppllp |
| 2221 | spfqqspsvp lnhlpgmpdt hlgighlnva akpemaalgg ggrlafetgp prlshlpvas |
| 2281 | gtstvlgsss ggalnftvgg stslngqcew lsrlqsgmvp nqynplrgsv apgplstqap |
| 2341 | slqhgmvgpl hsslaasals qmmsyqglps trlatqphlv qtqqvqpqnl qmqqqnlqpa |
| 2401 | niqqqqslqp pppppqphlg vssaasghlg rsflsgepsq advqplgpss lavhtilpqe |
| 2461 | spalptslps slvppvtaaq fltppsqhsy sspvdntpsh qlqvpehpfl tpspespdqw |
| 2521 | ssssphsnvs dwsegvsspp tsmqsqiari peafk |

By “Notch1 polynucleotide” is meant a nucleic acid molecule encoding a Notch1 polypeptide. An exemplary Notch1 polynucleotide sequence is provided at NCBI Reference Sequence: NM 017617.4, and reproduced herein below.

| | |
|---|---|
| 1 | atgccgccgc tcctggcgcc cctgctctgc ctggcgctgc tgcccgcgct cgccgcacga |
| 61 | ggcccgcgat gctcccagcc cggtgagacc tgcctgaatg gcgggaagtg tgaagcggcc |
| 121 | aatggcacgg aggcctgcgt ctgtggcggg gccttcgtgg gcccgcgatg ccaggacccc |
| 181 | aacccgtgcc tcagcacccc ctgcaagaac gccgggacat gccacgtggt ggaccgcaga |
| 241 | ggcgtggcag actatgcctg cagctgtgcc ctgggcttct ctgggcccct ctgcctgaca |
| 301 | cccctggaca atgcctgcct caccaacccc tgccgcaacg ggggcacctg cgacctgctc |
| 361 | acgctgacgg agtacaagtg ccgctgcccg cccggctggt cagggaaatc gtgccagcag |
| 421 | gctgacccgt gcgcctccaa cccctgcgcc aacggtggcc agtgcctgcc cttcgaggcc |
| 481 | tcctacatct gccactgccc acccagcttc catggcccca cctgccggca ggatgtcaac |
| 541 | gagtgtggcc agaagcccgg gctttgccgc cacggaggca cctgccacaa cgaggtcggc |
| 601 | tcctaccgct gcgtctgccg cgccacccac actggcccca actgcgagcg gccctacgtg |
| 661 | ccctgcagcc cctcgccctg ccagaacggg ggcacctgcc gccccacggg cgacgtcacc |
| 721 | cacgagtgtg cctgcctgcc aggcttcacc ggccagaact gtgaggaaaa tatcgacgat |
| 781 | tgtccaggaa acaactgcaa gaacgggggt gcctgtgtgg acggcgtgaa cacctacaac |
| 841 | tgccgctgcc cgccagagtg gacaggtcag tactgtaccg aggatgtgga cgagtgccag |
| 901 | ctgatgccaa atgcctgcca gaacggcggg acctgccaca acacccacgg tggctacaac |
| 961 | tgcgtgtgtg tcaacggctg gactggtgag gactgcagcg agaacattga tgactgtgcc |
| 1021 | agcgccgcct gcttccacgg cgccacctgc catgaccgtg tggcctcctt ctactgcgag |
| 1081 | tgtccccatg gccgcacagg tctgctgtgc cacctcaacg acgcatgcat cagcaacccc |
| 1141 | tgtaacgagg gctccaactg cgacaccaac cctgtcaatg gcaaggccat ctgcacctgc |
| 1201 | ccctcggggt acacgggccc ggcctgcagc caggacgtgg atgagtgctc gctgggtgcc |
| 1261 | aacccctgcg agcatgcggg caagtgcatc aacacgctgg gctccttcga gtgccagtgt |
| 1321 | ctgcagggct acacgggccc ccgatgcgag atcgacgtca acgagtgcgt ctcgaacccg |
| 1381 | tgccagaacg acgccacctg cctggaccag attggggagt tccagtgcat ctgcatgccc |
| 1441 | ggctacgagg gtgtgcactg cgaggtcaac acagacgagt gtgccagcag cccctgcctg |
| 1501 | cacaatggcc gctgcctgga caagatcaat gagttccagt gcgagtgccc cacgggcttc |
| 1561 | actgggcatc tgtgccagta cgatgtggac gagtgtgcca gcaccccctg caagaatggt |
| 1621 | gccaagtgcc tggacggacc caacacttac acctgtgtgt gcacggaagg gtacacgggg |
| 1681 | acgcactgcg aggtggacat cgatgagtgc gaccccgacc cctgccacta cggctcctgc |
| 1741 | aaggacggcg tcgccacctt cacctgcctc tgccgcccag gctacacggg ccaccactgc |
| 1801 | gagaccaaca tcaacgagtg ctccagccag ccctgccgcc acgggggcac ctgccaggac |
| 1861 | cgcgacaacg cctacctctg cttctgcctg aaggggacca caggacccaa ctgcgagatc |
| 1921 | aacctggatg actgtgccag cagcccctgc gactcgggca cctgtctgga caagatcgat |
| 1981 | ggctacgagt gtgcctgtga gccgggctac acagggagca tgtgtaacat caacatcgat |
| 2041 | gagtgtgcgg gcaacccctg ccacaacggg ggcacctgcg aggacggcat caatggcttc |
| 2101 | acctgccgct gccccgaggg ctaccacgac cccacctgcc tgtctgaggt caatgagtgc |
| 2161 | aacagcaacc cctgcgtcca cggggcctgc cgggacagcc tcaacgggta caagtgcgac |
| 2221 | tgtgaccctg ggtggagtgg gaccaactgt gacatcaaca acaatgagtg tgaatccaac |
| 2281 | ccttgtgtca acggcggcac ctgcaaagac atgaccagtg gctacgtgtg cacctgccgg |
| 2341 | gagggcttca gcggtcccaa ctgccagacc aacatcaacg agtgtgcgtc caacccatgt |
| 2401 | ctgaaccagg gcacgtgtat tgacgacgtt gccgggtaca agtgcaactg cctgctgccc |
| 2461 | tacacaggtg ccacgtgtga ggtggtgctg gccccgtgtg cccccagccc ctgcagaaac |
| 2521 | ggcggggagt gcaggcaatc cgaggactat gagagcttct cctgtgtctg ccccacgggc |
| 2581 | tggcaagggc agacctgtga ggtcgacatc aacgagtgcg ttctgagccc gtgccggcac |
| 2641 | ggcgcatcct gccagaacac ccacggcggc taccgctgcc actgccaggc cggctacagt |
| 2701 | gggcgcaact gcgagaccga catcgacgac tgccggccca acccgtgtca caacgggggc |
| 2761 | tcctgcacag acggcatcaa cacggccttc tgcgactgcc tgcccggctt ccggggcact |
| 2821 | ttctgtgagg aggacatcaa cgagtgtgcc agtgacccct gccgcaacgg ggccaactgc |
| 2881 | acggactgcg tggacagcta cacgtgcacc tgccccgcag gcttcagcgg gatccactgt |
| 2941 | gagaacaaca cgcctgactg cacagagagc tcctgcttca acggtggcac ctgcgtggac |
| 3001 | ggcatcaact cgttcacctg cctgtgtcca cccggcttca cgggcagcta ctgccagcac |
| 3061 | gatgtcaatg agtgcgactc acagccctgc ctgcatggcg gcacctgtca ggacggctgc |
| 3121 | ggctcctaca ggtgcacctg cccccagggc tacactggcc ccaactgcca gaaccttgtg |
| 3181 | cactggtgtg actcctcgcc ctgcaagaac ggcggcaaat gctggcagac ccacacccag |
| 3241 | taccgctgcg agtgccccag cggctggacc ggcctttact gcgacgtgcc cagcgtgtcc |
| 3301 | tgtgaggtgg ctgcgcagcg acaaggtgtt gacgttgccc gcctgtgcca gcatggaggg |
| 3361 | ctctgtgtgg acgcgggcaa cacgcaccac tgccgctgcc aggcgggcta cacaggcagc |
| 3421 | tactgtgagg acctggtgga cgagtgctca cccagcccct gccagaacgg ggccacctgc |
| 3481 | acggactacc tgggcggcta ctcctgcaag tgcgtggccg gctaccacgg ggtgaactgc |
| 3541 | tctgaggaga tcgacgagtg cctctcccac ccctgccaga acgggggcac ctgcctcgac |
| 3601 | ctccccaaca cctacaagtg ctcctgccca cggggcactc agggtgtgca ctgtgagatc |
| 3661 | aacgtggacg actgcaatcc ccccgttgac cccgtgtccc ggagccccaa gtgctttaac |
| 3721 | aacggcacct gcgtggacca ggtgggcggc tacagctgca cctgcccgcc gggcttcgtg |
| 3781 | ggtgagcgct gtgaggggga tgtcaacgag tgcctgtcca atccctgcga cgcccgtggc |
| 3841 | acccagaact gcgtgcagcg cgtcaatgac ttccactgcg agtgccgtgc tggtcacacc |
| 3901 | gggcgccgct gcgagtccgt catcaatggc tgcaaaggca agccctgcaa gaatgggggc |
| 3961 | acctgcgccg tggcctccaa caccgcccgc gggttcatct gcaagtgccc tgcgggcttc |
| 4021 | gagggcgcca cgtgtgagaa tgacgctcgt acctgcggca gcctgcgctg cctcaacggc |
| 4081 | ggcacatgca tctccggccc gcgcagcccc acctgcctgt gcctgggccc cttcacgggc |
| 4141 | cccgaatgcc agttcccggc cagcagcccc tgcctgggcg gcaacccctg ctacaaccag |
| 4201 | gggacctgtg agcccacatc cgagagcccc ttctaccgtt gcctgtgccc cgccaaattc |
| 4261 | aacgggctct tgtgccacat cctggactac agcttcgggg gtggggccgg gcgcgacatc |
| 4321 | cccccgccgc tgatcgagga ggcgtgcgag ctgcccgagt gccaggagga cgcgggcaac |
| 4381 | aaggtctgca gcctgcagtg caacaaccac gcgtgcggct gggacggcgg tgactgctcc |
| 4441 | ctcaacttca atgacccctg gaagaactgc acgcagtctc tgcagtgctg gaagtacttc |
| 4501 | agtgacggcc actgtgacag ccagtgcaac tcagccggct gcctcttcga cggctttgac |
| 4561 | tgccagcgtg cggaaggcca gtgcaacccc ctgtacgacc agtactgcaa ggaccacttc |
| 4621 | agcgacgggc actgcgacca gggctgcaac agcgcggagt gcgagtggga cgggctggac |
| 4681 | tgtgcggagc atgtacccga gaggctggcg gccggcacgc tggtggtggt ggtgctgatg |
| 4741 | ccgccggagc agctgcgcaa cagctccttc cacttcctgc gggagctcag ccgcgtgctg |
| 4801 | cacaccaacg tggtcttcaa gcgtgacgca cacggccagc agatgatctt cccctactac |
| 4861 | ggccgcgagg aggagctgcg caagcacccc atcaagcgtg ccgccgaggg ctgggccgca |
| 4921 | cctgacgccc tgctgggcca ggtgaaggcc tcgctgctcc ctggtggcag cgagggtggg |
| 4981 | cggcggcgga gggagctgga ccccatggac gtccgcggct ccatcgtcta cctggagatt |
| 5041 | gacaaccggc agtgtgtgca ggcctcctcg cagtgcttcc agagtgccac cgacgtggcc |
| 5101 | gcattcctgg gagcgctcgc ctcgctgggc agcctcaaca tcccctacaa gatcgaggcc |
| 5161 | gtgcagagtg agaccgtgga gccgcccccg ccggcgcagc tgcacttcat gtacgtggcg |
| 5221 | gcggccgcct ttgtgcttct gttcttcgtg ggctgcgggg tgctgctgtc ccgcaagcgc |
| 5281 | cggcggcagc atggccagct ctggttccct gagggcttca aagtgtctga ggccagcaag |
| 5341 | aagaagcggc gggagcccct cggcgaggac tccgtgggcc tcaagcccct gaagaacgct |
| 5401 | tcagacggtg ccctcatgga cgacaaccag aatgagtggg gggacgagga cctggagacc |
| 5461 | aagaagttcc ggttcgagga gcccgtggtt ctgcctgacc tggacgacca gacagaccac |
| 5521 | cggcagtgga ctcagcagca cctggatgcc gctgacctgc gcatgtctgc catggccccc |
| 5581 | acaccgcccc agggtgaggt tgacgccgac tgcatggacg tcaatgtccg cgggcctgat |
| 5641 | ggcttcaccc cgctcatgat cgcctcctgc agcgggggcg gcctggagac gggcaacagc |
| 5701 | gaggaagagg aggacgcgcc ggccgtcatc tccgacttca tctaccaggg cgccagcctg |
| 5761 | cacaaccaga cagaccgcac gggcgagacc gccttgcacc tggccgcccg ctactcacgc |
| 5821 | tctgatgccg ccaagcgcct gctggaggcc agcgcagatg ccaacatcca ggacaacatg |
| 5881 | ggccgcaccc cgctgcatgc ggctgtgtct gccgacgcac aaggtgtctt ccagatcctg |
| 5941 | atccggaacc gagccacaga cctggatgcc cgcatgcatg atggcacgac gccactgatc |
| 6001 | ctggctgccc gcctggccgt ggagggcatg ctggaggacc tcatcaactc acacgccgac |
| 6061 | gtcaacgccg tagatgacct gggcaagtcc gccctgcact gggccgccgc cgtgaacaat |
| 6121 | gtggatgccg cagttgtgct cctgaagaac ggggctaaca aagatatgca gaacaacagg |
| 6181 | gaggagacac ccctgtttct ggccgcccgg gagggcagct acgagaccgc caaggtgctg |
| 6241 | ctggaccact ttgccaaccg ggacatcacg gatcatatgg accgcctgcc gcgcgacatc |
| 6301 | gcacaggagc gcatgcatca cgacatcgtg aggctgctgg acgagtacaa cctggtgcgc |
| 6361 | agcccgcagc tgcacggagc cccgctgggg ggcacgccca ccctgtcgcc cccgctctgc |
| 6421 | tcgcccaacg gctacctggg cagcctcaag cccggcgtgc agggcaagaa ggtccgcaag |
| 6481 | cccagcagca aaggcctggc ctgtggaagc aaggaggcca aggacctcaa ggcacggagg |
| 6541 | aagaagtccc aggacggcaa gggctgcctg ctggacagct ccggcatgct ctcgcccgtg |
| 6601 | gactccctgg agtcacccca tggctacctg tcagacgtgg cctcgccgcc actgctgccc |
| 6661 | tccccgttcc agcagtctcc gtccgtgccc ctcaaccacc tgcctgggat gcccgacacc |
| 6721 | cacctgggca tcgggcacct gaacgtggcg gccaagcccg agatggcggc gctgggtggg |
| 6781 | ggcggccggc tggcctttga gactggccca cctcgtctct cccacctgcc tgtggcctct |
| 6841 | ggcaccagca ccgtcctggg ctccagcagc ggaggggccc tgaatttcac tgtgggcggg |
| 6901 | tccaccagtt tgaatggtca atgcgagtgg ctgtcccggc tgcagagcgg catggtgccg |
| 6961 | aaccaataca accctctgcg ggggagtgtg gcaccaggcc ccctgagcac acaggccccc |
| 7021 | tccctgcagc atggcatggt aggcccgctg cacagtagcc ttgctgccag cgccctgtcc |
| 7081 | cagatgatga gctaccaggg cctgcccagc acccggctgg ccacccagcc tcacctggtg |
| 7141 | cagacccagc aggtgcagcc acaaaactta cagatgcagc agcagaacct gcagccagca |
| 7201 | aacatccagc agcagcaaag cctgcagccg ccaccaccac caccacagcc gcaccttggc |
| 7261 | gtgagctcag cagccagcgg ccacctgggc cggagcttcc tgagtggaga gccgagccag |
| 7321 | gcagacgtgc agccactggg ccccagcagc ctggcggtgc acactattct gccccaggag |
| 7381 | agccccgccc tgcccacgtc gctgccatcc tcgctggtcc cacccgtgac cgcagcccag |
| 7441 | ttcctgacgc ccccctcgca gcacagctac tcctcgcctg tggacaacac ccccagccac |
| 7501 | cagctacagg tgcctgagca ccccttcctc accccgtccc ctgagtcccc tgaccagtgg |
| 7561 | tccagctcgt ccccgcattc caacgtctcc gactggtccg agggcgtctc cagccctccc |
| 7621 | accagcatgc agtcccagat cgcccgcatt ccggaggcct tcaagtaaac ggcgcgcccc |
| 7681 | acgagacccc ggcttccttt cccaagcctt cgggcgtctg tgtgcgctct gtggatgcca |
| 7741 | gggccgacca gaggagcctt tttaaaacac atgtttttat acaaaataag aacgaggatt |
| 7801 | ttaatttttt ttagtattta tttatgtact tttattttac acagaaacac tgccttttta |
| 7861 | tttatatgta ctgttttatc tggccccagg tagaaacttt tatctattct gagaaaacaa |
| 7921 | gcaagttctg agagccaggg ttttcctacg taggatgaaa agattcttct gtgtttataa |
| 7981 | aatataaaca aagattcatg atttataaat gccatttatt tattgattcc ttttttcaaa |
| 8041 | atccaaaaag aaatgatgtt ggagaaggga agttgaacga gcatagtcca aaaagctcct |
| 8101 | ggggcgtcca ggccgcgccc tttccccgac gcccacccaa ccccaagcca gcccggccgc |
| 8161 | tccaccagca tcacctgcct gttaggagaa gctgcatcca gaggcaaacg gaggcaaagc |
| 8221 | tggctcacct tccgcacgcg gattaatttg catctgaaat aggaaacaag tgaaagcata |
| 8281 | tgggttagat gttgccatgt gttttagatg gtttcttgca agcatgcttg tgaaaatgtg |
| 8341 | ttctcggagt gtgtatgcca agagtgcacc catggtacca atcatgaatc tttgtttcag |
| 8401 | gttcagtatt atgtagttgt tcgttggtta tacaagttct tggtccctcc agaaccaccc |
| 8461 | cggccccctg cccgttcttg aaatgtaggc atcatgcatg tcaaacatga gatgtgtgga |
| 8521 | ctgtggcact tgcctgggtc acacacggag gcatcctacc cttttctggg gaaagacact |
| 8581 | gcctgggctg accccggtgg cggccccagc acctcagcct gcacagtgtc ccccaggttc |
| 8641 | cgaagaagat gctccagcaa cacagcctgg gccccagctc gcgggacccg accccccgtg |
| 8701 | ggctcccgtg ttttgtagga gacttgccag agccgggcac attgagctgt gcaacgccgt |
| 8761 | gggctgcgtc ctttggtcct gtccccgcag ccctggcagg gggcatgcgg tcgggcaggg |
| 8821 | gctggaggga ggcgggggct gcccttgggc cacccctcct agtttgggag gagcagattt |
| 8881 | ttgcaatacc aagtatagcc tatggcagaa aaaatgtctg taaatatgtt tttaaaggtg |
| 8941 | gattttgttt aaaaaatctt aatgaatgag tctgttgtgt gtcatgccag tgagggacgt |
| 9001 | cagacttggc tcagctcggg gagccttagc cgcccatgca ctggggacgc tccgctgccg |
| 9061 | tgccgcctgc actcctcagg gcagcctccc ccggctctac gggggccgcg tggtgccatc |
| 9121 | cccagggggc atgaccagat gcgtcccaag atgttgattt ttactgtgtt ttataaaata |
| 9181 | gagtgtagtt tacagaaaaa gactttaaaa gtgatctaca tgaggaactg tagatgatgt |
| 9241 | atttttttca tcttttttgt taactgattt gcaataaaaa tgatactgat ggtgatctgg |
| 9301 | cttccaaaaa aaaaaaaaaa aa |

By “Neurogenic locus notch homolog protein 2 (Notch2) polypeptide” is meant a protein having at least about 85% amino acid identity to the sequence provided at NCBI Reference Sequence: AAG37073.1, or a fragment thereof, and having Notch receptor activity. An exemplary Notch2 amino acid sequence is provided below:

| | |
|---|---|
| 1 | mpalrpallw allalwlcca tpahalqcrd gyepcvnegm cvtyhngtgy ckcpegflge |
| 61 | ycqhrdpcek nrcqnggtcv aqamlgkatc rcasgftged cqystshpcf vsrpclnggt |
| 121 | chmlsrdtye ctcqvgftgk ecqwtdacls hpcangstct tvanqfsckc ltgftgqkce |
| 181 | tdvnecdipg hcqhggtcln lpgsyqcqcl qgftgqycds lyvpcapspc vnggtcrqtg |
| 241 | dftfecnclp gfegstcern iddcpnhrcq nggvcvdgvn tyncrcppqw tgqfctedvd |
| 301 | ecllqpnacq nggtcanrng gygcvcvngw sgddcsenid dcafasctpg stcidrvasf |
| 361 | scmcpegkag llchlddaci snpchkgalc dtnplngqyi ctcpqgykga dctedvdeca |
| 421 | mansnpceha gkcvntdgaf hceclkgyag prcemdinec hsdpcqndat cldkiggftc |
| 481 | lcmpgfkgvh celeinecqs npcvnngqcv dkvnrfqclc ppgftgpvcq ididdcsstp |
| 541 | clngakcidh pngyecqcat gftgvlceen idncdpdpch hgqcqdgids ytcicnpgym |
| 601 | gaicsdqide cysspclndg rcidlvngyq cncqpgtsgv nceinfddca snpcihgicm |
| 661 | dginryscvc spgftgqrcn ididecasnp crkgatcing vngfrcicpe gphhpscysq |
| 721 | vneclsnpci hgnctgglsg ykclcdagwv gincevdkne clsnpcqngg tcdnlvngyr |
| 781 | ctckkgfkgy ncqvnideca snpclnqgtc fddisgytch cvlpytgknc qtvlapcspn |
| 841 | pcenaavcke spnfesytcl capgwqgqrc tididecisk pcmnhglchn tqgsymcecp |
| 901 | pgfsgmdcee diddclanpc qnggscmdgv ntfsclclpg ftgdkcqtdm neclsepckn |
| 961 | ggtcsdyvns ytckcqagfd gvhcennine ctesscfngg tcvdginsfs clcpvgftgs |
| 1021 | fclheinecs shpclnegtc vdglgtyrcs cplgytgknc qtlvnlcsrs pcknkgtcvq |
| 1081 | kkaesqclcp sgwagaycdv pnvscdiaas rrgvlvehlc qhsgvcinag nthycqcplg |
| 1141 | ytgsyceeql decasnpcqh gatcsdfigg yrcecvpgyq gvnceyevde cqnqpcqngg |
| 1201 | tcidlvnhfk cscppgtrgl lceeniddca rgphclnggq cmdriggysc rclpgfager |
| 1261 | cegdinecls npcssegsld ciqltndylc vcrsaftgrh cetfvdvcpq mpclnggtca |
| 1321 | vasnmpdgfi crcppgfsga rcqsscgqvk crkgeqcvht asgprcfcps prdcesgcas |
| 1381 | spcqhggsch pqrqppyysc qcappfsgsr celytappst ppatclsqyc adkardgvcd |
| 1441 | eacnshacqw dggdcsltme npwancsspl pcwdyinnqc delcntvecl fdnfecqgns |
| 1501 | ktckydkyca dhfkdnhcdq gcnseecgwd gldcaadqpe nlaegtlviv vlmppeqllq |
| 1561 | darsflralg tllhtnlrik rdsqgelmvy pyygeksaam kkqrmtrrsl pgeqeqevag |
| 1621 | skvfleidnr qcvqdsdhcf kntdaaaall ashaiqgtls yplvsvvses ltpertqlly |
| 1681 | llavavviil fiillgvima krkrkhgslw lpegftlrrd asnhkrrepv gqdavglknl |
| 1741 | svqvseanli gtgtsehwvd degpqpkkvk aedeallsee ddpidrrpwt qqhleaadir |
| 1801 | rtpslaltpp qaeqevdvld vnvrgpdgct plmlaslrgg ssdlsdeded aedssaniit |
| 1861 | dlvyqgaslq aqtdrtgema lhlaarysra daakrlldag adanaqdnmg rcplhaavaa |
| 1921 | daqgvfqili rnrvtdldar mndgttplil aarlavegmv aelincqadv navddhgksa |
| 1981 | lhwaaavnnv eatllllkng anrdmqdnke etplflaare gsyeaakill dhfanrditd |
| 2041 | hmdrlprdva rdhmhhdivr lldeynvtps ppgtvltsal spvicgpnrs flslkhtpmg |
| 2101 | kksrrpsaks tmptslpnla keakdakgsr rkkslsekvq lsessvtlsp vdslesphty |
| 2161 | vsdttsspmi tspgilqasp npmlataapp apvhaqhals fsnlhemqpl ahgastvlps |
| 2221 | vsqllshhhi vspgsgsags lsrlhpvpvp adwmnrmevn etqynemfgm vlapaegthp |
| 2281 | giapqsrppe gkhittprep lppivtfqli pkgsiaqpag apqpqstcpp avagplptmy |
| 2341 | qipemarlps vafptammpq qdgqvaqtil payhpfpasv gkyptppsqh syassnaaer |
| 2401 | tpshsghlqg ehpyltpspe spdqwssssp hsasdwsdvt tsptpggagg gqrgpgthms |
| 2461 | epphnnmqvy a |

By “Notch2 polynucleotide” is meant a nucleic acid molecule encoding a Notch2 polypeptide. An exemplary Notch2 polynucleotide sequence is provided at NCBI Reference Sequence: AF315356.1, and reproduced herein below.

| | |
|---|---|
| 1 | gcgaccgaga agatgcccgc cctgcgcccc gctctgctgt gggcgctgct ggcgctctgg |
| 61 | ctgtgctgcg cgacccccgc gcatgcattg cagtgtcgag atggctatga accctgtgta |
| 121 | aatgaaggaa tgtgtgttac ctaccacaat ggcacaggat actgcaaatg tccagaaggc |
| 181 | ttcttggggg aatattgtca acatcgagac ccctgtgaga agaaccgctg ccagaatggt |
| 241 | gggacttgtg tggcccaggc catgctgggg aaagccacgt gccgatgtgc ctcagggttt |
| 301 | acaggagagg actgccagta ctcgacatct catccatgct ttgtgtctcg accctgcctg |
| 361 | aatggcggca catgccatat gctcagccgg gatacctatg agtgcacctg tcaagtcggg |
| 421 | tttacaggta aggagtgcca atggaccgat gcctgcctgt ctcatccctg tgcaaatgga |
| 481 | agtacctgta ccactgtggc caaccagttc tcctgcaaat gcctcacagg cttcacaggg |
| 541 | cagaaatgtg agactgatgt caatgagtgt gacattccag gacactgcca gcatggtggc |
| 601 | acctgcctca acctgcctgg ttcctaccag tgccagtgcc ttcagggctt cacaggccag |
| 661 | tactgtgaca gcctgtatgt gccctgtgca ccctcgcctt gtgtcaatgg aggcacctgt |
| 721 | cggcagactg gtgacttcac ttttgagtgc aactgccttc caggttttga agggagcacc |
| 781 | tgtgagagga atattgatga ctgccctaac cacaggtgtc agaatggagg ggtttgtgtg |
| 841 | gatggggtca acacttacaa ctgccgctgt cccccacaat ggacaggaca gttctgcaca |
| 901 | gaggatgtgg atgaatgcct gctgcagccc aatgcctgtc aaaatggggg cacctgtgcc |
| 961 | aaccgcaatg gaggctatgg ctgtgtatgt gtcaacggct ggagtggaga tgactgcagt |
| 1021 | gagaacattg atgattgtgc cttcgcctcc tgtactccag gctccacctg catcgaccgt |
| 1081 | gtggcctcct tctcttgcat gtgcccagag gggaaggcag gtctcctgtg tcatctggat |
| 1141 | gatgcatgca tcagcaatcc ttgccacaag ggggcactgt gtgacaccaa ccccctaaat |
| 1201 | gggcaatata tttgcacctg cccacaaggc tacaaagggg ctgactgcac agaagatgtg |
| 1261 | gatgaatgtg ccatggccaa tagcaatcct tgtgagcatg caggaaaatg tgtgaacacg |
| 1321 | gatggcgcct tccactgtga gtgtctgaag ggttatgcag gacctcgttg tgagatggac |
| 1381 | atcaatgagt gccattcaga cccctgccag aatgatgcta cctgtctgga taagattgga |
| 1441 | ggcttcacat gtctgtgcat gccaggtttc aaaggtgtgc attgtgaatt agaaataaat |
| 1501 | gaatgtcaga gcaacccttg tgtgaacaat gggcagtgtg tggataaagt caatcgtttc |
| 1561 | cagtgcctgt gtcctcctgg tttcactggg ccagtttgcc agattgatat tgatgactgt |
| 1621 | tccagtactc cgtgtctgaa tggggcaaag tgtatcgatc acccgaatgg ctatgaatgc |
| 1681 | cagtgtgcca caggtttcac tggtgtgttg tgtgaggaga acattgacaa ctgtgacccc |
| 1741 | gatccttgcc accatggtca gtgtcaggat ggtattgatt cctacacctg catctgcaat |
| 1801 | cccgggtaca tgggcgccat ctgcagtgac cagattgatg aatgttacag cagcccttgc |
| 1861 | ctgaacgatg gtcgctgcat tgacctggtc aatggctacc agtgcaactg ccagccaggc |
| 1921 | acgtcagggg ttaattgtga aattaatttt gatgactgtg caagtaaccc ttgtatccat |
| 1981 | ggaatctgta tggatggcat taatcgctac agttgtgtct gctcaccagg attcacaggg |
| 2041 | cagagatgta acattgacat tgatgagtgt gcctccaatc cctgtcgcaa gggtgcaaca |
| 2101 | tgtatcaacg gtgtgaatgg tttccgctgt atatgccccg agggacccca tcaccccagc |
| 2161 | tgctactcac aggtgaacga atgcctgagc aatccctgca tccatggaaa ctgtactgga |
| 2221 | ggtctcagtg gatataagtg tctctgtgat gcaggctggg ttggcatcaa ctgtgaagtg |
| 2281 | gacaaaaatg aatgcctttc gaatccatgc cagaatggag gaacttgtga caatctggtg |
| 2341 | aatggataca ggtgtacttg caagaagggc tttaaaggct ataactgcca ggtgaatatt |
| 2401 | gatgaatgtg cctcaaatcc atgcctgaac caaggaacct gctttgatga cataagtggc |
| 2461 | tacacttgcc actgtgtgct gccatacaca ggcaagaatt gtcagacagt attggctccc |
| 2521 | tgttccccaa acccttgtga gaatgctgct gtttgcaaag agtcaccaaa ttttgagagt |
| 2581 | tatacttgct tgtgtgctcc tggctggcaa ggtcagcggt gtaccattga cattgacgag |
| 2641 | tgtatctcca agccctgcat gaaccatggt ctctgccata acacccaggg cagctacatg |
| 2701 | tgtgaatgtc caccaggctt cagtggtatg gactgtgagg aggacattga tgactgcctt |
| 2761 | gccaatcctt gccagaatgg aggttcctgt atggatggag tgaatacttt ctcctgcctc |
| 2821 | tgccttccgg gtttcactgg ggataagtgc cagacagaca tgaatgagtg tctgagtgaa |
| 2881 | ccctgtaaga atggagggac ctgctctgac tacgtcaaca gttacacttg caagtgccag |
| 2941 | gcaggatttg atggagtcca ttgtgagaac aacatcaatg agtgcactga gagctcctgt |
| 3001 | ttcaatggtg gcacatgtgt tgatgggatt aactccttct cttgcttgtg ccctgtgggt |
| 3061 | ttcactggat ccttctgcct ccatgagatc aatgaatgca gctctcatcc atgcctgaat |
| 3121 | gagggaacgt gtgttgatgg cctgggtacc taccgctgca gctgccccct gggctacact |
| 3181 | gggaaaaact gtcagaccct ggtgaatctc tgcagtcggt ctccatgtaa aaacaaaggt |
| 3241 | acttgcgttc agaaaaaagc agagtcccag tgcctatgtc catctggatg ggctggtgcc |
| 3301 | tattgtgacg tgcccaatgt ctcttgtgac atagcagcct ccaggagagg tgtgcttgtt |
| 3361 | gaacacttgt gccagcactc aggtgtctgc atcaatgctg gcaacacgca ttactgtcag |
| 3421 | tgccccctgg gctatactgg gagctactgt gaggagcaac tcgatgagtg tgcgtccaac |
| 3481 | ccctgccagc acggggcaac atgcagtgac ttcattggtg gatacagatg cgagtgtgtc |
| 3541 | ccaggctatc agggtgtcaa ctgtgagtat gaagtggatg agtgccagaa tcagccctgc |
| 3601 | cagaatggag gcacctgtat tgaccttgtg aaccatttca agtgctcttg cccaccaggc |
| 3661 | actcggggcc tactctgtga agagaacatt gatgactgtg cccggggtcc ccattgcctt |
| 3721 | aatggtggtc agtgcatgga taggattgga ggctacagtt gtcgctgctt gcctggcttt |
| 3781 | gctggggagc gttgtgaggg agacatcaac gagtgcctct ccaacccctg cagctctgag |
| 3841 | ggcagcctgg actgtataca gctcaccaat gactacctgt gtgtttgccg tagtgccttt |
| 3901 | actggccggc actgtgaaac cttcgtcgat gtgtgtcccc agatgccctg cctgaatgga |
| 3961 | gggacttgtg ctgtggccag taacatgcct gatggtttca tttgccgttg tcccccggga |
| 4021 | ttttccgggg caaggtgcca gagcagctgt ggacaagtga aatgtaggaa gggggagcag |
| 4081 | tgtgtgcaca ccgcctctgg accccgctgc ttctgcccca gtccccggga ctgcgagtca |
| 4141 | ggctgtgcca gtagcccctg ccagcacggg ggcagctgcc accctcagcg ccagcctcct |
| 4201 | tattactcct gccagtgtgc cccaccattc tcgggtagcc gctgtgaact ctacacggca |
| 4261 | ccccccagca cccctcctgc cacctgtctg agccagtatt gtgccgacaa agctcgggat |
| 4321 | ggcgtctgtg atgaggcctg caacagccat gcctgccagt gggatggggg tgactgttct |
| 4381 | ctcaccatgg agaacccctg ggccaactgc tcctccccac ttccctgctg ggattatatc |
| 4441 | aacaaccagt gtgatgagct gtgcaacacg gtcgagtgcc tgtttgacaa ctttgaatgc |
| 4501 | caggggaaca gcaagacatg caagtatgac aaatactgtg cagaccactt caaagacaac |
| 4561 | cactgtgacc aggggtgcaa cagtgaggag tgtggttggg atgggctgga ctgtgctgct |
| 4621 | gaccaacctg agaacctggc agaaggtacc ctggttattg tggtattgat gccacctgaa |
| 4681 | caactgctcc aggatgctcg cagcttcttg cgggcactgg gtaccctgct ccacaccaac |
| 4741 | ctgcgcatta agcgggactc ccagggggaa ctcatggtgt acccctatta tggtgagaag |
| 4801 | tcagctgcta tgaagaaaca gaggatgaca cgcagatccc ttcctggtga acaagaacag |
| 4861 | gaggtggctg gctctaaagt ctttctggaa attgacaacc gccagtgtgt tcaagactca |
| 4921 | gaccactgct tcaagaacac ggatgcagca gcagctctcc tggcctctca cgccatacag |
| 4981 | gggaccctgt cataccctct tgtgtctgtc gtcagtgaat ccctgactcc agaacgcact |
| 5041 | cagctcctct atctccttgc tgttgctgtt gtcatcattc tgtttattat tctgctgggg |
| 5101 | gtaatcatgg caaaacgaaa gcgtaagcat ggctctctct ggctgcctga aggtttcact |
| 5161 | cttcgccgag atgcaagcaa tcacaagcgt cgtgagccag tgggacagga tgctgtgggg |
| 5221 | ctgaaaaatc tctcagtgca agtctcagaa gctaacctaa ttggtactgg aacaagtgaa |
| 5281 | cactgggtcg atgatgaagg gccccagcca aagaaagtaa aggctgaaga tgaggcctta |
| 5341 | ctctcagaag aagatgaccc cattgatcga cggccatgga cacagcagca ccttgaagct |
| 5401 | gcagacatcc gtaggacacc atcgctggct ctcacccctc ctcaggcaga gcaggaggtg |
| 5461 | gatgtgttag atgtgaatgt ccgtggccca gatggctgca ccccattgat gttggcttct |
| 5521 | ctccgaggag gcagctcaga tttgagtgat gaagatgaag atgcagagga ctcttctgct |
| 5581 | aacatcatca cagacttggt ctaccagggt gccagcctcc aggcccagac agaccggact |
| 5641 | ggtgagatgg ccctgcacct tgcagcccgc tactcacggg ctgatgctgc caagcgtctc |
| 5701 | ctggatgcag gtgcagatgc caatgcccag gacaacatgg gccgctgtcc actccatgct |
| 5761 | gcagtggcag ctgatgccca aggtgtcttc cagattctga ttcgcaaccg agtaactgat |
| 5821 | ctagatgcca ggatgaatga tggtactaca cccctgatcc tggctgcccg cctggctgtg |
| 5881 | gagggaatgg tggcagaact gatcaactgc caagcggatg tgaatgcagt ggatgaccat |
| 5941 | ggaaaatctg ctcttcactg ggcagctgct gtcaataatg tggaggcaac tcttttgttg |
| 6001 | ttgaaaaatg gggccaaccg agacatgcag gacaacaagg aagagacacc tctgtttctt |
| 6061 | gctgcccggg aggggagcta tgaagcagcc aagatcctgt tagaccattt tgccaatcga |
| 6121 | gacatcacag accatatgga tcgtcttccc cgggatgtgg ctcgggatca catgcaccat |
| 6181 | gacattgtgc gccttctgga tgaatacaat gtgaccccaa gccctccagg caccgtgttg |
| 6241 | acttctgctc tctcacctgt catctgtggg cccaacagat ctttcctcag cctgaagcac |
| 6301 | accccaatgg gcaagaagtc tagacggccc agtgccaaga gtaccatgcc tactagcctc |
| 6361 | cctaaccttg ccaaggaggc aaaggatgcc aagggtagta ggaggaagaa gtctctgagt |
| 6421 | gagaaggtcc aactgtctga gagttcagta actttatccc ctgttgattc cctagaatct |
| 6481 | cctcacacgt atgtttccga caccacatcc tctccaatga ttacatcccc tgggatctta |
| 6541 | caggcctcac ccaaccctat gttggccact gccgcccctc ctgccccagt ccatgcccag |
| 6601 | catgcactat ctttttctaa ccttcatgaa atgcagcctt tggcacatgg ggccagcact |
| 6661 | gtgcttccct cagtgagcca gttgctatcc caccaccaca ttgtgtctcc aggcagtggc |
| 6721 | agtgctggaa gcttgagtag gctccatcca gtcccagtcc cagcagattg gatgaaccgc |
| 6781 | atggaggtga atgagaccca gtacaatgag atgtttggta tggtcctggc tccagctgag |
| 6841 | ggcacccatc ctggcatagc tccccagagc aggccacctg aagggaagca cataaccacc |
| 6901 | cctcgggagc ccttgccccc cattgtgact ttccagctca tccctaaagg cagtattgcc |
| 6961 | caaccagcgg gggctcccca gcctcagtcc acctgccctc cagctgttgc gggccccctg |
| 7021 | cccaccatgt accagattcc agaaatggcc cgtttgccca gtgtggcttt ccccactgcc |
| 7081 | atgatgcccc agcaggacgg gcaggtagct cagaccattc tcccagccta tcatcctttc |
| 7141 | ccagcctctg tgggcaagta ccccacaccc ccttcacagc acagttatgc ttcctcaaat |
| 7201 | gctgctgagc gaacacccag tcacagtggt cacctccagg gtgagcatcc ctacctgaca |
| 7261 | ccatccccag agtctcctga ccagtggtca agttcatcac cccactctgc ttctgactgg |
| 7321 | tcagatgtga ccaccagccc tacccctggg ggtgctggag gaggtcagcg gggacctggg |
| 7381 | acacacatgt ctgagccacc acacaacaac atgcaggttt atgcgtgaga gagtccacct |
| 7441 | ccagtgtaga gacataactg acttttgtaa atgctgctga ggaacaaatg aaggtcatcc |
| 7501 | gggagagaaa tgaagaaatc tctggagcca gcttctagag gtaggaaaga gaagatgttc |
| 7561 | ttattcagat aatgcaagag aagcaattcg tcagtttcac tgggtatctg caaggcttat |
| 7621 | tgattattct aatctaataa gacaagtttg tggaaatgca agatgaatac aagccttggg |
| 7681 | tccatgttta ctctcttcta tttggagaat aagatggatg cttattgaag cccagacatt |
| 7741 | cttgcagctt ggactgcatt ttaagccctg caggcttctg ccatatccat gagaagattc |
| 7801 | tacactagcg tcctgttggg aattatgccc tggaattctg cctgaattga cctacgcatc |
| 7861 | tcctcctcct tggacattct tttgtcttca tttggtgctt ttggttttgc acctctccgt |
| 7921 | gattgtagcc ctaccagcat gttatagggc aagacctttg tgcttttgat cattctggcc |
| 7981 | catgaaagca actttggtct cctttcccct cctgtcttcc cggtatccct tggagtctca |
| 8041 | caaggtttac tttggtatgg ttctcagcac aaacctttca agtatgttgt ttctttggaa |
| 8101 | aatggacata ctgtattgtg ttctcctgca tatatcattc ctggagagag aaggggagaa |
| 8161 | gaatactttt cttcaacaaa ttttgggggc aggagatccc ttcaagaggc tgcaccttaa |
| 8221 | tttttcttgt ctgtgtgcag gtcttcatat aaactttacc aggaagaagg gtgtgagttt |
| 8281 | gttgtttttc tgtgtatggg cctggtcagt gtaaagtttt atccttgata gtctagttac |
| 8341 | tatgaccctc cccacttttt taaaaccaga aaaaggtttg gaatgttgga atgaccaaga |
| 8401 | gacaagttaa ctcgtgcaag agccagttac ccacccacag gtccccctac ttcctgccaa |
| 8461 | gcattccatt gactgcctgt atggaacaca tttgtcccag atctgagcat tctaggcctg |
| 8521 | tttcactcac tcacccagca tatgaaacta gtcttaactg ttgagccttt cctttcatat |
| 8581 | ccacagaaga cactgtctca aatgttgtac ccttgccatt taggactgaa ctttccttag |
| 8641 | cccaagggac ccagtgacag ttgtcttccg tttgtcagat gatcagtctc tactgattat |
| 8701 | cttgctgctt aaaggcctgc tcaccaatct ttctttcaca ccgtgtggtc cgtgttactg |
| 8761 | gtatacccag tatgttctca ctgaagacat ggactttata tgttcaagtg caggaattgg |
| 8821 | aaagttggac ttgttttcta tgatccaaaa cagccctata agaaggttgg aaaaggagga |
| 8881 | actatatagc agcctttgct attttctgct accatttctt ttcctctgaa gcggccatga |
| 8941 | cattcccttt ggcaactaac gtagaaactc aacagaacat tttcctttcc tagagtcacc |
| 9001 | ttttagatga taatggacaa ctatagactt gctcattgtt cagactgatt gcccctcacc |
| 9061 | tgaatccact ctctgtattc atgctcttgg caatttcttt gactttcttt taagggcaga |
| 9121 | agcattttag ttaattgtag ataaagaata gttttcttcc tcttctcctt gggccagtta |
| 9181 | ataattggtc catggctaca ctgcaacttc cgtccagtgc tgtgatgccc atgacacctg |
| 9241 | caaaataagt tctgcctggg cattttgtag atattaacag gtgaattccc gactcttttg |
| 9301 | gtttgaatga cagttctcat tccttctatg gctgcaagta tgcatcagtg cttcccactt |
| 9361 | acctgatttg tctgtcggtg gccccatatg gaaaccctgc gtgtctgttg gcataatagt |
| 9421 | ttacaaatgg ttttttcagt cctatccaaa tttattgaac caacaaaaat aattacttct |
| 9481 | gccctgagat aagcagatta agtttgttca ttctctgctt tattctctcc atgtggcaac |
| 9541 | attctgtcag cctctttcat agtgtgcaaa cattttatca ttctaaatgg tgactctctg |
| 9601 | cccttggacc catttattat tcacagatgg ggagaaccta tctgcatgga cctctgtgga |
| 9661 | ccacagcgta cctgcccctt tctgccctcc tgctccagcc ccacttctga aagtatcagc |
| 9721 | tactgatcca gccactggat attttatatc ctcccttttc cttaagcaca atgtcagacc |
| 9781 | aaattgcttg tttctttttc ttggactact ttaatttgga tcctttgggt ttggagaaag |
| 9841 | ggaatgtgaa agctgtcatt acagacaaca ggtttcagtg atgaggagga caacactgcc |
| 9901 | tttcaaactt tttactgatc tcttagattt taagaactct tgaattgtgt ggtatctaat |
| 9961 | aaaagggaag gtaagatgga taatcacttt ctcatttggg ttctgaattg gagactcagt |
| 10021 | ttttatgaga cacatctttt atgccatgta tagatcctcc cctgctattt ttggtttatt |
| 10081 | tttattgtta taaatgcttt ctttctttga ctcctcttct gcctgccttt ggggataggt |
| 10141 | ttttttgttt gtttatttgc ttcctctgtt ttgttttaag catcattttc ttatgtgagg |
| 10201 | tggggaaggg aaaggtatga gggaaagaga gtctgagaat taaaatattt tagtataagc |
| 10261 | aattggctgt gatgctcaaa tccattgcat cctcttattg aatttgccaa tttgtaattt |
| 10321 | ttgcataata aagaaccaaa ggtgtaatgt tttgttgaga ggtggtttag ggattttggc |
| 10381 | cctaaccaat acattgaatg tatgatgact atttgggagg acacatttat gtacccagag |
| 10441 | gcccccacta ataagtggta ctatggttac ttccttgtgt acatttctct taaaagtgat |
| 10501 | attatatctg tttgtatgag aaacccagta accaataaaa tgaccgcata ttcctgacta |
| 10561 | aacgtagtaa ggaaaatgca cactttgttt ttacttttcc gtttcattct aaaggtagtt |
| 10621 | aagatgaaat ttatatgaaa gcatttttat cacaaaataa aaaaggtttg ccaagctcag |
| 10681 | tggtgttgta ttttttattt tccaatactg catccatggc ctggcagtgt tacctcatga |
| 10741 | tgtcataatt tgctgagaga gcaaattttc ttttctttct gaatcccaca aagcctagca |
| 10801 | ccaaacttct ttttttcttc ctttaattag atcataaata aatgatcctg gggaaaaagc |
| 10861 | atctgtcaaa taggaaacat cacaaaactg agcactcttc tgtgcactag ccatagctgg |
| 10921 | tgacaaacag atggttgctc agggacaagg tgccttccaa tggaaatgcg aagtagttgc |
| 10981 | tatagcaaga attgggaact gggatataag tcataatatt aattatgctg ttatgtaaat |
| 11041 | gattggtttg taacattcct taagtgaaat ttgtgtagaa cttaatatac aggattataa |
| 11101 | aataatattt tgtgtataaa tttgttataa gttcacattc atacatttat ttataaagtc |
| 11161 | agtgagatat ttgaacatga aaaaaaaaa |

By “Neurogenic locus notch homolog protein 3 (Notch3) polypeptide” is meant a protein having at least about 85% amino acid identity to the sequence provided at NCBI Reference Sequence: AAB91371.1, or a fragment thereof, and having Notch receptor activity. An exemplary Notch3 amino acid sequence is provided below:

| | |
|---|---|
| 1 | mgpgargrrr rrrpmspppp pppvralpll lllagpgaaa ppcldgspca nggrctqlps |
| 61 | reaaclcppg wvgercqled pchsgpcagr gvcqssvvag tarfscrcpr gfrgpdcslp |
| 121 | dpclsspcah garcsvgpdg rflcscppgy qgrscrsdvd ecrvgepcrh ggtclntpgs |
| 181 | frcqcpagyt gplcenpavp capspcrngg tcrqsgdlty dcaclpgfeg qncevnvddc |
| 241 | pghrclnggt cvdgvntync qcppewtgqf ctedvdecql qpnachnggt cfntlgghsc |
| 301 | vcvngwtges csqniddcat avcfhgatch drvasfycac pmgktgllch lddacvsnpc |
| 361 | hedaicdtnp vngraictcp pgftggacdq dvdecsigan pcehlgrcvn tqgsflcqcg |
| 421 | rgytgprcet dvneclsgpc rnqatcldri gqftcicmag ftgtycevdi decqsspcvn |
| 481 | ggvckdrvng fsctcpsgfs gstcqldvde castpcrnga kcvdqpdgye crcaegfegt |
| 541 | lcdrnvddcs pdpchhgrcv dgiasfscac apgytgtrce sqvdecrsqp crhggkcldl |
| 601 | vdkylcrcps gttgvncevn iddcasnpct fgvcrdginr ydcvcqpgft gplcnveine |
| 661 | casspcgegg scvdgengfr clcppgslpp lclppshpca hepcshgicy dapggfrcvc |
| 721 | epgwsgprcs qslardaces qpcraggtcs sdgmgfhctc ppgvqgrqce llspctpnpc |
| 781 | ehggrcesap gqlpvcscpq gwqgprcqqd vdecagpapc gphgictnla gsfsctchgg |
| 841 | ytgpscdqdi ndcdpnpcln ggscqdgvgs fscsclpgfa gprcardvde clsnpcgpgt |
| 901 | ctdhvasftc tcppgyggfh ceqdlpdcsp sscfnggtcv dgvnsfsclc rpgytgahcq |
| 961 | headpclsrp clhggvcsaa hpgfrctcle sftgpqcqtl vdwcsrqpcq nggrcvqtga |
| 1021 | yclcppgwsg rlcdirslpc reaaaqigvr leqlcqaggq cvdedsshyc vcpegrtgsh |
| 1081 | ceqevdpcla qpcqhggtcr gymggymcec lpgyngdnce ddvdecasqp cqhggscidl |
| 1141 | varylcscpp gtlgvlcein eddcgpgppl dsgprclhng tcvdlvggfr ctcppgytgl |
| 1201 | rceadinecr sgachaahtr dclqdpgggf rclchagfsg prcqtvlspc esqpcqhggq |
| 1261 | crpspgpggg ltftchcaqp fwgprcerva rscrelqcpv gvpcqqtprg prcacppgls |
| 1321 | gpscrsfpgs ppgasnasca aapclhggsc rpaplapffr cacaqgwtgp rceapaaape |
| 1381 | vseeprcpra acqakrgdqr cdrecnspgc gwdggdcsls vgdpwrqcea lqcwrlfnns |
| 1441 | rcdpacsspa clydnfdcha ggrertcnpv yekycadhfa dgrcdqgcnt eecgwdgldc |
| 1501 | asevpallar gvlvltvllp peellrssad flqrlsailr tslrfrldah gqamvfpyhr |
| 1561 | pspgseprar relapevigs vvmleidnrl clqspendhc fpdaqsaady lgalsaverl |
| 1621 | dfpyplrdvr gepleppeps vpllpllvag avlllvilvl gvmvarrkre hstlwfpegf |
| 1681 | slhkdvasgh kgrrepvgqd algmknmakg eslmgevatd wmdtecpeak rlkveepgmg |
| 1741 | aeeavdcrqw tqhhlvaadi rvapamaltp pqgdadadgm dvnvrgpdgf tplmlasfcg |
| 1801 | galepmptee deaddtsasi isdlicqgaq lgartdrtge talhlaarya radaakrlld |
| 1861 | agadtnaqdh sgrtplhtav tadaqgvfqi lirnrstdld armadgstal ilaarlaveg |
| 1921 | mveeliasha dvnavdelgk salhwaaavn nveatlallk ngankdmqds keetplflaa |
| 1981 | regsyeaakl lldhfanrei tdhldrlprd vaqerlhqdi vrlldqpsgp rsppgphglg |
| 2041 | pllcppgafl pglkaaqsgs kksrrppgka glgpqgprgr gkkltlacpg pladssvtls |
| 2101 | pvdsldsprp fggppaspgg fplegpyaaa tatavslaql ggpgraglgr qppggcvlsl |
| 2161 | gllnpvavpl dwarlpppap pgpsfllpla pgpqllnpgt pvspqerppp ylavpghgee |
| 2221 | ypvagahssp pkarflrvps ehpyltpspe spehwaspsp pslsdwsest pspatatgam |
| 2281 | atttgalpaq plplsvpssl aqaqtqlgpq pevtpkrqvl a |

By “Notch3 polynucleotide” is meant a nucleic acid molecule encoding a Notch3 polypeptide. An exemplary Notch3 polynucleotide sequence is provided at NCBI Reference Sequence: U97669.1, and reproduced herein below.

| | |
|---|---|
| 1 | acgcggcgcg gaggctggcc cgggacgcgc ccggagccca gggaaggagg gaggagggga |
| 61 | gggtcgcggc cggccgccat ggggccgggg gcccgtggcc gccgccgccg ccgtcgcccg |
| 121 | atgtcgccgc caccgccacc gccacccgtg cgggcgctgc ccctgctgct gctgctagcg |
| 181 | gggccggggg ctgcagcccc cccttgcctg gacggaagcc cgtgtgcaaa tggaggtcgt |
| 241 | tgcacccagc tgccctcccg ggaggctgcc tgcctgtgcc cgcctggctg ggtgggtgag |
| 301 | cggtgtcagc tggaggaccc ctgtcactca ggcccctgtg ctggccgtgg tgtctgccag |
| 361 | agttcagtgg tggctggcac cgcccgattc tcatgccggt gcccccgtgg cttccgaggc |
| 421 | cctgactgct ccctgccaga tccctgcctc agcagccctt gtgcccacgg tgcccgctgc |
| 481 | tcagtggggc ccgatggacg cttcctctgc tcctgcccac ctggctacca gggccgcagc |
| 541 | tgccgaagcg acgtggatga gtgccgggtg ggtgagccct gccgccatgg tggcacctgc |
| 601 | ctcaacacac ctggctcctt ccgctgccag tgtccagctg gctacacagg gccactatgt |
| 661 | gagaaccccg cggtgccctg tgcgccctca ccatgccgta acgggggcac ctgcaggcag |
| 721 | agtggcgacc tcacttacga ctgtgcctgt cttcctgggt ttgagggtca gaattgtgaa |
| 781 | gtgaacgtgg acgactgtcc aggacaccga tgtctcaatg gggggacatg cgtggatggc |
| 841 | gtcaacacct ataactgcca gtgccctcct gagtggacag gccagttctg cacggaggac |
| 901 | gtggatgagt gtcagctgca gcccaacgcc tgccacaatg ggggtacctg cttcaacacg |
| 961 | ctgggtggcc acagctgcgt gtgtgtcaat ggctggacag gtgagagctg cagtcagaat |
| 1021 | atcgatgact gtgccacagc cgtgtgcttc catggggcca cctgccatga ccgcgtggct |
| 1081 | tctttctact gtgcctgccc catgggcaag actggcctcc tgtgtcacct ggatgacgcc |
| 1141 | tgtgtcagca acccctgcca cgaggatgct atctgtgaca caaatccggt gaacggccgg |
| 1201 | gccatttgca cctgtcctcc cggcttcacg ggtggggcat gtgaccagga tgtggacgag |
| 1261 | tgctctatcg gcgccaaccc ctgcgagcac ttgggcaggt gcgtgaacac gcagggctcc |
| 1321 | ttcctgtgcc agtgcggtcg tggctacact ggacctcgct gtgagaccga tgtcaacgag |
| 1381 | tgtctgtcgg ggccctgccg aaaccaggcc acgtgcctcg accgcatagg ccagttcacc |
| 1441 | tgtatctgta tggcaggctt cacaggaacc tattgcgagg tggacattga cgagtgtcag |
| 1501 | agtagcccct gtgtcaacgg tggggtctgc aaggaccgag tcaatggctt cagctgcacc |
| 1561 | tgcccctcgg gcttcagcgg ctccacgtgt cagctggacg tggacgaatg cgccagcacg |
| 1621 | ccctgcagga atggcgccaa atgcgtggac cagcccgatg gctacgagtg ccgctgtgcc |
| 1681 | gagggctttg agggcacgct gtgtgatcgc aacgtggacg actgctcccc tgacccatgc |
| 1741 | caccatggtc gctgcgtgga tggcatcgcc agcttctcat gtgcctgtgc tcctggctac |
| 1801 | acgggcacac gctgcgagag ccaggtggac gaatgccgca gccagccctg ccgccatggc |
| 1861 | ggcaaatgcc tagacctggt ggacaagtac ctctgccgct gcccttctgg gaccacaggt |
| 1921 | gtgaactgcg aagtgaacat tgacgactgt gccagcaacc cctgcacctt tggagtctgc |
| 1981 | cgtgatggca tcaaccgcta cgactgtgtc tgccaacctg gcttcacagg gcccctttgt |
| 2041 | aacgtggaga tcaatgagtg tgcttccagc ccatgcggcg agggaggttc ctgtgtggat |
| 2101 | ggggaaaatg gcttccgctg cctctgcccg cctggctcct tgcccccact ctgcctcccc |
| 2161 | ccgagccatc cctgtgccca tgagccctgc agtcacggca tctgctatga tgcacctggc |
| 2221 | gggttccgct gtgtgtgtga gcctggctgg agtggccccc gctgcagcca gagcctggcc |
| 2281 | cgagacgcct gtgagtccca gccgtgcagg gccggtggga catgcagcag cgatggaatg |
| 2341 | ggtttccact gcacctgccc gcctggtgtc cagggacgtc agtgtgaact cctctccccc |
| 2401 | tgcaccccga acccctgtga gcatgggggc cgctgcgagt ctgcccctgg ccagctgcct |
| 2461 | gtctgctcct gcccccaggg ctggcaaggc ccacgatgcc agcaggatgt ggacgagtgt |
| 2521 | gctggccccg caccctgtgg ccctcatggt atctgcacca acctggcagg gagtttcagc |
| 2581 | tgcacctgcc atggagggta cactggccct tcctgtgatc aggacatcaa tgactgtgac |
| 2641 | cccaacccat gcctgaacgg tggctcgtgc caagacggcg tgggctcctt ttcctgctcc |
| 2701 | tgcctccctg gtttcgccgg cccacgatgc gcccgcgatg tggatgagtg cctgagcaac |
| 2761 | ccctgcggcc cgggcacctg taccgaccac gtggcctcct tcacctgcac ctgcccgccg |
| 2821 | ggctacggag gcttccactg cgaacaggac ctgcccgact gcagccccag ctcctgcttc |
| 2881 | aatggcggga cctgtgtgga cggcgtgaac tcgttcagct gcctgtgccg tcccggctac |
| 2941 | acaggagccc actgccaaca tgaggcagac ccctgcctct cgcggccctg cctacacggg |
| 3001 | ggcgtctgca gcgccgccca ccctggcttc cgctgcacct gcctcgagag cttcacgggc |
| 3061 | ccgcagtgcc agacgctggt ggattggtgc agccgccagc cttgtcaaaa cgggggtcgc |
| 3121 | tgcgtccaga ctggggccta ttgcctttgt ccccctggat ggagcggacg cctctgtgac |
| 3181 | atccgaagct tgccctgcag ggaggccgca gcccagatcg gggtgcggct ggagcagctg |
| 3241 | tgtcaggcgg gtgggcagtg tgtggatgaa gacagctccc actactgcgt gtgcccagag |
| 3301 | ggccgtactg gtagccactg tgagcaggag gtggacccct gcttggccca gccctgccag |
| 3361 | catgggggga cctgccgtgg ctatatgggg ggctacatgt gtgagtgtct tcctggctac |
| 3421 | aatggtgata actgtgagga cgacgtggac gagtgtgcct cccagccctg ccagcacggg |
| 3481 | ggttcatgca ttgacctcgt ggcccgctat ctctgctcct gtcccccagg aacgctgggg |
| 3541 | gtgctctgcg agattaatga ggatgactgc ggcccaggcc caccgctgga ctcagggccc |
| 3601 | cggtgcctac acaatggcac ctgcgtggac ctggtgggtg gtttccgctg cacctgtccc |
| 3661 | ccaggataca ctggtttgcg ctgcgaggca gacatcaatg agtgtcgctc aggtgcctgc |
| 3721 | cacgcggcac acacccggga ctgcctgcag gacccaggcg gaggtttccg ttgcctttgt |
| 3781 | catgctggct tctcaggtcc tcgctgtcag actgtcctgt ctccctgcga gtcccagcca |
| 3841 | tgccagcatg gaggccagtg ccgtcctagc ccgggtcctg ggggtgggct gaccttcacc |
| 3901 | tgtcactgtg cccagccgtt ctggggtccg cgttgcgagc gggtggcgcg ctcctgccgg |
| 3961 | gagctgcagt gcccggtggg cgtcccatgc cagcagacgc cccgcgggcc gcgctgcgcc |
| 4021 | tgccccccag ggttgtcggg accctcctgc cgcagcttcc cggggtcgcc gccgggggcc |
| 4081 | agcaacgcca gctgcgcggc cgccccctgt ctccacgggg gctcctgccg ccccgcgccg |
| 4141 | ctcgcgccct tcttccgctg cgcttgcgcg cagggctgga ccgggccgcg ctgcgaggcg |
| 4201 | cccgccgcgg cacccgaggt ctcggaggag ccgcggtgcc cgcgcgccgc ctgccaggcc |
| 4261 | aagcgcgggg accagcgctg cgaccgcgag tgcaacagcc caggctgcgg ctgggacggc |
| 4321 | ggcgactgct cgctgagcgt gggcgacccc tggcggcaat gcgaggcgct gcagtgctgg |
| 4381 | cgcctcttca acaacagccg ctgcgacccc gcctgcagct cgcccgcctg cctctacgac |
| 4441 | aacttcgact gccacgccgg tggccgcgag cgcacttgca acccggtgta cgagaagtac |
| 4501 | tgcgccgacc actttgccga cggccgctgc gaccagggct gcaacacgga ggagtgcggc |
| 4561 | tgggatgggc tggattgtgc cagcgaggtg ccggccctgc tggcccgcgg cgtgctggtg |
| 4621 | ctcacagtgc tgctgccgcc ggaggagcta ctgcgttcca gcgccgactt tctgcagcgg |
| 4681 | ctcagcgcca tcctgcgcac ctcgctgcgc ttccgcctgg acgcgcacgg ccaggccatg |
| 4741 | gtcttccctt accaccggcc tagtcctggc tccgaacccc gggcccgtcg ggagctggcc |
| 4801 | cccgaggtga tcggctcggt agtaatgctg gagattgaca accggctctg cctgcagtcg |
| 4861 | cctgagaatg atcactgctt ccccgatgcc cagagcgccg ctgactacct gggagcgttg |
| 4921 | tcagcggtgg agcgcctgga cttcccgtac ccactgcggg acgtgcgggg ggagccgctg |
| 4981 | gagcctccag aacccagcgt cccgctgctg ccactgctag tggcgggcgc tgtcttgctg |
| 5041 | ctggtcattc tcgtcctggg tgtcatggtg gcccggcgca agcgcgagca cagcaccctc |
| 5101 | tggttccctg agggcttctc actgcacaag gacgtggcct ctggtcacaa gggccggcgg |
| 5161 | gaacccgtgg gccaggacgc gctgggcatg aagaacatgg ccaagggtga gagcctgatg |
| 5221 | ggggaggtgg ccacagactg gatggacaca gagtgcccag aggccaagcg gctaaaggta |
| 5281 | gaggagccag gcatgggggc tgaggaggct gtggattgcc gtcagtggac tcaacaccat |
| 5341 | ctggttgctg ctgacatccg cgtggcacca gccatggcac tgacaccacc acagggcgac |
| 5401 | gcagatgctg atggcatgga tgtcaatgtg cgtggcccag atggcttcac cccgctaatg |
| 5461 | ctggcttcct tctgtggggg ggctctggag ccaatgccaa ctgaagagga tgaggcagat |
| 5521 | gacacatcag ctagcatcat ctccgacctg atctgccagg gggctcagct tggggcacgg |
| 5581 | actgaccgta ctggcgagac tgctttgcac ctggctgccc gttatgcccg tgctgatgca |
| 5641 | gccaagcggc tgctggatgc tggggcagac accaatgccc aggaccactc aggccgcact |
| 5701 | cccctgcaca cagctgtcac agccgatgcc cagggtgtct tccagattct catccgaaac |
| 5761 | cgctctacag acttggatgc ccgcatggca gatggctcaa cggcactgat cctggcggcc |
| 5821 | cgcctggcag tagagggcat ggtggaagag ctcatcgcca gccatgctga tgtcaatgct |
| 5881 | gtggatgagc ttgggaaatc agccttacac tgggctgcgg ctgtgaacaa cgtggaagcc |
| 5941 | actttggccc tgctcaaaaa tggagccaat aaggacatgc aggatagcaa ggaggagacc |
| 6001 | cccctattcc tggccgcccg cgagggcagc tatgaggctg ccaagctgct gttggaccac |
| 6061 | tttgccaacc gtgagatcac cgaccacctg gacaggctgc cgcgggacgt agcccaggag |
| 6121 | agactgcacc aggacatcgt gcgcttgctg gatcaaccca gtgggccccg cagccccccc |
| 6181 | ggtccccacg gcctggggcc tctgctctgt cctccagggg ccttcctccc tggcctcaaa |
| 6241 | gcggcacagt cggggtccaa gaagagcagg aggccccccg ggaaggcggg gctggggccg |
| 6301 | caggggcccc gggggcgggg caagaagctg acgctggcct gcccgggccc cctggctgac |
| 6361 | agctcggtca cgctgtcgcc cgtggactcg ctggactccc cgcggccttt cggtgggccc |
| 6421 | cctgcttccc ctggtggctt cccccttgag gggccctatg cagctgccac tgccactgca |
| 6481 | gtgtctctgg cacagcttgg tggcccaggc cgggcaggtc tagggcgcca gccccctgga |
| 6541 | ggatgtgtac tcagcctggg cctgctgaac cctgtggctg tgcccctcga ttgggcccgg |
| 6601 | ctgcccccac ctgcccctcc aggcccctcg ttcctgctgc cactggcgcc gggaccccag |
| 6661 | ctgctcaacc cagggacccc cgtctccccg caggagcggc ccccgcctta cctggcagtc |
| 6721 | ccaggacatg gcgaggagta cccggtggct ggggcacaca gcagcccccc aaaggcccgc |
| 6781 | ttcctgcggg ttcccagtga gcacccttac ctgaccccat cccccgaatc ccctgagcac |
| 6841 | tgggccagcc cctcacctcc ctccctctca gactggtccg aatccacgcc tagcccagcc |
| 6901 | actgccactg gggccatggc caccaccact ggggcactgc ctgcccagcc acttcccttg |
| 6961 | tctgttccca gctcccttgc tcaggcccag acccagctgg ggccccagcc ggaagttacc |
| 7021 | cccaagaggc aagtgttggc ctgagacgct cgtcagttct tagatcttgg gggcctaaag |
| 7081 | agacccccgt cctgcctcct ttctttctct gtctcttcct tccttttagt ctttttcatc |
| 7141 | ctcttctctt tccaccaacc ctcctgcatc cttgccttgc agcgtgaccg agataggtca |
| 7201 | tcagcccagg gcttcagtct tcctttattt ataatgggtg ggggctacca cccaccctct |
| 7261 | cagtcttgtg aagagtctgg gacctccttc ttccccactt ctctcttccc tcattccttt |
| 7321 | ctctctcctt ctggcctctc atttccttac actctgacat gaatgaatta ttattatttt |
| 7381 | tctttttctt ttttttttta cattttgtat agaaacaaat tcatttaaac aaacttatta |
| 7441 | ttattatttt ttacaaaata tatatatgga gatgctccct ccccctgtga accccccagt |
| 7501 | gcccccgtgg ggctgagtct gtgggcccat tcggccaagc tggattctgt gtacctagta |
| 7561 | cacaggcatg actgggatcc cgtgtaccga gtacacgacc caggtatgta ccaagtaggc |
| 7621 | acccttgggc gcacccactg gggccagggg tcgggggagt gttgggagcc tcctccccac |
| 7681 | cccacctccc tcacttcact gcattccaga ttggacatgt tccatagcct tgctggggaa |
| 7741 | gggcccactg ccaactccct ctgccccagc cccacccttg gccatctccc tttgggaact |
| 7801 | agggggctgc tggtgggaaa tgggagccag ggcagatgta tgcattcctt tatgtccctg |
| 7861 | taaatgtggg actacaagaa gaggagctgc ctgagtggta ctttctcttc ctggtaatcc |
| 7921 | tctggcccag ccttatggca gaatagaggt atttttaggc tatttttgta atatggcttc |
| 7981 | tggtcaaaat ccctgtgtag ctgaattccc aagccctgca ttgtacagcc ccccactccc |
| 8041 | ctcaccacct aataaaggaa tagttaacac tcaaaaaaaa aaaaaaaaaa a |

By “Neurogenic locus notch homolog protein 4 (Notch4) polypeptide” is meant a protein having at least about 85% amino acid identity to the sequence provided at NCBI Reference Sequence: AAC32288.1, or a fragment thereof, and having Notch receptor activity. An exemplary Notch4 amino acid sequence is provided below:

| | |
|---|---|
| 1 | mqppslllll llllllcvsv vrprgllcgs fpepcanggt clslslgqgt cqcapgflge |
| 61 | tcqfpdpcqn aqlcqnggsc qallpaplgl psspspltps flctclpgft gercqakled |
| 121 | pcppsfcskr grchiqasgr pqcscmpgwt geqcqlrdfc sanpcvnggv clatypqiqc |
| 181 | hcppgfegha cerdvnecfq dpgpcpkgts chntlgsfqc lcpvgqegpr celragpcpp |
| 241 | rgcsnggtcq lmpekdstfh lclcppgfig pdcevnpdnc vshqcqnggt cqdgldtytc |
| 301 | lcpetwtgwd csedvdecet qgpphcrngg tcqnsagsfh cvcvsgwggt sceenlddci |
| 361 | aatcapgstc idrvgsfscl cppgrtgllc hledmclsqp chgdaqcstn pltgstlclc |
| 421 | qpgysgptch qdldeclmaq qgpspcehgg sclntpgsfn clcppgytgs rceadhnecl |
| 481 | sqpchpgstc ldllatfhcl cppglegqlc evetnecasa pclnhadchd llngfqcicl |
| 541 | pgfsgtrcee didecrsspc anggqcqdqp gafhckclpg fegprcqtev declsdpcpv |
| 601 | gascldlpga ffclcpsgft gqlcevplca pnlcqpkqic kdqkdkancl cpdgspgcap |
| 661 | pednctchhg hcqrsscvcd vgwtgpecea elggcisapc ahggtcypqp sgynctcptg |
| 721 | ytgptcseem tachsgpcln ggscnpspgg yyctcppsht gpqcqtstdy cvsapcfngg |
| 781 | tcvnrpgtfs clcamgfqgp rcegklrpsc adspcrnrat cqdspqgprc lcptgytggs |
| 841 | cqtlmdlcaq kpcprnshcl qtgpsfhclc lqgwtgplcn lplsscqkaa lsqgidvssl |
| 901 | chngglcvds gpsyfchcpp gfqgslcqdh vnpcesrpcq ngatcmaqps gylcqcapgy |
| 961 | dgqncskeld acqsqpchnh gtctpkpggf hcacppgfvg lrcegdvdec ldqpchptgt |
| 1021 | aachslanaf ycqclpghtg qwceveidpc hsqpcfhggt ceatagsplg fichcpkgfe |
| 1081 | gptcshraps cgfhhchhgg lclpspkpgf pprcaclsgy ggpdcltppa pkgcgppspc |
| 1141 | lyngscsett glggpgfrcs cphsspgprc qkpgakgceg rsgdgacdag csgpggnwdg |
| 1201 | gdcslgvpdp wkgcpshsrc wllfrdgqch pqcdseeclf dgydcetppa ctpaydqych |
| 1261 | dhfhnghcek gcntaecgwd ggdcrpedgd pewgpslall vvlsppaldq qlfalarvls |
| 1321 | ltlrvglwvr kdrdgrdmvy pypgaraeek lggtrdptyq eraapqtqpl gketdslsag |
| 1381 | fvvvmgvdls rcgpdhpasr cpwdpglllr flaamaavga lepllpgpll avhphagtap |
| 1441 | panqlpwpvl cspvagvill algallvlql irrrrrehga lwlppgftrr prtqsaphrr |
| 1501 | rpplgedsig lkalkpkaev dedgvvmcsg peegeevgqa eetgppstcq lwslsggcga |
| 1561 | lpqaamltpp qesemeapdl dtrgpdgvtp lmsavccgev qsgtfqgawl gcpepwepll |
| 1621 | dggacpqaht vgtgetplhl aarfsrptaa rrlleaganp nqpdragrtp lhaavaadar |
| 1681 | evcqlllrsr qtavdarted gttplmlaar lavedlveel iaaqadvgar dkwgktalhw |
| 1741 | aaavnnaraa rsllqagadk daqdnreqtp lflaaregav evaqlllglg aarelrdqag |
| 1801 | lapadvahqr nhwdlltlle gagppearhk atpgreagpf prartvsysv pphgggalpr |
| 1861 | crtlsagagp rgggaclqar twsvdlaarg ggayshcrsl sgvgagggpt prgrrfsagm |
| 1921 | rgprpnpaim rgrygvaagr ggrvstddwp cdwvalgacg sasnipippp cltpspergs |
| 1981 | pqldcgppal qempinqgge gkk |

By “Notch4 polynucleotide” is meant a nucleic acid molecule encoding a Notch4 polypeptide. An exemplary Notch4 polynucleotide sequence is provided at NCBI Reference Sequence: U95299.1, and reproduced herein below.

| | |
|---|---|
| 1 | gccggccgcg tcgaccctgc cccagtgaga gctctgaggg tccctgcctg aagagggaca |
| 61 | gggaccgggg cttggagaag gggctgtgga atgcagcccc cttcactgct gctgctgctg |
| 121 | ctgctgctgc tgctgctatg tgtctcagtg gtcagaccca gagggctgct gtgtgggagt |
| 181 | ttcccagaac cctgtgccaa tggaggcacc tgcctgagcc tgtctctggg acaagggacc |
| 241 | tgccagtgtg cccctggctt cctgggtgag acgtgccagt ttcctgaccc ctgccagaac |
| 301 | gcccagctct gccaaaatgg aggcagctgc caagccctgc ttcccgctcc cctagggctc |
| 361 | cccagctctc cctctccatt gacacccagc ttcttgtgca cttgcctccc tggcttcact |
| 421 | ggtgagagat gccaggccaa gcttgaagac ccttgtcctc cctccttctg ttccaaaagg |
| 481 | ggccgctgcc acatccaggc ctcgggccgc ccacagtgct cctgcatgcc tggatggaca |
| 541 | ggtgagcagt gccagcttcg ggacttctgt tcagccaacc catgtgttaa tggaggggtg |
| 601 | tgtctggcca cataccccca gatccagtgc cactgcccac cgggcttcga gggccatgcc |
| 661 | tgtgaacgtg atgtcaacga gtgcttccag gacccaggac cctgccccaa aggcacctcc |
| 721 | tgccataaca ccctgggctc cttccagtgc ctctgccctg tggggcagga gggtccacgt |
| 781 | tgtgagctgc gggcaggacc ctgccctcct aggggctgtt cgaatggggg cacctgccag |
| 841 | ctgatgccag agaaagactc cacctttcac ctctgcctct gtcccccagg tttcataggc |
| 901 | ccagactgtg aggtgaatcc agacaactgt gtcagccacc agtgtcagaa tgggggcact |
| 961 | tgccaggatg ggctggacac ctacacctgc ctctgcccag aaacctggac aggctgggac |
| 1021 | tgctccgaag atgtggatga gtgtgagacc cagggtcccc ctcactgcag aaacgggggc |
| 1081 | acctgccaga actctgctgg tagctttcac tgcgtgtgtg tgagtggctg gggcggcaca |
| 1141 | agctgtgagg agaacctgga tgactgtatt gctgccacct gtgccccggg atccacctgc |
| 1201 | attgaccggg tgggctcttt ctcctgcctc tgcccacctg gacgcacagg actcctgtgc |
| 1261 | cacttggaag acatgtgtct gagccagccg tgccatgggg atgcccaatg cagcaccaac |
| 1321 | cccctcacag gctccacact ctgcctgtgt cagcctggct attcggggcc cacctgccac |
| 1381 | caggacctgg acgagtgtct gatggcccag caaggcccaa gtccctgtga acatggcggt |
| 1441 | tcctgcctca acactcctgg ctccttcaac tgcctctgtc cacctggcta cacaggctcc |
| 1501 | cgttgtgagg ctgatcacaa tgagtgcctc tcccagccct gccacccagg aagcacctgt |
| 1561 | ctggacctac ttgccacctt ccactgcctc tgcccgccag gcttagaagg gcagctctgt |
| 1621 | gaggtggaga ccaacgagtg tgcctcagct ccctgcctga accacgcgga ttgccatgac |
| 1681 | ctgctcaacg gcttccagtg catctgcctg cctggattct ccggcacccg atgtgaggag |
| 1741 | gatatcgatg agtgcagaag ctctccctgt gccaatggtg ggcagtgcca ggaccagcct |
| 1801 | ggagccttcc actgcaagtg tctcccaggc tttgaagggc cacgctgtca aacagaggtg |
| 1861 | gatgagtgcc tgagtgaccc atgtcccgtt ggagccagct gccttgatct tccaggagcc |
| 1921 | ttcttttgcc tctgcccctc tggtttcaca ggccagctct gtgaggttcc cctgtgtgct |
| 1981 | cccaacctgt gccagcccaa gcagatatgt aaggaccaga aagacaaggc caactgcctc |
| 2041 | tgtcctgatg gaagccctgg ctgtgcccca cctgaggaca actgcacctg ccaccacggg |
| 2101 | cactgccaga gatcctcatg tgtgtgtgac gtgggttgga cggggccaga gtgtgaggca |
| 2161 | gagctagggg gctgcatctc tgcaccctgt gcccatgggg ggacctgcta cccccagccc |
| 2221 | tctggctaca actgcacctg ccctacaggc tacacaggac ccacctgtag tgaggagatg |
| 2281 | acagcttgtc actcagggcc atgtctcaat ggcggctcct gcaaccctag ccctggaggc |
| 2341 | tactactgca cctgccctcc aagccacaca gggccccagt gccaaaccag cactgactac |
| 2401 | tgtgtgtctg ccccgtgctt caatgggggt acctgtgtga acaggcctgg caccttctcc |
| 2461 | tgcctctgtg ccatgggctt ccagggcccg cgctgtgagg gaaagctccg ccccagctgt |
| 2521 | gcagacagcc cctgtaggaa tagggcaacc tgccaggaca gccctcaggg tccccgctgc |
| 2581 | ctctgcccca ctggctacac cggaggcagc tgccagactc tgatggactt atgtgcccag |
| 2641 | aagccctgcc cacgcaattc ccactgcctc cagactgggc cctccttcca ctgcttgtgc |
| 2701 | ctccagggat ggaccgggcc tctctgcaac cttccactgt cctcctgcca gaaggctgca |
| 2761 | ctgagccaag gcatagacgt ctcttccctt tgccacaatg gaggcctctg tgtcgacagc |
| 2821 | ggcccctcct atttctgcca ctgcccccct ggattccaag gcagcctgtg ccaggatcac |
| 2881 | gtgaacccat gtgagtccag gccttgccag aacggggcca cctgcatggc ccagcccagt |
| 2941 | gggtatctct gccagtgtgc cccaggctac gatggacaga actgctcaaa ggaactcgat |
| 3001 | gcttgtcagt cccaaccctg tcacaaccat ggaacctgta ctcccaaacc tggaggattc |
| 3061 | cactgtgcct gccctccagg ctttgtgggg ctacgctgtg agggagacgt ggacgagtgt |
| 3121 | ctggaccagc cctgccaccc cacaggcact gcagcctgcc actctctggc caatgccttc |
| 3181 | tactgccagt gtctgcctgg acacacaggc cagtggtgtg aggtggagat agacccctgc |
| 3241 | cacagccaac cctgctttca tggagggacc tgtgaggcca cagcaggatc acccctgggt |
| 3301 | ttcatctgcc actgccccaa gggttttgaa ggccccacct gcagccacag ggccccttcc |
| 3361 | tgcggcttcc atcactgcca ccacggaggc ctgtgtctgc cctcccctaa gccaggcttc |
| 3421 | ccaccacgct gtgcctgcct cagtggctat gggggtcctg actgcctgac cccaccagct |
| 3481 | cctaaaggct gtggccctcc ctccccatgc ctatacaatg gcagctgctc agagaccacg |
| 3541 | ggcttggggg gcccaggctt tcgatgctcc tgccctcaca gctctccagg gccccggtgt |
| 3601 | cagaaacccg gagccaaggg gtgtgagggc agaagtggag atggggcctg cgatgctggc |
| 3661 | tgcagtggcc cgggaggaaa ctgggatgga ggggactgct ctctgggagt cccagacccc |
| 3721 | tggaagggct gcccctccca ctctcggtgc tggcttctct tccgggacgg gcagtgccac |
| 3781 | ccacagtgtg actctgaaga gtgtctgttt gatggctacg actgtgagac ccctccagcc |
| 3841 | tgcactccag cctatgacca gtactgccat gatcacttcc acaacgggca ctgtgagaaa |
| 3901 | ggctgcaaca ctgcagagtg tggctgggat ggaggtgact gcaggcctga agatggggac |
| 3961 | ccagagtggg ggccctccct ggccctgctg gtggtactga gccccccagc cctagaccag |
| 4021 | cagctgtttg ccctggcccg ggtgctgtcc ctgactctga gggtaggact ctgggtaagg |
| 4081 | aaggatcgtg atggcaggga catggtgtac ccctatcctg gggcccgggc tgaagaaaag |
| 4141 | ctaggaggaa ctcgggaccc cacctatcag gagagagcag cccctcaaac gcagcccctg |
| 4201 | ggcaaggaga ccgactccct cagtgctggg ttcgtggtgg tcatgggtgt ggatttgtcc |
| 4261 | cgctgtggcc ctgaccaccc ggcatcccgc tgtccctggg accctgggct tctactccgc |
| 4321 | ttccttgctg cgatggctgc agtgggagcc ctggagcccc tgctgcctgg accactgctg |
| 4381 | gctgtccacc ctcatgcagg gaccgcaccc cctgccaacc agcttccctg gcctgtgctg |
| 4441 | tgctccccag tggccggggt gattctcctg gccctagggg ctcttctcgt cctccagctc |
| 4501 | atccggcgtc gacgccgaga gcatggagct ctctggctgc cccctggttt cactcgacgg |
| 4561 | cctcggactc agtcagctcc ccaccgacgc cggcccccac taggcgagga cagcattggt |
| 4621 | ctcaaggcac tgaagccaaa ggcagaagtt gatgaggatg gagttgtgat gtgctcaggc |
| 4681 | cctgaggagg gagaggaggt gggccaggct gaagaaacag gcccaccctc cacgtgccag |
| 4741 | ctctggtctc tgagtggtgg ctgtggggcg ctccctcagg cagccatgct aactcctccc |
| 4801 | caggaatctg agatggaagc ccctgacctg gacacccgtg gacctgatgg ggtgacaccc |
| 4861 | ctgatgtcag cagtttgctg tggggaagta cagtccggga ccttccaagg ggcatggttg |
| 4921 | ggatgtcctg agccctggga acctctgctg gatggagggg cctgtcccca ggctcacacc |
| 4981 | gtgggcactg gggagacccc cctgcacctg gctgcccgat tctcccggcc aaccgctgcc |
| 5041 | cgccgcctcc ttgaggctgg agccaacccc aaccagccag accgggcagg gcgcacaccc |
| 5101 | cttcatgctg ctgtggctgc tgatgctcgg gaggtctgcc agcttctgct ccgtagcaga |
| 5161 | caaactgcag tggacgctcg cacagaggac gggaccacac ccttgatgct ggctgccagg |
| 5221 | ctggcggtgg aagacctggt tgaagaactg attgcagccc aagcagacgt gggggccaga |
| 5281 | gataaatggg ggaaaactgc gctgcactgg gctgctgccg tgaacaacgc ccgagccgcc |
| 5341 | cgctcgcttc tccaggccgg agccgataaa gatgcccagg acaacaggga gcagacgccg |
| 5401 | ctattcctgg cggcgcggga aggagcggtg gaagtagccc agctactgct ggggctgggg |
| 5461 | gcagcccgag agctgcggga ccaggctggg ctagcgccgg cggacgtcgc tcaccaacgt |
| 5521 | aaccactggg atctgctgac gctgctggaa ggggctgggc caccagaggc ccgtcacaaa |
| 5581 | gccacgccgg gccgcgaggc tgggcccttc ccgcgcgcac ggacggtgtc agtaagcgtg |
| 5641 | cccccgcatg ggggcggggc tctgccgcgc tgccggacgc tgtcagccgg agcaggccct |
| 5701 | cgtgggggcg gagcttgtct gcaggctcgg acttggtccg tagacttggc tgcgcggggg |
| 5761 | ggcggggcct attcgcattg ccggagcctc tcgggagtag gagcaggagg aggcccgacc |
| 5821 | cctcgcggcc gtaggttttc tgcaggcatg cgcgggcctc ggcccaaccc tgcgataatg |
| 5881 | cgaggaagat acggagtggc tgccgggcgc ggaggcaggg tctcaacgga tgactggccc |
| 5941 | tgtgattggg tggccctggg agcttgcggt tctgcctcca acattccgat cccgcctcct |
| 6001 | tgccttactc cgtccccgga gcggggatca cctcaacttg actgtggtcc cccagccctc |
| 6061 | caagaaatgc ccataaacca aggaggagag ggtaaaaaat agaagaatac atggtaggga |
| 6121 | gg |

By “Notch inhibitor” is meant an agent capable of inhibiting the expression or activity of a Notch protein. Notch proteins include, but are not limited to, Notch1, Notch2, Notch3 and/or Notch4. In one embodiment, a Notch inhibitor reduces Notch signaling, for example by disrupting the receptor: ligand interaction or any other signaling event downstream of the Notch1, Notch2, Notch3 and/or Notch4 receptor, such as proteolytic cleavage of the Notch protein. In one embodiment, the Notch inhibitor is a gamma-secretase inhibitor (GSI). Notch inhibitors can include, for example, MK-0752, PF03084014, RO-4929097, DAPT, N-[N-(3,5-difluorophenacetyl)-L-alanyl]-S-phenylglycine t-butyl ester, tetralin imidazole PF-03084014, LY3039478 and BMS906-024. In some embodiments, inhibition is by at least about 10%, 25%, 50%, 75% or more. In another embodiment, a Notch inhibitor is any inhibitory nucleic acid that inhibits, for example, the expression of a Notch protein. In another embodiment, a Notch inhibitor is an antibody against Notch that inhibits Notch activity. Exemplary inhibitory Notch antibodies are known in the art, and include, for example, anti-Notch 1 (OMP-52M521) and anti-delta-like-4. In another embodiment, a

Notch inhibitor is a CRISPR-based therapeutic that depletes Notch (e.g., results in the conditional depletion of Notch).

By “B cell receptor inhibitor” is meant an agent capable of reducing B cell receptor signaling, including signaling by downstream pathways that are functionally regulated by B cell receptor signaling. In one embodiment, the B cell receptor inhibitor interrupts the receptor: ligand interaction or any other signaling event downstream of the B cell receptor. In one embodiment, the inhibitor is a Bruton tyrosine kinase (BTK) inhibitor. B cell receptor inhibitors can include, for example, ibrutinib (PCI-32765), acalabrutinib (ACP-196), ONO-4059 (e.g., GS-4059 or NCT02457598), spebrutinib (e.g., AVL-292, CC-292), and BGB-3111. In some embodiments, inhibition is by at least about 10%, 25%, 50%, 75% or more.

In another embodiment, a B cell receptor inhibitor is any inhibitory nucleic acid that inhibits, for example, the expression of a B cell receptor component, e.g., any protein that forms a functional part of the B cell receptor. In another embodiment, a B cell receptor inhibitor is an antibody that inhibits B cell receptor activity. In another embodiment, a B cell receptor inhibitor is a CRISPR-based therapeutic that depletes a B cell receptor component (e.g., results in the conditional depletion of a B cell receptor component).

By “Neural precursor cell expressed developmentally down-regulated protein 9 (Nedd9) polypeptide” is meant a protein having at least about 85% amino acid identity to the sequence provided at NCBI Reference Sequence: AAH40207.1, or a fragment thereof, and having cell cycle or growth regulatory activity. An exemplary Nedd9 amino acid sequence is provided below:

| | |
|---|---|
| 1 | mkyknlmara lydnvpecae elafrkgdil tvieqntggl egwwlcslhg rqgivpgnrv |
| 61 | klligpmqet assheqpasg lmqqtfgqqk lyqvpnpqaa prdtiyqvpp syqnqgiyqv |
| 121 | ptghgtqeqe vyqvppsvqr siggtsgphv gkkvitpvrt ghgyvyeyps ryqkdvydip |
| 181 | pshttqgvyd ippssakgpv fsvpvgeikp qgvydipptk gvyaippsac rdeaglrekd |
| 241 | ydfpppmrqa grpdlrpegv ydipptctkp agkdlhvkyn cdipgaaepv arrhqslspn |
| 301 | hpppqlgqsv gsqndaydvp rgvqfleppa etsekanpqe rdgvydvplh nppdakgsrd |
| 361 | lvdginrlsf sstgstrsnm stsstsskes slsaspaqdk rlfldpdtai erlqrlqqal |
| 421 | emgvsslmal vttdwrcygy merhineirt avdkvelflk eylhfvkgav anaaclpeli |
| 481 | lhnkmkrelq rvedshqils qtshdlnecs wslnilaink pqnkcddldr fvmvaktvpd |
| 541 | dakqltttin tnaealfrpg pgslhlkngp esimnsteyp hggsqgqllh pgdhkaqahn |
| 601 | kalppglske qapdcsssdg serswmddyd yvhlqgkeef erqqkellek enimkqnkmq |
| 661 | lehhqlsqfq lleqeitkpv endiskwkps qslpttnsgv saqdrqllcf yydqcethfi |
| 721 | sllnaidalf scvssaqppr ifvahskfvi lsahklvfig dtltrqvtaq dirnkvmnss |
| 781 | nqlceqlkti vmatkmaalh ypsttalqem vhqvtdlsrn aqlfkrslle matf |

By “Nedd9 polynucleotide” is meant a nucleic acid molecule encoding a Nedd9 polypeptide. An exemplary Nedd9 polynucleotide sequence is provided at NCBI Reference Sequence BC040207.1, and reproduced herein below.

| | |
|---|---|
| 1 | agtgacttga gggaggcgct gcgactgaca agcggctctg cccgggacct tctcgctttc |
| 61 | atctagcgct gcactcaatg gaggggcggg caccgcagtg cttaatgctg tcttaactag |
| 121 | tgtaggaaaa cggctcaacc caccgctgcc gaaatgaagt ataagaatct tatggcaagg |
| 181 | gccttatatg acaatgtccc agagtgtgcc gaggaactgg cctttcgcaa gggagacatc |
| 241 | ctgaccgtca tagagcagaa cacaggggga ctggaaggat ggtggctgtg ctcgttacac |
| 301 | ggtcggcaag gcattgtccc aggcaaccgg gtgaagcttc tgattggtcc catgcaggag |
| 361 | actgcctcca gtcacgagca gcctgcctct ggactgatgc agcagacctt tggccaacag |
| 421 | aagctctatc aagtgccaaa cccacaggct gctccccgag acaccatcta ccaagtgcca |
| 481 | ccttcctacc aaaatcaggg aatttaccaa gtccccactg gccacggcac ccaagaacaa |
| 541 | gaggtatatc aggtgccacc atcagtgcag agaagcattg ggggaaccag tgggccccac |
| 601 | gtgggtaaaa aggtgataac ccccgtgagg acaggccatg gctacgtata cgagtaccca |
| 661 | tccagatacc aaaaggacgt ctatgatatc cctccttctc ataccactca aggggtatac |
| 721 | gacatccctc cctcatcagc aaaaggccct gtgttttcag ttccagtggg agagataaaa |
| 781 | cctcaagggg tgtatgacat cccgcctaca aaaggggtat atgccattcc gccctctgct |
| 841 | tgccgggatg aagcagggct tagggaaaaa gactatgact tcccccctcc catgagacaa |
| 901 | gctggaaggc cggacctcag accggagggg gtttatgaca ttcctccaac ctgcaccaag |
| 961 | ccagcaggga aggaccttca tgtaaaatac aactgtgaca ttccaggagc tgcagaaccg |
| 1021 | gtggctcgaa ggcaccagag cctgtccccg aatcacccac ccccgcaact cggacagtca |
| 1081 | gtgggctctc agaacgacgc atatgatgtc ccccgaggcg ttcagtttct tgagccacca |
| 1141 | gcagaaacca gtgagaaagc aaacccccag gaaagggatg gtgtttatga tgtccctctg |
| 1201 | cataacccgc cagatgctaa aggctctcgg gacttggtgg atgggatcaa ccgattgtct |
| 1261 | ttctccagta caggcagcac ccggagtaac atgtccacgt cttccacctc ctccaaggag |
| 1321 | tcctcactgt cagcctcccc agctcaggac aaaaggctct tcctggatcc agacacagct |
| 1381 | attgagagac ttcagcggct ccagcaggcc cttgagatgg gtgtctccag cctaatggca |
| 1441 | ctggtcacta ccgactggcg gtgttacgga tatatggaaa gacacatcaa tgaaatacgc |
| 1501 | acagcagtgg acaaggtgga gctgttcctg aaggagtacc tccactttgt caagggagct |
| 1561 | gttgcaaatg ctgcctgcct cccggaactc atcctccaca acaagatgaa gcgggagctg |
| 1621 | caacgagttg aagactccca ccagatcctg agtcaaacca gccatgactt aaatgagtgc |
| 1681 | agctggtccc tgaatatctt ggccatcaac aagccccaga acaagtgtga cgatctggac |
| 1741 | cggtttgtga tggtggcaaa gacggtgccc gatgacgcca agcagctcac cacaaccatc |
| 1801 | aacaccaacg cagaggccct cttcagaccc ggccctggca gcttgcatct gaagaatggg |
| 1861 | ccggagagca tcatgaactc aacggagtac ccacacggtg gctcccaggg acagctgctg |
| 1921 | catcctggtg accacaaggc ccaggcccac aacaaggcac tgcccccagg cctgagcaag |
| 1981 | gagcaggccc ctgactgtag cagcagtgat ggttctgaga ggagctggat ggatgactac |
| 2041 | gattacgtcc acctacaggg taaggaggag tttgagaggc aacagaaaga gctattggaa |
| 2101 | aaagagaata tcatgaaaca gaacaagatg cagctggaac atcatcagct gagccagttc |
| 2161 | cagctgttgg aacaagagat tacaaagccc gtggagaatg acatctcgaa gtggaagccc |
| 2221 | tctcagagcc tacccaccac aaacagtggc gtgagtgctc aggatcggca gttgctgtgc |
| 2281 | ttctactatg accaatgtga gacccatttc atttcccttc tcaacgccat tgacgcactc |
| 2341 | ttcagttgtg tcagctcagc ccagcccccg cgaatcttcg tggcacacag caagtttgtc |
| 2401 | atcctcagtg cacacaaact ggtgttcatt ggagacacgc tgacacggca ggtgactgcc |
| 2461 | caggacattc gcaacaaagt catgaactcc agcaaccagc tctgcgagca gctcaagacc |
| 2521 | atagtcatgg caaccaagat ggccgccctc cattacccca gcaccacggc cctgcaggaa |
| 2581 | atggtgcacc aagtgacaga cctttctaga aatgcccagc tgttcaagcg ctctttgctg |
| 2641 | gagatggcaa cgttctgaga agaaaaaaaa gaggaagggg actgcgttaa cggttactaa |
| 2701 | ggaaaactgg aaatactgtc tggtttttgt aaatgttatc tatttttgta gatattttat |
| 2761 | ataaaaatga aatattttaa cattttatgg gtcagtcaac tttcagaaat tcagggagct |
| 2821 | ggagagggaa atcttttttt ttccccctga gtggttctta tgtacataga ggtatctgag |
| 2881 | acataaactg tacagaaaac ttgtccacgt gcttttgtat gcccatgtat tcatgtttgt |
| 2941 | ttgtagatgt ttgtctgatg catttcatta aaaaaaaaac catgaattac gaagcacctt |
| 3001 | agtaagcacc tcctaatgct gcattttttt tgttgttgtt aaaaacatac cagctggtta |
| 3061 | taatattgtt ctccacgtcc ttgtgatgat tctgagcctg gcactcccaa atctgggaag |
| 3121 | catagtttat ttgcaagtgt tcaccttcca aatcatgagg catagcatga cttattcttg |
| 3181 | tttggaaaac tcttttcaaa actgaccatc ttaaacacat gatggccaag tgcccaaaag |
| 3241 | ccctcttgcg gagcaaattt cagaatatat atgtggatcc aagctctgat agttcaggtg |
| 3301 | ctggagggaa gagagacctg tgtgtttaga ggccaggacc acagttagga ttgggttgtt |
| 3361 | tcaatactga gagacagcta caataaaagg agagcaattg cctccctggg gctgttcaat |
| 3421 | cttctgcatt tgtgagtggt tcagtcatga ggttttccaa aagatgtttt tagagttgta |
| 3481 | aaaaccatat ttgcagcaaa gatttacaaa ggcgtatcag actatgattg ttcaccaaaa |
| 3541 | taggggaatg gtttgatccg ccagttgcaa gtagaggcct ttctgactct taatattcac |
| 3601 | tttggtgcta ctacccccat tacctgaggg aaactggcca ggtccttgat catggaacta |
| 3661 | tagagctacc aggacatatc ctgctctcta agggaattta ttgctatctt gcaccttctt |
| 3721 | taaaactcac atatgcagac ctgacactca agagtggcta gctacacaga gtccatctaa |
| 3781 | tttttgcaac ttcctgtggc cagtgtgtat aaccccttcc actatctcac agatagtcac |
| 3841 | agcgtccatt ccatagtctg tctcctcaca tctgttagta ttgacacagc acagacacca |
| 3901 | caagccatca ggttcttcat ggggcaggtg aaatacttct accccatggg taaatgtatt |
| 3961 | cacatattac caagagaaga agcacattat ctatgatctt ttggcccagt tcttatttag |
| 4021 | catttttatt ccagcctact tggaaacatg tttttatttg caatatatgc ctgactgaat |
| 4081 | taagcttgct tgttttaaac aaccaaatca ttggaacaga aaaggattta aaaaacaaga |
| 4141 | atgcatgatc tcagagtgat taaaaaaaaa tcagtggaaa taaatgatca tagaaggtgc |
| 4201 | ttttcaaaac aactgctatt ataattctca aagtcctact ctgccaaaag aagattaaaa |
| 4261 | gtcatacatt acattacaag gaaatgttca tgtgggaaga gggttgctga aaatcaacaa |
| 4321 | cgcttgaagt taaaaagtgt gtctttgtag atttcattgt ataatgtgta tttcttagga |
| 4381 | gatggctgac ttgattgatc tacgctaagt ggagacattt cacattttta aaaccaaatg |
| 4441 | ttcaatctgt attactcttt gccgtcttgt atgtagaggc tatttttaaa tcattaaatt |
| 4501 | tttagatctc tgttttcaaa aaaaaaaaaa aa |

By “Phospholipase C Gamma 2, (PLCG2, 1-Phosphatidylinositol-4,5-bisphosphate phosphodiesterase gamma-2) polypeptide” is meant a protein having at least about 85% amino acid identity to the sequence provided at NCBI Reference Sequence: AAQ76815.1, or a fragment thereof, and having phospholipase activity. An exemplary PLCG2 amino acid sequence is provided below:

| | |
|---|---|
| 1 | msttvnvdsl aeyeksqikr alelgtvmtv fsfrkstper rtvqvimetr qvawsktadk |
| 61 | iegfldimei keirpgknsk dferakavrq kedccftily gtqfvlstls laadskedav |
| 121 | nwlsglkilh qeamnastpt iieswlrkqi ysvdqtrrns islrelktil plinfkvssa |
| 181 | kflkdkfvei gahkdelsfe qfhlfykklm feqqksilde fkkdssvfil gntdrpdasa |
| 241 | vylhdfqrfl iheqqehwaq dlnkvrermt kfiddtmret aepflfvdef ltylfsrens |
| 301 | iwdekydavd mqdmnnplsh ywissshnty ltgdqlrses speayirclr mgcrcieldc |
| 361 | wdgpdgkpvi yhgwtrttki kfddvvqaik dhafvtssfp vilsieehcs veqqrhmaka |
| 421 | fkevfgdlll tkpteasadq lpspsqlrek iiikhkklgp rgdvdvnmed kkdehkqqge |
| 481 | lymwdsidqk wtrhycaiad aklsfsddie qtmeeevpqd ipptelhfge kwfhkkvekr |
| 541 | tsaekllqey cmetggkdgt flvresetfp ndytlsfwrs grvqhcrirs tmeggtlkyy |
| 601 | ltdnlrfrrm yaliqhyret hlpcaefelr ltdpvpnpnp heskpwyyds lsrgeaedml |
| 661 | mriprdgafl irkregsdsy aitfrargkv khcrinrdgr hfvlgtsayf eslvelvsyy |
| 721 | ekhslyrkmr lrypvtpell erynterdin slydvsrmyv dpseinpsmp qrtvkalydy |
| 781 | kakrsdelsf crgalihnvs kepggwwkgd ygtriqqyfp snyvedista dfeelekqii |
| 841 | ednplgslcr gildlntynv vkapqgknqk sfvfilepke qgdppvefat drveelfewf |
| 901 | qsireitwki dskennmkyw eknqsiaiel sdlvvyckpt sktkdnlenp dfreirsfve |
| 961 | tkadsiirqk pvdllkynqk gltrvypkgq rvdssnydpf rlwlcgsqmv alnfqtadky |
| 1021 | mgmnhalfsl ngrtgyvlqp esmrtekydp mppesqrkil mtltvkvlga rhlpklgrsi |
| 1081 | acpfveveic gaeygnnkfk ttvvndngls piwaptqekv tfeiydpnla flrfvvyeed |
| 1141 | mfsdpnflah atypikavks gfrsvplkng ysedielasl lvfcemrpvl eseeelyssc |
| 1201 | rqlrrrqeel nnqlflydth qnlrnanrda lvkefsvnen hssctrrnat rg |

By “PLCG2 polynucleotide” is meant a nucleic acid molecule encoding a PLCG2 polypeptide. An exemplary PLCG2 polynucleotide sequence is provided at NCBI Reference Sequence: NM 002661.4, and reproduced herein below.

| | |
|---|---|
| 1 | gaggatcacg tggcgcggcg ccgcggccga agcagaagta gcgagcgccg gcggcggagg |
| 61 | gcgtgagcgg cgctgagtga cccgagtcgg gacgcgggct gcgcgcgcgg gaccccggag |
| 121 | cccaaacccg gggcaggcgg gcagctgtgc ccgggcggca cggccagctt cctgatttct |
| 181 | cccgattcct tccttctccc tggagcggcc gacaatgtcc accacggtca atgtagattc |
| 241 | ccttgcggaa tatgagaaga gccagatcaa gagagccctg gagctgggga cggtgatgac |
| 301 | tgtgttcagc ttccgcaagt ccacccccga gcggagaacc gtccaggtga tcatggagac |
| 361 | gcggcaggtg gcctggagca agaccgctga caagatcgag ggcttcttgg atatcatgga |
| 421 | aataaaagaa atccgcccag ggaagaactc caaagatttc gagcgagcaa aagcagttcg |
| 481 | ccagaaagaa gactgctgct tcaccatcct atatggcact cagttcgtcc tcagcacgct |
| 541 | cagcttggca gctgactcta aagaggatgc agttaactgg ctctctggct tgaaaatctt |
| 601 | acaccaggaa gcgatgaatg cgtccacgcc caccattatc gagagttggc tgagaaagca |
| 661 | gatatattct gtggatcaaa ccagaagaaa cagcatcagt ctccgagagt tgaagaccat |
| 721 | cttgcccctg atcaacttta aagtgagcag tgccaagttc cttaaagata agtttgtgga |
| 781 | aataggagca cacaaagatg agctcagctt tgaacagttc catctcttct ataaaaaact |
| 841 | tatgtttgaa cagcaaaaat cgattctcga tgaattcaaa aaggattcgt ccgtgttcat |
| 901 | cctggggaac actgacaggc cggatgcctc tgctgtttac ctgcatgact tccagaggtt |
| 961 | tctcatacat gaacagcagg agcattgggc tcaggatctg aacaaagtcc gtgagcggat |
| 1021 | gacaaagttc attgatgaca ccatgcgtga aactgctgag cctttcttgt ttgtggatga |
| 1081 | gttcctcacg tacctgtttt cacgagaaaa cagcatctgg gatgagaagt atgacgcggt |
| 1141 | ggacatgcag gacatgaaca accccctgtc tcattactgg atctcctcgt cacataacac |
| 1201 | gtaccttaca ggtgaccagc tgcggagcga gtcgtcccca gaagcttaca tccgctgcct |
| 1261 | gcgcatgggc tgtcgctgca ttgaactgga ctgctgggac gggcccgatg ggaagccggt |
| 1321 | catctaccat ggctggacgc ggactaccaa gatcaagttt gacgacgtcg tgcaggccat |
| 1381 | caaagaccac gcctttgtta cctcgagctt cccagtgatc ctgtccatcg aggagcactg |
| 1441 | cagcgtggag caacagcgtc acatggccaa ggccttcaag gaagtatttg gcgacctgct |
| 1501 | gttgacgaag cccacggagg ccagtgctga ccagctgccc tcgcccagcc agctgcggga |
| 1561 | gaagatcatc atcaagcata agaagctggg cccccgaggc gatgtggatg tcaacatgga |
| 1621 | ggacaagaag gacgaacaca agcaacaggg ggagctgtac atgtgggatt ccattgacca |
| 1681 | gaaatggact cggcactact gcgccattgc cgatgccaag ctgtccttca gtgatgacat |
| 1741 | tgaacagact atggaggagg aagtgcccca ggatataccc cctacagaac tacattttgg |
| 1801 | ggagaaatgg ttccacaaga aggtggagaa gaggacgagt gccgagaagt tgctgcagga |
| 1861 | atactgcatg gagacggggg gcaaggatgg caccttcctg gttcgggaga gcgagacctt |
| 1921 | ccccaatgac tacaccctgt ccttctggcg gtcaggccgg gtccagcact gccggatccg |
| 1981 | ctccaccatg gagggcggga ccctgaaata ctacttgact gacaacctca ccttcagcag |
| 2041 | catctatgcc ctcatccagc actaccgcga gacgcacctg cgctgcgccg agttcgagct |
| 2101 | gcggctcacg gaccctgtgc ccaaccccaa cccccacgag tccaagccgt ggtactatga |
| 2161 | cagcctgagc cgcggagagg cagaggacat gctgatgagg attccccggg acggggcctt |
| 2221 | cctgatccgg aagcgagagg ggagcgactc ctatgccatc accttcaggg ctaggggcaa |
| 2281 | ggtaaagcat tgtcgcatca accgggacgg ccggcacttt gtgctgggga cctccgccta |
| 2341 | ttttgagagt ctggtggagc tcgtcagtta ctacgagaag cattcactct accgaaagat |
| 2401 | gagactgcgc taccccgtga cccccgagct cctggagcgc tacaatatgg aaagagatat |
| 2461 | aaactccctc tacgacgtca gcagaatgta tgtggatccc agtgaaatca atccgtccat |
| 2521 | gcctcagaga accgtgaaag ctctgtatga ctacaaagcc aagcgaagcg atgagctgag |
| 2581 | cttctgccgt ggtgccctca tccacaatgt ctccaaggag cccgggggct ggtggaaagg |
| 2641 | agactatgga accaggatcc agcagtactt cccatccaac tacgtcgagg acatctcaac |
| 2701 | tgcagacttc gaggagctag aaaagcagat tattgaagac aatcccttag ggtctctttg |
| 2761 | cagaggaata ttggacctca atacctataa cgtcgtgaaa gcccctcagg gaaaaaacca |
| 2821 | gaagtccttt gtcttcatcc tggagcccaa gcagcagggc gatcctccgg tggagtttgc |
| 2881 | cacagacagg gtggaggagc tctttgagtg gtttcagagc atccgagaga tcacctggaa |
| 2941 | gattgacacc aaggagaaca acatgaagta ctgggagaag aaccagtcca tcgccatcga |
| 3001 | gctctctgac ctggttgtct actgcaaacc aaccagcaaa accaaggaca acttagaaaa |
| 3061 | tcctgacttc cgagaaatcc gctcctttgt ggagacgaag gctgacagca tcatcagaca |
| 3121 | gaagcccgtc gacctcctga agtacaatca aaagggcctg acccgcgtct acccaaaggg |
| 3181 | acaaagagtt gactcttcaa actacgaccc cttccgcctc tggctgtgcg gttctcagat |
| 3241 | ggtggcactc aatttccaga cggcagataa gtacatgcag atgaatcacg cattgttttc |
| 3301 | tctcaatggg cgcacgggct acgttctgca gcctgagagc atgaggacag agaaatatga |
| 3361 | cccgatgcca cccgagtccc agaggaagat cctgatgacg ctgacagtca aggttctcgg |
| 3421 | tgctcgccat ctccccaaac ttggacgaag tattgcctgt ccctttgtag aagtggagat |
| 3481 | ctgtggagcc gagtatgaca acaacaagtt caagacgacg gttgtgaatg ataatggcct |
| 3541 | cagccctatc tgggctccaa cacaggagaa ggtgacattt gaaatttatg acccaaacct |
| 3601 | ggcatttctg cgctttgtgg tttatgaaga agatatgttc agcgatccca actttcttgc |
| 3661 | tcatgccact taccccatta aagcagtcaa atcaggattc aggtccgttc ctctgaagaa |
| 3721 | tgggtacagc gaggacatag agctggcttc cctcctggtt ttctgtgaga tgcggccagt |
| 3781 | cctggagagc gaagaggaac tttactcctc ctgtcgccag ctgaggaggc ggcaagaaga |
| 3841 | actgaacaac cagctctttc tgtatgacac acaccagaac ttgcgcaatg ccaaccggga |
| 3901 | tgccctggtt aaagagttca gtgttaatga gaaccagctc cagctgtacc aggagaaatg |
| 3961 | caacaagagg ttaagagaga agagagtcag caacagcaag ttttactcat agaagctggg |
| 4021 | gtatgtgtgt aagggtattg tgtgtgtgcg catgtgtgtt tgcatgtagg agaacgtgcc |
| 4081 | ctattcacac tctgggaaga cgctaatctg tgacatcttt tcttcaagcc tgccatcaag |
| 4141 | gacatttctt aagacccaac tggcatgagt tggggtaatt tcctattatt ttcatcttgg |
| 4201 | acaactttct taacttatat tctttataga ggattcccca aaatgtgctc ctcatttttg |
| 4261 | gcctctcatg ttccaaacct cattgaataa aagcaatgaa aaccttgatc aattaagcct |
| 4321 | tctgttgcac gacctgtgca gtgaacagga tttcttttct ggccaagaag attctacctc |
| 4381 | taatgatcca ggtaactgat gtccatggag gatgagctgg aaatgtaaga aactattcat |
| 4441 | gagattctga aaaggatttt aactcaaagg caaatgattc cataagggcc caaagagaag |
| 4501 | ccctacccac aggcagcctg ctcagttcaa tgtactttaa ctaccaccgg ctgcctgctg |
| 4561 | cagtccacaa gaaaatggct gagtgatggg atctgttcat taagacaatt tctaattaat |
| 4621 | ggtgacagct tgttttgtga ctagagttac tgggatggag ggtaggaatc ttggggcctc |
| 4681 | tttgttttaa aaagcccatc agagagacca gagccgtgct gcaggggcag gttctcactt |
| 4741 | gcccctggct ctgccagctg ctgggaggct ctggccccac tagtccctca tggccctact |
| 4801 | gaactggctg ggaggctgct ggaatggccc ttggtccaca gctctccaca ggcaagaggt |
| 4861 | caactgctgc ttgaaagagg tagacaaaag ttaggttgat ggcgaaatgt ctctgggtta |
| 4921 | cccagtcttc tggagcagca agctgagctt taatgggcta agcattaggg tgttacagaa |
| 4981 | aatttcaaat gcagccatct cccttggggc agatctacct agttcatgac agtatgtgcg |
| 5041 | gctggccagg gctttacacc tctgcatctt aagttgttaa tacataccaa taatgtaata |
| 5101 | tggcttttta aaggagagga gagtgctggg ttgggaaggg aggtggttgg tagagtcaca |
| 5161 | acttctcaat gagtgaattt acagctgatg ggaaaaggag tgtaactgtg aaaaacgatg |
| 5221 | gctgtggtgg ggaagaacaa accagcagta agcctgatgt ttgatgtgga tggaactggc |
| 5281 | ccctagaaac ccatctgacc ctcctcttgt tacccgaaat gctgggctta gtatgcatgt |
| 5341 | actgctgaaa agcagggcag aacaaatcag gctctgacca gaagatcctt ctggtccctt |
| 5401 | cactctacaa aaacttactg atcacctcca catgccaaat acagtgccaa gatttggggg |
| 5461 | tgtggatgtt taaacaaaaa gctgtgggtc tcatcaatca tctccatcca caagctccta |
| 5521 | aaagaaagcc atttacctcg cttgaagcca ggaacacagg gaacagcagt ctggccaagg |
| 5581 | aagggctgtt atctggtgct atcactccag ttactcctcc aactgggagc tgctatttta |
| 5641 | tttggcagtc agcaactgaa gaaagaacat tcctcttagt ggcagatgtt caaagcaact |
| 5701 | ttcaagaaag gctaggtgag aaaggcactg ggatgagtgc tgcaggcact ctgtagccag |
| 5761 | ggccccatta gcctttggcc aggtagccac cagaacctat ttattgcacc tggcatctcc |
| 5821 | cccaacccct ctcagctctg ttaggacttc cacacagcag agctcaggtg ttgctgtcat |
| 5881 | tacctccttt cagctcctca cttcattcta ctttaaagcc acagtgctaa ggcctgcatc |
| 5941 | ccctttctgc ccaaatgggt tttttgctac catatcaaag aacctgacat atggcggcat |
| 6001 | aggaagcaga agctaagcct ctctccagct gctgctgtgt aaaatccatg cgtggccaaa |
| 6061 | gagaagtcag gggattatga cataaatggt gctgggaaga accctctgcc taaaactgtc |
| 6121 | tccttctcct ggtgctacaa ccggaatcca ccatgagaga gtactttctt cggttctttc |
| 6181 | ctcctgtcct tgacagagta acacgttaat ctggttcttg gtggtgttag ggactgattc |
| 6241 | tctcaggaaa ggcacacatg gtatgatggc tcttcccaga gtctatgtga tgctacataa |
| 6301 | cttcagtatc tagctgagac atgcttccta catgactgtt aaagcacagc caatccaggc |
| 6361 | caagaagact agtaacaggc acattctgaa agatggaagc agcactgata gatcaaaacc |
| 6421 | accactgcat atgtattaca ctgtttttgt tcaccatttt cctaagtgtg ttatttagaa |
| 6481 | tattggttat tacaaggaaa aataaagtgg ggaggctggt taggccttgt gagtttggga |
| 6541 | aacttaggtt ataaaaacta aataaagttt ttctactgtg agactagatg tgcaggagtg |
| 6601 | aaaggtgtag agggtcttgt tttccaaatt cgatctcaga atctttttgc cagaagtgtc |
| 6661 | tcatgggact tatctatagt ggaacacatt tgaagaccta ctgctctatt aagaaggcag |
| 6721 | ccggacaaca tgttctaata cttcgtatgc tttgtgacct agttaaaatc taaacttaag |
| 6781 | tcgccatggc cagtggcctt tagattaagc tagccttacc cctgggagta taccagagct |
| 6841 | ttccaaggaa tacacagact ccagtactct caggggagca gtgttcagag cctcatcttc |
| 6901 | ctgttatatt cttctctaag attcatctgc ctgagaaaat gcccttttct caccttacaa |
| 6961 | aagaaaatat ggctgtctcc acctctagtc ttactgtaga gcatgtccca aggtgtaaaa |
| 7021 | attcaaaatg tggatatttg gaaagtgaaa gacttatcaa cagggcacaa atctttttgc |
| 7081 | aaatggattt tccaagtttt tctggtggtt ccaaattttt tgctttcaac aaagtgggag |
| 7141 | gaacagcctg tagatttctg agtctcttag catgtaacta caaaggggtt ggaagaattc |
| 7201 | agtgattctg ctatcataaa gcttccgttc ccattgatgt atctgtgtga acaaggatca |
| 7261 | acatctccat aaatgaaatt gaaaacggaa aatagaattg atgatgaact ttggctcaat |
| 7321 | cttaagatgt tatcaatcta catagatgaa ataattgtgg agaaaagccc tctttatctc |
| 7381 | attaagtgat acatttccaa agaagtttta ctatgtttaa taatttagtg aaatttgggc |
| 7441 | tatgtgttta ttgattcagc tcaatccaga ggaaaatttt aaaggcttac agccttagga |
| 7501 | ttataggata ctatataata cttttggtac agagatagaa ttaaataaca taaaaatcaa |
| 7561 | aaatttatta ggctaaaatt ttgagggaga agtggtatga aaatacaaat tcaaggagta |
| 7621 | aaaggaaaag tggggcattc cttgctacta aaaattgcct tgttccaggt aagactgatc |
| 7681 | ataaaaaaat ggccctgttc ataaaatttt taaaaagatc atagtatcta tcaaataact |
| 7741 | tatattaaga acctcctggg ctaaatttaa aaagtaatac aacagtttta tttaaacatg |
| 7801 | tagtgtctac ggtatgccag cactttgcag ctatttataa tgagaaattt tagatgtcaa |
| 7861 | tatagcaatg tgcaagaaga tagagatttt caaaattcac ttaagagtat ctgagcataa |
| 7921 | aatgttaaga ttgctgatcg gatgtgaggg cgatctggct gcgacatctg tcaccccatt |
| 7981 | gatcgccagg gttgattcgg ctgatctggc tggctaggtg ggtgtcccct tcctacctca |
| 8041 | ccgctccatg tgcgtccctc ccgaagctgc gcgctccgtc gaagaggacg accaaccccg |
| 8101 | atagaggagg accggtcttc ggtcaagggt atacgagtag ctgcgctccc ctgctggaac |
| 8161 | ctccaaacaa gctctcaaga ttgctgatct agggccacta agtgatgaat tgtatttgga |
| 8221 | agcaaaaagg atggctaaaa aggacctcaa cccttttgac tttaaaagga aaatagctta |
| 8281 | accttcaacc tgtgtgacat ttaacttttt gaacccaacc gtaaaagcta tcttctaacc |
| 8341 | aacaaaaagt taataattag atttggaatt atacagaatt agaaaattgg catttaaaaa |
| 8401 | tactcaataa tttgtccctg gtttttaatt ttcaaaatat tttctttttg aagagccaga |
| 8461 | ttccagtgat cctgcctctc agaaatttcc acatttctta tttttcatta ggccttaaga |
| 8521 | agctgcattt gtaaacttgt gtttcattat taaagcttaa tttatttttt atataaatag |
| 8581 | tatgtgcttt gtgtacatag agaattaagt gaatgagtca cacagatgtt ggctgttgtt |
| 8641 | aatgtgaaaa ttaaacagct gtatcacatt ttgaaaaata aaagtttcat ctgaatgaat |
| 8701 | atagcaa |

By “recombining binding protein suppressor of hairless isoform 1 (RBPJ) polypeptide” is meant a protein having at least about 85% amino acid identity to the sequence provided at NCBI Reference Sequence: NP 005340.2, or a fragment thereof, and having transcriptional regulatory activity. An exemplary RBPJ amino acid sequence is provided below:

| | |
|---|---|
| 1 | mdhtegspae eppahapspg kfgerpppkr ltreamrnyl kergdqtvli lhakvaqksy |
| 61 | gnekrffcpp pcvylmgsgw kkkkeqmerd gcseqesqpc afigignsdq emqqlnlegk |
| 121 | nyctaktlyi sdsdkrkhfm lsvkmfygns ddigvflskr ikviskpskk kqslknadlc |
| 181 | iasgtkvalf nrlrsqtvst rylhveggnf hassqqwgaf fihlldddes egeeftvrdg |
| 241 | yihygqtvkl vcsvtgmalp rliirkvdkq talldaddpv sqlhkcafyl kdtermylcl |
| 301 | sqeriiqfqa tpcpkepnke mindgaswti istdkaeytf yegmgpvlap vtpvpvvesl |
| 361 | qlngggdvam leltgqnftp nlrvwfgdve aetmyrcges mlcvvpdisa fregwrwvrq |
| 421 | pvqvpvtlvr ndgiiystsl tftytpepgp rphcsaagai lranssqvpp nesntnsegs |
| 481 | ytnastnsts vtsstatvvs |

By “RBPJ polynucleotide” is meant a nucleic acid molecule encoding a RBPJ polypeptide. An exemplary RBPJ polynucleotide sequence is provided at NCBI Reference Sequence NM 014276.3, and reproduced herein below.

| | |
|---|---|
| 1 | gtgtgcaggg ttccagcgac agcagcactg gactcgtcca gagggcggcg ggtgagcggc |
| 61 | tggggccccg tggagccacc atggaccccg caggggcagc agacccctca gtgcctccca |
| 121 | atcctttgac tcacctgagc ctgcaggaca gatcagagat gcagctgcag agcgaagccg |
| 181 | acaggcggag cctcccgggc acttggacca ggtcatcccc agagcacacc accattctga |
| 241 | ggggaggcgt gcgcaggtgc ctgcagcaac agtgtgaaca gactgtgcgg atcctgcatg |
| 301 | ccaaggtggc ccagaaatca tacggaaatg agaagcggtt cttctgcccc ccgccctgtg |
| 361 | tctacctctc ggggcctggc tggagggtga agccagggca ggatcaagct caccaggcgg |
| 421 | gggaaacggg gcccacggtc tgcggttaca tgggactgga cagcgcgtcc ggcagcgcca |
| 481 | ctgagacgca gaagctgaat ttcgagcagc agccggactc cagggaattc ggctgcgcca |
| 541 | agaccctgta catctcagat gcagacaaga ggaagcactt tcggctggtg ctgcggctgg |
| 601 | tgctgcgcgg gggccgggag ctgggtacct tccacagccg ccttatcaag gtcatctcga |
| 661 | agccctcgca gaagaagcag tcgctgaaaa acaccgatct gtgcatatcc tccggctcaa |
| 721 | aggtctccct cttcaaccgc ctgcgctctc agacggtctc cacacgctac ctctctgtgg |
| 781 | aggatggggc ctttgtggcc agtgcacgac agtgggctgc cttcacgctc cacctggctg |
| 841 | atgggcactc tgcccaagga gacttcccac cgcgagaggg ctacgttcgc tatggctccc |
| 901 | tggtgcagct cgtctgcacg gtcaccggca tcacactacc tcccatgatc atccgtaaag |
| 961 | tagcaaaaca gtgtgcgctc cttgatgtgg atgagcccat ctcccagctg cacaagtgtg |
| 1021 | cattccagtt tccaggcagt cccccaggag ggggtggcac ctacttatgc cttgccacag |
| 1081 | agaaggtggt gcaatttcag gcctctccct gccccaagga ggcgaacagg gctctgctta |
| 1141 | acgacagctc ttgctggacc atcatcggca ccgagtcggt ggaattttcc ttcagcacca |
| 1201 | gcctggcgtg taccctggag ccggtcactc cggtgcctct catcagcacc ctagagctga |
| 1261 | gcggcggggg cgacgtggcc acgctggagc tccacggaga gaacttccac gcggggctca |
| 1321 | aggtgtggtt tggggacgtg gaggcagaaa ccatgtacag gagcccgcgg tccctggtgt |
| 1381 | gcgtggtgcc ggacgtggcg gccttctgca gcgactggcg ctggctgcgc gctcccatca |
| 1441 | caatccccat gagcctggtg cgcgccgacg ggctcttcta ccctagtgcc ttctccttca |
| 1501 | cctacacccc ggaatacagc gtgcggccgg gtcaccccgg cgtccccgag cccgccaccg |
| 1561 | acgccgacgc gctcctggag agcatccatc aggagttcac gcgcaccaac ttccacctct |
| 1621 | tcatccagac ttaggcgcgc ccggtagccc cggctgccca ccctggaggg ctgcgcccgc |
| 1681 | gccaggcgcg gggacgtgtt tctgggttct aggccctgct tccttgcccc tttgctgcag |
| 1741 | aagggcagct gaaggctcac cctagaaacc gggcctggtg ggtcttaccc ggctcactcc |
| 1801 | ctcccttgtc cttacacata caggaagaca agacctgagt ggtgctgtct ttgtgtccgt |
| 1861 | cgtgtatggc tctccctgtc ttcatttctt ctcactctgt ctctaaacct ctctctctct |
| 1921 | cccttccccc tcagtactta gtctacagac ctatgtgcgt gtccctatcc ttctgtcctt |
| 1981 | ttctctcttc agctctccct gcctctcaca cacaatttta catgccccga ggagccaagt |
| 2041 | ttgggacatt taccctccag gcatctgtgt cccctcttga agagaaaaca cacagcttca |
| 2101 | cacatccagg catagggggc aagctcttgg ggcatcagga ccctggagca ccaggtcctt |
| 2161 | cctggaatat tagatccacc tggagcaccg ggtctctcta agtctcacct ggggaattcg |
| 2221 | gtcccacctg gggcaccagt tcccacctag agcactgtgt cctgccctag agcacaaaga |
| 2281 | cctgctcctc ccgagactct ctctgactgc agccaggcat agtacctttg cctgtgtttg |
| 2341 | ctccctggtc cacagatttg gtggctgggc aggtgcctgg acagtgatga ggtcttgccg |
| 2401 | ccttaactgt cccccccagt cacttctccc acaggcccag caggacgcag tcctgaggat |
| 2461 | cagggattct acagctgcat taaaatcaat cctatccaa |

By “agent” is meant a small compound, polynucleotide, or polypeptide.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression or activity levels, a 25% change, a 40% change, a 50% change, or an even greater change in expression or activity levels (i.e., 75%, 80%, 85%, 90%).

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

The term “co-administration” or “combined administration” as used herein is defined to encompass the administration of the selected therapeutic agents to a single patient, and are intended to include treatment regimens in which the agents are not necessarily administered by the same route of administration or at the same time.

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “disease” is meant any condition or disorder that damages, or interferes with the normal function of a cell, tissue, or organ. Examples of diseases include cancer, including but not limited to small B-cell lymphomas, such as mantle cell lymphoma, or chronic lymphocytic leukemia (e.g., small lymphocytic lymphoma), diffuse large B cell lymphoma, splenic marginal zone lymphoma, follicular lymphoma, splenic red pulp lymphoma, MALT lymphoma and leukemias such as chronic lymphocytic leukemia, B cell acute lymphoblastic leukemia, T-cell acute lymphoblastic leukemia, and early T cell acute lymphoblastic leukemia).

By “effective amount” is meant the amount of an agent required to ameliorate the symptoms of a disease relative to an untreated patient. In one embodiment, an effective amount of an agent of the invention reduces or stabilizes the growth or proliferation of a neoplastic cell. In other embodiments, an effective amount of an agent of the invention reduces the survival of a neoplastic cell. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “jointly therapeutically active” or “joint therapeutic effect” as used herein means that the therapeutic agents may be given separately (in a chronologically staggered manner, especially a sequence-specific manner) in such time intervals as are preferable, in the subject, especially human subject, to be treated, and show an additive or greater effect. In a preferred embodiment, the joint therapeutic effect is an effect greater than the combined effect that each of the compounds would be expected to provide when administered on its own.

By “marker” is meant any protein or polynucleotide having an alteration in expression level or activity that is associated with a disease or disorder.

By “neoplasia” is meant abnormal cell proliferation. A neoplasm is a collection of cells characterized by increased cell division, poor cellular differentiation, and that is potentially cancerous.

As used herein, “obtaining” as in “obtaining an agent” includes synthesizing, purchasing, or otherwise acquiring the agent.
By “reduces” is meant a negative alteration of at least 10%, 25%, 50%, 75%, or 100%.

By “reference” is meant a standard or controlled condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, a siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those of ordinary skill in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to a person of ordinary skill in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to a person of ordinary skill in the art. Hybridization techniques are well known to a person of ordinary skill in the art and are described, for example, in Benton and Davis (Science 196:180, 1977); Grunstein and Hogness (Proc. Natl. Acad. Sci., USA 72:3961, 1975); Ausubel et al. (Current Protocols in

Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

The term “synergistic effect” as used herein refers to action of two therapeutic agents such as, for example, an agent that inhibits Notch signaling and an agent that inhibits B cell receptor signaling producing an effect, for example, slowing the symptomatic progression of a proliferative disease, particularly cancer, or symptoms thereof, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1A depicts in schematic form a transcript identified using RNASeq analysis, where the transcript includes the first exon of HLA-DMB and exons 24-30 of NOTCH4.

FIG. 1B provides a Western blot showing free (i.e., gamma secretase-cleaved) ICN-1 expression in MCL cell lines grown in the presence or absence of immobilized recombinant Notch ligand (DLL^{ext}-IgG) or control protein (IgG) at various times following exposure.

FIG. 2 provides graphs showing the effect of a gamma secretase inhibitor (GSI) on four clones (numbered 3, 4, 5, and 7) engineered to express GFP and tet activator from a constitutive transgene promoter, and MYC from a doxycycline-inducible promoter. The construct is called pINDUCER-22-MYC. in the presence of doxycycline.

FIG. 3A provides a schematic diagram of wild-type and mutants Notch proteins expressed in specific MCL cell lines (indicated in bold type).

FIG. 3B provides a western blot for cleaved ICN-1 in Mino cells plated on DLL l^{ext}-IgG-coated plates for the indicated time period.

FIG. 3C provides a schematic diagram of GSI-washout experiments in MCL lines with ligand-independent (top) and ligand-dependent (bottom) Notch signaling.

FIG. 3D provides a Western blot showing modulation of ICN-1 levels by GSI-washout in Mino and Rec-1 cells.

FIG. 4 provides a graph showing that myc enhancers are bound in enhancer 1 and enhancer RBPJ.

FIG. 5A shows the targeted epigenetic repression of 5′ enhancers inhibits MYC expression in Notch-dependent and EBV and MCL lines.

FIG. 5B shows flow cytometry quantification of the ratio of mCherry+versus GFP+cells relative to cells infected with a control gRNA

FIG. 5C shows a graph indicating decreased proliferation of the dCas9-KRAB-E2F-mCherry population for Granta-519, but little effect was seen for SP-49.

FIGS. 6A-6F show that GSI-sensitive MCL is driven by a Notch-dependent MYC program shared with other Notch-dependent cancers. FIG. 6A shows heatmaps indicating significantly up-regulated genes identified in GSI-washout versus mock-washout experiments in at least 2 of 3 MCL lines (Mino, Sp-49 and Rec-1). Heatmap clusters were defined and numbered as shown in the Venn diagram at the lower right of the figure, and are sorted within clusters by mean change in expression in GSI-washout experiments conducted in T-cell acute lymphoblastic leukemia (T-ALL) cell line CUTLL1 and TNBC cell line HCC-1599.

Canonical Notch target genes are labeled in grey text (NRARP, HES1, HEY1, NOTCH3, HES4, HEY2, and DTX1).

FIG. 6B shows gene sets from the MSigDB Hallmark (‘H’) and Reactome (‘R’) databases enriched in genes activated by GSI-washout in both GSI-sensitive and GSI-insensitive MCL cell lines (FIG. 6A, groups 1-3). FDR q-values are for combined analysis of both gene set collections.

FIG. 6C shows gene sets from the MSigDB Hallmark (‘H’) and Reactome (‘R’) databases enriched in genes activated by GSI-washout in GSI-sensitive MCL cell lines only (FIG. 6A, group 4). FDR q-values are for combined analysis of both gene set collections.

FIG. 6D provides a western blot for Notch and MYC proteins in MCL cell lines treated for three days with GSI or DMSO. It should be noted that the NOTCH4 band in GSI-treated SP-49 has a slightly increased molecular weight.

FIG. 6E provides a Western blot showing rescue of MYC expression in single-cell-derived clones of SP-49 transduced with pINDUCER-22-MYC, or parental SP-49, treated with GSI or GSI +100 ng/ml doxycycline.

FIG. 6F provides a graph showing growth of parental SP-49 and pINDUCER-22-MYC clones treated with GSI or GSI +doxycycline. Doxycycline doses were as follows: Clones 3 & 7−33.6 ng/ml, Clone 4 and parental−100 ng/ml.

FIGS. 7A-7E show data illustrating that Notch-rearranged and EBV+, but not MYC-rearranged MCL/CLL lines show acetylation and RBPJ binding at B cell-specific 5′ MYC enhancers.

FIG. 7A shows H3K27ac ChIP-Seq data showing mutually exclusive acetylation of 5′ MYC enhancers in Notch-dependent MCL and 3′ MYC enhancer in Notch-dependent T-ALL cell lines. Arrows indicate previously described looping interactions with the MYC promoter in MCL (Ryan et al., 2015) and T-ALL (Herranz et al., 2014; Yashiro-Ohtani et al., 2014).

FIG. 7B shows H3K27ac ChIP-Seq data for 5′ MYC enhancers and CD79A promoter regions in CLL (Me) and MCL (Jv, Gr, Re, Sp, Mi, Je, Z1, Ma, Hb, and Up) cell lines. The cell line abbreviations used are: Me=Mec-1, Jv=JVM2, Gr=Granta-519, Re=Rec-1, Sp=SP-49, Mi=Mino, Je=Jeko-1, Z1=Z138, Ma=MAVER1, Hb=HBL-2, and Up=UPN-1.

FIG. 7C provides a Western blot showing expression of EBNA2 and c-MYC in nuclear extracts from CLL and MCL lines.

FIG. 7D provides a graph showing ChIP-PCR showing binding of RBPJ at 5′ MYC enhancer E-2 in CLL and MCL cell lines.

FIG. 7E provides a graph showing ChIP-PCR showing binding of EBNA2 at 5′ MYC enhancer E-2 in CLL and MCL cell lines.

FIGS. 8A-8E provide data showing that ChIP-Seq and CRISPR-Cas9 validation of Notch-dependent 5′ MYC enhancers confirms the role of Notch in MYC expression and MCL proliferation.

FIG. 8A provides ChIP-Seq data showing the dynamics of ICN-1 and RBPJ binding, and H3K27ac modification at the 5′ B cell Notch-dependent MYC enhancers (BNDME) sites. Mino cells in the top two rows were plated on DLL 1^{e}-IgG for 48 hours. The bottom six rows depict ChIP-Seq data for the indicated marker after GSI-washout experiments conducted as in FIG. 1C. Washout=‘on’, grey track; Mock washout=‘off’, black overlay track.

FIG. 8B shows ICN-1 and RBPJ binding at BNDME sites after GSI-washout, as well as Phastcons 46-vertebrate conservation score (‘conservation’). Consensus RBPJ logos are aligned to the position of conserved RBPJ motifs in each enhancer. The positions of specific gRNAs are indicated.

FIG. 8C provides a graph showing qRT-PCR measurement of MYC expression after transduction of dCAS9-KRAB:E2A:mCherry-expressing EBV+(Granta-519), Notch-rearranged (SP-49), and MYC-rearranged/amplified (Jeko-1) MCL cell lines with guideRNAs targeting the BNDME sites, or non-targeting controls (GFP).

FIG. 8D provides a series of graphs showing qRT-PCR measurement of MYC expression after transduction of Cas9 nuclease-expressing MCL lines with gRNAs against BNDME sites, or non-targeting controls.

FIG. 8E provides a series of graphs showing growth of indicated Cas9 nuclease-expressing MCL cell lines after transduction with gRNAs as in (FIG. 8D).

FIGS. 9A-9E shows genes activated by Notch independently of MYC are highly enriched for direct Notch regulatory targets, and include B cell signaling pathway regulators.

FIG. 9A provides a graph showing fraction of Notch-activated genes identified in MCL models that show ICN-1 binding in Rec-1 to the gene promoter, or to a distal site linked to the gene promoter by 3D looping in EBV+B cells (GM12878 Pol2 ChIA-PET). Gene groups are defined as in FIG. 6A, with genes in groups 1-3 showing activation in a cell line (Mino) that lacks Notch-dependent MYC activation (“MYC-independent”). “Rnd” is a randomly selected group of expressed genes that do not show Notch-dependent differential expression.

FIG. 9B shows representative known and novel direct Notch target genes with promoter-proximal ICN-1 binding in Rec-1. H3K27 acetylation shown for Rec-1 and for NOTCH/-mutant MCL and CLL lymph node biopsies.

FIG. 9C-1-9C-6 shows representative direct Notch target genes with ICN-1 binding to promoter-distal sites. GM12878 Pol2 ChIA-PET data shows loop interactions between ICN1-bound distal sites and Notch-activated gene promoters.

FIG. 9D shows CRISPR-Cas9-mediated validation of representative ICN1+regulatory sites for CR2 and IL6R.

FIGS. 10A-10F show Notch-dependent activation of target genes and pathways in primary CLL cells.

FIG. 10A shows immunohistochemistry for ICN-1 in representative cases of ICN1-high and ICN-1-low CLL.

FIG. 10B shows a heatmap indicating relative expression of genes (RNA-Seq) significantly upregulated by gamma-secretase inhibitor-washout in MCL, and in ICN1-high versus ICN1-low MCL.

FIG. 10C shows ChIP-Seq data from MCL cell lines and primary CLL and MCL samples, demonstrating ICN-1 and RBPJ binding at enhancers of genes validated as direct Notch targets in MCL cell lines and primary CLL samples.

FIG. 10D shows a schematic diagram of primary CLL/HS-5 co-culture experiments.

FIG. 10E provides a graph showing the relative expression of MYC (qRT-PCR) in CD19+CD5+CLL cells sorted following three-day HS-5-DLL-1 co culture in the presence of GSI or vehicle.

FIG. 10F provides a series of a graphs showing the phosphorylation-specific flow analysis of specified epitopes in primary CLL cells (CLL-015) co-cultured for three days with HS-5-DLL1 cells in the presence of GSI or vehicle. Indicated samples were treated for the stated time with F(ab) anti-IgG/IgM to crosslink B-cell receptors. Dotted line marks the mode of fluorescence intensity in the un-stimulated/GSI-treated sample for each epitope.

FIG. 11 shows a schematic wherein Notch drives potentiation of B-cell receptor and cytokine signaling via MYC-independent targets, as well as a MYC-dependent metabolic shift. The diagram depicts direct Notch target gene products as well as their relationship to B cell-receptor signaling and other pathways. Solid lines indicate direct regulatory relationships, while dotted lines indicate presence of one or more intermediaries. Phosphorylation of active B-cell receptor (BCR) signaling mediators is potentiated by Notch-dependent increases in expression of SRC-family kinases and signaling adaptor proteins, while another direct Notch target gene product, c-MYC, controls expression of critical metabolic regulators. Both the BCR and MYC pathways drive signaling events that regulate mTORC1 activity. NF-KB activation downstream of BCR signaling may activate additional genes in the setting of Notch activation, or may confer synergistic activation of direct Notch target genes.

FIG. 12A shows a schematic of CLL HS-5 co-culture experiments performed in the presence of CpG-rich oligodideoxynucleotides.

FIG. 12B shows quantification of CLL HS-5 co-culture experiments.

FIG. 12C shows quantification of Notch target cell surface proteins in MCL cells within the spleen, bone marrow and blood.

### DETAILED DESCRIPTION OF THE INVENTION

The invention generally provides therapeutic compositions comprising a combination of an agent that inhibits the activity of or decreases the levels of a Notch protein and an agent that inhibits B-cell receptor (BCR) signalling, and methods of using such combinations to treat cancer (e.g., small B-cell lymphomas, such as mantle cell lymphoma, or chronic lymphocytic leukemia (e.g., small lymphocytic lymphoma), diffuse large B cell lymphoma, splenic marginal zone lymphoma, follicular lymphoma, splenic red pulp lymphoma, MALT lymphoma and leukemias, such as chronic lymphocytic leukemia, B cell acute lymphoblastic leukemia, T-cell acute lymphoblastic leukemia, and early T cell acute lymphoblastic leukemia).

Recurrent gain-of-function mutations in genes encoding Notch receptors are associated with poor clinical outcome in two small B-cell lymphoma subtypes, mantle cell lymphoma (MCL) and chronic lymphocytic leukemia (CLL; also known as small lymphocytic lymphoma, SLL), but functional targets of Notch signaling in B cells have not been systematically characterized. As described herein, a gamma-secretase washout strategy was used to rapidly activate Notch signaling in Notch-dependent and -independent MCL lines, and to identify direct Notch regulatory targets through genome-wide expression profiling and chromatin immunoprecipitation (ChIP-Seq) of Notch transcriptional complex (NTC) components.

The invention is based, at least in part, on the discovery that proliferation of Notch-dependent mantle cell lymphoma (MCL) lines was driven by activation of the oncogene MYC via Notch transcriptional complex binding at B-cell-specific 5′ enhancer elements, resulting in secondary activation of MYC target genes and a metabolic program associated with mTORC1 activation. These studies identified novel Notch regulatory targets in B-cell lymphomas associated with NTC binding to proximal and distal regulatory elements, that activate genes encoding cytokine receptors (IL6R, IL 10R, IL21R), as well as SRC-family kinases (FYN, LYN, BLK) and signaling adaptor proteins (BLNK, NEDD9, SH2B2, PIK3AP 1) involved in activation of pathways downstream of B-cell receptor (BCR) signaling. Genome-wide profiling analysis of lymphoma biopsies, plus functional studies of patient-derived lymphoma cells in vitro and in vivo were utilized to validate Notch-dependent regulation of MYC and oncogenic BCR signaling in primary human CLL and MCL.

Genome-wide profiling of mRNA, histone acetylation, and NTC binding in MCL was used to identify differential regulation of enhancers and genes that represent the direct targets of Notch signaling in B cell lymphoma. The findings indicated that Notch signaling drives two distinct oncogenic programs in lymphoma cell lines and primary tumors. First, ICN binds and activates B-cell-specific 5′ MYC enhancers, resulting in activation of a MYC-dependent metabolic program that is shared with other Notch-dependent tumor types. Second, Notch directly activates the expression of cytokine receptors and B cell receptor signaling intermediates, thus potentiating the response of lymphoma cells to activating stimuli. Notably, the data indicated a Notch-dependent increase in B cell-receptor-dependent phosphorylation of PLC2G and downstream activation of NF-KB, a pathway that is known to be central to the proliferation and survival of small B cell lymphomas.

Building on these findings, the invention provides novel therapeutic compositions and methods combining direct B cell receptor inhibition (expected to block B cell receptor signaling and to drive cancerous B cells towards apoptosis and/or disrupts tumor formation) with Notch inhibition (expected to both cease the activation of MYC and to also cease B cell receptor potentiation). In taking both approaches towards B cell inhibition in concert, cancerous B cells are specifically targeted and have increased difficulty escaping the treatment by mutation.

Accordingly, the invention provides therapeutic compositions comprising an agent (e.g., polypeptides, inhibitory nucleic acids, and small molecules) that inhibits a Notch polypeptide (e.g., Notch1, Notch2, Notch3, Notch4) expression or activity and an agent that inhibits B Cell Receptor (BCR) signaling, and methods of using such compositions to inhibit the growth or proliferation of a neoplastic cell. Compositions of the invention are useful for the treatment of cancer (e.g., e.g., small B-cell lymphomas, such as mantle cell lymphoma, or chronic lymphocytic leukemia (e.g., small lymphocytic lymphoma), diffuse large B cell lymphoma, splenic marginal zone lymphoma, follicular lymphoma, splenic red pulp lymphoma, MALT lymphoma and leukemias such as chronic lymphocytic leukemia, B cell acute lymphoblastic leukemia, T-cell acute lymphoblastic leukemia, and early T cell acute lymphoblastic leukemia).

### Notch

Notch proteins are expressed as trans-membrane receptors that undergo sequential proteolytic cleavage upon interaction with Notch ligands expressed on neighboring cells, resulting in gamma secretase-dependent release of the intracellular notch (ICN) fragment. ICN then traffics to the nucleus, where it binds to transcriptional regulatory elements in a Notch transcriptional complex (NTC) with the DNA sequence-specific transcription factor RBPJ, mastermind-like (MAML) proteins, and other co-factors. Nearly all Notch gene mutations reported in CLL and MCL result in frameshift-mediated truncation of the C-terminal PEST domain, which mediates ubiquitination and degradation of ICN. Notch PEST domain truncations have been extensively studied in T-cell acute lymphoblastic leukemia (T-ALL), where they enhance the nuclear accumulation of ICN, but do not confer active signaling in the absence of ligand. This contrasts with Notch gene heterodimerization domain mutations and rearrangements, which do confer ligand-independent signaling, and are common in T-ALL, but are extremely rare in CLL and MCL patients. Immunohistochemistry (IHC) with an antibody that specifically recognizes the gamma-secretase-cleaved NOTCH1 ICN (ICN-1) was previously used to demonstrate NOTCH1 activation in >80% of CLL lymph node biopsies. Strong and diffuse ICN-1 staining was significantly, but not exclusively, associated with cases bearing NOTCH1 PEST mutations. These findings suggested that activation of Notch signaling in lymphoma cells via interaction with ligand-presenting cells in the lymph node microenvironment may be a broadly important feature of this disease.

In vitro models for the study of Notch signaling in B-cell lymphoma have been limited. Two MCL cell lines, Rec-1 and SP-49, were reported to show marked growth inhibition upon treatment with gamma-secretase inhibitors (GSI) or expression of a Notch-inhibiting transgene, suggesting dependency of these lines on ligand-independent Notch signaling (Kridel et al., 2012). Subsequently, ICN-1 activation in Rec-1 was found to be due to a genomic deletion encompassing most of the exons encoding the NOTCH1 extracellular domain, and that this allele confers ligand-independent Notch signaling that is sensitive to GSI inhibition.

### Therapeutic Compositions Comprising Notch and B Cell Receptor Inhibitors

The present invention features compositions comprising one or more agents that inhibit Notch signaling and one or more agents that inhibit B cell receptor signaling. Such agents include small molecules, polypeptides, and polynucleotides described herein.

Small molecules capable of inhibiting Notch include gamma-secretase inhibitors (GSI). Exemplary gamma-secretase inhibitors are known in the art, and include, for example, Compound E, MK-0752, PF03084014, RO-4929097, DAPT, N-[N-(3,5-difluorophenacetyl)- L-alanyl]-S-phenylglycine t-butyl ester, tetralin imidazole PF-03084014, LY3039478 and BMS906-024.

Further examples of compounds suitable as Notch inhibitors can include the compounds listed in U.S. Pat. Nos. 8,377,886, 6,756,511, 6,890,956, 6,984,626, 7,049,296, 7,101,895, 7,138,400, 7,144,910, and 7,183,303, incorporated by reference herein in their entirety.

Other Notch inhibitors include antibodies that specifically bind Notch and inhibit or disrupt its activity, or deplete its levels. Exemplary inhibitory Notch antibodies are known in the art, and include, for example, anti-Notch 1 (OMP-52M521) and anti-delta-like-4.

Further examples of antibodies suitable for inhibiting Notch and Notch signaling pathway include the antibodies listed in U.S. Pat. Nos. 9,090,690, 8,945,547, 8,945,873, 7,534,868 and International Patent Application Nos. WO 2008150525, WO 2010059543, WO 2011041336, incorporated by reference herein in their entirety.

Examples of compounds suitable as B cell receptor (BCR) inhibitors can include Bruton tyrosine kinase (BTK) inhibitors, SRC family kinase inhibitors, SYK inhibitors, or protein kinase C inhibitors, and PI3 Kinase inhibitors.

Exemplary B cell receptor inhibitors include, for example, ibrutinib (PC1-32765), acalabrutinib (ACP-ONO-4059 (e.g., GS-4059 or NCT02457598), spebrutinib (e.g., AVL-292, CC-292), and BGB-3111.

Further examples of compounds suitable as BCR inhibitors can include the compounds listed in U.S. Pat. Nos. 8,227,433, 6,306,897, 8,999,999 and International Patent Application Nos. WO2015110923, WO1999054286 (incorporated by reference in their entirety).

Small molecules capable of inhibiting signaling mediated by B cell receptors or Notch can include SRC family kinase inhibitors. Exemplary SRC family kinase inhibitors are known in the art, and include, for example, dasatinib (BMS-354825), KX2-391, bosutinib (SKI-606), and saracatinib (AZD-0530).

Small molecules capable of inhibiting signaling mediated by B cell receptors or Notch can include spleen tyrosine kinase (SYK) inhibitors. Exemplary SYK inhibitors are known in the art, and include, for example, fostamatinib (R788), piceatannol, entospletinib (GS-9973), and GSK2646264.

Small molecules capable of inhibiting signaling mediated by B cell receptors or Notch can include protein kinase C (PKC) inhibitors. Exemplary PKC inhibitors are known in the art, and include, for example, midostaurin (PKC412), enzastaurin (LY317615), sotrastaurin (AEB071), and ruboxistaurin (LY333531).

Small molecules capable of inhibiting signaling mediated by B cell receptors or Notch can include phosphoinositol-3-kinase (PI3K) inhibitors. Exemplary PI3K inhibitors are known in the art, and include, for example, idelalisib (e.g., zydelig, GS-1101, CAL-101), alpelisib (13Y⁻1-719), AEZS-136, buparlisib (BKM120), copanlisib (BAY 80-6946), CA1,263, CU⁻DC-907, dactolisib (e.g., NNT-BEZ235, BEZ-235), duvelisib (1PI-145), GNE-477, GSM 059615, 1087114, 1P1-549, INK1117, palomid 529, perifosine (KRX-0401), pictilisib (GDC-0941), ME-401, PI-103, PWT33597, PX-866, RP6503, RP6530, SF⁻1126, TGR 1202, wortniannin, demethoxyviridin, X1,147 (SAR245408), XL765 (SAR245409), ZSIK474.

Further examples of compounds suitable as PI3K inhibitors can include the compounds listed in U.S. Pat. Nos. 9,403,779, 9,150,579, 9,126,948, 8,940,752, 8,759,359, 8,440,651, U.S. Patent Application Nos. 20140364447, 20100056523, 20100029693, and International Patent Application Nos. WO 2016051374, WO 2015181728, WO 2015160986, WO 2014195888, WO 2011123751 (incorporated by reference herein in their entirety).

In accordance with the present invention, a therapeutically effective amount of each of the combination partners (e.g., an agent that inhibits Notch signaling and an agent that inhibits B cell receptor signaling) may be administered simultaneously or sequentially and in any order, and the components may be administered separately or as a fixed combination. For example, the method of treating a neoplasia according to the invention may comprise (i) administration of the first agent (a) in free or pharmaceutically acceptable salt form and (ii) administration of an agent (b) in free or pharmaceutically acceptable salt form, simultaneously or sequentially in any order, in jointly therapeutically effective amounts, preferably in synergistically effective amounts, e.g. in daily or intermittently dosages corresponding to the amounts described herein. The individual combination partners may be administered separately at different times during the course of therapy or concurrently in divided or single combination forms. Furthermore, the term “administering” also encompasses the use of a pro-drug of a combination partner that converts in vivo to the combination partner as such. The invention is therefore to be understood as embracing all such regimens of simultaneous or alternating treatment and the term “administering” is to be interpreted accordingly.

The effective dosage of each of the combination partners employed in the methods of the invention may vary depending on the particular compound or pharmaceutical composition employed, the mode of administration, the condition being treated, and the severity of the condition being treated. Thus, the dosage regimen is selected in accordance with a variety of factors including the route of administration and the renal and hepatic function of the patient. A clinician or physician of ordinary skill in the art can readily determine and prescribe the effective amount of the single therapeutic agents required to alleviate, counter or arrest the progress of the condition.

The optimum ratios, individual and combined dosages, and concentrations of the combination partners that yield efficacy without toxicity are based on the kinetics of the therapeutic agents' availability to target sites, and are determined using methods known to those of skill in the art.

The effective dosage of each of the combination partners may require more frequent administration of one of the agents in the combination. Therefore, to permit appropriate dosing, packaged pharmaceutical products may contain one or more dosage forms that contain the combination of compounds, and one or more dosage forms that contain one of the combination of compounds, but not the other compound(s) of the combination.

When the combination partners are employed or as marketed as single drugs, their dosage and mode of administration can be in accordance with the information provided on the package insert of the respective marketed drug, if not mentioned herein otherwise.

The optimal dosage of each combination partner for treatment of a proliferative disease can be determined empirically for each individual using known methods and will depend upon a variety of factors, including, though not limited to, the degree of advancement of the disease; the age, body weight, general health, gender and diet of the individual; the time and route of administration; and other medications the individual is taking optimal dosages may be established using routine testing and procedures that are well known in the art.

The amount of each combination partner that may be combined with the carrier materials to produce a single dosage form will vary depending upon the individual treated and the particular mode of administration. In some embodiments the unit dosage forms containing the combination of agents as described herein will contain the amounts of each agent of the combination that are typically administered when the agents are administered alone.

Frequency of dosage may vary depending on the compound used and the particular condition to be treated or prevented. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

The present invention relates to a method of treating a subject having a proliferative disease comprising administering to said subject a combination of an agent that inhibits Notch signaling and an agent that inhibits B cell receptor signaling in a quantity which is jointly therapeutically effective against a neoplastic disease. In particular, the neoplastic disease to be treated is a leukemia or lymphoma.

The present invention further provides a commercial package comprising as therapeutic agents an agent that inhibits Notch signaling and an agent that inhibits B cell receptor signaling, optionally together with instructions for simultaneous, separate or sequential administration thereof for use in the delay of progression or treatment of a proliferative disease in a subject in need thereof.

### Inhibitory Nucleic Acids

The invention further provides inhibitory nucleic acids (e.g., antisense molecules, siRNA, shRNA) that inhibit the expression of a Notch polypeptide (e.g., Notch 1, Notch 2, Notch 3, Notch4). In addition, the invention provides inhibitory nucleic acids (e.g., antisense molecules, siRNA, shRNA) that inhibit the expression of a functional component of the B cell receptor. Such oligonucleotides include single and double stranded nucleic acid molecules (e.g., DNA, RNA, and analogs thereof) that bind a nucleic acid molecule that encodes a Notch polypeptide, as well as nucleic acid molecules that bind directly to the polypeptide to modulate its biological activity (e.g., aptamers).

siRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of a siRNA approach in mammals was demonstrated in vivo by McCaffrey et al. (Nature 418: 38-39.2002).

Given the sequence of a target gene, siRNAs may be designed to inactivate that gene. Such siRNAs, for example, could be administered directly to an affected tissue, or administered systemically. The nucleic acid sequence of a gene can be used to design small interfering RNAs (siRNAs). The 21 to 25 nucleotide siRNAs may be used, for example, as therapeutics to treat cancer (e.g., small B-cell lymphomas, such as mantle cell lymphoma, or chronic lymphocytic leukemia (e.g., small lymphocytic lymphoma), diffuse large B cell lymphoma, splenic marginal zone lymphoma, follicular lymphoma, splenic red pulp lymphoma, MALT lymphoma and leukemias such as chronic lymphocytic leukemia, B cell acute lymphoblastic leukemia, T-cell acute lymphoblastic leukemia, and early T cell acute lymphoblastic leukemia).

The inhibitory nucleic acid molecules of the present invention may be employed as double-stranded RNAs for RNA interference (RNAi)-mediated knock-down of expression of a Notch polypeptide. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the invention, a double-stranded RNA (dsRNA) molecule is made that includes between eight and nineteen consecutive nucleobases of a nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

Small hairpin RNAs (shRNAs) comprise an RNA sequence having a stem-loop structure. A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). The term “hairpin” is also used herein to refer to stem-loop structures. Such structures are well known in the art and the term is used consistently with its known meaning in the art. As is known in the art, the secondary structure does not require exact base-pairing. Thus, the stem can include one or more base mismatches or bulges. Alternatively, the base-pairing can be exact, i.e. not include any mismatches. The multiple stem-loop structures can be linked to one another through a linker, such as, for example, a nucleic acid linker, a miRNA flanking sequence, other molecule, or some combination thereof.

As used herein, the term “small hairpin RNA” includes a conventional stem-loop shRNA, which forms a precursor miRNA (pre-miRNA). While there may be some variation in range, a conventional stem-loop shRNA can comprise a stem ranging from 19 to 29 bp, and a loop ranging from 4 to 30 bp. “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. In some instances the precursor miRNA molecule can include more than one stem-loop structure. MicroRNAs are endogenously encoded RNA molecules that are about 22-nucleotides long and generally expressed in a highly tissue- or developmental-stage-specific fashion and that post-transcriptionally regulate target genes. More than 200 distinct miRNAs have been identified in plants and animals. These small regulatory RNAs are believed to serve important biological functions by two prevailing modes of action: (1) by repressing the translation of target mRNAs, and (2) through RNA interference (RNAi), that is, cleavage and degradation of mRNAs. In the latter case, miRNAs function analogously to small interfering RNAs (siRNAs). Thus, one can design and express artificial miRNAs based on the features of existing miRNA genes.

shRNAs can be expressed from DNA vectors to provide sustained silencing and high yield delivery into almost any cell type. In some embodiments, the vector is a viral vector. Exemplary viral vectors include retroviral, including lentiviral, adenoviral, baculoviral and avian viral vectors, and including such vectors allowing for stable, single-copy genomic integrations. Retroviruses from which the retroviral plasmid vectors can be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus. A retroviral plasmid vector can be employed to transduce packaging cell lines to form producer cell lines. Examples of packaging cells which can be transfected include, but are not limited to, the PE501, PA317, R-2, R-AM, PA12, T19-14x, VT-19-17-H2, RCRE, RCRIP, GP+E-86, GP+envAm12, and DAN cell lines as described in Miller, Human Gene Therapy 1:5-14 (1990), which is incorporated herein by reference in its entirety. The vector can transduce the packaging cells through any means known in the art. A producer cell line generates infectious retroviral vector particles which include polynucleotide encoding a DNA replication protein. Such retroviral vector particles then can be employed, to transduce eukaryotic cells, either in vitro or in vivo. The transduced eukaryotic cells will express a DNA replication protein.

Examples of delivery methods suitable to deliver siRNA and shRNA molecules of the present invention are disclosed in Nature Materials Vol 12, 2013, pages 967-977, incorporated by reference in its entirety.

Catalytic RNA molecules or ribozymes that include an antisense sequence of the present invention can be used to inhibit expression of a nucleic acid molecule in vivo (e.g., a nucleic acid encoding any component of the Notch signaling pathway (e.g., Notch 1, Notch 2, Notch 3, Notch, 4, canonical Notch signaling modalities) and B Cell receptor (BCR) signaling (e.g. phospholipase C gamma 2, LYN, FYN, PI3K, NF-KB transcription factor pathway). The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference.

Accordingly, the invention also features a catalytic RNA molecule that includes, in the binding arm, an antisense RNA having between eight and nineteen consecutive nucleobases. In preferred embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

Essentially any method for introducing a nucleic acid construct into cells can be employed. Physical methods of introducing nucleic acids include injection of a solution containing the construct, bombardment by particles covered by the construct, soaking a cell, tissue sample or organism in a solution of the nucleic acid, or electroporation of cell membranes in the presence of the construct. A viral construct packaged into a viral particle can be used to accomplish both efficient introduction of an expression construct into the cell and transcription of the encoded shRNA. Other methods known in the art for introducing nucleic acids to cells can be used, such as lipid-mediated carrier transport, chemical mediated transport, such as calcium phosphate, and the like. Thus the shRNA-encoding nucleic acid construct can be introduced along with components that perform one or more of the following activities: enhance RNA uptake by the cell, promote annealing of the duplex strands, stabilize the annealed strands, or otherwise increase inhibition of the target gene.

For expression within cells, DNA vectors, for example plasmid vectors comprising either an RNA polymerase II or RNA polymerase III promoter can be employed. Expression of endogenous miRNAs is controlled by RNA polymerase II (Pol II) promoters and in some cases, shRNAs are most efficiently driven by Pol II promoters, as compared to RNA polymerase III promoters (Dickins et al., 2005, Nat. Genet. 39: 914-921). In some embodiments, expression of the shRNA can be controlled by an inducible promoter or a conditional expression system, including, without limitation, RNA polymerase type II promoters. Examples of useful promoters in the context of the invention are tetracycline-inducible promoters (including TRE-tight), IPTG-inducible promoters, tetracycline transactivator systems, and reverse tetracycline transactivator (rtTA) systems. Constitutive promoters can also be used, as can cell- or tissue-specific promoters. Many promoters will be ubiquitous, such that they are expressed in all cell and tissue types. A certain embodiment uses tetracycline-responsive promoters, one of the most effective conditional gene expression systems in in vitro and in vivo studies. See International Patent Application PCT/US2003/030901 (Publication No. WO 2004-029219 A2) and Fewell et al., 2006, Drug Discovery Today 11: 975-982, for a description of inducible shRNA.

### Delivery of Polynucleotides

Naked polynucleotides, or analogs thereof, are capable of entering mammalian cells and inhibiting expression of a gene of interest. Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference). Inhibitory nucleic acid molecule can be delivered using a nanoparticle. Nanoparticle compositions suitable for use with inhibitory nucleic acid molecules are known in the art and described for example by Kanasty et al., Nature materials 12: 967-977, 2013, which is incorporated herein by reference. Such nanoparticle delivery compositions include cyclodextrin polymer (CDP)-based nanoparticles, lipid nanoparticles, cationic or ionizable lipid, lipid-anchored PEG, PEGylated nanoparticles, oligonucleotide nanoparticles (ONPs), and siRNA-polymer conjugate delivery systems (e.g., Dynamic PolyConjugate, Triantennary GalNAc-siRNA).

### Chemotherapeutic Agents

The invention further provides for the use of a combination of the invention (e.g., an agent that inhibits Notch signaling and an agent that inhibits B cell receptor signaling) in combination with another therapeutic agent, such as a conventional chemotherapeutic agent, or agent that mitigates a side effect associated with an agent of the invention. Chemotherapeutic agents can be used with the methods of the present invention including, but are not limited to alkylating agents. Without intending to be limited to any particular theory, alkylating agents directly damage DNA to keep the cell from reproducing. Alkylating agents work in all phases of the cell cycle and are used to treat many different cancers (e.g., small B-cell lymphomas, such as mantle cell lymphoma, or chronic lymphocytic leukemia (e.g., small lymphocytic lymphoma), diffuse large B cell lymphoma, splenic marginal zone lymphoma, follicular lymphoma, splenic red pulp lymphoma, MALT lymphoma and leukemias such as chronic lymphocytic leukemia, B cell acute lymphoblastic leukemia, T-cell acute lymphoblastic leukemia, and early T cell acute lymphoblastic leukemia). Alkylating agents are divided into different classes, including, but not limited to: (i) nitrogen mustards, such as, for example mechlorethamine (nitrogen mustard), chlorambucil, cyclophosphamide (Cytoxan®), ifosfamide, and melphalan; (ii) nitrosoureas, such as, for example, streptozocin, carmustine (BCNU), and lomustine; (iii) alkyl sulfonates, such as, for example, busulfan; (iv) riazines, such as, for example, dacarbazine (DTIC) and temozolomide (Temodar®); (v) ethylenimines, such as, for example, thiotepa and altretamine (hexamethylmelamine); and (v) platinum drugs, such as, for example, cisplatin, carboplatin, and oxalaplatin.

Uses of Notch and B Cell Receptor Inhibitors

The invention features methods for inhibiting the proliferation, growth, or viability of a neoplastic cell by contacting the cell with a Notch inhibitor and an agent that inhibits B Cell Receptor signaling. In general, the method includes a step of contacting a neoplastic cell with an effective amount of a compound of the invention. The present method can be performed on cells in culture, e.g., in vitro or ex vivo, or can be performed on cells present in an animal subject, e.g., as part of an in vivo therapeutic protocol. The therapeutic regimen can be carried out on a human or other subject.

The compounds of the invention or otherwise described herein can be tested initially in vitro for their inhibitory effects on the proliferation or survival of neoplastic cells. Examples of cell lines that can be used are any of the MCL cell lines described herein or any other suitable cell line known in the art. Alternatively, the antineoplastic activity of compounds of the invention can be tested in vivo using various animal models known in the art. For example, xenographs of human neoplastic cells or cell lines are injected into immunodeficient mice (e.g., nude or SCID) mice. Compounds of the invention are then administered to the mice and the growth and/or metastasis of the tumor is compared in mice treated with a compound of the invention relative to untreated control mice. Agents that reduce the growth or metastasis of a tumor or increase mice survival are identified as useful in the methods of the invention.

The methods discussed herein can be used to inhibit the proliferation of virtually any neoplastic cell. The invention provides methods for treating a subject having a neoplasia by administering to the subject an effective amount of an agent that inhibits Notch signaling and an agent that inhibits B cell receptor signaling as described herein. In certain embodiments, the subject is a mammal, in particular a human.

Agents which are determined to be effective for the prevention or treatment of neoplasias in animals, e.g., dogs, rodents, may also be useful in treatment of neoplasias in humans. Those skilled in the art of treating neoplasias in humans will know, based upon the data obtained in animal studies, the dosage and route of administration of the compound to humans. In general, the dosage and route of administration in humans is expected to be similar to that in animals.

The identification of those patients who are in need of prophylactic treatment for hyperplastic/neoplastic disease states is well within the ability and knowledge of one skilled in the art. Certain of the methods for identification of patients who are at risk of developing neoplastic disease states which can be treated by the subject method are appreciated in the medical arts, such as family history of the development of a particular disease state and the presence of risk factors associated with the development of that disease state in the subject patient. A clinician skilled in the art can readily identify such candidate patients, by the use of, for example, clinical tests, physical examination and medical/family history.

### Pharmaceutical Compositions

The invention provides pharmaceutical compositions for the treatment of a neoplasia, comprising an effective amount of an agent that inhibits Notch activity or decreases Notch levels, an agent that inhibits B Cell Receptor signaling and a pharmaceutically acceptable carrier. In particular embodiments, compositions of the invention comprise an agent or combination of agents described herein in combination with a conventional chemotherapeutic agent. In still other embodiments, such compositions are labeled for the treatment of cancer. In a further embodiment, the effective amount is effective to reduce the growth, proliferation, or survival of a neoplastic cell or to otherwise treat or prevent a neoplasia in a subject, as described herein.

In an embodiment, the agent is administered to the subject using a pharmaceutically-acceptable formulation. In certain embodiments, these pharmaceutical compositions are suitable for oral or parenteral administration to a subject. In still other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound. In certain embodiments, the subject is a mammal, e.g., a primate, e.g., a human.

The methods of the invention further include administering to a subject a therapeutically effective amount of a compound in combination with a pharmaceutically acceptable excipient. The phrase “pharmaceutically acceptable” refers to those compounds of the invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable excipient” includes pharmaceutically-acceptable material, composition or vehicle, such as a liquid or solid filler, diluent, carrier, solvent or encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a compound(s) include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred per cent, this amount will range from about 1 per cent to about ninety-nine percent of active ingredient, preferably from about 5 per cent to about 70 per cent, most preferably from about 10 per cent to about 30 per cent.

Methods of preparing these compositions include the step of bringing into association a agent(s) with the carrier and, optionally, one or more accessory ingredients. In general, the formulations are prepared by uniformly and intimately bringing into association a compound with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound(s) as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compound(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants, such as wetting agents, emulsifying and suspending agents, sweetening, flavoring, coloring, perfuming and preservative agents.

Suspensions, in addition to the active compound(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more compound(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a compound(s) include powders, sprays, ointments, pastes, creams, lotions, gels, solutions, patches and inhalants. The active compound(s) may be mixed under sterile conditions with a pharmaceutically-acceptable carrier, and with any preservatives, buffers, or propellants which may be required.

The ointments, pastes, creams and gels may contain, in addition to compound(s) of the present invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound(s), excipients, such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The compound(s) can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids, such as glycine, buffers, salts, sugars or sugar alcohols. Aerosols generally are prepared from isotonic solutions.

Transdermal patches have the added advantage of providing controlled delivery of a compound(s) to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compound(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of compound(s) in biodegradable polymers, such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compound(s) are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, the compound(s), which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of this invention may be varied so as to obtain an amount of the active ingredient which is effective to achieve the desired therapeutic response for a particular patient, composition, and mode of administration, without being toxic to the patient. An exemplary dose range is from about 0.1 μg to 20 milligram per kilogram of body weight per day (mg/kg/day) (e.g., 0.1 μg/kg to 10 mg/kg, 0.1-10 μg/kg, 0.1-1 mg/kg). In other embodiments, the amount varies from about 0.1 mg/kg/day to about 100 mg/kg/day. In still other embodiments, the amount varies from about 0.001 μg to about 100 μg/kg (e.g., of body weight). Ranges intermediate to the above-recited values are also intended to be part of the invention.

### Kits

The invention provides kits for the treatment or prevention of cancer. In some embodiments, the kit includes a therapeutic or prophylactic composition containing an effective amount of an agent that inhibits the activity of or decreases the levels of a Notch protein and an effective amount of an agent that inhibits B cell receptor signaling. In one embodiment, the invention provides a commercial package comprising as therapeutic agents a combination comprising a first agent (e.g., an agent that inhibits Notch signaling) or a pharmaceutically acceptable salt thereof, and at least one second agent (e.g., an agent that inhibits B cell receptor signaling) or a pharmaceutically acceptable salt thereof, together with instructions for simultaneous, separate or sequential administration thereof for use in the delay of progression or treatment of a neoplasia.

In particular embodiments, each agent is provided in unit dosage form in a sterile container. Such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

The kit optionally includes instructions for administering the pharmaceutical composition to a subject having or at risk of contracting or developing cancer. The instructions will generally include information about the use of the composition for the treatment or prevention of cancer. In other embodiments, the instructions include at least one of the following: description of the therapeutic/prophylactic agent; dosage schedule and administration for treatment or prevention of cancer or symptoms thereof; precautions; warnings; indications; counter-indications; over dosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### A novel HLA-DMB/NOTCH4 Rearrangement in the MCL Cell Line SP-49.

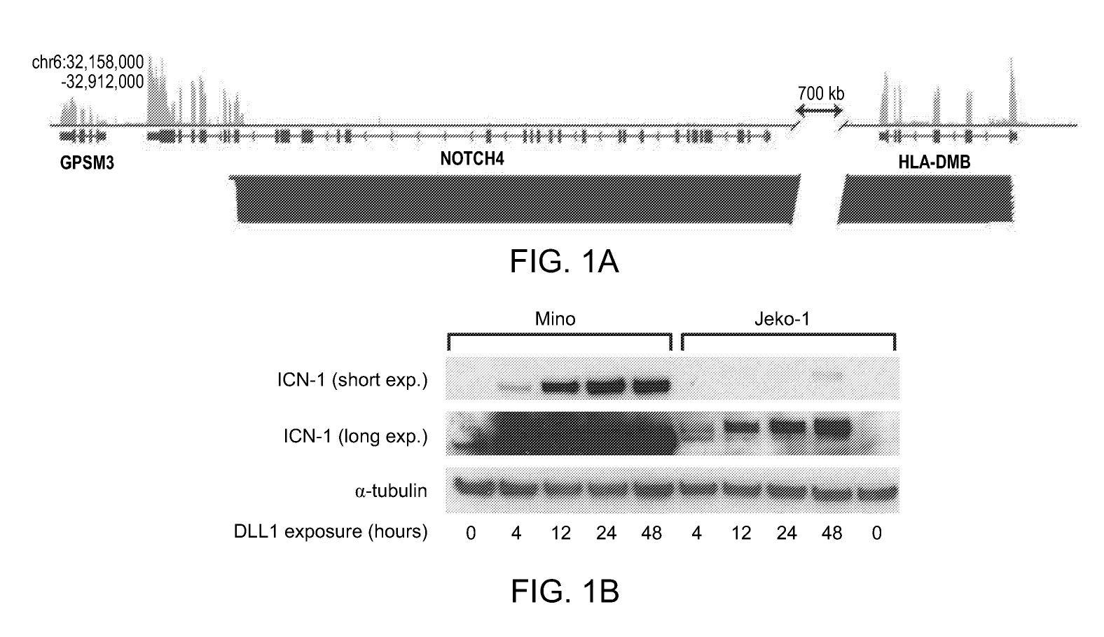

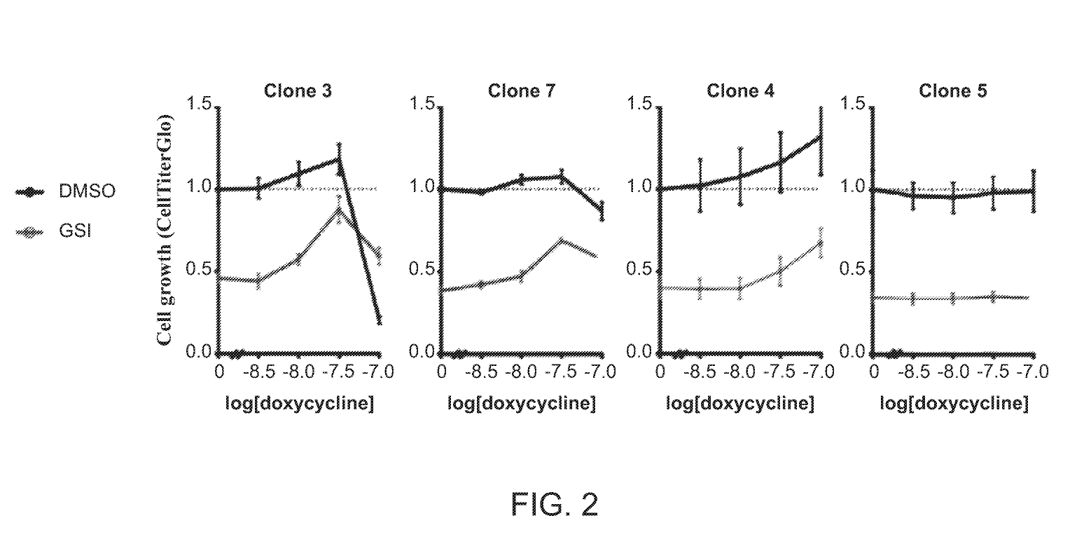

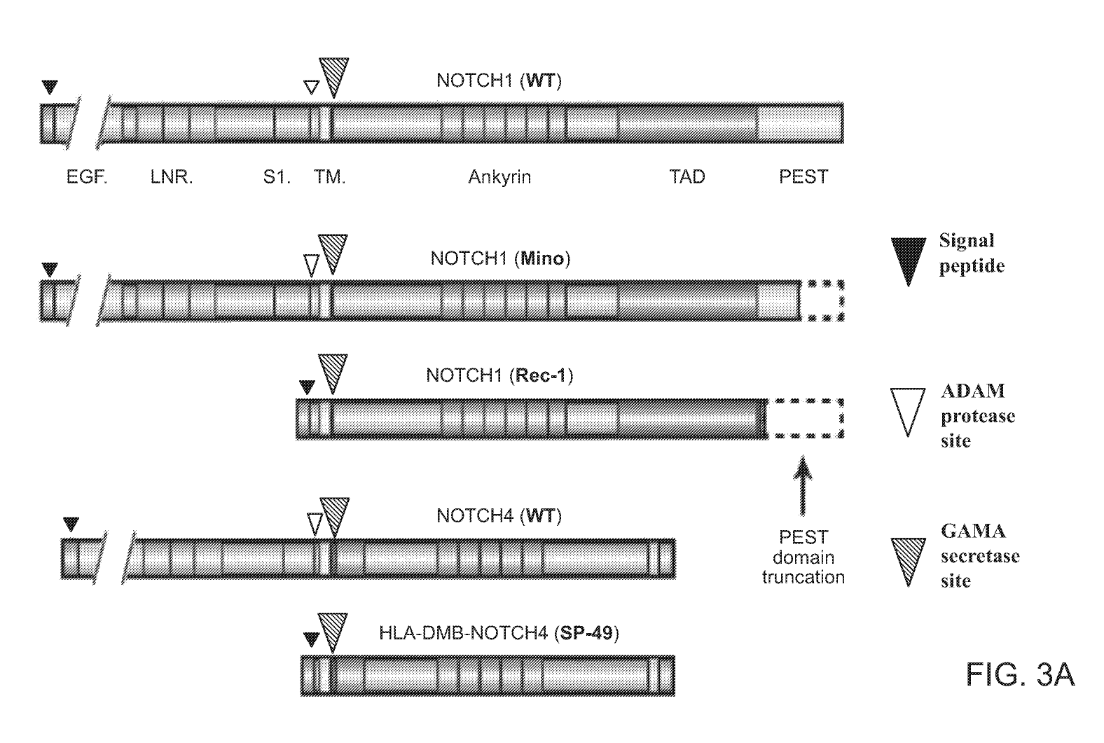

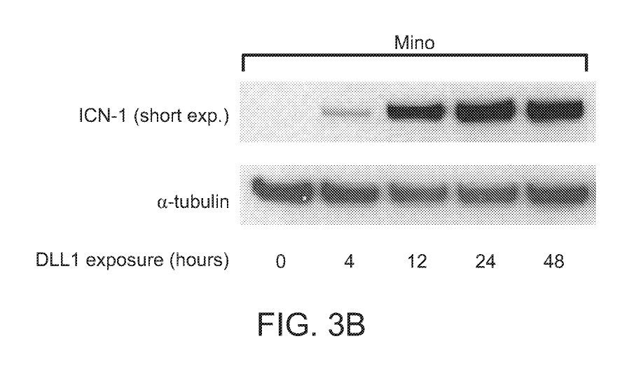

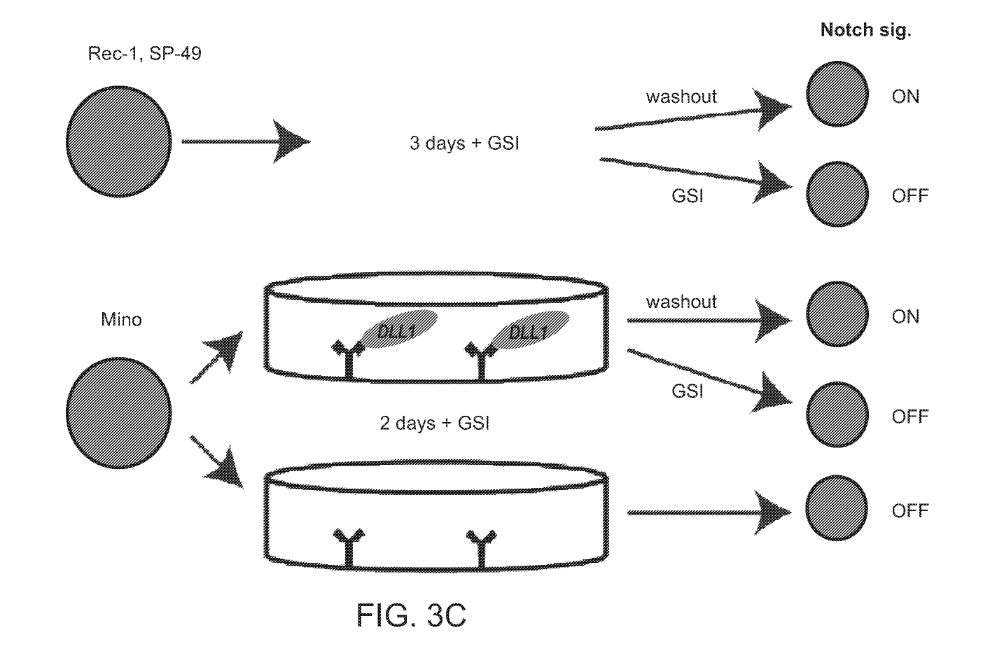

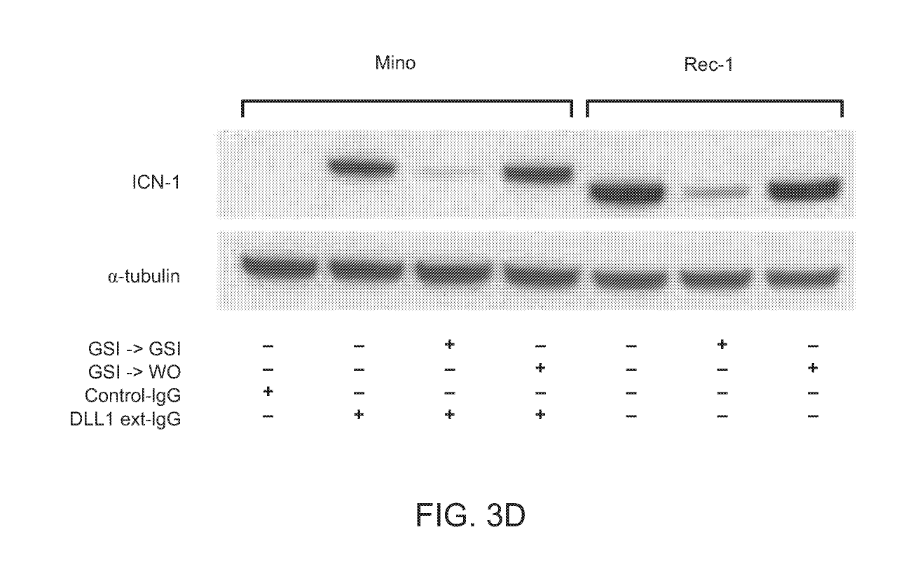

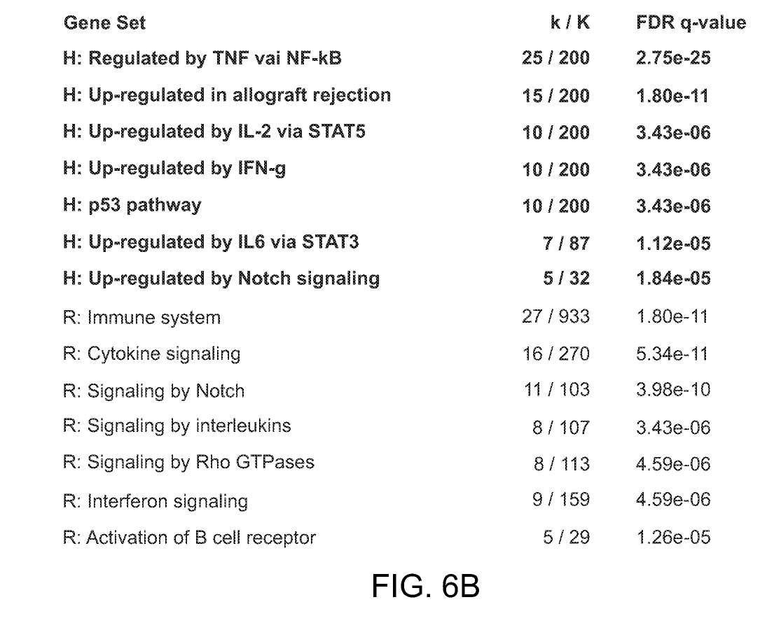

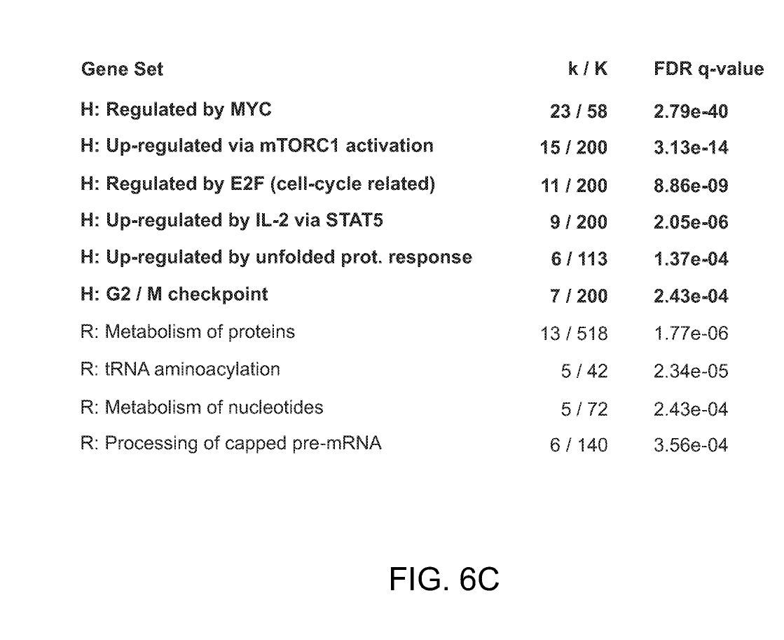

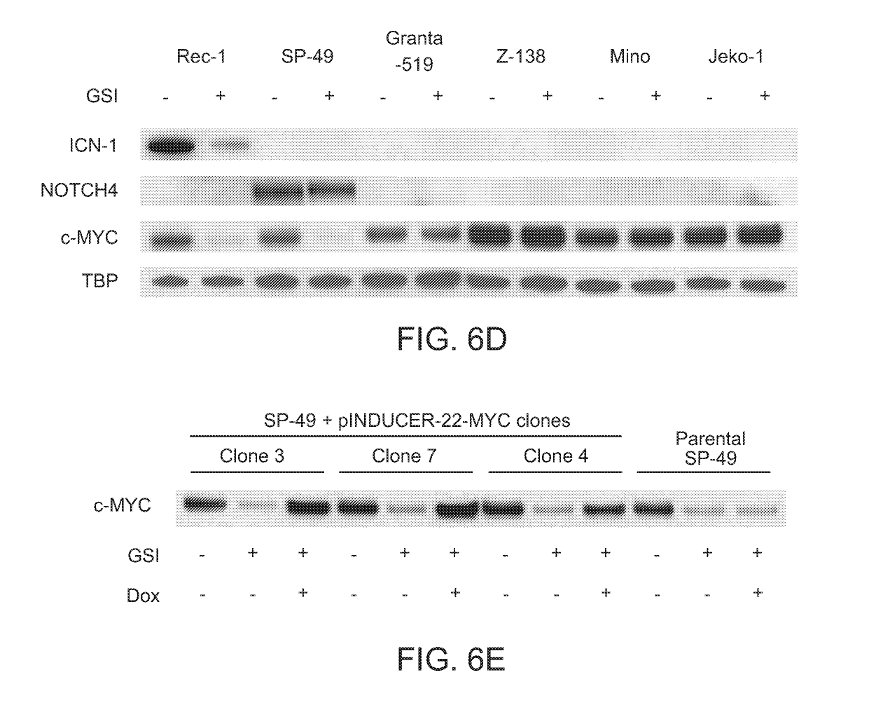

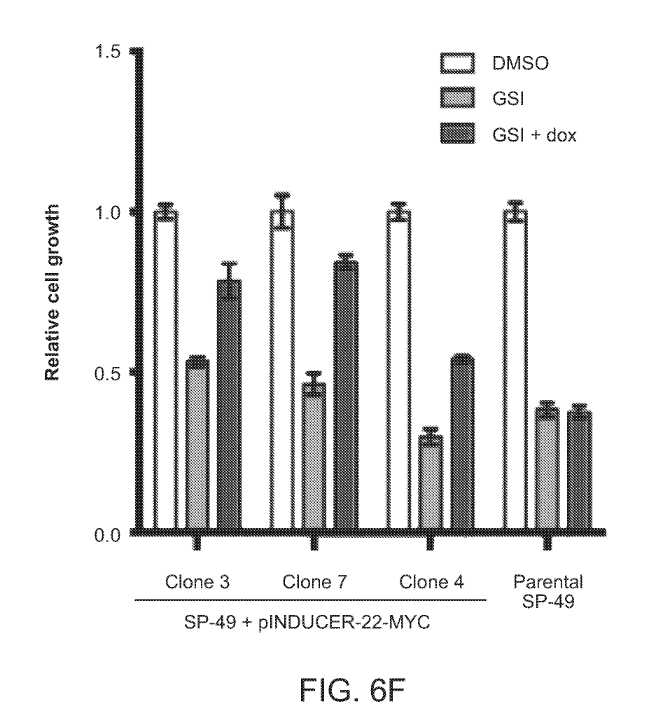

Rec-1 and SP-49 are the only known MCL cell lines that demonstrate substantial growth inhibition upon treatment with GSI (Kridel et al., 2012) (FIG. 2). To understand the basis of GSI-sensitivity in SP-49, paired-end RNA-Seq data was analyzed from that line. The analysis detected a highly expressed, aberrant transcript consisting of the first exon of HLA-DMB and exons 24-30 of NOTCH4 (FIG. 1A) resulting from an approximately 700 kb deletion on chromosome 6 that juxtaposes the corresponding portions of the HLA-DMB and NOTCH4 genes. Exon 1 of HLA-DMB encodes a signal peptide similar to that found at the N-terminal of normal Notch precursor proteins and the truncated Rec-1 NOTCH1 allele, while exons 24-30 of NOTCH4 encode the trans-membrane and intracellular portions of NOTCH4, as well as the gamma-secretase protease site that is required for release of the intracellular NOTCH4 transcription factor from the membrane (FIG. 3A). Thus, the predicted protein product of this fusion transcript resembles other constitutively active aberrant Notch proteins, such as those reported in Rec-1 and T-cell acute lymphoblastic leukemia (T-ALL). Indeed, western blot of CLL and MCL cell line nuclear extracts with a NOTCH4 antibody revealed a band at the predicted size of intracellular NOTCH4 (ICN-4) that was exclusive to SP-49 (FIG. 6D).

### Example 2

### Genome-Wide Identification of Functional Notch Target Genes

To model ligand-dependent Notch activation, MCL cell lines on immobilized recombinant Notch ligand (DLL1^{ext}-IgG) or control protein (IgG) were grown. Analysis by Western blot with an antibody specific for free (gamma secretase-cleaved) ICN-1 demonstrated a time-dependent accumulation of ICN-1 expression in both Mino (FIG. 1B and FIG. 3B), and Jeko-1 (FIG. 1B). ICN-1 accumulation was stronger and more rapid in Mino, consistent with the predicted stabilizing effects of the PEST-truncating mutation in that line (NOTCH1 Q2487*) (FIG. 3B).

To identify Notch-regulated genes and enhancers genome-wide, a GSI-washout strategy in three MCL cell lines was employed (FIG. 3C). Rec-1 and SP-49 were treated for three days with GSI (1 μM compound E), to eliminate intracellular Notch proteins. Subsequently, the media was replaced and a four-hour incubation was performed with media containing vehicle only (washout), or GSI (mock-washout). To rapidly activate Notch in the Mino line, Mino cells were grown in the presence of both DLL 1^{ext}-IgG stimulation and GSI over a 48-hour period, during which time Notch receptors on the cell surface can undergo ligand- and ADAM-protease-dependent S2 cleavage, but not the gamma-secretase-dependent S3 cleavage event that releases ICN. This was then followed by a four-hour GSI-washout or mock-washout procedure identical to that employed for Rec-1 and SP-49. Both the ligand-independent and ligand-dependent procedures lead to rapid Notch activation as measured by ICN-1 accumulation in the NOTCH1-mutant cell lines (FIG. 3D).

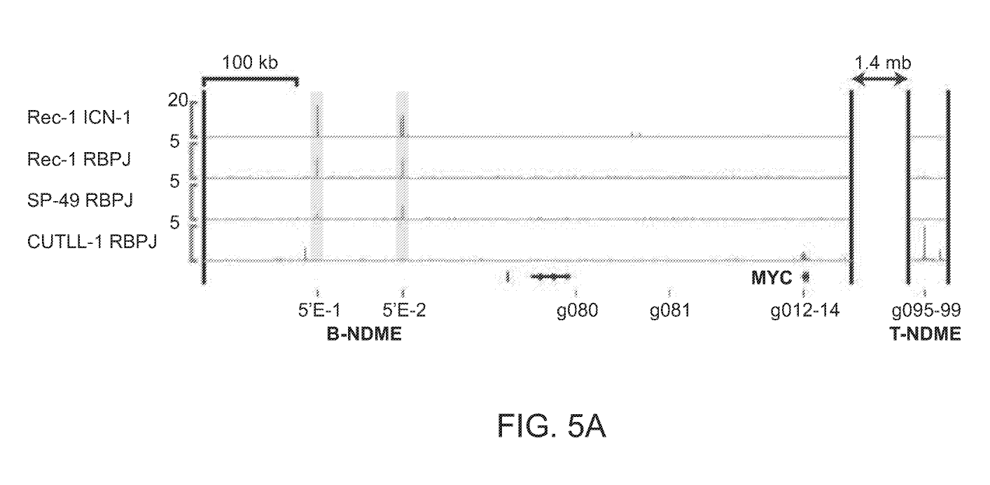

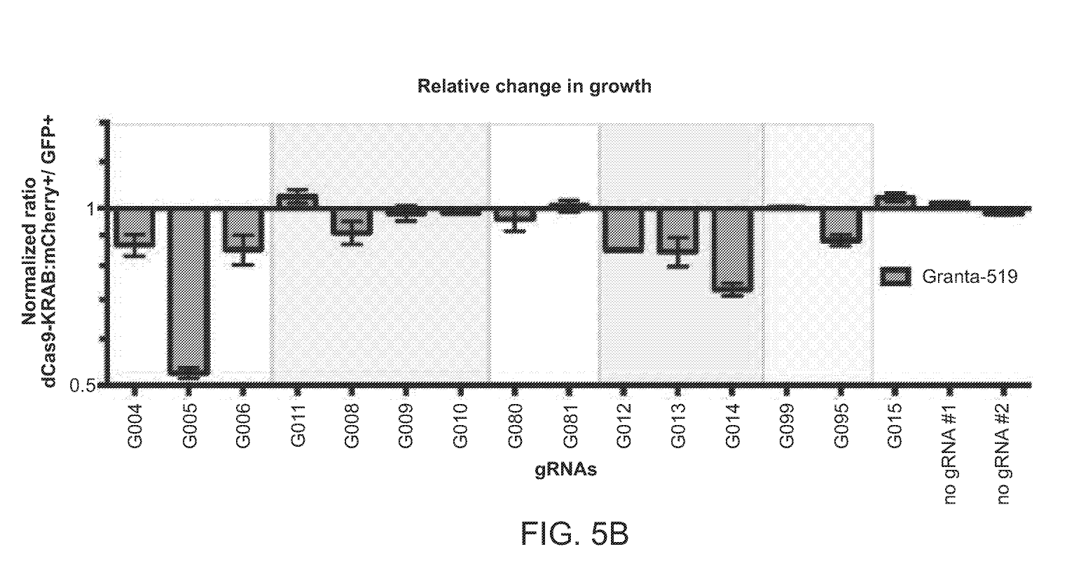

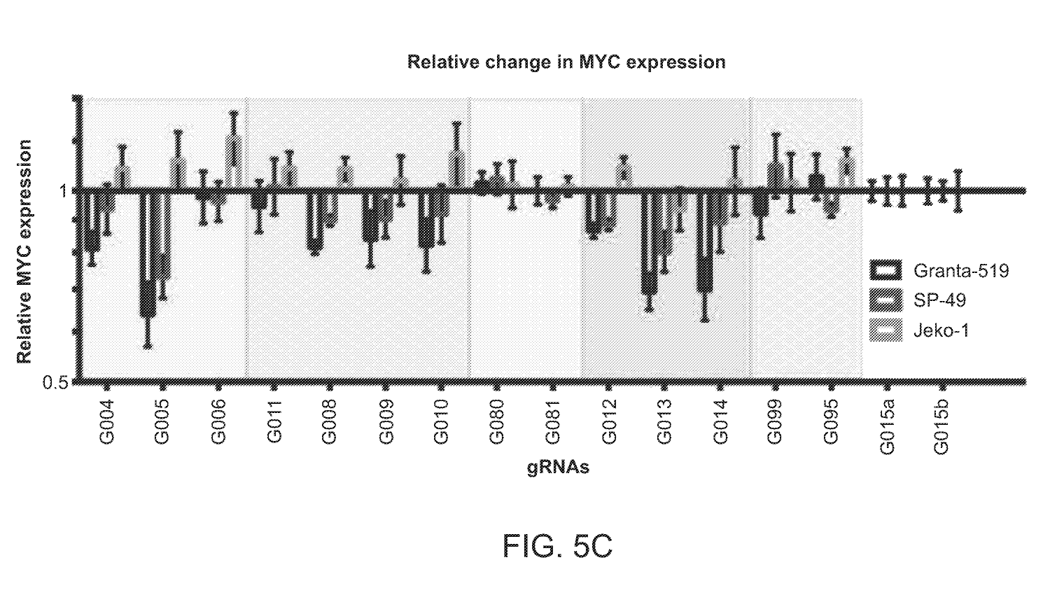

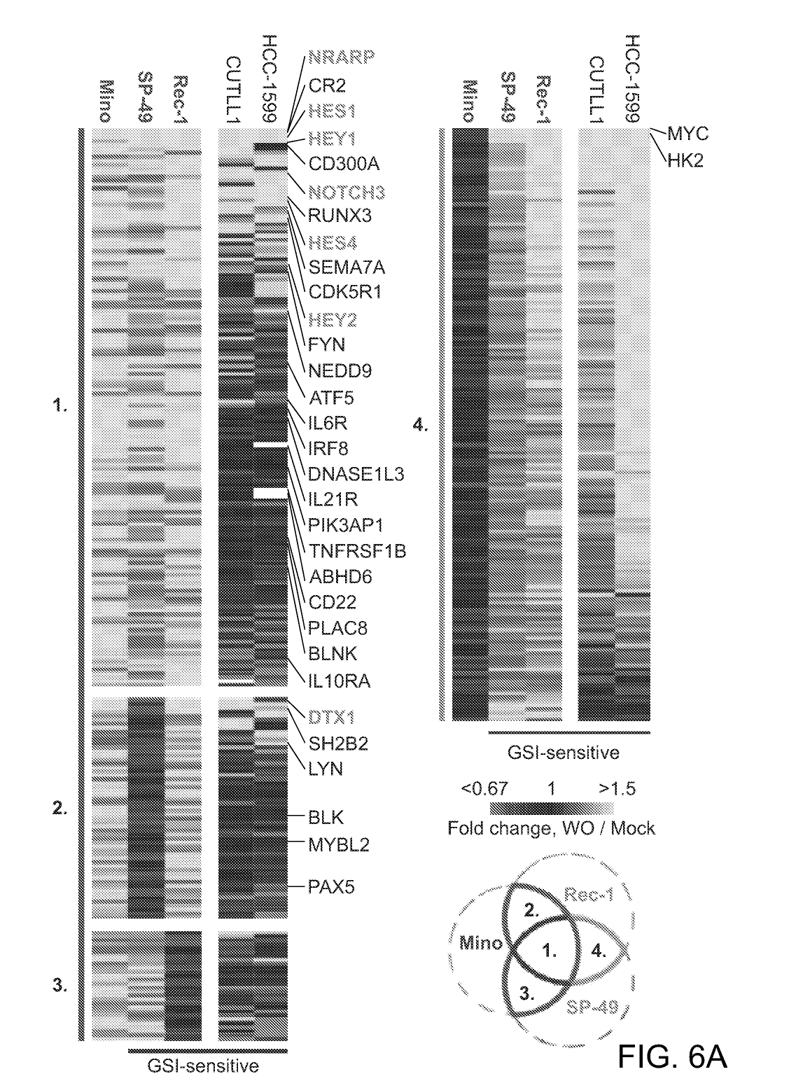

Analysis of triplicate RNA-Seq datasets in each state for the three MCL lines revealed primarily gene activation rather than gene repression, consistent with the known role of intracellular Notch proteins as transcriptional activators (FIG. 5). A total of 377 genes showed independently significant activation in at least two of the three lines (FIG. 6A). Significant Notch-activated genes were further clustered into genes up-regulated in all three, or only two of three MCL lines, and were compared to RNA-Seq data from comparable GSI-washout experiments performed in two other Notch-dependent cancer lines: the T-ALL cell line CUTLL1 and the triple-negative breast cancer line HCC-1599 (Stoeck et al., 2014). Most targets showed less activation in SP-49 compared in Mino and Rec-1, possibly due to altered dynamics or transactivation potential of ICN-4 compared to PEST-truncated ICN-1.

The set of genes up-regulated in all three MCL lines (n=142) included many canonical Notch target genes (HES1, HES4, HEY1, HEY2, NRARP, and NOTCH3), which were also strongly up-regulated in CUTLL1 and HCC-1599. However, a large proportion of genes up-regulated by Notch activation in all MCL lines showed unchanged, or even reduced expression upon Notch activation in CUTLL1 and HCC-1599, indicating that these may represent context-specific Notch targets. A similar pattern was seen in the set of activated genes common to Mino and Rec-1, but not SP-49 (n=56), which included the canonical Notch target gene DTX1 as well as many apparently tissue-specific target genes. Gene set analysis of all genes activated by Notch in at least one GSI-sensitive MCL line and the GSI-insensitive Mino line revealed significant enrichment for gene sets associated with Notch signaling in the mSigDB Hallmark and Reactome collections (FIG. 6B), but also for gene sets related to lymphocyte or B-cell biology, including interleukin, interferon, and B-cell receptor signaling, as well as a signature of NF-KB target gene activation.

In contrast, a very different pattern was observed in the large set of genes (n=151) that were activated by Notch signaling in both of the GSI-sensitive MCL lines SP-49 and Rec-1, but not in GSI-insensitive Mino. The vast majority of these genes were also Notch-activated in CUTLL1 and HCC-1599 (FIG. 6A), indicating that these may represent a gene expression module associated with Notch-dependent growth across cancer types. Indeed, the most strongly up-regulated of these genes in all four GSI-sensitive lines was the oncogene MYC, which is known to be a critical direct Notch target in T-ALL. Furthermore, comparison of genes uniquely activated in GSI-sensitive MCL to the curated mSigDB Hallmark and Reactome collections (FIG. 6C) revealed strong enrichment for MYC target genes, and MYC-regulated biological processes, including nucleotide metabolism, transcriptional processing, protein synthesis, and cell cycle control, indicating that many genes in this set may be secondarily or cooperatively activated by Notch-dependent MYC activation. Genes associated with mTORC1 activation were also enriched in this set, consistent with prior data linking mTORC1 to MYC upregulation in T-ALL (Chan et al., 2007) and in mature T cell activation (Wang et al., 2011).

Treatment of MCL cell lines with GSI revealed a substantial decrease in c-Myc protein levels for Rec-1 and SP-49 only (FIG. 6D), supporting MYC as a Notch-activated target in GSI-sensitive MCL. Given the broad role of MYC in normal and neoplastic lymphocyte proliferation, these findings indicated that loss of MYC expression might explain the proliferation defect seen in GSI-treated Rec-1 and SP-49. To test this, single-cell clones were derived from SP-49 transduced with a lentiviral vector encoding a MYC transgene under the control of a doxycycline-inducible promoter (pINDUCER-22-MYC). Indeed, clones that demonstrated effective MYC induction showed a doxycycline dose-dependent rescue of cell growth in the presence of GSI (FIGS. 6E -6F). Thus, Notch-dependent regulation of MYC expression explains much of the dependency of Recl and SP-49 on constitutive Notch signaling. Interestingly, expression of MYC at levels higher than that seen in parental SP-49 cells was associated with reduced cell viability, indicating that Notch-dependent MCL cells are highly sensitive to either excessive or insufficient MYC levels.

### Example 3

### Intracellular Notch or Viral Surrogates Drive MYC Via 5′ Enhancers in MCL Cell Lines.

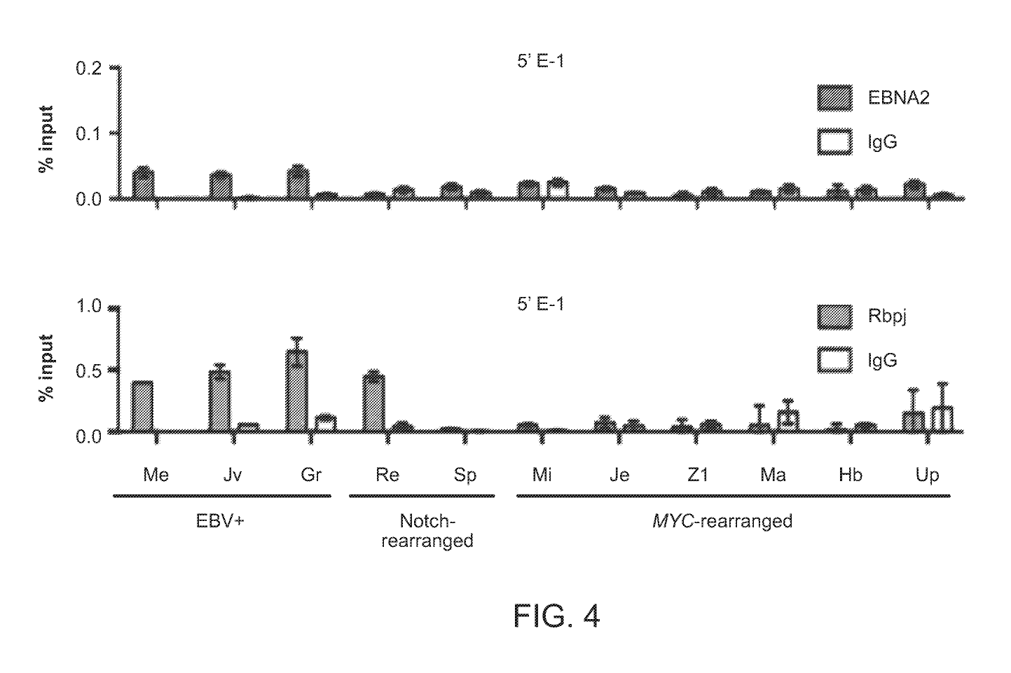

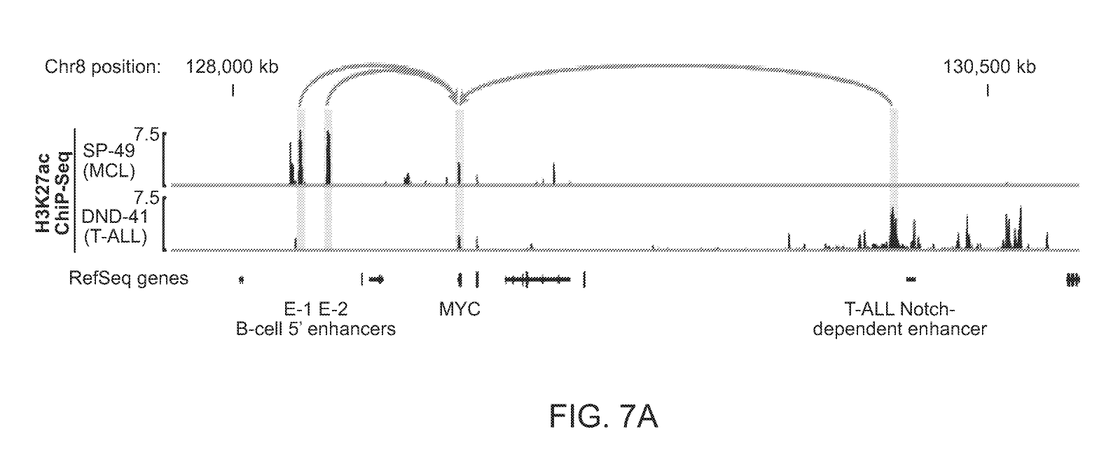

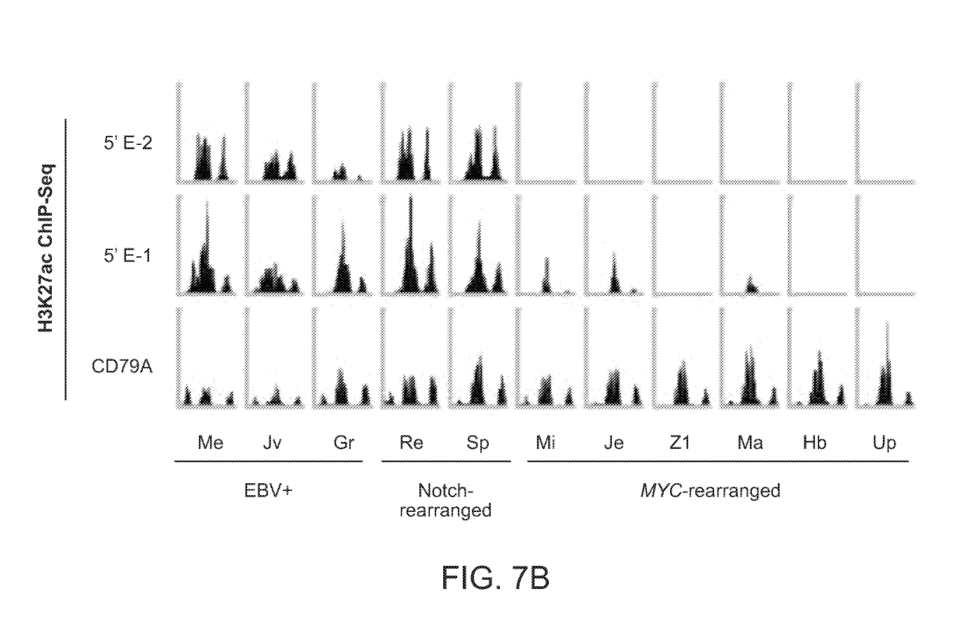

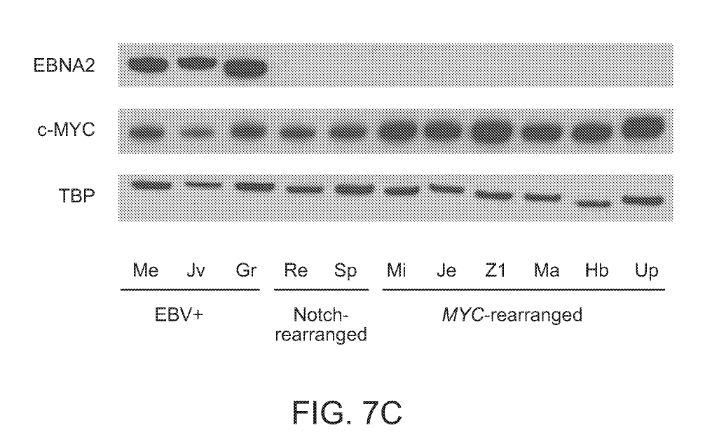

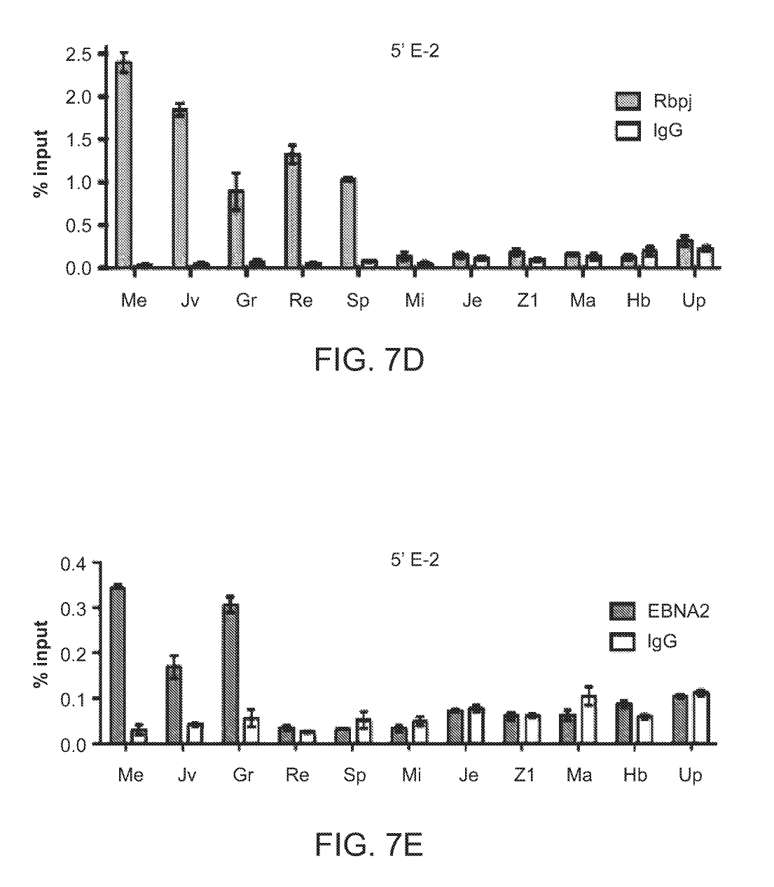

Additional studies to understand the genomic mechanism by which Notch signaling regulates MYC expression in MCL were undertaken. Prior studies across diverse tissues and cancer types have implicated highly tissue-specific distal enhancer elements in MYC activation, including the Notch-dependent 3′ MYC enhancer identified in immature T cells and T-lymphoblastic leukemia (hereafter TNDME). Lymph node biopsies from CLL and MCL showed no evidence of T-NDME acetylation, but do show strong acetylation of enhancer-like elements on the 5′ side of the MYC gene (Ryan et al., 2015). ChIP-Seq was performed for histone H3 Lysine 27 acetylation (H3K27ac) in one CLL and ten MCL cell lines, and noted strong acetylation at the 5′ MYC enhancers in only five lines, including the two Notch gene-rearranged lines Rec-1 and SP-49 (FIGS. 7A-7B). EBV+-transformed human B cells show acetylation of these same elements, which are bound by RBPJ and the EBV-encoded RBPJ cofactor EBNA2 (Zhao et al., 2011). Three of the CLL and MCL cell lines are known to be positive for EBV infection and showed EBNA2 protein expression by Western blot (FIG. 7C and FIG. 4), and all three show strong 5′ enhancer acetylation. Thus, all CLL/MCL lines showing acetylation of 5′ enhancers express either constitutively active intracellular Notch, or a viral Notch surrogate protein, indicating that these elements represent B cell-specific Notch-dependent MYC enhancers (hereafter BNDME sites E1 and E2). Indeed, ChIP-PCR demonstrated binding of EBNA2 at the two 5′ enhancers in the EBV+lines, while RBPJ was exclusively bound to 5′ enhancers in the EBV+and Notch-rearranged lines (FIGS. 7D-7E). Importantly, analysis of all 11 cell lines with MYC break-apart and MYC/IGH dual fusion FISH, as well as published conventional karyotyping and other analyses convincingly demonstrate the presence of genomic MYC locus rearrangements in all six MCL lines that lack both EBNA2 expression and an activating Notch gene rearrangement, thus explaining the high levels of Notch-independent MYC expression in these lines, including Mino (FIG. 7C).

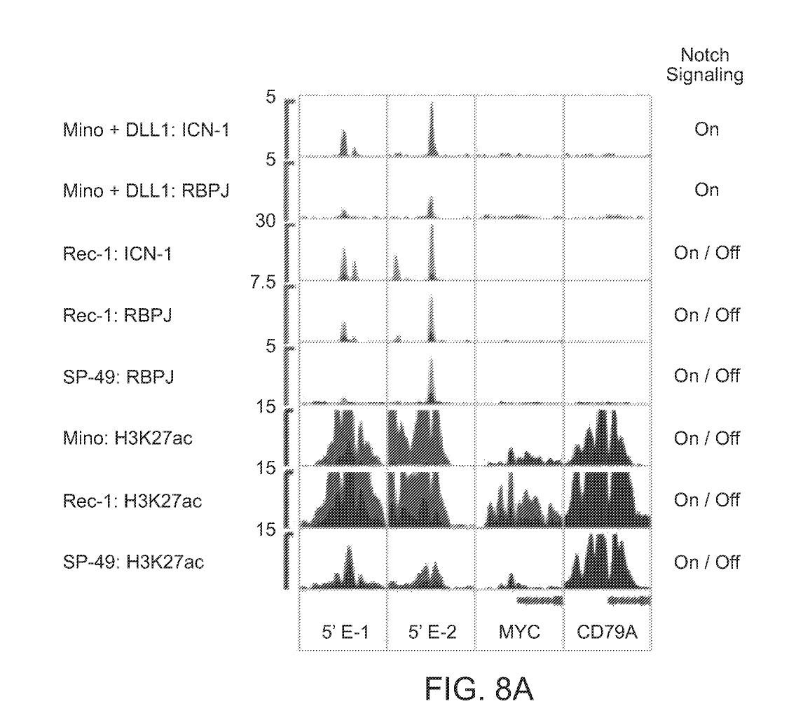

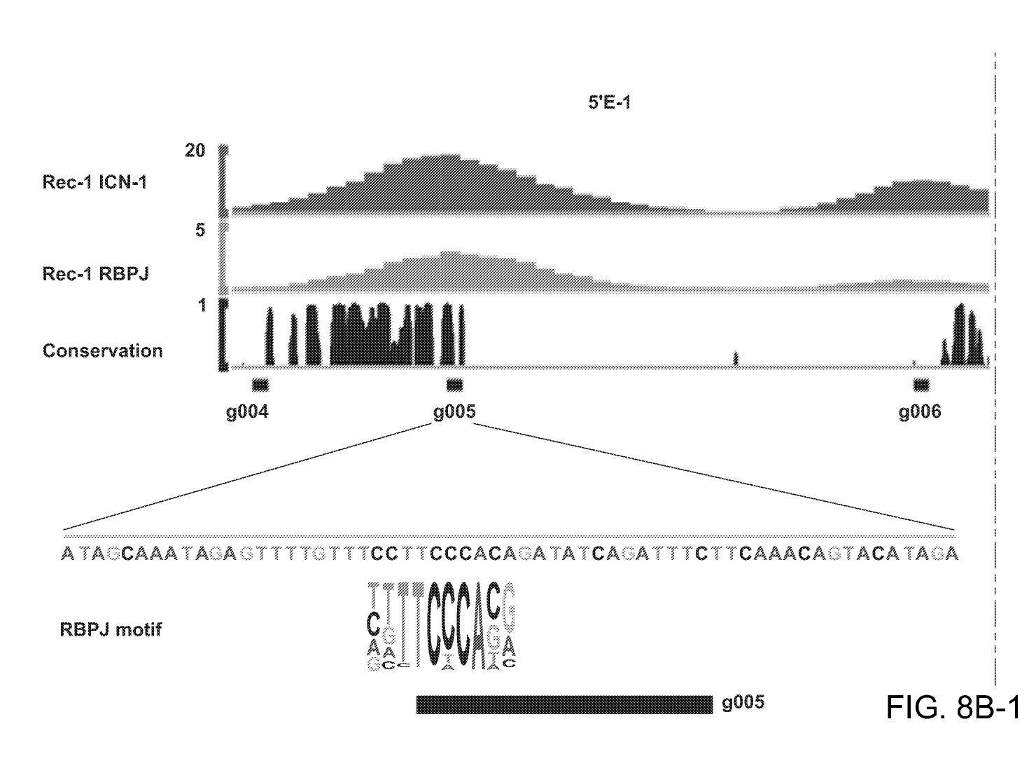

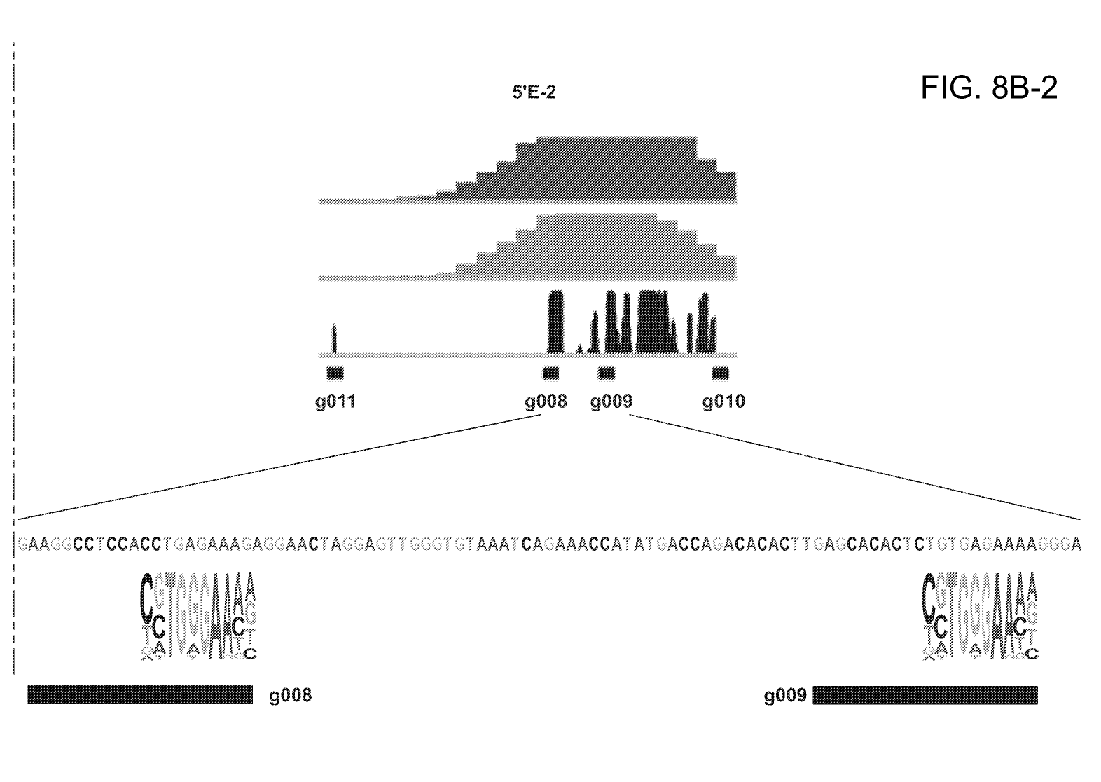

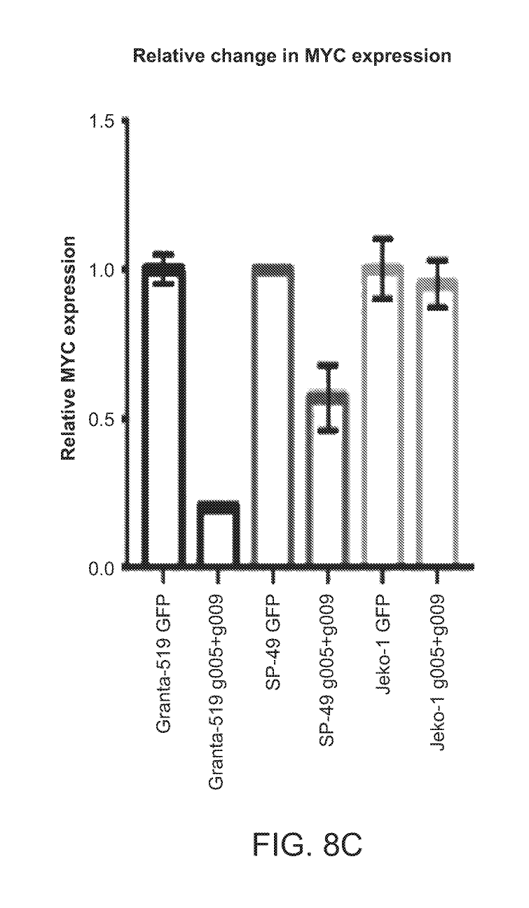

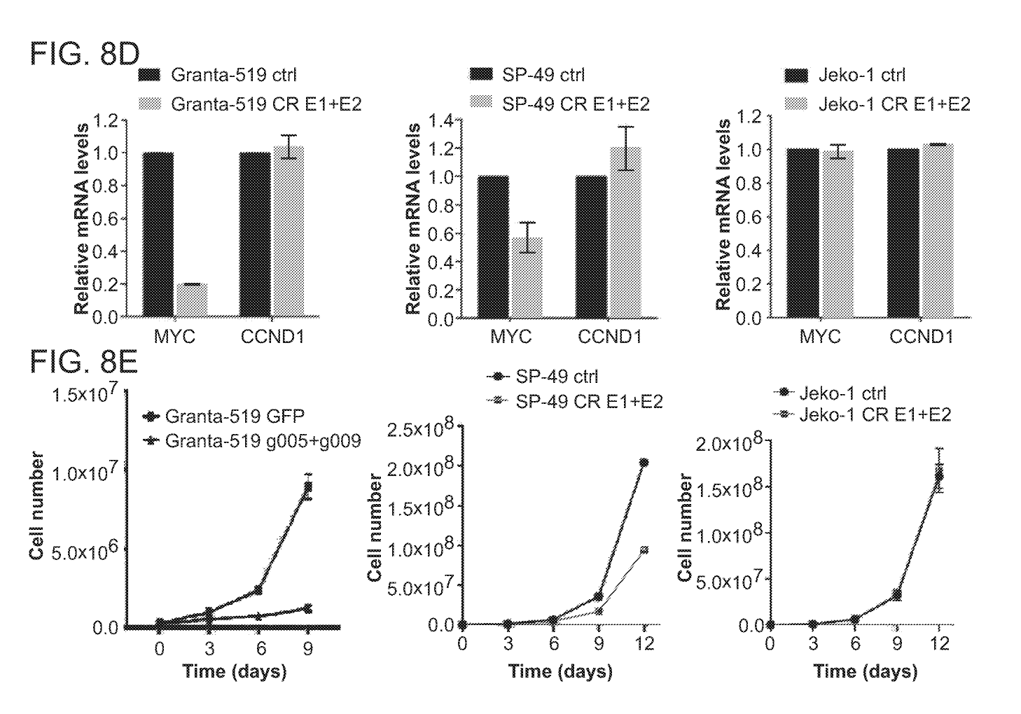

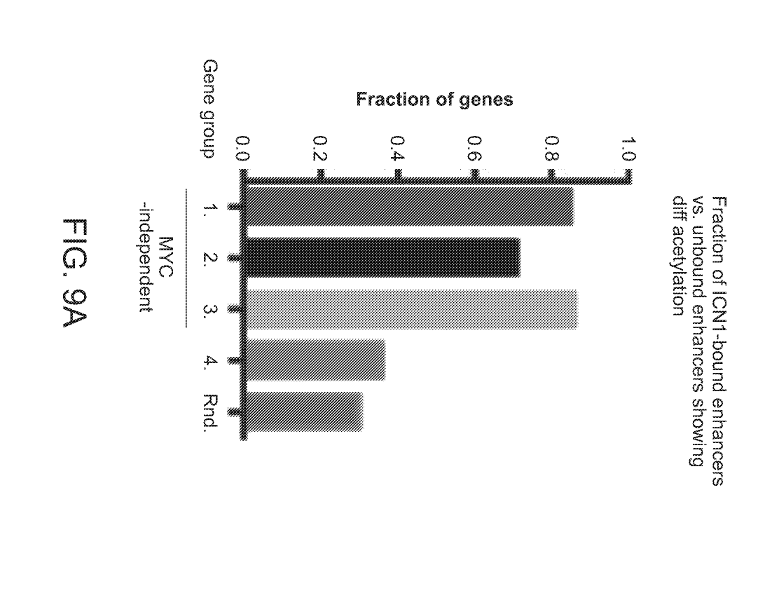

To directly evaluate enhancer regulation by Notch transcription complex, ChIP-Seq was performed for H3K27ac, RBPJ, and ICN-1 in Notch-rearranged MCL cell lines following GSI-washout and mock-washout experiments. Specific peaks of RBPJ and (in Rec-1) ICN-1 binding at the BNDME sites were noted in the washout (‘notch-on’) samples which were absent or markedly reduced in the mock-washout (notch off) state (FIG. 8A). BNDME sites also showed markedly stronger acetylation in the Notch-on state. Mino cells stimulated with recombinant DLL1 also showed binding of NTC proteins and activation of BDME acetylation, despite decoupling of MYC expression from Notch activity in the setting of a MYC-IGH genomic rearrangement. Motif analysis of DNA sequence within each BNDME site revealed the presence of one evolutionarily conserved RBPJ motif in E1 and two conserved motifs in E2 (FIG. 8B). Importantly, no evidence of ICN-1 or RBPJ binding at the T-NMDE was observed in any MCL line, while conversely, published RBPJ binding data in CUTLL1 showed strong binding at the T-NDME, but not at the B-NDME sites, indicating that additional tissue-specific factors must be necessary to facilitate tissue-specific binding of the NTC to each enhancer in a tissue-specific manner.

To prove that the BNDME sites are bona fide MYC enhancers, lentiviral guideRNA constructs targeting 15 distinct sites across the MYC locus were designed, including the MYC promoter, RBPJ motifs with the T-NDME and both B-NDME sites, as well as the MYC promoter and other intergenic sites (FIG. 8B and FIG. 5A), plus a non-targeting control guideRNA. Populations were generated of SP-49 (Notch-rearranged), Granta-519 (EBV+), and Jeko-1 (MYC-rearranged and amplified) stably expressing a dCas9-KRAB-E2A-mCherry transgene, which encodes a nuclease-dead Cas9-KRAB fusion protein that mediates local epigenetic repression. Transduction of dCas9-KRAB-E2A-mCherry stable lines with MYC locus gRNAs led to a substantial decrease in MYC expression in Granta-519 and SP-49 for guides targeting the MYC promoter or central RBPJ of E1, a modest but significant decrease for gRNAs targeting the E2 RBPJ sites, and no change in MYC expression for guides targeting the T-NDME or intergenic regions (FIG. 5C). Next, dCas9-KRAB-E2A-mCherry stable lines were simultaneously infected with E1- and E2-targeting guideRNA lentiviruses encoding distinct fluorescent proteins, sorted doubly-transduced cells, and measured MYC expression, revealing a substantially greater decrease in MYC expression for Granta-519 and SP-49 (FIG. 8C) when both enhancers were targeted compared to targeting of E1 or E2 alone. To test the effect of these guides on MCL proliferation, the original 16 guideRNAs were utilized to infect a mixture of dCas9-KRAB-E2F-mCherry-expressing cells and cells transduced with a vector expressing GFP alone (FIG. 5B). After 7 days, flow cytometry was used to measure the ratio of mCherry+versus GFP+cells relative to cells infected with a control gRNA. Guides targeting the MYC promoter and E1 were associated with decreased proliferation of the dCas9-KRAB-E2F-mCherry population for Granta-519, but little effect was seen for SP-49 (FIG. 5C). However, both MYC expression (FIG. 8D) and proliferation (FIG. 8E) markedly suppressed in both Granta-519 and SP-49 (but not Jeko-1) with a combination of E1- and E2-targeting guides in cells stably expressing Cas9 nuclease. Together, these findings demonstrate that the BNDME sites drive MYC expression and proliferation in EBV+and Notch-dependent MCL lines.

### Example 4

### Direct Notch Targets Include Regulators of B Cell Signaling and Differentiation

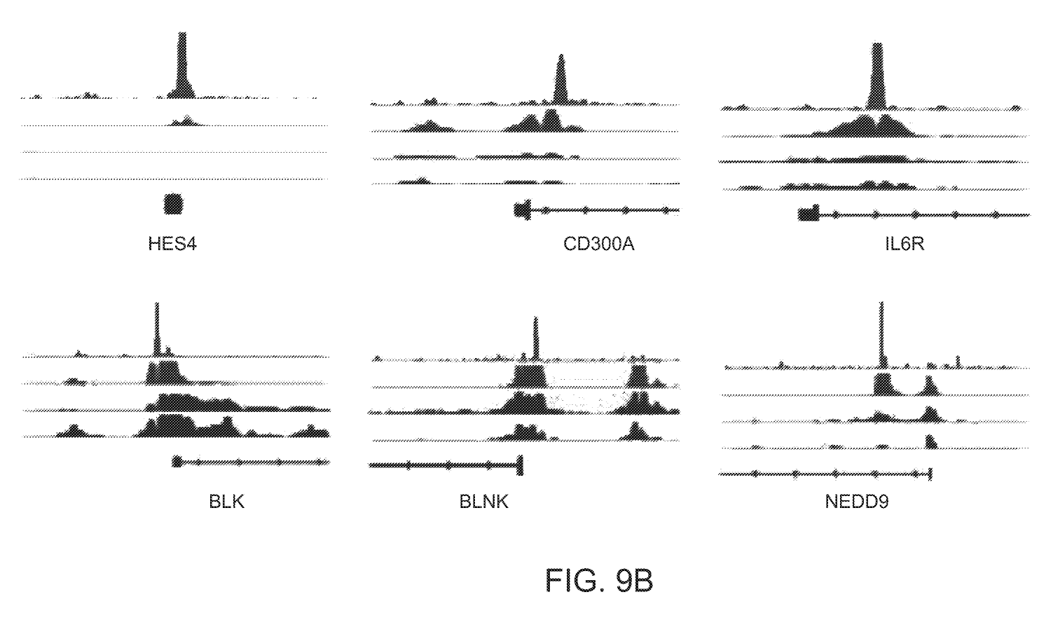

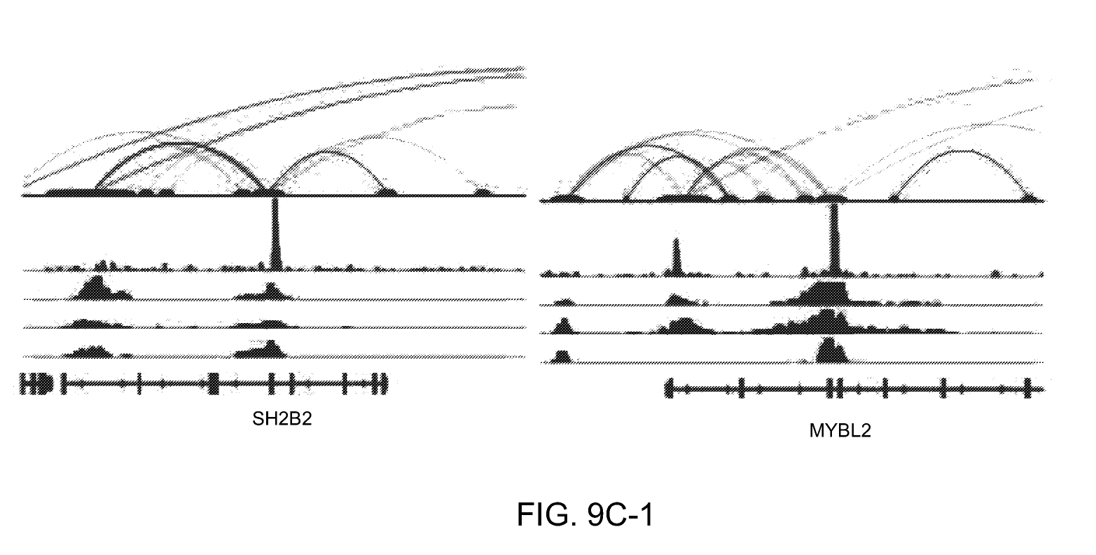

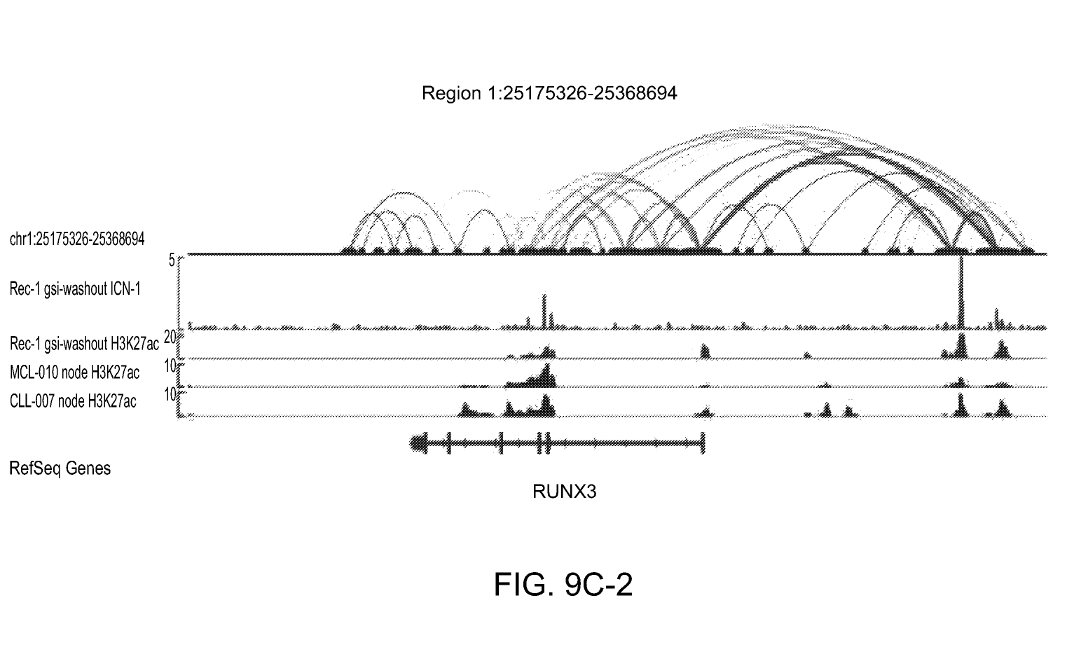

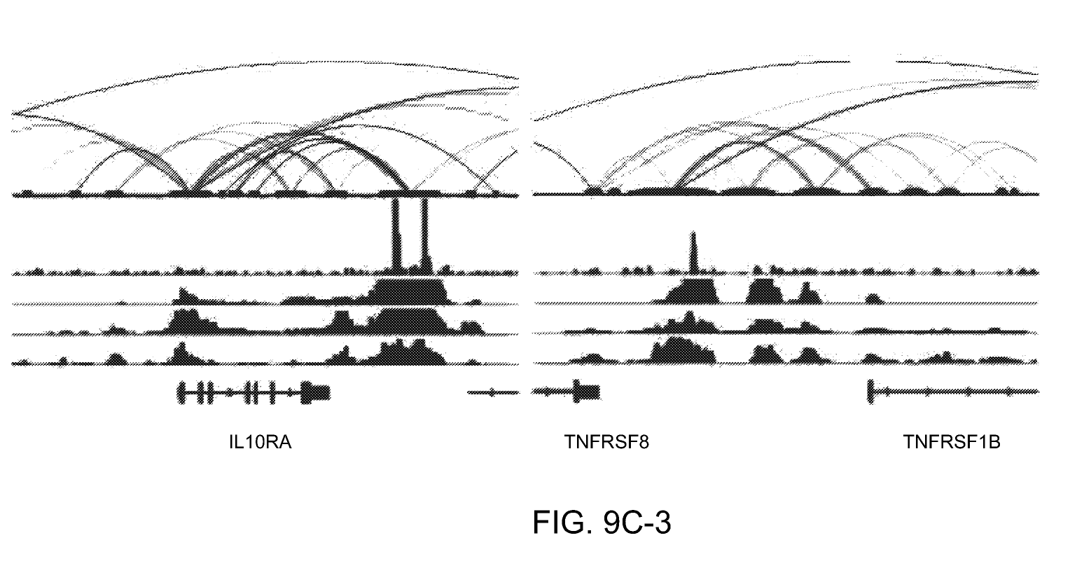

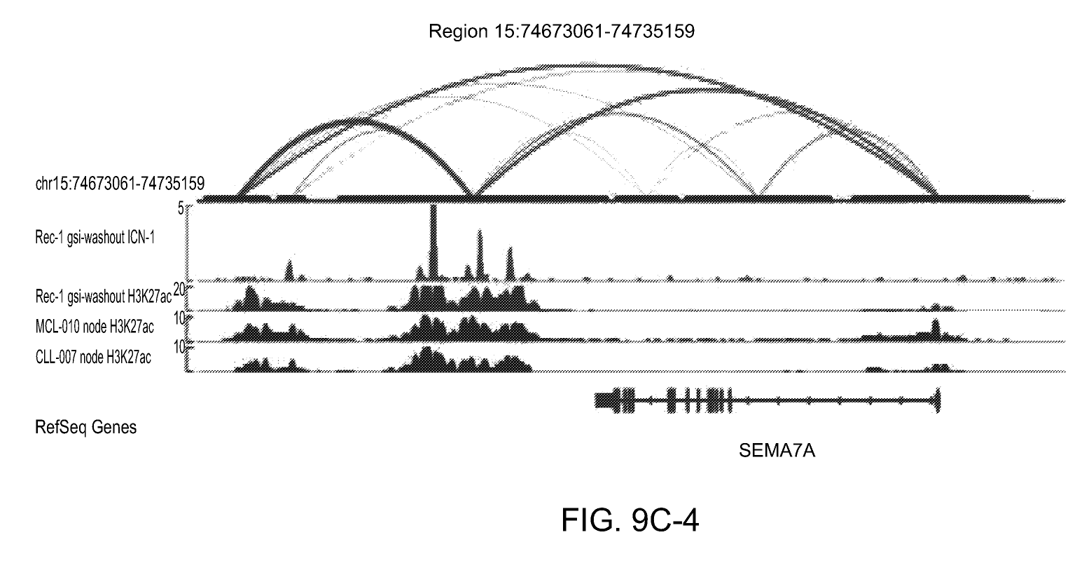

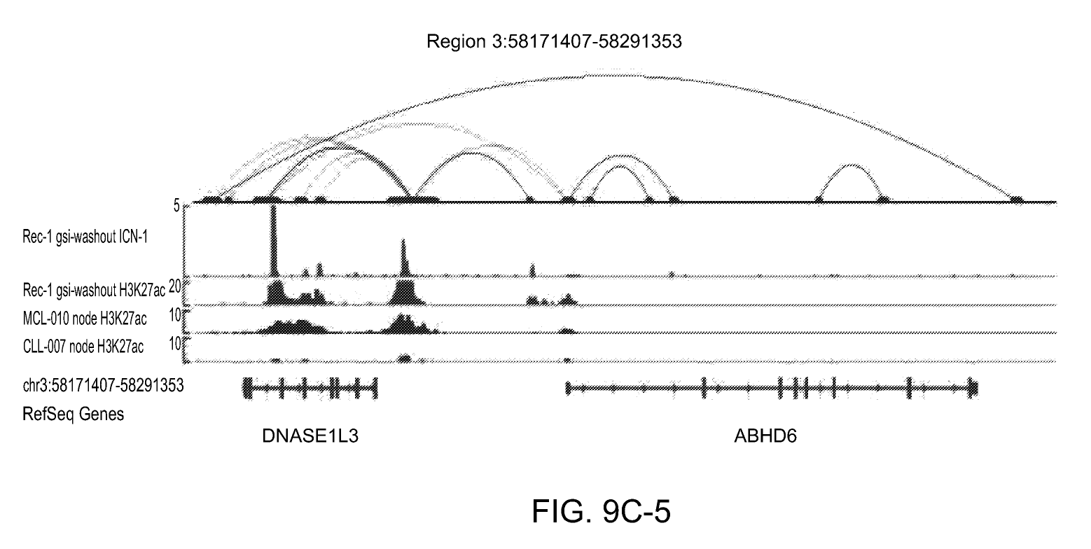

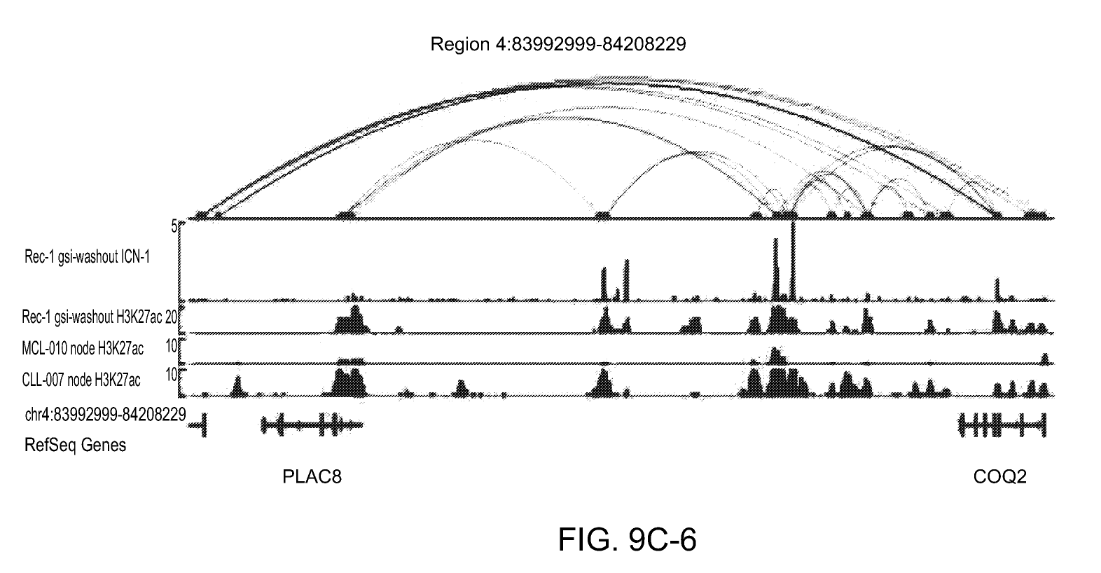

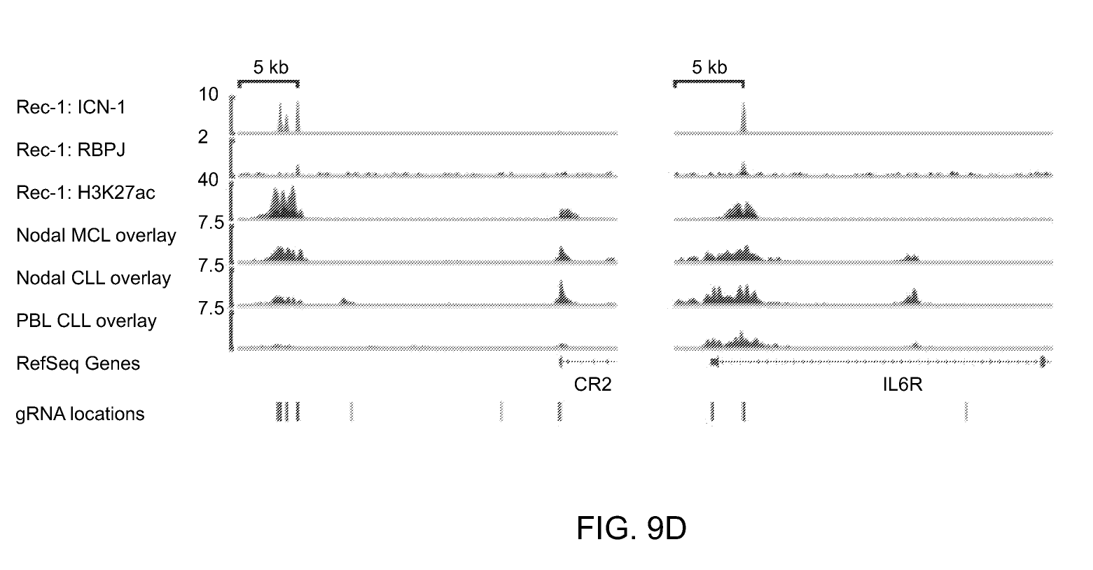

Additional studies were undertaken to identify other direct Notch target genes that might play an important role in MCL and CLL biology. Only a small fraction of Notch-activated genes identified in the GSI-washout analysis showed ICN-1 and RBPJ binding, raising the possibility that many of these genes, like MYC, might be activated by Notch-dependent distal elements. To identify such elements, published genome-wide maps were utilized of 3-dimensional genomic interactions associated with RNA Polymerase II via Chromatin Interaction Analysis by Paired-End Tag sequencing (PolII ChIA-PET) in the EBV-immortalized B-lymphoblastoid cell line (LCL) GM12878 (Tang et al., 2015). In support of this approach, strong interactions between both B-NDME sites and the MYC promoter were observed in the GM12878 PolII ChIA-PET data (FIG. 7A). Strikingly, the majority of genes activated by GSI-washout in both GSI-sensitive and -insensitive MCL models showed either ICN-1-bound enhancers linked via ChIA-PET analysis or ICN-1 bound promoters (FIG. 9A), strongly supporting these genes as direct Notch regulatory targets. This association was highly significant compared to randomly selected gene sets, or to the set of genes activated by Notch in GSI-sensitive MCL only, consistent with most of the latter genes being secondary targets up-regulated via Notch-dependent MYC activation. Because the regulatory state of some true Notch target genes in MCL might be different in EBV+LCLs, a secondary linkage analysis was performed based on the presence on a gene promoter and ICN-1 binding site within the same CTCF-mediated chromatin contact domains (CCD), which are thought to be relatively invariant between related cell types. This analysis yielded an even higher proportion of candidate direct Notch targets among Notch-activated genes in

GSI-sensitive and -insensitive MCL, and highly significant enrichment over GSI sensitive-only and random gene sets. Notch-activated enhancers identified in these analyses showed properties consistent with Notch target enhancers in other tissues, including dynamic ICN-1 and RBPJ binding in the presence or absence of GSI, and increased H3K27ac signal in the notch-on state.

In total, the combined functional and epigenetic analysis revealed high-confidence direct Notch target genes with linked regulatory elements in the MCL models presented herein. Only a minority of these genes also showed Notch-dependent activation in T-ALL (CUTLL-1) and TNBC (HCC-1599) cell lines, and most have not been previously identified as Notch target genes in any tissue, although all of the canonical Notch target genes identified in the gene expression analysis presented herein was correctly supported as direct ICN-1 targets via promoter binding or ChIA-PET linkage. The positions of ICN-1 peaks with respect to novel target gene promoters were diverse, reflecting a similar diversity seen in canonical Notch target genes (FIG. 9B, FIGS. 9C-1-9C-6, FIG. 9D). Some targets showed only a single ICN-1 peak at or just proximal to the gene promoter (e.g. HES4, BLK, BLNK), while a substantial number of genes showed an ICN-1 peak within the proximal first intron (NOTCH3, CD300A, IL6R, NEDD9) a region often associated with regulation of RNA polymerase pause-release. Other genes showed ChIA-PET-linked ICN-1 binding sites more distally within the gene body (SH2B2, MYBL2, LYN), at intergenic sites upstream (RUNX3, CR2) or downstream (SEMA7A, IL10RA, IKZF3) of the target gene, or within the gene body of an adjacent gene (NRARP, CDK5R1). Some genes showed both strong promoter-proximal and -distal ICN-1 peaks (HES1, IL21R), while others showed multiple distal peaks (BATF, POU2AF1, PAX5, PIK3AP1). Finally, there were several loci that contained multiple Notch-activated genes commonly linked to adjacent ICN-1 binding sites, likely representing multi-gene regulatory units (DNASE1L3/ABHD6 and PLAC8/COQ2). To validate the linkage analysis, three strongly Notch-regulated genes were selected, that encode cell surface proteins that were associated with a first intron ICN-1 binding site (IL6R), a 5′ distal enhancer (CR2), and a 3′ distal enhancer (SEMA7A) and demonstrated knockdown of cell surface expression in SP-49 by dCas9-KRAB using guideRNAs designed to target the corresponding regulatory sites (FIG. 9D).

Next, the set of identified direct Notch target genes for association with pathways identified in the gene set analysis of the RNA-Seq data was examined. Notably, genes involved in cytokine/interleukin signaling (IL6R, IL10RA, IL2 IR) and B cell receptor activation (FYN, LYN, BLK, BLNK, PIK3AP1, SH2B2, NEDD9) were identified as direct Notch targets, indicating that these pathways may be directly modulated by Notch-dependent gene activation. Functional analysis of the set of direct Notch targets with the Ingenuity system predicted a significant activatory effect of Notch-regulated genes on B cell receptor signaling. The large number of transcription factor genes that were predicted to be direct

Notch targets was striking, indicating a broad effect of Notch in activating or reinforcing diverse transcriptional regulatory programs in MCL lines. Interestingly, the NF-KB target gene signature noted in the Notch-activated genes was substantially driven by genes that were not associated with ICN1 peaks, indicating that secondary activation of NF-KB and NF-KB target genes may be an early feature of Notch activation in B-cell lymphoma cells, similar to the phenomenon observed with MYC.

### Example 5

### Direct Targets are Regulated by Notch in Primary CLL and MCL

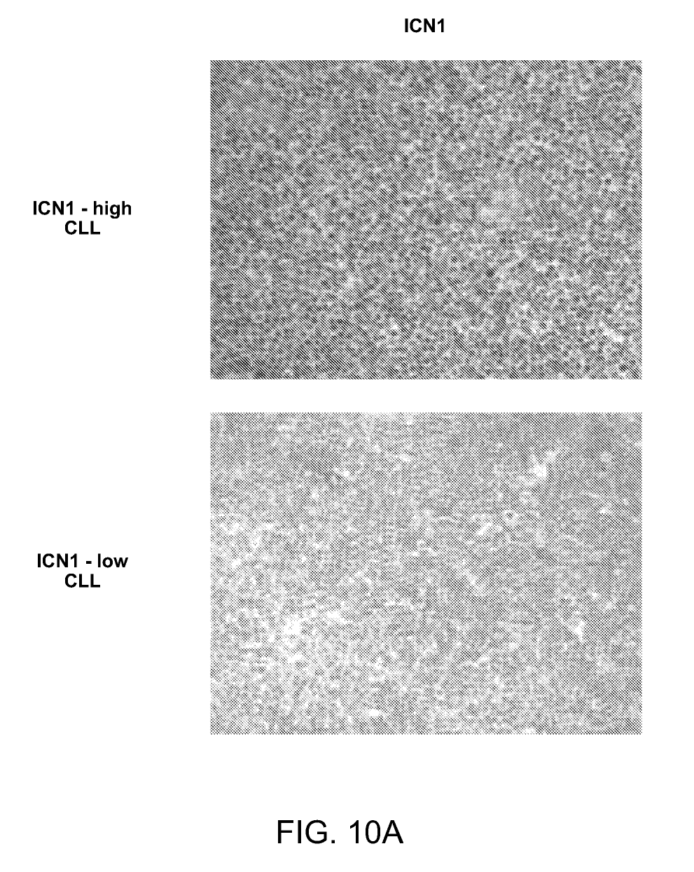

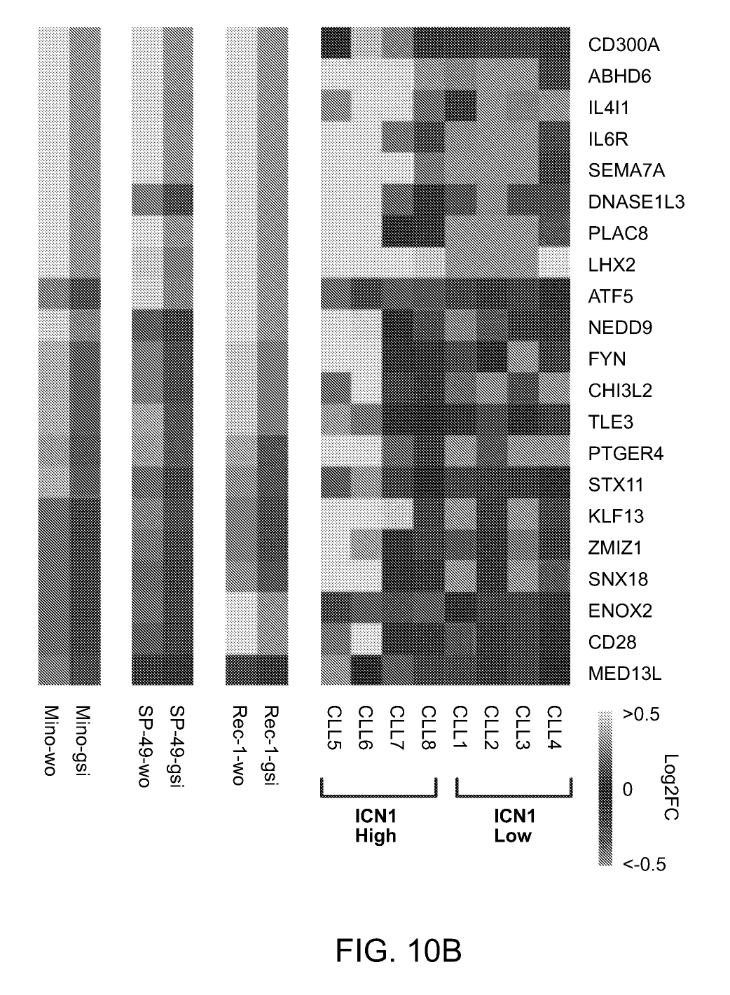

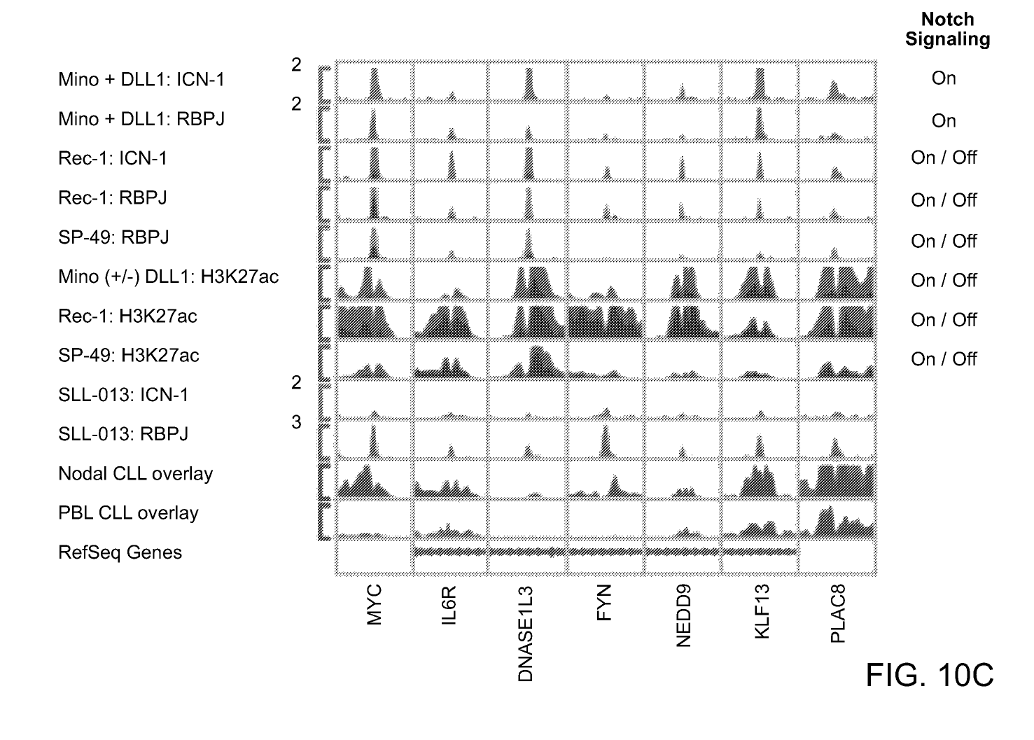

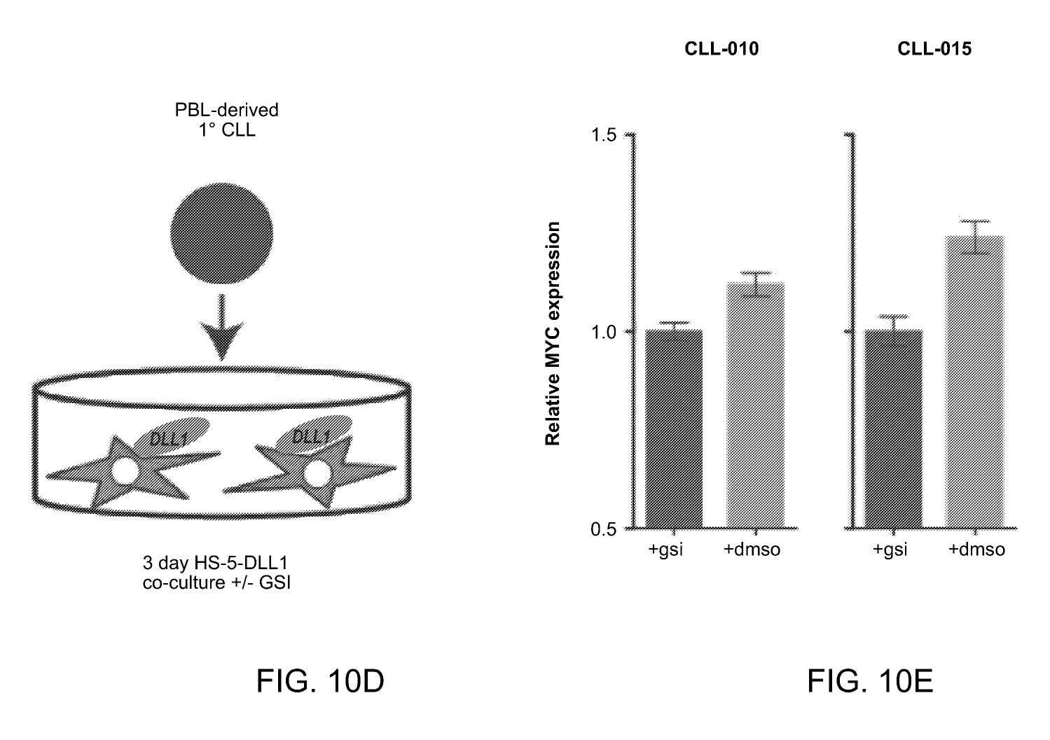

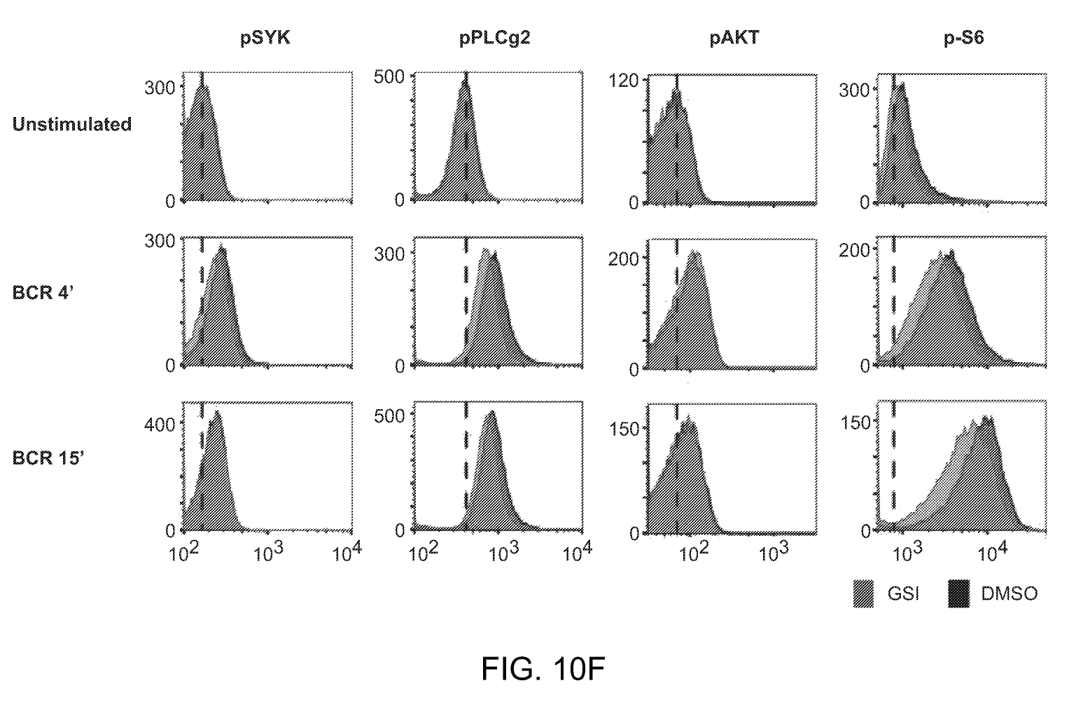

Since rapidly proliferating MCL cell lines show important biological differences from relatively low-grade MCL and CLL cells in vivo, experiments were conducted to validate the activity of Notch target genes and enhancers in primary CLL and MCL cells. RNA-Seq was performed on CLL lymph node biopsies with strong, diffuse ICN-1 staining by IHC and compared it to data from CLL lymph node biopsies with low ICN-1 staining (0 of 4 with NOTCH1 PEST domain mutations). Genome-wide analysis revealed significantly increased expression in the ICN1-high biopsies of many of the strongest Notch target genes identified in the cell line analysis (FIG. 10A), including genes implicated in B-cell receptor (BCR) signaling (FYN) and cytokine (IL6R) signaling, or associated with B cell activation (SEMA7A). As in the cell line models, GSEA analysis revealed up-regulation of MYC and NF-KB target gene signatures in ICN1-high versus ICN1-low CLL lymph nodes (Suppl), although MYC itself did not show a significant difference in expression.

Next, ChIP-Seq was performed for ICN1, RBPJ, and H3K27ac in CLL and MCL biopsies. One CLL (CLL-013) and one MCL (MCL-010) biopsy yielded a dramatically higher number of significant RBPJ peaks compared to the others, and both contained NOTCH1 PEST domain mutations (FIG. 7B). ICN1 enrichment was relatively poor in the primary samples, but again, the largest number of peaks were seen in CLL-013 and MCL-010. Both cases showed enrichment for ICN1 and RBPJ binding at enhancers linked to MYC and other Notch target genes (FIG. 10C and FIG. 7B). Furthermore, enhancers linked to Notch-regulated genes were acetylated in most primary CLL and MCL lymph node biopsies, but showed reduced acetylation in peripheral blood CLL samples, consistent with microenvironment-dependent activation.

To functionally demonstrate Notch-dependent activation of Notch target genes in primary CLL and MCL cells, a co-culture model with the immortalized human bone-marrow stromal cell line HS-5 was utilized, which has been widely employed to support the survival of CLL cells in vitro (FIG. 10D). Peripheral blood mononuclear cells from CLL patients were co-cultured for three days with HS-5 cells stably transduced with a DLL1-IRES-GFP transgene (HS5-DLL1) in the presence of GSI or vehicle, and then sorted CD19+CDS+CLL cells for analysis. Co-cultured CLL cells showed a significant and reproducible, albeit modest, increase in expression of MYC and other Notch target genes by qRT-PCR (FIG. 10E), while flow analysis showed a significant increase in cell surface proteins encoded by Notch target genes.

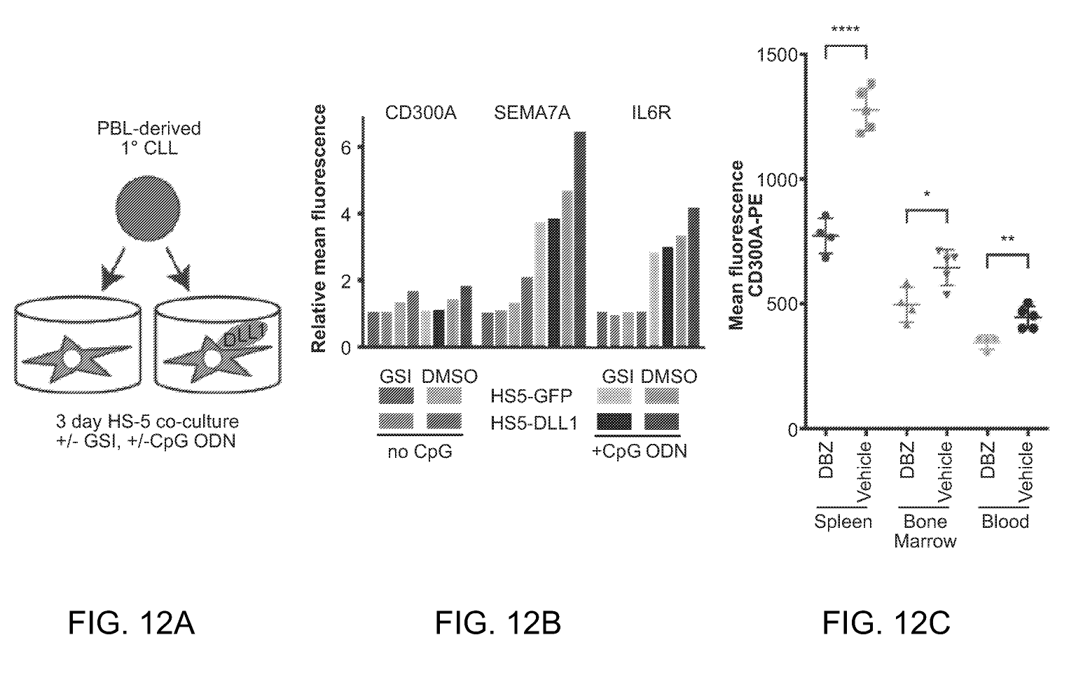

Next, the same model was used to evaluate the effect of Notch activation on the activity of signaling pathways linked to lymphoma proliferation and survival. CLL PBMC's were harvested following three days of co-culture with HS5-DLL1 with or without GSI, and then performed an additional brief incubation in the presence of absence of B-cell receptor (BCR)-crosslinking antibodies, followed by flow cytometric analysis of phosphoepitopes associated with BCR signaling and downstream pathways (FIG. 10F and FIG. 12A). As expected, BCR crosslinking was associated with a rapid increase in phosphorylation of proximal signaling mediators (p-SYK, p-PLCg2), MAP kinases (p-ERK, p-p38), pSTAT5, and mediators downstream of PI3 kinase and mTOR (pAKT, p-S6). Of all phospho-proteins evaluated, only ribosomal protein S6, a target of p70-S6 kinase downstream of mTORC1, showed a substantial notch-dependent increase in phosphorylation in the absence of BCR signaling. This Notch-dependent increase in S6 phosphorylation was still maintained in the setting of a 10-fold increase in S6 phosphorylation seen at 15 minutes after BCR crosslinking. A Notch-dependent difference in AKT phosphorylation was not detected either at rest or upon PI3K-AKT activation by BCR crosslinking, indicating that Notch activates S6 phosphorylation through a pathway independent of BCR signaling or PI3K-AKT activation.

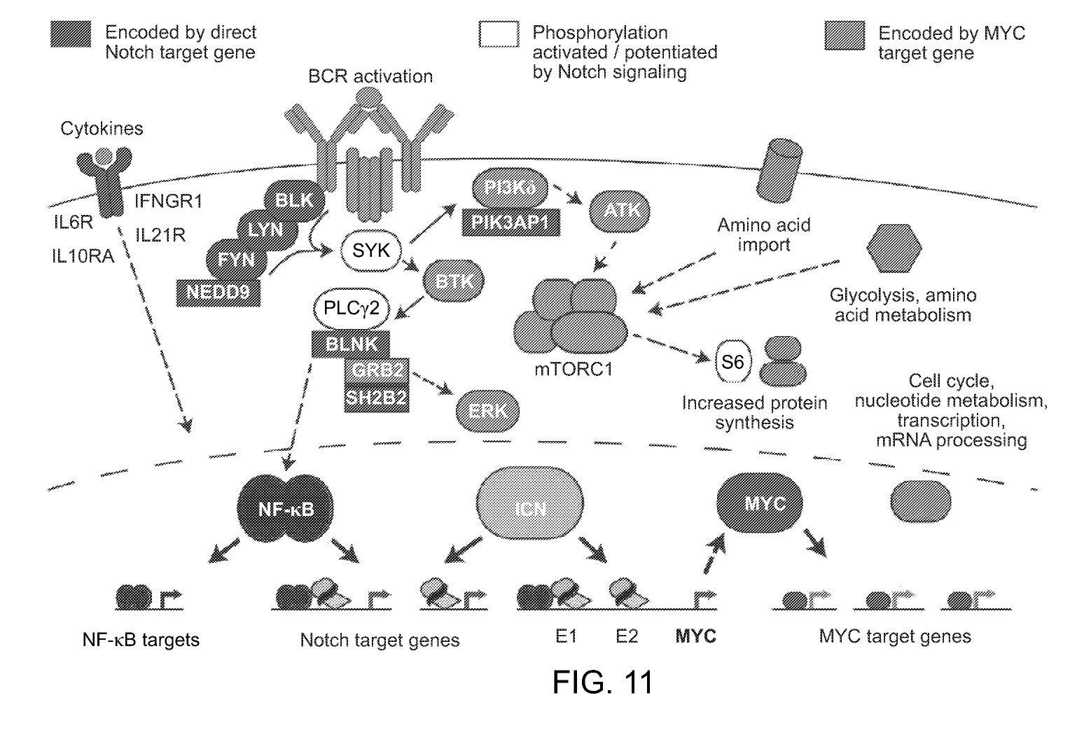

Proximal BCR signaling mediators did not show a notch-dependent difference in phosphorylation in the absence of stimulation, but significantly greater phosphorylation of SYK and PLCg2 were noted in Notch-on CLL cells upon BCR crosslinking. These findings indicate that Notch potentiates BCR signaling via up-regulation of proximal pathway regulators, resulting in increased NF-KB activity upon initiation of BCR signaling (FIG. 10F, FIG. 11).

NF-KB is known to be a strong activator of enhancer-mediated gene expression, and in fact, published ChIP-Seq datasets from LCLs show NF-KB protein binding at many ICN-1 bound enhancers, indicating that NF-KB and Notch may act cooperatively to activate many target genes. To test this, additional CLL HS-5 co-culture experiments were performed in the presence of CpG-rich oligodideoxynucleotides, which act as a strong agonist of Toll-like receptor 9 (TLR9) signaling (FIG. 12A). The toll-like receptor signaling pathway activates NF-KB independent of the BCR signaling pathway, and is mutationally activated in a minority of CLL cases. CLL surface expression of CD300A was increased by Notch signaling, but unaffected by TLR activation, while SEMA7A showed additive increases in expression due to Notch and TLR signaling, and the activation of IL6R expression by Notch was detectable only in the presence of concomitant TLR activation, indicating a synergistic effect (FIG. 12B)

### Example 6

### Notch Target Genes Show Microenvironment-Specific Activation in MCL in Vivo

Implicit in the present investigation of CLL and MCL lymph node biopsies, as well as co-culture model described herein, is the assumption that Notch activation occurs due to interaction of lymphoma cells with Notch ligand-expressing cells within the lymph node microenvironment. To support this in vivo, a patient-derived xenograft (PDX) model derived from a case of MCL with a NOTCH1 PEST domain mutation was utilized.

Immunohistochemistry showed strong expression of ICN1 in MCL cells within the spleen, but minimal staining in three different, NOTCH1 wild-type MCL PDX models. PDX-XXX mice were treated for five days with either the gamma-secretase inhibitor DBZ or vehicle. Flow cytometry revealed the highest expression of Notch target cell surface proteins in MCL cells within the spleen compared to bone marrow or blood, with substantially decreased expression seen in GSI-treated animals (FIG. 12C).

Since the initial discovery of recurrent Notch gene mutations in CLL and MCL, it has been clear that aberrant Notch signaling plays a role in the etiology of small B cell lymphomas, but the specific mechanisms by which Notch signaling drives B cell lymphoma growth, and its interaction with other oncogenic signaling pathways have remained largely obscure. The present study reported herein represents a substantial advance by defining a set of direct Notch regulatory targets in B cell lymphoma that is distinct from those identified in other tissue types, indicating unique mechanisms by which small B-cell lymphomas may utilize this pathway to drive malignant biology.

The data presented herein provides the first demonstration of MYC as a critical and direct regulatory target of enhancer activation by ICN/RBPJ in small B cell lymphomas, and the findings reported herein are consistent with other recent data linking Notch signaling to MYC activation in CLL. The BNDME sites are recurrently amplified in a small subset of CLL cases, and an enhancer-like element immediately adjacent to BNDME1 contains a germline polymorphism linked by genome-wide association studies (GWAS) to hereditary risk for CLL, further supporting the central role of these elements in CLL pathogenesis. MYC is a pivotal regulator of cellular growth, directly activating genes responsible for nutrient import, metabolic pathway activation, nucleotide synthesis and core components of the transcriptional and translational machinery. MYC is essential for the proliferation of normal mature B and T cells, as well as most, if not all B-cell lymphomas, and activating genomic rearrangements of the MYC locus are frequently seen in aggressive B cell lymphomas, including blastic transformation of MCL and large-cell transformation of CLL (Richter syndrome), where NOTCH1 mutations and MYC-activating genomic lesions show near-complete mutual exclusivity. Notch-dependent activation of MYC and MYC target genes appears to be a common feature of Notch-dependent cell lines across at least three cancer types (B-cell lymphoma, T-ALL, and TNBC), although the specific distal regulatory elements through which Notch activates MYC in B-cell lymphomas are not utilized in T-ALL. The data presented herein indicates that inhibition of Notch-dependent MYC expression is the primary mechanism by which GSI inhibits growth of Notch-dependent MCL cell lines, since a similar loss of MYC expression and proliferation could be demonstrated via direct CRISPR-Cas9 targeting of the 5′ BNDME sites, while conversely, GSI sensitivity could be largely rescued via expression of a MYC transgene (FIG. 2).

CLL and MCL are considered to be low-grade lymphomas, and it is important to note that the growth cycle of these tumors in vivo is different from that of the rapidly proliferating MCL cell lines utilized in the present study (doubling time 24-36 hours). Clinical and biological observations demonstrate that most cases of MCL show slow tumor growth for years after initial presentation, while the majority of CLL cells in most patients are in a quiescent state in both peripheral blood and secondary lymphoid organs, with bursts of proliferation limited to a small subset of cells in proliferation centers. However, the data presented herein, and the findings others, supports an important role for Notch-dependent MYC activation in driving a shift toward anabolic metabolism in primary CLL cells, which may facilitate subsequent cellular growth and proliferation. Co-culture of CLL cells with Notch ligand-expressing stromal cells has been shown to activate expression of hexokinase II and other MYC-activated metabolic regulators, resulting in activation of glycolysis. During activation of normal T cells, MYC is required for initiation of glycolysis and altered amino acid transport and metabolism, resulting in activation of p70-S6 kinase and other mTORC-regulated drivers of protein synthesis. The data presented herein from both proliferating cell lines and non-proliferating primary CLL cells is consistent with an analogous model in which Notch-dependent MYC activation leads to up-regulation of nutrient transporters, as well as HK2 and other metabolic gatekeepers, leading to activation of mTORC1 and S6 phosphorylation. This mechanism could play an important role in the growth of CLL and MCL cells during either proliferation or a pre-proliferative state.

In addition to activating MYC, the data indicated that Notch directly activates genes that encode regulators of B-cell receptor (BCR) signaling, including all three of the SRC family kinases implicated in proximal BCR activation (LYN, BLK, and FYN), as well as signaling adaptor proteins associated with PI3 kinase (PIK3AP; encodes BCAP) and phospholipase C gamma 2 (BLNK). While many details about the oncogenic role of BCR signaling in CLL and MCL are still unclear, phosphorylation of PLCy2 by Bruton tyrosine kinase (BTK) appears to be a critical step, since treatment with the BTK inhibitor ibrutinib drives sustained clinical remission in many CLL and MCL patients, while acquired ibrutinib resistance in lymphoma is often associated with mutations in BTK or PLCG2. A reproducibly stronger increase was observed in PLCy2 phosphorylation upon BCR signaling activation in “notch on” versus GSI-treated CLL cells from HS-5-DLL1 co-cultures, demonstrating that Notch activation potentiates this step of the BCR signaling cascade, likely through increased expression of one or more of the Notch target genes described above.

The validation studies were focused on the MYC and BCR signaling pathways, this work also identified genes encoding a striking array of cell surface signaling receptors as direct Notch targets, including receptors for IL6, IL10, and IL21, interferon gamma, TNF, and others, indicating that Notch may also potentiate signaling through these pathways. IL6R is a particularly strong Notch target, and has been implicated in the pathogenesis of both small B cell lymphomas and several autoimmune disorders. IL6R was among the Notch target genes that showed significantly increased expression in ICN1-high CLL (FIG. 10B), and given the availability of an FDA-approved antibody inhibitor of IL6R, the potential value of anti-IL6R therapy in Notch-mutant CLL could be worth further investigation. It is likely that many of the direct Notch target genes identified in this study may be regulated by Notch in normal immunity or autoimmune disease, and in this context it is interesting to note that several direct Notch target genes lie in loci that have been linked by genome wide association studies to immunological disorders. Notch is known to play a critical role in the development of specific B cell subsets, since B cell-specific deletion of Rbpj or Notch2 results in absence of splenic marginal zone B cells (MZB) in mice. Interestingly, mice with homozygous inactivation of Nedd9, the human homolog of which was identified as a direct Notch target in this study, also results in absence of MZB, indicating that Notch-dependent activation of Nedd9 may play a critical role in development of this subset. The protein product of Nedd9 (also known as HEF1 or CAS-L) encodes a signaling adaptor known to play an important role in motility and mitosis. In B cells, NEDD9 associates with LYN or FYNto convey active integrin- or B-cell receptor signals to CRKL, which activates downstream effectors involved in cytoskeletal regulation and motility. Interference with BCR- and integrin-mediated trafficking signals has been cited as an important therapeutic mechanism of action for ibrutinib in CLL (De Rooij et al., 2012). Given that the data presented herein identification of NEDD9 and FYN as strong direct Notch targets in MCL cell lines, and as significantly up-regulated genes in ICN1-high CLL, the role of Notch signaling in regulation of lymphoma adhesion and trafficking merits further study.

The findings presented herein have important implications for the potential use of Notch inhibitors in the treatment of small B cell lymphomas. Notch signaling in lymphomas with wild-type or PEST domain-mutated Notch receptors is predicted to be largely or entirely ligand-dependent, and thus Notch inhibitors might be expected to have little effect on circulating lymphoma cells outside of secondary lymphoid organs, or other microenvironments that support Notch signaling activation. However, there is precedent for selectively targeting lymphoma within a tissue niche, as clinically efficacious agents that inhibit BCR-related signaling, including ibrutinib and the PI3K6 inhibitor idelalisib, show minimal toxicity to circulating CLL cells, and in fact, treatment with these agents is frequently associated with sustained tumor lymphocytosis, despite dramatic shrinkage of lymphadenopathy and eventual clinical remission. BCR signaling-mediated activation of NF-KB, as well as up-regulation of MYC and MYC target genes, are believed to be critical drivers of lymphoma proliferation and survival in the lymph node microenvironment. The potential of Notch inhibitor therapy to target both of these pathways by a single unique mechanism may provide an advantage over existing agents, either alone or in combination therapy. Mutations or rearrangements predicted to yield ligand-independent Notch signaling, as observed in Notch-dependent MCL lines, are essentially absent in low-grade CLL and MCL, although development of a NOTCH1 heterodimerization domain mutation has been observed following large cell (Richter) transformation of CLL. Such patients might represent particularly appealing candidates for Notch-targeting therapy. However, the data presented herein indicates that MYC-activating genomic rearrangements, which are relatively common following high-grade transformation of CLL or MCL, would be likely to show Notch-independent MYC expression and thus reduced susceptibility to Notch inhibitor therapy, indicating that clinical investigators might consider excluding such patients from future trials of Notch-targeting drugs.

The results described herein above, were obtained using the following methods and materials.

### Cell Lines and Specimen Collection

MCL-derived cell lines were kindly provided by Dr. Randy Gascoyne, BC Cancer Agency, Canada (Z-138, Maver-1, JVM-2, Granta-519, HBL-2, and UPN-1). The cell lines SP-49, Jeko-1 and Mino were kind gift of Dr. Mariusz Wasik, University of Pennsylvania. Rec-1 and HEK293T cell lines were purchased from the American Type Culture Collection. Mec-1 cells were obtained. All cell lines were authenticated by short tandem repeat (STR) profiling analysis. This study was approved by the Institutional Review Board and MCL and CLL patient samples were collected.

### Cell Culture and GSI Washout Assay

All cell lines were grown in RPMI medium 1640 (Invitrogen) supplemented with 10% FCS, 100 IU per 100 μg per mL penicillin/streptomycin, 1% nonessential amino acids, 1 mM sodium pyruvate and 5μM 2-mercaptoethanol. In GSI washout studies, Rec-1, Mino and SP-49 cells were treated with the GSI compound E (1 μM) (Shelton et al., 2009) for 48-72 hours, washed, and then replated in either 1 μM GSI (washout control) or in DMSO for 4 h (washout) as described in Weng et al., 2006. To activate Notch signaling Mino and Jeko-1 cells were cultured on either immobilized recombinant Notch ligand (DLL1^{ext}-IgG) or control protein (IgG) for 48 hours supplemented with either DMSO or 1 μM GSI, following mock or GSI washout for 4 hours.

### Western Blotting

Cells were lysed in 50 mM Tris, pH 8.0, containing 150 mM NaCl, 1% NP40, 0.5% sodium deoxycholate, 0.1% SDS, 1 mM EDTA and supplemented with protease inhibitors. Total protein was determined. Samples were mixed with sample buffer containing 5% f3-mercaptoethanol, separated by 4% to 12% NuPAGE Tris-Acetate gel (Life technologies) and transferred to a nitrocellulose membrane that was blocked for 1 hour in 5% non fat dry milk/BSA in TBST (20 mmol/L Tris-HCl, 0.5 mol/L NaCl, and 0.1% Tween 20). The membrane was probed and incubated with a primary antibody overnight at 4° C. Following washes with TBST, the membrane was incubated with horseradish peroxidase-conjugated secondary antibody (Ref) and detected with ECL developing solution (Thermo Scientific). Primary antibodies used are a monoclonal rabbit antibody against the cleaved Notch1 (Val1744, CST; #4147) in 1:1000 dilution, c-MYC and TBP.

### Quantitative Real-Time PCR

RNA was isolated using the RNeasy Plus Mini Kit (Qiagen). cDNA was synthetized with the SuperScript III kit (Invitrogen). qRT-PCR was carried out using 1 μL cDNA, SYBR Green PCR Master Mix (ABI) and gene-specific primers (supplementary table 1) on an ABI ViiA 7 real-time PCR System. cDNA was used as template for each pair of primers in triplicate PCR reactions and resulting qPCR data were analyzed using the ΔΔCₜ relative quantification protocol.

### Chromatin Immunoprecipitation Assay

ChIP-qPCR and ChIP-Seq were performed as previously described (Ref). Briefly, chromatin samples prepared from fixed cells were immunoprecipitated with rabbit IgG (Santa Cruz Biotechnology, sc-3888), rabbit monoclonal anti-Rbpj (CST, #5313), rabbit polyclonal anti-H3K27ac (Active Motif, #39133) and mouse monoclonal anti-EBNA2(PE2) antibody (Abcam, ab90543). Antibody-chromatin complexes were captured with protein G-conjugated agarose beads, washed several times, and eluted. Following reversal of cross-links, RNase and proteinase K treatment, DNA was purified with QIAquick PCR Purification Kit (Qiagen). Input sample was prepared in parallel without immunoprecipitation. Real-time PCR was performed in triplicates for indicated regions using primers listed in supplementary table 2. For ChIP-Seq two replicates were used per experimental condition and libraries were prepared using NEBNext® Ultra™ DNA Library Prep Kit for Illumina according to the manufacturer's instructions. Indexed libraries were validated for quality and size distribution using the Agilent 2100 Bioanalyzer. High-throughput sequencing was performed by using the HiSeq 2500 Illumina Genome Analyzer. ChIP-Seq reads were aligned to the human genome (hg19).

### Lentiviral Infection and Cell Sorting

Lentiviral particles were generated with the use of standard procedures (Ref). Briefly, lentivirus was produced in HEK293T cells that were transfected with transfection mix containing 3.9 pg of gRNA expression vectors (Addgene, #57822, #57823, #52963) or pHR-SFFV-KRAB-dCas9-P2A-mCherry (Addgene, #60954), 1.3 μg of pCMV-VSV-G and 2.6 μg pCMV-delta and FuGENE HD (Promega). Viral supernatant was harvested 48 hours post-transfection. Cell lines were transduced with lentiviral supernatants by spinfection for 90 minutes in the presence of 12 μg/ml of polybrene at 37° C. 3 days after infection, transduced cells were selected either with puromycin (3 days), or were selected by fluorescent marker with cell sorting on a BD FACSAria II SORP. Selected cells were used for RNA extraction and proliferation assay.

### RNA-Seq

RNA-Seq was performed using three replicates per experimental condition. RNA was isolated with RNeasy Plus Mini Kit (Qiagen) from SP-49 cells treated with GSI for 3 days to establish a Notch-off state or cells where Notch was re-activated by GSI washout as described in GSI washout assay or from Mino cells that were cultured with the following modification: supplemented with either immobilized recombinant Notch ligand (DLL1^{ext}-IgG) or control protein (IgG) for 48 hours of purified mRNA was used as template for cDNA synthesis and library construction. Indexed libraries were validated for quality and size distribution using the Agilent 2100 Bioanalyzer and were sequenced on the HiSeq 2500 Illumina Genome Analyzer.

### MYC Rescue Experiment

SP-49 cells were stably transduced with pINDUCER-22-MYC (Ref) and single cell clones were isolated by limiting dilution with plating 0.3 cells/well in 96 well plates. Selected clones were treated with DMSO or GSI for 5 days and then MYC expression was induced by increasing concentration of doxycycline for 2 days and cell growth was measured using the CellTiter-Glo Luminescent Cell viability assay (Promega) as recommended by the manufacturer.

### Proliferation Assay After Silencing CR2 and CD300A Regulatory Elements

SP-49 and Granta-519 were engineered to stably express SFFV-KRAB-dCas9-P2A-mCherry or pLX-304-GFP. GFP+and dCas9-KRAB-mCherry+cells derived from SP-49 or Granta-519 were mixed in 1:1 ratio and transduced with gRNA lentiviruses designed against CD300A and CR2 regulatory regions (gRNA sequences are provided in supplementary table 3), following the puromycin selection for 3 days. Flow antibodies against CR2 and CD300A (Ref) were used to detect the expression in GFP+(negative control) and dCas9-KRAB-mCherry+populations following the epigenetic silencing of CR2 and CD300A.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.
Aster, J. C., 2014. In brief: Notch signalling in health and disease. J. Pathol. 232, 1-3. doi:10.1002/path.4291
Astier, a, Manié, S. N., Law, S. F., Canty, T., Haghayghi, N., Druker, B. J., Salgia, R., Golemis, E. a, Freedman, a S., 1997. Association of the Cas-like molecule HEF1 with CrkL following integrin and antigen receptor signaling in human B-cells: potential relevance to neoplastic lymphohematopoietic cells. Leuk. Lymphoma 28, 65-72. doi:10.3109/10428199709058332
Beá, S., Valdés-Mas, R., Navarro, A., Salaverria, I., Martin-Garcia, D., Jares, P., Giné, E., Pinyol, M., Royo, C., Nadeu, F., Conde, L., Juan, M., Clot, G., Vizán, P., Di Croce, L., Puente, D. a, López-Guerra, M., Moros, A., Roue, G., Aymerich, M., Villamor, N., Colomo, L., Martinez, A., Valera, A., Martin-Subero, J. I., Amador, V., Hernández, L., Rozman, M., Enjuanes, A., Forcada, P., Muntanñola, A., Hartmann, E. M., Calasanz, M. J., Rosenwald, A., Ott, G., Hernández-Rivas, J. M., Klapper, W., Siebert, R., Wiestner, A., Wilson, W. H., Colomer, D., López-Guillermo, A., López-Otin, C., Puente, X. S., Campo, E., 2013. Landscape of somatic mutations and clonal evolution in mantle cell lymphoma. Proc. Natl. Acad. Sci. U. S. A. 110,18250-5. doi: 10.1073/pnas.1314608110
Bo, M. D., Del Principe, M. I., Pozzo, F., Ragusa, D., Bulian, P., Rossi, D., Capelli, G., Rossi, F. M., Niscola, P., Buccisano, F., Bomben, R., Zucchetto, A., Maurillo, L., de Fabritiis, P., Amadori, S., Gaidano, G., Gattei, V., Del Poeta, G., 2014. NOTCH1 mutations identify a chronic lymphocytic leukemia patient subset with worse prognosis in the setting of a rituximab-based induction and consolidation treatment. Ann. Hematol. 93,1765-1774. doi:10.1007/s00277-014-2117-x
Chan, S. M., Weng, A. P., Tibshirani, R., Aster, J. C., Utz, P. J., 2007. Notch signals positively regulate activity of the mTOR pathway in T-cell acute lymphoblastic leukemia. Blood 110,278-286. doi:10.1182/blood-2006-08-039883
De Rooij, M. F. M., Kuil, A., Geest, C. R., Eldering, E., Chang, B. Y., Buggy, J. J., Pals, S. T., Spaargaren, M., 2012. The clinically active BTK inhibitor PCI-32765 targets B-cell receptor- and chemokine-controlled adhesion and migration in chronic lymphocytic leukemia. Blood 119, 2590-2594. doi:10.1182/blood-2011-11-390989
Fabbri, G., Khiabanian, H., Holmes, A. B., Wang, J., Messina, M., Mullighan, C. G., Pasqualucci, L., Rabadan, R., Dalla-Favera, R., 2013. Genetic lesions associated with chronic lymphocytic leukemia transformation to Richter syndrome. J. Exp. Med. 210,2273-88. doi:10.1084/jem.20131448
Fabbri, G., Rasi, S., Rossi, D., Trifonov, V., Khiabanian, H., Ma, J., Grunn, A., Fangazio, M., Capello, D., Monti, S., Cresta, S., Gargiulo, E., Forconi, F., Guarini, A., Arcaini, L., Paulli, M., Laurenti, L., Larocca, L. M., Marasca, R., Gattei, V., Oscier, D., Bertoni, F., Mullighan, C. G., Foá, R., Pasqualucci, L., Rabadan, R., Dalla-Favera, R., Gaidano, G., 2011. Analysis of the chronic lymphocytic leukemia coding genome: role of NOTCH1 mutational activation. J. Exp. Med. 208,1389-401. doi:10.1084/jem.20110921
Herishanu, Y., Perez-Galan, P., Liu, D., Biancotto, a., Pittaluga, S., Vire, B., Gibellini, F., Njuguna, N., Lee, E., Stennett, L., Raghavachari, N., Liu, P., McCoy, J. P., Raffeld, M., Stetler-Stevenson, M., Yuan, C., Sherry, R., Arthur, D. C., Maric, I., White, T., Marti, G. E., Munson, P., Wilson, W. H., Wiestner, a., 2011. The lymph node microenvironment promotes B-cell receptor signaling, NF-B activation, and tumor proliferation in chronic lymphocytic leukemia. Blood 117,563-574. doi:10.1182/blood-2010-05-284984
Herranz, D., Ambesi-Impiombato, A., Palomero, T., Schnell, S., Belver, L., Wendorff, A., Xu, L., Castillo-Martin, M., Llobet-Navás, D., Cordon-Cardo, C., Clappier, E., Soulier, J., Ferrando, A. a, 2014. A NOTCH1-driven MYC enhancer promotes T cell development, transformation and acute lymphoblastic leukemia. Nat. Med. 20,1130-1137. doi:10.1038/nm.3665
Jitschin, R., Braun, M., Qorraj, M., Saul, D., Blanc, K. Le, Zenz, T., 2015. Stromal cell-mediated glycolytic switch in CLL cells involves Notch-c-Myc signaling. Blood 125,3432-3437. doi:10.1182/blood-2014-10-607036.The
Kluk, M. J., Ashworth, T., Wang, H., Knoechel, B., Mason, E. F., Morgan, E. A., Dorfman, D., Pinkus, G., Weigert, O., Hornick, J. L., Chirieac, L. R., Hirsch, M., Oh, D. J., South, A. P., Leigh, I. M., Pourreyron, C., Cassidy, A. J., Deangelo, D. J., Weinstock, D. M., Krop, I. E., Dillon, D., Brock, J. E., Lazar, A. J. F., Peto, M., Cho, R. J., Stoeck, A., Haines, B. B., Sathayanrayanan, S., Rodig, S., Aster, J. C., 2013. Gauging NOTCH1 Activation in Cancer Using Immunohistochemistry. PLoS One 8, e67306. doi:10.1371/journal.pone.0067306
Kridel, R., Meissner, B., Rogic, S., Boyle, M., Telenius, A., Woolcock, B., Gunawardana, J., Jenkins, C., Cochrane, C., Ben-Neriah, S., Tan, K., Morin, R. D., Opat, S., Sehn, L. H., Connors, J. M., Marra, M. a, Weng, A. P., Steidl, C., Gascoyne, R. D., 2012. Whole transcriptome sequencing reveals recurrent NOTCH1 mutations in mantle cell lymphoma. Blood 119,1963-71. doi:10.1182/blood-2011-11-391474
Mania, S. N., Beck, A. R. P., Astier, A., Law, S. F., Canty, T., Hirai, H., Druker, B. J., Avraham, H., Haghayeghi, N., Sattler, M., Salgia, R., Griffin, J. D., Golemis, E. A., Freedman, A. S., 1997. Involvement of p130(Cas) and p105(HEF1), a novel Cas-like docking protein, in a cytoskeleton-dependent signaling pathway initiated by ligation of integrin or antigen receptor on human B cells. J. Biol. Chem. 272, 4230-4236. doi:10.1074/jbc.272.7.4230
Minegishi, M., Tachibana, K., Sato, T., Iwata, S., Nojima, Y., Morimoto, C., 1996. Structure and function of Cas-L, a 105-kD Crk-associated substrate-related protein that is involved in beta 1 integrin-mediated signaling in lymphocytes. J. Exp. Med. 184, 1365-75. doi:0.1084/jem.184.4.1365
Puente, X. S., Beá, S., Valdés-Mas, R., Villamor, N., Gutiérrez-Abril, J., Martin-Subero, J. I., Munar, M., Rubio-Perez, C., Jares, P., Aymerich, M., Baumann, T., Beekman, R., Belver, L., Carrio, A., Castellano, G., Clot, G., Colado, E., Colomer, D., Costa, D., Delgado, J., Enjuanes, A., Estivill, X., Ferrando, A. a., Gelpi, J. L., González, B., González, S., González, M., Gut, M., Hernández-Rivas, J. M., López-Guerra, M., Martin-Garcia, D., Navarro, A., Nicolás, P., Orozco, M., Payer, Á. R., Pinyol, M., Pisano, D. G., Puente, D. a., Queirós, A. C., Quesada, V., Romeo-Casabona, C. M., Royo, C., Royo, R., Rozman, M., Russifiol, N., Salaverria, I., Stamatopoulos, K., Stunnenberg, H.G., Tamborero, D., Terol, M.J., Valencia, A., López-Bigas, N., Torrents, D., Gut, I., López-Guillermo, A., López-Otin, C., Campo, E., 2015. Non-coding recurrent mutations in chronic lymphocytic leukaemia. Nature. doi:10.1038/nature14666
Puente, X. S., Pinyol, M., Quesada, V., Conde, L., Ordòñez, G. R., Villamor, N., Escaramis, G., Jares, P., Beá, S., González-Diaz, M., Bassaganyas, L., Baumann, T., Juan, M., López-Guerra, M., Colomer, D., Tubio, J. M. C., López, C., Navarro, A., Tornador, C., Aymerich, M., Rozman, M., Hernández, J. M., Puente, D. A., Freije, J. M. P., Velasco, G., Gutiérrez-Fernandez, A., Costa, D., Carrió, A., Guijarro, S., Enjuanes, A., Hernández, L., Yagüe, J., Nicolás, P., Romeo-Casabona, C. M., Himmelbauer, H., Castillo, E., Dohm, J. C., de Sanjosé, S., Pins, M. A., de Alava, E., Miguel, J. S., Royo, R., Gelpi, J. L., Torrents, D., Orozco, M., Pisano, D. G., Valencia, A., Guigó, R., Bayés, M., Heath, S., Gut, M., Klatt, P., Marshall, J., Raine, K., Stebbings, L. A., Futreal, P. A., Stratton, M. R., Campbell, P. J., Gut, I., López-Guillermo, A., Estivill, X., Montserrat, E., López-Otin, C., Campo, E., 2011. Whole-genome sequencing identifies recurrent mutations in chronic lymphocytic leukaemia. Nature 475, 101-105. doi:10.1038/nature10113
Pugacheva, E. N., Golemis, E. A., 2005. The focal adhesion scaffolding protein HEF1 regulates activation of the Aurora-A and Nek2 kinases at the centrosome. Nat. Cell Biol. 7, 937-46. doi:10.1038/ncb1309
Rossi, D., Rasi, S., Fabbri, G., Spina, V., Fangazio, M., Forconi, F., Marasca, R., Laurenti, L., Bruscaggin, A., Cerri, M., Monti, S., Cresta, S., Famá, R., De Paoli, L., Bulian, P., Gattei, V., Guarini, A., Deaglio, S., Capello, D., Rabadan, R., Pasqualucci, L., Dalla-Favera, R., Foá, R., Gaidano, G., 2011. Mutations of NOTCH1 are an independent predictor of survival in chronic lymphocytic leukemia. Blood 521-529. doi:10.1182/blood-2011-09-379966
Ryan, R. J. H., Drier, Y., Whitton, H., Cotton, M. J., Kaur, J., Issner, R., Gillespie, S., Epstein, C. B., Nardi, V., Sohani, A. R., Hochberg, E. P., Bernstein, B. E., 2015. Detection of Enhancer-Associated Rearrangements Reveals Mechanisms of Oncogene Dysregulation in B-cell Lymphoma. Cancer Discov. 5, 1058-1071. doi:10.1158/2159-8290.CD-15-0370
Seo, S., Asai, T., Saito, T., Suzuki, T., Morishita, Y., Nakamoto, T., Ichikawa, M., Yamamoto, G., Kawazu, M., Yamagata, T., Sakai, R., Mitani, K., Ogawa, S., Kurokawa, M., Chiba, S., Hirai, H., 2005. Crk-Associated Substrate Lymphocyte Type Is Required for Lymphocyte Trafficking and Marginal Zone B Cell Maintenance. J. Immunol. 175, 3492-3501. doi:10.4049/jimmuno1.175.6.3492
Singh, M. K., Cowell, L., Seo, S., O'Neill, G. M., Golemis, E. A., 2007. Molecular basis for HEF1/NEDD9/Cas-L action as a multifunctional co-ordinator of invasion, apoptosis and cell cycle. Cell Biochem. Biophys. 48, 54-72. doi:10.1007/s12013-007-0036-3
Stoeck, A., Lejnine, S., Truong, A., Pan, L., Wang, H., Zang, C., Yuan, J., Ware, C., MacLean, J., Garrett-Engele, P. W., Kluk, M., Laskey, J., Haines, B. B., Moskaluk, C., Zawel, L., Fawell, S., Gilliland, G., Zhang, T., Kremer, B. E., Knoechel, B., Bernstein, B. E., Pear, W. S., Liu, X. S., Aster, J. C., Sathyanarayanan, S., 2014. Discovery of biomarkers predictive of GSI response in triple-negative breast cancer and adenoid cystic carcinoma. Cancer Discov. 4, 1154-67. doi: 10.1158/2159-8290.CD-13-0830
Swerdlow, S. H., Campo, E., Harris, N. L., Jaffe, E. S., Pileri, S. A., Stein, H., Thiele, J., Vardiman, J. W. (Eds.), 2008. WHO Classification of Tumors of Haematopoietic and Lymphoid Tissues, 4th ed. International Agency for Research on Cancer, Lyon.
Tang, Z., Luo, O. J., Li, X., Zheng, M., Zhu, J. J., Szalaj, P., Trzaskoma, P., Magalska, A., Wlodarczyk, J., Ruszczycki, B., Michalski, P., Piecuch, E., Wang, P., Wang, D., Tian, S. Z., Penrad-Mobayed, M., Sachs, L. M., Ruan, X., Wei, C. L., Liu, E. T., Wilczynski, G. M., Plewczynski, D., Li, G., Ruan, Y., 2015. CTCF-Mediated Human 3D Genome Architecture Reveals Chromatin Topology for Transcription. Cell 163, 1611-1627. doi: 10.1016/j.ce11.2015.11.024
Tanigaki, K., Han, H., Yamamoto, N., Tashiro, K., Ikegawa, M., Kuroda, K., Suzuki, A., Nakano, T., Honjo, T., 2002. Notch-RBP-J signaling is involved in cell fate determination of marginal zone B cells. Nat. Immunol. 3, 443-450. doi:10.1038/ni793
Wang, H., Zang, C., Liu, X. S., Aster, J. C., 2015. The Role of Notch Receptors in Transcriptional Regulation. J. Cell. Physiol. 230, 982-988. doi:10.1002/jcp.24872
Wang, R., Dillon, C. P., Shi, L. Z., Milasta, S., Carter, R., Finkelstein, D., McCormick, L. L., Fitzgerald, P., Chi, H., Munger, J., Green, D. R., 2011. The Transcription Factor Myc Controls Metabolic Reprogramming upon T Lymphocyte Activation. Immunity 35, 871-882. doi:10.1016/j.immuni.2011.09.021
Weng, A. P., Ferrando, A. A., Lee, W., Morris, J. P., Silverman, L. B., Sanchez-Irizarry, C., Blacklow, S. C., Look, A. T., Aster, J. C., 2004. Activating mutations of NOTCH1 in human T cell acute lymphoblastic leukemia. Science 306, 269-271. doi:10.1126/science.1102160
Yashiro-Ohtani, Y., Wang, H., Zang, C., Arnett, K. L., Bailis, W., Ho, Y., Knoechel, B., Lanauze, C., Louis, L., Forsyth, K. S., Chen, S., Chung, Y., Schug, J., Blobel, G. a., Liebhaber, S. a., Bernstein, B. E., Blacklow, S. C., Liu, X. S., Aster, J. C., Pear, W. S., 2014. Long-range enhancer activity determines Myc sensitivity to Notch inhibitors in T cell leukemia. Proc. Natl. Acad. Sci. 111, E4946-E4953. doi:10.1073/pnas.1407079111
Zhao, B., Zou, J., Wang, H., Johannsen, E., Peng, C. -w., Quackenbush, J., Mar, J. C., Morton, C. C., Freedman, M. L., Blacklow, S. C., Aster, J. C., Bernstein, B. E., Kieff, E., 2011. Epstein-Barr virus exploits intrinsic B-lymphocyte transcription programs to achieve immortal cell growth. Proc. Natl. Acad. Sci. 108, 14902-14907. doi:10.1073/pnas.1108892108

1-16. (canceled)

17. A method of inhibiting the survival or proliferation of a neoplastic cell, the method comprising contacting the cell with an agent that inhibits expression or activity of a Notch polynucleotide or polypeptide and an effective amount of an agent that inhibits expression or activity of a functional component of a B cell receptor polypeptide or polynucleotide

18. The method of claim 17, wherein the agent that inhibits Notch expression or activity is a gamma secretase inhibitor, a Notch signaling pathway inhibitory antibody, or an anti-Notch1 antibody.

19. The method of claim 17, wherein the gamma secretase inhibitor is selected from the group consisting of Compound E, MK-0752, PF03084014, RO-4929097, DAPT, N-[N-(3,5-difluorophenacetyl)-L-alanyl]-S-phenylglycine t-butyl ester, tetralin imidazole PF-03084014, LY3039478, and BMS906-024.

20. The method of claim 17, wherein the anti-Notch1 antibody is OMP-52M521 and the Notch signaling pathway inhibitory antibody is an anti-Delta-like-4 antibody.

21. The method of claim 17, wherein the agent that inhibits Notch expression or activity is an inhibitory nucleic acid molecule.

22. The method of claim 17, wherein the agent that inhibits B cell receptor expression or activity is a PI3 kinase inhibitor, inhibitory nucleic acid molecule, BTK inhibitor, SRC family kinase inhibitor, SYK inhibitor, or a protein kinase C inhibitor.

23. The method of claim 22, wherein the BTK inhibitor is selected from the group consisting of ibrutinib, ACP-196, ONO/GS-4059, BGB-3111, and CC-292.

24. The method of claim 22, wherein the SRC family kinase inhibitor is Dasatinib and the PI3 kinase inhibitor is idelalisib.

25. The method of claim 22, wherein the SYK inhibitor is Fostamatinib.

26. The method of claim 22, wherein the protein kinase C inhibitor is Midostaurin, Enzastuarin, or Sotrasturin.

27. The method of claim 22, further comprising administration of one or more additional therapeutic agents.

28-56. (canceled)


SEQUENCE LISTING (22 sequences)

SEQ ID NO: 1 - protein, 226 aa, Homo sapiens
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 2 - protein, 229 aa, Homo sapiens
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 3 - protein, 659 aa, Homo sapiens
MAAVILESIFLKRSQQKKKTSPLNFKKRLFLLTVHKLSYYEYDFERGRRGSKKGSIDVEKITCVETVVPEKNPPPERQIPRRGEESSEMEQISIIERFPYPFQVVYDEGPLYVFSPTEELRKRWIHQLKNVIRYNSDLVQKYHPCFWIDGQYLCCSQTAKNAMGCQILENRNGSLKPGSSHRKTKKPLPPTPEEDQILKKPLPPEPAAAPVSTSELKKVVALYDYMPMNANDLQLRKGDEYFILEESNLPWWRARDKNGQEGYIPSNYVTEAEDSIEMYEWYSKHMTRSQAEQLLKQEGKEGGFIVRDSSKAGKYTVSVFAKSTGDPQGVIRHYVVCSTPQSQYYLAEKHLFSTIPELINYHQHNSAGLISRLKYPVSQQNKNAPSTAGLGYGSWEIDPKDLTFLKELGTGQFGVVKYGKWRGQYDVAIKMIKEGSMSEDEFIEEAKVMMNLSHEKLVQLYGVCTKQRPIFIITEYMANGCLLNYLREMRHRFQTQQLLEMCKDVCEAMEYLESKQFLHRDLAARNCLVNDQGVVKVSDFGLSRYVLDDEYTSSVGSKFPVRWSPPEVLMYSKFSSKSDIWAFGVLMWEIYSLGKMPYERFTNSETAEHIAQGLRLYRPHLASEKVYTIMYSCWHEKADERPTFKILLSNILDVMDEES

SEQ ID NO: 4 - DNA, 2611 bp, Homo sapiens
aactgagtggctgtgaaagggtggggtttgctcagactgtccttcctctctggactgtaagaatatgtctccagggccagtgtctgctgcgatcgagtcccaccttccaagtcctggcatctcaatgcatctgggaagctacctgcattaagtcaggactgagcacacaggtgaactccagaaagaagaagctatggccgcagtgattctggagagcatctttctgaagcgatcccaacagaaaaagaaaacatcacctctaaacttcaagaagcgcctgtttctcttgaccgtgcacaaactctcctactatgagtatgactttgaacgtgggagaagaggcagtaagaagggttcaatagatgttgagaagatcacttgtgttgaaacagtggttcctgaaaaaaatcctcctccagaaagacagattccgagaagaggtgaagagtccagtgaaatggagcaaatttcaatcattgaaaggttcccttatcccttccaggttgtatatgatgaagggcctctctacgtcttctccccaactgaagaactaaggaagcggtggattcaccagctcaaaaacgtaatccggtacaacagtgatctggttcagaaatatcacccttgcttctggatcgatgggcagtatctctgctgctctcagacagccaaaaatgctatgggctgccaaattttggagaacaggaatggaagcttaaaacctgggagttctcaccggaagacaaaaaagcctcttcccccaacgcctgaggaggaccagatcttgaaaaagccactaccgcctgagccagcagcagcaccagtctccacaagtgagctgaaaaaggttgtggccctttatgattacatgccaatgaatgcaaatgatctacagctgcggaagggtgatgaatattttatcttggaggaaagcaacttaccatggtggagagcacgagataaaaatgggcaggaaggctacattcctagtaactatgtcactgaagcagaagactccatagaaatgtatgagtggtattccaaacacatgactcggagtcaggctgagcaactgctaaagcaagaggggaaagaaggaggtttcattgtcagagactccagcaaagctggcaaatatacagtgtctgtgtttgctaaatccacaggggaccctcaaggggtgatacgtcattatgttgtgtgttccacacctcagagccagtattacctggctgagaagcaccttttcagcaccatccctgagctcattaactaccatcagcacaactctgcaggactcatatccaggctcaaatatccagtgtctcaacaaaacaagaatgcaccttccactgcaggcctgggatacggatcatgggaaattgatccaaaggacctgaccttcttgaaggagctggggactggacaatttggggtagtgaagtatgggaaatggagaggccagtacgacgtggccatcaagatgatcaaagaaggctccatgtctgaagatgaattcattgaagaagccaaagtcatgatgaatctttcccatgagaagctggtgcagttgtatggcgtctgcaccaagcagcgccccatcttcatcatcactgagtacatggccaatggctgcctcctgaactacctgagggagatgcgccaccgcttccagactcagcagctgctagagatgtgcaaggatgtctgtgaagccatggaatacctggagtcaaagcagttccttcaccgagacctggcagctcgaaactgtttggtaaacgatcaaggagttgttaaagtatctgatttcggcctgtccaggtatgtcctggatgatgaatacacaagctcagtaggctccaaatttccagtccggtggtccccaccggaagtcctgatgtatagcaagttcagcagcaaatctgacatttgggcttttggggttttgatgtgggaaatttactccctggggaagatgccatatgagagatttactaacagtgagactgctgaacacattgcccaaggcctacgtctctacaggcctcatctggcttcagagaaggtatataccatcatgtacagttgctggcatgagaaagcagatgagcgtcccactttcaaaattcttctgagcaatattctagatgtcatggatgaagaatcctgagctcgccaataagcttcttggttctacttctcttctccacaagccccaatttcactttctcagaggaaatcccaagcttaggagccctggagcctttgtgctcccactcaatacaaaaaggcccctctctacatctgggaatgcacctcttctttgattccctgggatagtggcttctgagcaaaggccaagaaattattgtgcctgaaatttcccgagagaattaagacagactgaatttgcgatgaaaatattttttaggagggaggatgtaaatagccgcacaaaggggtccaacagctctttgagtaggcatttggtagagcttgggggtgtgtgtgtgggggtggaccgaatttggcaagaatgaaatggtgtcataaagatgggaggggagggtgttttgataaaataaaattactagaaagcttgaaagtc

SEQ ID NO: 5 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 6 - DNA, 2121 bp, Homo sapiens
ctgctcgcggccgccaccgccgggccccggccgtccctggctcccctcctgcctcgagaagggcagggcttctcagaggcttggcgggaaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgcttgatcccccaggccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaa

SEQ ID NO: 7 - protein, 2555 aa, Homo sapiens
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPGLCRHGGTCHNEVGSYRCVCRATHTGPNCERPYVPCSPSPCQNGGTCRPTGDVTHECACLPGFTGQNCEENIDDCPGNNCKNGGACVDGVNTYNCRCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCICMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRHGGTCQDRDNAYLCFCLKGTTGPNCEINLDDCASSPCDSGTCLDKIDGYECACEPGYTGSMCNINIDECAGNPCHNGGTCEDGINGFTCRCPEGYHDPTCLSEVNECNSNPCVHGACRDSLNGYKCDCDPGWSGTNCDINNNECESNPCVNGGTCKDMTSGYVCTCREGFSGPNCQTNINECASNPCLNQGTCIDDVAGYKCNCLLPYTGATCEVVLAPCAPSPCRNGGECRQSEDYESFSCVCPTGWQGQTCEVDINECVLSPCRHGASCQNTHGGYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHDVNECDSQPCLHGGTCQDGCGSYRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 8 - DNA, 9322 bp, Homo sapiens
atgccgccgctcctggcgcccctgctctgcctggcgctgctgcccgcgctcgccgcacgaggcccgcgatgctcccagcccggtgagacctgcctgaatggcgggaagtgtgaagcggccaatggcacggaggcctgcgtctgtggcggggccttcgtgggcccgcgatgccaggaccccaacccgtgcctcagcaccccctgcaagaacgccgggacatgccacgtggtggaccgcagaggcgtggcagactatgcctgcagctgtgccctgggcttctctgggcccctctgcctgacacccctggacaatgcctgcctcaccaacccctgccgcaacgggggcacctgcgacctgctcacgctgacggagtacaagtgccgctgcccgcccggctggtcagggaaatcgtgccagcaggctgacccgtgcgcctccaacccctgcgccaacggtggccagtgcctgcccttcgaggcctcctacatctgccactgcccacccagcttccatggccccacctgccggcaggatgtcaacgagtgtggccagaagcccgggctttgccgccacggaggcacctgccacaacgaggtcggctcctaccgctgcgtctgccgcgccacccacactggccccaactgcgagcggccctacgtgccctgcagcccctcgccctgccagaacgggggcacctgccgccccacgggcgacgtcacccacgagtgtgcctgcctgccaggcttcaccggccagaactgtgaggaaaatatcgacgattgtccaggaaacaactgcaagaacgggggtgcctgtgtggacggcgtgaacacctacaactgccgctgcccgccagagtggacaggtcagtactgtaccgaggatgtggacgagtgccagctgatgccaaatgcctgccagaacggcgggacctgccacaacacccacggtggctacaactgcgtgtgtgtcaacggctggactggtgaggactgcagcgagaacattgatgactgtgccagcgccgcctgcttccacggcgccacctgccatgaccgtgtggcctccttctactgcgagtgtccccatggccgcacaggtctgctgtgccacctcaacgacgcatgcatcagcaacccctgtaacgagggctccaactgcgacaccaaccctgtcaatggcaaggccatctgcacctgcccctcggggtacacgggcccggcctgcagccaggacgtggatgagtgctcgctgggtgccaacccctgcgagcatgcgggcaagtgcatcaacacgctgggctccttcgagtgccagtgtctgcagggctacacgggcccccgatgcgagatcgacgtcaacgagtgcgtctcgaacccgtgccagaacgacgccacctgcctggaccagattggggagttccagtgcatctgcatgcccggctacgagggtgtgcactgcgaggtcaacacagacgagtgtgccagcagcccctgcctgcacaatggccgctgcctggacaagatcaatgagttccagtgcgagtgccccacgggcttcactgggcatctgtgccagtacgatgtggacgagtgtgccagcaccccctgcaagaatggtgccaagtgcctggacggacccaacacttacacctgtgtgtgcacggaagggtacacggggacgcactgcgaggtggacatcgatgagtgcgaccccgacccctgccactacggctcctgcaaggacggcgtcgccaccttcacctgcctctgccgcccaggctacacgggccaccactgcgagaccaacatcaacgagtgctccagccagccctgccgccacgggggcacctgccaggaccgcgacaacgcctacctctgcttctgcctgaaggggaccacaggacccaactgcgagatcaacctggatgactgtgccagcagcccctgcgactcgggcacctgtctggacaagatcgatggctacgagtgtgcctgtgagccgggctacacagggagcatgtgtaacatcaacatcgatgagtgtgcgggcaacccctgccacaacgggggcacctgcgaggacggcatcaatggcttcacctgccgctgccccgagggctaccacgaccccacctgcctgtctgaggtcaatgagtgcaacagcaacccctgcgtccacggggcctgccgggacagcctcaacgggtacaagtgcgactgtgaccctgggtggagtgggaccaactgtgacatcaacaacaatgagtgtgaatccaacccttgtgtcaacggcggcacctgcaaagacatgaccagtggctacgtgtgcacctgccgggagggcttcagcggtcccaactgccagaccaacatcaacgagtgtgcgtccaacccatgtctgaaccagggcacgtgtattgacgacgttgccgggtacaagtgcaactgcctgctgccctacacaggtgccacgtgtgaggtggtgctggccccgtgtgcccccagcccctgcagaaacggcggggagtgcaggcaatccgaggactatgagagcttctcctgtgtctgccccacgggctggcaagggcagacctgtgaggtcgacatcaacgagtgcgttctgagcccgtgccggcacggcgcatcctgccagaacacccacggcggctaccgctgccactgccaggccggctacagtgggcgcaactgcgagaccgacatcgacgactgccggcccaacccgtgtcacaacgggggctcctgcacagacggcatcaacacggccttctgcgactgcctgcccggcttccggggcactttctgtgaggaggacatcaacgagtgtgccagtgacccctgccgcaacggggccaactgcacggactgcgtggacagctacacgtgcacctgccccgcaggcttcagcgggatccactgtgagaacaacacgcctgactgcacagagagctcctgcttcaacggtggcacctgcgtggacggcatcaactcgttcacctgcctgtgtccacccggcttcacgggcagctactgccagcacgatgtcaatgagtgcgactcacagccctgcctgcatggcggcacctgtcaggacggctgcggctcctacaggtgcacctgcccccagggctacactggccccaactgccagaaccttgtgcactggtgtgactcctcgccctgcaagaacggcggcaaatgctggcagacccacacccagtaccgctgcgagtgccccagcggctggaccggcctttactgcgacgtgcccagcgtgtcctgtgaggtggctgcgcagcgacaaggtgttgacgttgcccgcctgtgccagcatggagggctctgtgtggacgcgggcaacacgcaccactgccgctgccaggcgggctacacaggcagctactgtgaggacctggtggacgagtgctcacccagcccctgccagaacggggccacctgcacggactacctgggcggctactcctgcaagtgcgtggccggctaccacggggtgaactgctctgaggagatcgacgagtgcctctcccacccctgccagaacgggggcacctgcctcgacctccccaacacctacaagtgctcctgcccacggggcactcagggtgtgcactgtgagatcaacgtggacgactgcaatccccccgttgaccccgtgtcccggagccccaagtgctttaacaacggcacctgcgtggaccaggtgggcggctacagctgcacctgcccgccgggcttcgtgggtgagcgctgtgagggggatgtcaacgagtgcctgtccaatccctgcgacgcccgtggcacccagaactgcgtgcagcgcgtcaatgacttccactgcgagtgccgtgctggtcacaccgggcgccgctgcgagtccgtcatcaatggctgcaaaggcaagccctgcaagaatgggggcacctgcgccgtggcctccaacaccgcccgcgggttcatctgcaagtgccctgcgggcttcgagggcgccacgtgtgagaatgacgctcgtacctgcggcagcctgcgctgcctcaacggcggcacatgcatctccggcccgcgcagccccacctgcctgtgcctgggccccttcacgggccccgaatgccagttcccggccagcagcccctgcctgggcggcaacccctgctacaaccaggggacctgtgagcccacatccgagagccccttctaccgttgcctgtgccccgccaaattcaacgggctcttgtgccacatcctggactacagcttcgggggtggggccgggcgcgacatccccccgccgctgatcgaggaggcgtgcgagctgcccgagtgccaggaggacgcgggcaacaaggtctgcagcctgcagtgcaacaaccacgcgtgcggctgggacggcggtgactgctccctcaacttcaatgacccctggaagaactgcacgcagtctctgcagtgctggaagtacttcagtgacggccactgtgacagccagtgcaactcagccggctgcctcttcgacggctttgactgccagcgtgcggaaggccagtgcaaccccctgtacgaccagtactgcaaggaccacttcagcgacgggcactgcgaccagggctgcaacagcgcggagtgcgagtgggacgggctggactgtgcggagcatgtacccgagaggctggcggccggcacgctggtggtggtggtgctgatgccgccggagcagctgcgcaacagctccttccacttcctgcgggagctcagccgcgtgctgcacaccaacgtggtcttcaagcgtgacgcacacggccagcagatgatcttcccctactacggccgcgaggaggagctgcgcaagcaccccatcaagcgtgccgccgagggctgggccgcacctgacgccctgctgggccaggtgaaggcctcgctgctccctggtggcagcgagggtgggcggcggcggagggagctggaccccatggacgtccgcggctccatcgtctacctggagattgacaaccggcagtgtgtgcaggcctcctcgcagtgcttccagagtgccaccgacgtggccgcattcctgggagcgctcgcctcgctgggcagcctcaacatcccctacaagatcgaggccgtgcagagtgagaccgtggagccgcccccgccggcgcagctgcacttcatgtacgtggcggcggccgcctttgtgcttctgttcttcgtgggctgcggggtgctgctgtcccgcaagcgccggcggcagcatggccagctctggttccctgagggcttcaaagtgtctgaggccagcaagaagaagcggcgggagcccctcggcgaggactccgtgggcctcaagcccctgaagaacgcttcagacggtgccctcatggacgacaaccagaatgagtggggggacgaggacctggagaccaagaagttccggttcgaggagcccgtggttctgcctgacctggacgaccagacagaccaccggcagtggactcagcagcacctggatgccgctgacctgcgcatgtctgccatggcccccacaccgccccagggtgaggttgacgccgactgcatggacgtcaatgtccgcgggcctgatggcttcaccccgctcatgatcgcctcctgcagcgggggcggcctggagacgggcaacagcgaggaagaggaggacgcgccggccgtcatctccgacttcatctaccagggcgccagcctgcacaaccagacagaccgcacgggcgagaccgccttgcacctggccgcccgctactcacgctctgatgccgccaagcgcctgctggaggccagcgcagatgccaacatccaggacaacatgggccgcaccccgctgcatgcggctgtgtctgccgacgcacaaggtgtcttccagatcctgatccggaaccgagccacagacctggatgcccgcatgcatgatggcacgacgccactgatcctggctgcccgcctggccgtggagggcatgctggaggacctcatcaactcacacgccgacgtcaacgccgtagatgacctgggcaagtccgccctgcactgggccgccgccgtgaacaatgtggatgccgcagttgtgctcctgaagaacggggctaacaaagatatgcagaacaacagggaggagacacccctgtttctggccgcccgggagggcagctacgagaccgccaaggtgctgctggaccactttgccaaccgggacatcacggatcatatggaccgcctgccgcgcgacatcgcacaggagcgcatgcatcacgacatcgtgaggctgctggacgagtacaacctggtgcgcagcccgcagctgcacggagccccgctggggggcacgcccaccctgtcgcccccgctctgctcgcccaacggctacctgggcagcctcaagcccggcgtgcagggcaagaaggtccgcaagcccagcagcaaaggcctggcctgtggaagcaaggaggccaaggacctcaaggcacggaggaagaagtcccaggacggcaagggctgcctgctggacagctccggcatgctctcgcccgtggactccctggagtcaccccatggctacctgtcagacgtggcctcgccgccactgctgccctccccgttccagcagtctccgtccgtgcccctcaaccacctgcctgggatgcccgacacccacctgggcatcgggcacctgaacgtggcggccaagcccgagatggcggcgctgggtgggggcggccggctggcctttgagactggcccacctcgtctctcccacctgcctgtggcctctggcaccagcaccgtcctgggctccagcagcggaggggccctgaatttcactgtgggcgggtccaccagtttgaatggtcaatgcgagtggctgtcccggctgcagagcggcatggtgccgaaccaatacaaccctctgcgggggagtgtggcaccaggccccctgagcacacaggccccctccctgcagcatggcatggtaggcccgctgcacagtagccttgctgccagcgccctgtcccagatgatgagctaccagggcctgcccagcacccggctggccacccagcctcacctggtgcagacccagcaggtgcagccacaaaacttacagatgcagcagcagaacctgcagccagcaaacatccagcagcagcaaagcctgcagccgccaccaccaccaccacagccgcaccttggcgtgagctcagcagccagcggccacctgggccggagcttcctgagtggagagccgagccaggcagacgtgcagccactgggccccagcagcctggcggtgcacactattctgccccaggagagccccgccctgcccacgtcgctgccatcctcgctggtcccacccgtgaccgcagcccagttcctgacgcccccctcgcagcacagctactcctcgcctgtggacaacacccccagccaccagctacaggtgcctgagcaccccttcctcaccccgtcccctgagtcccctgaccagtggtccagctcgtccccgcattccaacgtctccgactggtccgagggcgtctccagccctcccaccagcatgcagtcccagatcgcccgcattccggaggccttcaagtaaacggcgcgccccacgagaccccggcttcctttcccaagccttcgggcgtctgtgtgcgctctgtggatgccagggccgaccagaggagcctttttaaaacacatgtttttatacaaaataagaacgaggattttaattttttttagtatttatttatgtacttttattttacacagaaacactgcctttttatttatatgtactgttttatctggccccaggtagaaacttttatctattctgagaaaacaagcaagttctgagagccagggttttcctacgtaggatgaaaagattcttctgtgtttataaaatataaacaaagattcatgatttataaatgccatttatttattgattccttttttcaaaatccaaaaagaaatgatgttggagaagggaagttgaacgagcatagtccaaaaagctcctggggcgtccaggccgcgccctttccccgacgcccacccaaccccaagccagcccggccgctccaccagcatcacctgcctgttaggagaagctgcatccagaggcaaacggaggcaaagctggctcaccttccgcacgcggattaatttgcatctgaaataggaaacaagtgaaagcatatgggttagatgttgccatgtgttttagatggtttcttgcaagcatgcttgtgaaaatgtgttctcggagtgtgtatgccaagagtgcacccatggtaccaatcatgaatctttgtttcaggttcagtattatgtagttgttcgttggttatacaagttcttggtccctccagaaccaccccggccccctgcccgttcttgaaatgtaggcatcatgcatgtcaaacatgagatgtgtggactgtggcacttgcctgggtcacacacggaggcatcctacccttttctggggaaagacactgcctgggctgaccccggtggcggccccagcacctcagcctgcacagtgtcccccaggttccgaagaagatgctccagcaacacagcctgggccccagctcgcgggacccgaccccccgtgggctcccgtgttttgtaggagacttgccagagccgggcacattgagctgtgcaacgccgtgggctgcgtcctttggtcctgtccccgcagccctggcagggggcatgcggtcgggcaggggctggagggaggcgggggctgcccttgggccacccctcctagtttgggaggagcagatttttgcaataccaagtatagcctatggcagaaaaaatgtctgtaaatatgtttttaaaggtggattttgtttaaaaaatcttaatgaatgagtctgttgtgtgtcatgccagtgagggacgtcagacttggctcagctcggggagccttagccgcccatgcactggggacgctccgctgccgtgccgcctgcactcctcagggcagcctcccccggctctacgggggccgcgtggtgccatccccagggggcatgaccagatgcgtcccaagatgttgatttttactgtgttttataaaatagagtgtagtttacagaaaaagactttaaaagtgatctacatgaggaactgtagatgatgtatttttttcatcttttttgttaactgatttgcaataaaaatgatactgatggtgatctggcttccaaaaaaaaaaaaaaaaa

SEQ ID NO: 9 - protein, 2471 aa, Homo sapiens
MPALRPALLWALLALWLCCATPAHALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCLQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCMDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCDQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVIMAKRKRKHGSLWLPEGFTLRRDASNHKRREPVGQDAVGLKNLSVQVSEANLIGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDHMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 10 - DNA, 11189 bp, Homo sapiens
gcgaccgagaagatgcccgccctgcgccccgctctgctgtgggcgctgctggcgctctggctgtgctgcgcgacccccgcgcatgcattgcagtgtcgagatggctatgaaccctgtgtaaatgaaggaatgtgtgttacctaccacaatggcacaggatactgcaaatgtccagaaggcttcttgggggaatattgtcaacatcgagacccctgtgagaagaaccgctgccagaatggtgggacttgtgtggcccaggccatgctggggaaagccacgtgccgatgtgcctcagggtttacaggagaggactgccagtactcgacatctcatccatgctttgtgtctcgaccctgcctgaatggcggcacatgccatatgctcagccgggatacctatgagtgcacctgtcaagtcgggtttacaggtaaggagtgccaatggaccgatgcctgcctgtctcatccctgtgcaaatggaagtacctgtaccactgtggccaaccagttctcctgcaaatgcctcacaggcttcacagggcagaaatgtgagactgatgtcaatgagtgtgacattccaggacactgccagcatggtggcacctgcctcaacctgcctggttcctaccagtgccagtgccttcagggcttcacaggccagtactgtgacagcctgtatgtgccctgtgcaccctcgccttgtgtcaatggaggcacctgtcggcagactggtgacttcacttttgagtgcaactgccttccaggttttgaagggagcacctgtgagaggaatattgatgactgccctaaccacaggtgtcagaatggaggggtttgtgtggatggggtcaacacttacaactgccgctgtcccccacaatggacaggacagttctgcacagaggatgtggatgaatgcctgctgcagcccaatgcctgtcaaaatgggggcacctgtgccaaccgcaatggaggctatggctgtgtatgtgtcaacggctggagtggagatgactgcagtgagaacattgatgattgtgccttcgcctcctgtactccaggctccacctgcatcgaccgtgtggcctccttctcttgcatgtgcccagaggggaaggcaggtctcctgtgtcatctggatgatgcatgcatcagcaatccttgccacaagggggcactgtgtgacaccaaccccctaaatgggcaatatatttgcacctgcccacaaggctacaaaggggctgactgcacagaagatgtggatgaatgtgccatggccaatagcaatccttgtgagcatgcaggaaaatgtgtgaacacggatggcgccttccactgtgagtgtctgaagggttatgcaggacctcgttgtgagatggacatcaatgagtgccattcagacccctgccagaatgatgctacctgtctggataagattggaggcttcacatgtctgtgcatgccaggtttcaaaggtgtgcattgtgaattagaaataaatgaatgtcagagcaacccttgtgtgaacaatgggcagtgtgtggataaagtcaatcgtttccagtgcctgtgtcctcctggtttcactgggccagtttgccagattgatattgatgactgttccagtactccgtgtctgaatggggcaaagtgtatcgatcacccgaatggctatgaatgccagtgtgccacaggtttcactggtgtgttgtgtgaggagaacattgacaactgtgaccccgatccttgccaccatggtcagtgtcaggatggtattgattcctacacctgcatctgcaatcccgggtacatgggcgccatctgcagtgaccagattgatgaatgttacagcagcccttgcctgaacgatggtcgctgcattgacctggtcaatggctaccagtgcaactgccagccaggcacgtcaggggttaattgtgaaattaattttgatgactgtgcaagtaacccttgtatccatggaatctgtatggatggcattaatcgctacagttgtgtctgctcaccaggattcacagggcagagatgtaacattgacattgatgagtgtgcctccaatccctgtcgcaagggtgcaacatgtatcaacggtgtgaatggtttccgctgtatatgccccgagggaccccatcaccccagctgctactcacaggtgaacgaatgcctgagcaatccctgcatccatggaaactgtactggaggtctcagtggatataagtgtctctgtgatgcaggctgggttggcatcaactgtgaagtggacaaaaatgaatgcctttcgaatccatgccagaatggaggaacttgtgacaatctggtgaatggatacaggtgtacttgcaagaagggctttaaaggctataactgccaggtgaatattgatgaatgtgcctcaaatccatgcctgaaccaaggaacctgctttgatgacataagtggctacacttgccactgtgtgctgccatacacaggcaagaattgtcagacagtattggctccctgttccccaaacccttgtgagaatgctgctgtttgcaaagagtcaccaaattttgagagttatacttgcttgtgtgctcctggctggcaaggtcagcggtgtaccattgacattgacgagtgtatctccaagccctgcatgaaccatggtctctgccataacacccagggcagctacatgtgtgaatgtccaccaggcttcagtggtatggactgtgaggaggacattgatgactgccttgccaatccttgccagaatggaggttcctgtatggatggagtgaatactttctcctgcctctgccttccgggtttcactggggataagtgccagacagacatgaatgagtgtctgagtgaaccctgtaagaatggagggacctgctctgactacgtcaacagttacacttgcaagtgccaggcaggatttgatggagtccattgtgagaacaacatcaatgagtgcactgagagctcctgtttcaatggtggcacatgtgttgatgggattaactccttctcttgcttgtgccctgtgggtttcactggatccttctgcctccatgagatcaatgaatgcagctctcatccatgcctgaatgagggaacgtgtgttgatggcctgggtacctaccgctgcagctgccccctgggctacactgggaaaaactgtcagaccctggtgaatctctgcagtcggtctccatgtaaaaacaaaggtacttgcgttcagaaaaaagcagagtcccagtgcctatgtccatctggatgggctggtgcctattgtgacgtgcccaatgtctcttgtgacatagcagcctccaggagaggtgtgcttgttgaacacttgtgccagcactcaggtgtctgcatcaatgctggcaacacgcattactgtcagtgccccctgggctatactgggagctactgtgaggagcaactcgatgagtgtgcgtccaacccctgccagcacggggcaacatgcagtgacttcattggtggatacagatgcgagtgtgtcccaggctatcagggtgtcaactgtgagtatgaagtggatgagtgccagaatcagccctgccagaatggaggcacctgtattgaccttgtgaaccatttcaagtgctcttgcccaccaggcactcggggcctactctgtgaagagaacattgatgactgtgcccggggtccccattgccttaatggtggtcagtgcatggataggattggaggctacagttgtcgctgcttgcctggctttgctggggagcgttgtgagggagacatcaacgagtgcctctccaacccctgcagctctgagggcagcctggactgtatacagctcaccaatgactacctgtgtgtttgccgtagtgcctttactggccggcactgtgaaaccttcgtcgatgtgtgtccccagatgccctgcctgaatggagggacttgtgctgtggccagtaacatgcctgatggtttcatttgccgttgtcccccgggattttccggggcaaggtgccagagcagctgtggacaagtgaaatgtaggaagggggagcagtgtgtgcacaccgcctctggaccccgctgcttctgccccagtccccgggactgcgagtcaggctgtgccagtagcccctgccagcacgggggcagctgccaccctcagcgccagcctccttattactcctgccagtgtgccccaccattctcgggtagccgctgtgaactctacacggcaccccccagcacccctcctgccacctgtctgagccagtattgtgccgacaaagctcgggatggcgtctgtgatgaggcctgcaacagccatgcctgccagtgggatgggggtgactgttctctcaccatggagaacccctgggccaactgctcctccccacttccctgctgggattatatcaacaaccagtgtgatgagctgtgcaacacggtcgagtgcctgtttgacaactttgaatgccaggggaacagcaagacatgcaagtatgacaaatactgtgcagaccacttcaaagacaaccactgtgaccaggggtgcaacagtgaggagtgtggttgggatgggctggactgtgctgctgaccaacctgagaacctggcagaaggtaccctggttattgtggtattgatgccacctgaacaactgctccaggatgctcgcagcttcttgcgggcactgggtaccctgctccacaccaacctgcgcattaagcgggactcccagggggaactcatggtgtacccctattatggtgagaagtcagctgctatgaagaaacagaggatgacacgcagatcccttcctggtgaacaagaacaggaggtggctggctctaaagtctttctggaaattgacaaccgccagtgtgttcaagactcagaccactgcttcaagaacacggatgcagcagcagctctcctggcctctcacgccatacaggggaccctgtcataccctcttgtgtctgtcgtcagtgaatccctgactccagaacgcactcagctcctctatctccttgctgttgctgttgtcatcattctgtttattattctgctgggggtaatcatggcaaaacgaaagcgtaagcatggctctctctggctgcctgaaggtttcactcttcgccgagatgcaagcaatcacaagcgtcgtgagccagtgggacaggatgctgtggggctgaaaaatctctcagtgcaagtctcagaagctaacctaattggtactggaacaagtgaacactgggtcgatgatgaagggccccagccaaagaaagtaaaggctgaagatgaggccttactctcagaagaagatgaccccattgatcgacggccatggacacagcagcaccttgaagctgcagacatccgtaggacaccatcgctggctctcacccctcctcaggcagagcaggaggtggatgtgttagatgtgaatgtccgtggcccagatggctgcaccccattgatgttggcttctctccgaggaggcagctcagatttgagtgatgaagatgaagatgcagaggactcttctgctaacatcatcacagacttggtctaccagggtgccagcctccaggcccagacagaccggactggtgagatggccctgcaccttgcagcccgctactcacgggctgatgctgccaagcgtctcctggatgcaggtgcagatgccaatgcccaggacaacatgggccgctgtccactccatgctgcagtggcagctgatgcccaaggtgtcttccagattctgattcgcaaccgagtaactgatctagatgccaggatgaatgatggtactacacccctgatcctggctgcccgcctggctgtggagggaatggtggcagaactgatcaactgccaagcggatgtgaatgcagtggatgaccatggaaaatctgctcttcactgggcagctgctgtcaataatgtggaggcaactcttttgttgttgaaaaatggggccaaccgagacatgcaggacaacaaggaagagacacctctgtttcttgctgcccgggaggggagctatgaagcagccaagatcctgttagaccattttgccaatcgagacatcacagaccatatggatcgtcttccccgggatgtggctcgggatcacatgcaccatgacattgtgcgccttctggatgaatacaatgtgaccccaagccctccaggcaccgtgttgacttctgctctctcacctgtcatctgtgggcccaacagatctttcctcagcctgaagcacaccccaatgggcaagaagtctagacggcccagtgccaagagtaccatgcctactagcctccctaaccttgccaaggaggcaaaggatgccaagggtagtaggaggaagaagtctctgagtgagaaggtccaactgtctgagagttcagtaactttatcccctgttgattccctagaatctcctcacacgtatgtttccgacaccacatcctctccaatgattacatcccctgggatcttacaggcctcacccaaccctatgttggccactgccgcccctcctgccccagtccatgcccagcatgcactatctttttctaaccttcatgaaatgcagcctttggcacatggggccagcactgtgcttccctcagtgagccagttgctatcccaccaccacattgtgtctccaggcagtggcagtgctggaagcttgagtaggctccatccagtcccagtcccagcagattggatgaaccgcatggaggtgaatgagacccagtacaatgagatgtttggtatggtcctggctccagctgagggcacccatcctggcatagctccccagagcaggccacctgaagggaagcacataaccacccctcgggagcccttgccccccattgtgactttccagctcatccctaaaggcagtattgcccaaccagcgggggctccccagcctcagtccacctgccctccagctgttgcgggccccctgcccaccatgtaccagattccagaaatggcccgtttgcccagtgtggctttccccactgccatgatgccccagcaggacgggcaggtagctcagaccattctcccagcctatcatcctttcccagcctctgtgggcaagtaccccacacccccttcacagcacagttatgcttcctcaaatgctgctgagcgaacacccagtcacagtggtcacctccagggtgagcatccctacctgacaccatccccagagtctcctgaccagtggtcaagttcatcaccccactctgcttctgactggtcagatgtgaccaccagccctacccctgggggtgctggaggaggtcagcggggacctgggacacacatgtctgagccaccacacaacaacatgcaggtttatgcgtgagagagtccacctccagtgtagagacataactgacttttgtaaatgctgctgaggaacaaatgaaggtcatccgggagagaaatgaagaaatctctggagccagcttctagaggtaggaaagagaagatgttcttattcagataatgcaagagaagcaattcgtcagtttcactgggtatctgcaaggcttattgattattctaatctaataagacaagtttgtggaaatgcaagatgaatacaagccttgggtccatgtttactctcttctatttggagaataagatggatgcttattgaagcccagacattcttgcagcttggactgcattttaagccctgcaggcttctgccatatccatgagaagattctacactagcgtcctgttgggaattatgccctggaattctgcctgaattgacctacgcatctcctcctccttggacattcttttgtcttcatttggtgcttttggttttgcacctctccgtgattgtagccctaccagcatgttatagggcaagacctttgtgcttttgatcattctggcccatgaaagcaactttggtctcctttcccctcctgtcttcccggtatcccttggagtctcacaaggtttactttggtatggttctcagcacaaacctttcaagtatgttgtttctttggaaaatggacatactgtattgtgttctcctgcatatatcattcctggagagagaaggggagaagaatacttttcttcaacaaattttgggggcaggagatcccttcaagaggctgcaccttaatttttcttgtctgtgtgcaggtcttcatataaactttaccaggaagaagggtgtgagtttgttgtttttctgtgtatgggcctggtcagtgtaaagttttatccttgatagtctagttactatgaccctccccacttttttaaaaccagaaaaaggtttggaatgttggaatgaccaagagacaagttaactcgtgcaagagccagttacccacccacaggtccccctacttcctgccaagcattccattgactgcctgtatggaacacatttgtcccagatctgagcattctaggcctgtttcactcactcacccagcatatgaaactagtcttaactgttgagcctttcctttcatatccacagaagacactgtctcaaatgttgtacccttgccatttaggactgaactttccttagcccaagggacccagtgacagttgtcttccgtttgtcagatgatcagtctctactgattatcttgctgcttaaaggcctgctcaccaatctttctttcacaccgtgtggtccgtgttactggtatacccagtatgttctcactgaagacatggactttatatgttcaagtgcaggaattggaaagttggacttgttttctatgatccaaaacagccctataagaaggttggaaaaggaggaactatatagcagcctttgctattttctgctaccatttcttttcctctgaagcggccatgacattccctttggcaactaacgtagaaactcaacagaacattttcctttcctagagtcaccttttagatgataatggacaactatagacttgctcattgttcagactgattgcccctcacctgaatccactctctgtattcatgctcttggcaatttctttgactttcttttaagggcagaagcattttagttaattgtagataaagaatagttttcttcctcttctccttgggccagttaataattggtccatggctacactgcaacttccgtccagtgctgtgatgcccatgacacctgcaaaataagttctgcctgggcattttgtagatattaacaggtgaattcccgactcttttggtttgaatgacagttctcattccttctatggctgcaagtatgcatcagtgcttcccacttacctgatttgtctgtcggtggccccatatggaaaccctgcgtgtctgttggcataatagtttacaaatggttttttcagtcctatccaaatttattgaaccaacaaaaataattacttctgccctgagataagcagattaagtttgttcattctctgctttattctctccatgtggcaacattctgtcagcctctttcatagtgtgcaaacattttatcattctaaatggtgactctctgcccttggacccatttattattcacagatggggagaacctatctgcatggacctctgtggaccacagcgtacctgcccctttctgccctcctgctccagccccacttctgaaagtatcagctactgatccagccactggatattttatatcctcccttttccttaagcacaatgtcagaccaaattgcttgtttctttttcttggactactttaatttggatcctttgggtttggagaaagggaatgtgaaagctgtcattacagacaacaggtttcagtgatgaggaggacaacactgcctttcaaactttttactgatctcttagattttaagaactcttgaattgtgtggtatctaataaaagggaaggtaagatggataatcactttctcatttgggttctgaattggagactcagtttttatgagacacatcttttatgccatgtatagatcctcccctgctatttttggtttatttttattgttataaatgctttctttctttgactcctcttctgcctgcctttggggataggtttttttgtttgtttatttgcttcctctgttttgttttaagcatcattttcttatgtgaggtggggaagggaaaggtatgagggaaagagagtctgagaattaaaatattttagtataagcaattggctgtgatgctcaaatccattgcatcctcttattgaatttgccaatttgtaatttttgcataataaagaaccaaaggtgtaatgttttgttgagaggtggtttagggattttggccctaaccaatacattgaatgtatgatgactatttgggaggacacatttatgtacccagaggcccccactaataagtggtactatggttacttccttgtgtacatttctcttaaaagtgatattatatctgtttgtatgagaaacccagtaaccaataaaatgaccgcatattcctgactaaacgtagtaaggaaaatgcacactttgtttttacttttccgtttcattctaaaggtagttaagatgaaatttatatgaaagcatttttatcacaaaataaaaaaggtttgccaagctcagtggtgttgtattttttattttccaatactgcatccatggcctggcagtgttacctcatgatgtcataatttgctgagagagcaaattttcttttctttctgaatcccacaaagcctagcaccaaacttctttttttcttcctttaattagatcataaataaatgatcctggggaaaaagcatctgtcaaataggaaacatcacaaaactgagcactcttctgtgcactagccatagctggtgacaaacagatggttgctcagggacaaggtgccttccaatggaaatgcgaagtagttgctatagcaagaattgggaactgggatataagtcataatattaattatgctgttatgtaaatgattggtttgtaacattccttaagtgaaatttgtgtagaacttaatatacaggattataaaataatattttgtgtataaatttgttataagttcacattcatacatttatttataaagtcagtgagatatttgaacatgaaaaaaaaaa

SEQ ID NO: 11 - protein, 2321 aa, Homo sapiens
MGPGARGRRRRRRPMSPPPPPPPVRALPLLLLLAGPGAAAPPCLDGSPCANGGRCTQLPSREAACLCPPGWVGERCQLEDPCHSGPCAGRGVCQSSVVAGTARFSCRCPRGFRGPDCSLPDPCLSSPCAHGARCSVGPDGRFLCSCPPGYQGRSCRSDVDECRVGEPCRHGGTCLNTPGSFRCQCPAGYTGPLCENPAVPCAPSPCRNGGTCRQSGDLTYDCACLPGFEGQNCEVNVDDCPGHRCLNGGTCVDGVNTYNCQCPPEWTGQFCTEDVDECQLQPNACHNGGTCFNTLGGHSCVCVNGWTGESCSQNIDDCATAVCFHGATCHDRVASFYCACPMGKTGLLCHLDDACVSNPCHEDAICDTNPVNGRAICTCPPGFTGGACDQDVDECSIGANPCEHLGRCVNTQGSFLCQCGRGYTGPRCETDVNECLSGPCRNQATCLDRIGQFTCICMAGFTGTYCEVDIDECQSSPCVNGGVCKDRVNGFSCTCPSGFSGSTCQLDVDECASTPCRNGAKCVDQPDGYECRCAEGFEGTLCDRNVDDCSPDPCHHGRCVDGIASFSCACAPGYTGTRCESQVDECRSQPCRHGGKCLDLVDKYLCRCPSGTTGVNCEVNIDDCASNPCTFGVCRDGINRYDCVCQPGFTGPLCNVEINECASSPCGEGGSCVDGENGFRCLCPPGSLPPLCLPPSHPCAHEPCSHGICYDAPGGFRCVCEPGWSGPRCSQSLARDACESQPCRAGGTCSSDGMGFHCTCPPGVQGRQCELLSPCTPNPCEHGGRCESAPGQLPVCSCPQGWQGPRCQQDVDECAGPAPCGPHGICTNLAGSFSCTCHGGYTGPSCDQDINDCDPNPCLNGGSCQDGVGSFSCSCLPGFAGPRCARDVDECLSNPCGPGTCTDHVASFTCTCPPGYGGFHCEQDLPDCSPSSCFNGGTCVDGVNSFSCLCRPGYTGAHCQHEADPCLSRPCLHGGVCSAAHPGFRCTCLESFTGPQCQTLVDWCSRQPCQNGGRCVQTGAYCLCPPGWSGRLCDIRSLPCREAAAQIGVRLEQLCQAGGQCVDEDSSHYCVCPEGRTGSHCEQEVDPCLAQPCQHGGTCRGYMGGYMCECLPGYNGDNCEDDVDECASQPCQHGGSCIDLVARYLCSCPPGTLGVLCEINEDDCGPGPPLDSGPRCLHNGTCVDLVGGFRCTCPPGYTGLRCEADINECRSGACHAAHTRDCLQDPGGGFRCLCHAGFSGPRCQTVLSPCESQPCQHGGQCRPSPGPGGGLTFTCHCAQPFWGPRCERVARSCRELQCPVGVPCQQTPRGPRCACPPGLSGPSCRSFPGSPPGASNASCAAAPCLHGGSCRPAPLAPFFRCACAQGWTGPRCEAPAAAPEVSEEPRCPRAACQAKRGDQRCDRECNSPGCGWDGGDCSLSVGDPWRQCEALQCWRLFNNSRCDPACSSPACLYDNFDCHAGGRERTCNPVYEKYCADHFADGRCDQGCNTEECGWDGLDCASEVPALLARGVLVLTVLLPPEELLRSSADFLQRLSAILRTSLRFRLDAHGQAMVFPYHRPSPGSEPRARRELAPEVIGSVVMLEIDNRLCLQSPENDHCFPDAQSAADYLGALSAVERLDFPYPLRDVRGEPLEPPEPSVPLLPLLVAGAVLLLVILVLGVMVARRKREHSTLWFPEGFSLHKDVASGHKGRREPVGQDALGMKNMAKGESLMGEVATDWMDTECPEAKRLKVEEPGMGAEEAVDCRQWTQHHLVAADIRVAPAMALTPPQGDADADGMDVNVRGPDGFTPLMLASFCGGALEPMPTEEDEADDTSASIISDLICQGAQLGARTDRTGETALHLAARYARADAAKRLLDAGADTNAQDHSGRTPLHTAVTADAQGVFQILIRNRSTDLDARMADGSTALILAARLAVEGMVEELIASHADVNAVDELGKSALHWAAAVNNVEATLALLKNGANKDMQDSKEETPLFLAAREGSYEAAKLLLDHFANREITDHLDRLPRDVAQERLHQDIVRLLDQPSGPRSPPGPHGLGPLLCPPGAFLPGLKAAQSGSKKSRRPPGKAGLGPQGPRGRGKKLTLACPGPLADSSVTLSPVDSLDSPRPFGGPPASPGGFPLEGPYAAATATAVSLAQLGGPGRAGLGRQPPGGCVLSLGLLNPVAVPLDWARLPPPAPPGPSFLLPLAPGPQLLNPGTPVSPQERPPPYLAVPGHGEEYPVAGAHSSPPKARFLRVPSEHPYLTPSPESPEHWASPSPPSLSDWSESTPSPATATGAMATTTGALPAQPLPLSVPSSLAQAQTQLGPQPEVTPKRQVLA

SEQ ID NO: 12 - DNA, 8091 bp, Homo sapiens
acgcggcgcggaggctggcccgggacgcgcccggagcccagggaaggagggaggaggggagggtcgcggccggccgccatggggccgggggcccgtggccgccgccgccgccgtcgcccgatgtcgccgccaccgccaccgccacccgtgcgggcgctgcccctgctgctgctgctagcggggccgggggctgcagcccccccttgcctggacggaagcccgtgtgcaaatggaggtcgttgcacccagctgccctcccgggaggctgcctgcctgtgcccgcctggctgggtgggtgagcggtgtcagctggaggacccctgtcactcaggcccctgtgctggccgtggtgtctgccagagttcagtggtggctggcaccgcccgattctcatgccggtgcccccgtggcttccgaggccctgactgctccctgccagatccctgcctcagcagcccttgtgcccacggtgcccgctgctcagtggggcccgatggacgcttcctctgctcctgcccacctggctaccagggccgcagctgccgaagcgacgtggatgagtgccgggtgggtgagccctgccgccatggtggcacctgcctcaacacacctggctccttccgctgccagtgtccagctggctacacagggccactatgtgagaaccccgcggtgccctgtgcgccctcaccatgccgtaacgggggcacctgcaggcagagtggcgacctcacttacgactgtgcctgtcttcctgggtttgagggtcagaattgtgaagtgaacgtggacgactgtccaggacaccgatgtctcaatggggggacatgcgtggatggcgtcaacacctataactgccagtgccctcctgagtggacaggccagttctgcacggaggacgtggatgagtgtcagctgcagcccaacgcctgccacaatgggggtacctgcttcaacacgctgggtggccacagctgcgtgtgtgtcaatggctggacaggtgagagctgcagtcagaatatcgatgactgtgccacagccgtgtgcttccatggggccacctgccatgaccgcgtggcttctttctactgtgcctgccccatgggcaagactggcctcctgtgtcacctggatgacgcctgtgtcagcaacccctgccacgaggatgctatctgtgacacaaatccggtgaacggccgggccatttgcacctgtcctcccggcttcacgggtggggcatgtgaccaggatgtggacgagtgctctatcggcgccaacccctgcgagcacttgggcaggtgcgtgaacacgcagggctccttcctgtgccagtgcggtcgtggctacactggacctcgctgtgagaccgatgtcaacgagtgtctgtcggggccctgccgaaaccaggccacgtgcctcgaccgcataggccagttcacctgtatctgtatggcaggcttcacaggaacctattgcgaggtggacattgacgagtgtcagagtagcccctgtgtcaacggtggggtctgcaaggaccgagtcaatggcttcagctgcacctgcccctcgggcttcagcggctccacgtgtcagctggacgtggacgaatgcgccagcacgccctgcaggaatggcgccaaatgcgtggaccagcccgatggctacgagtgccgctgtgccgagggctttgagggcacgctgtgtgatcgcaacgtggacgactgctcccctgacccatgccaccatggtcgctgcgtggatggcatcgccagcttctcatgtgcctgtgctcctggctacacgggcacacgctgcgagagccaggtggacgaatgccgcagccagccctgccgccatggcggcaaatgcctagacctggtggacaagtacctctgccgctgcccttctgggaccacaggtgtgaactgcgaagtgaacattgacgactgtgccagcaacccctgcacctttggagtctgccgtgatggcatcaaccgctacgactgtgtctgccaacctggcttcacagggcccctttgtaacgtggagatcaatgagtgtgcttccagcccatgcggcgagggaggttcctgtgtggatggggaaaatggcttccgctgcctctgcccgcctggctccttgcccccactctgcctccccccgagccatccctgtgcccatgagccctgcagtcacggcatctgctatgatgcacctggcgggttccgctgtgtgtgtgagcctggctggagtggcccccgctgcagccagagcctggcccgagacgcctgtgagtcccagccgtgcagggccggtgggacatgcagcagcgatggaatgggtttccactgcacctgcccgcctggtgtccagggacgtcagtgtgaactcctctccccctgcaccccgaacccctgtgagcatgggggccgctgcgagtctgcccctggccagctgcctgtctgctcctgcccccagggctggcaaggcccacgatgccagcaggatgtggacgagtgtgctggccccgcaccctgtggccctcatggtatctgcaccaacctggcagggagtttcagctgcacctgccatggagggtacactggcccttcctgtgatcaggacatcaatgactgtgaccccaacccatgcctgaacggtggctcgtgccaagacggcgtgggctccttttcctgctcctgcctccctggtttcgccggcccacgatgcgcccgcgatgtggatgagtgcctgagcaacccctgcggcccgggcacctgtaccgaccacgtggcctccttcacctgcacctgcccgccgggctacggaggcttccactgcgaacaggacctgcccgactgcagccccagctcctgcttcaatggcgggacctgtgtggacggcgtgaactcgttcagctgcctgtgccgtcccggctacacaggagcccactgccaacatgaggcagacccctgcctctcgcggccctgcctacacgggggcgtctgcagcgccgcccaccctggcttccgctgcacctgcctcgagagcttcacgggcccgcagtgccagacgctggtggattggtgcagccgccagccttgtcaaaacgggggtcgctgcgtccagactggggcctattgcctttgtccccctggatggagcggacgcctctgtgacatccgaagcttgccctgcagggaggccgcagcccagatcggggtgcggctggagcagctgtgtcaggcgggtgggcagtgtgtggatgaagacagctcccactactgcgtgtgcccagagggccgtactggtagccactgtgagcaggaggtggacccctgcttggcccagccctgccagcatggggggacctgccgtggctatatggggggctacatgtgtgagtgtcttcctggctacaatggtgataactgtgaggacgacgtggacgagtgtgcctcccagccctgccagcacgggggttcatgcattgacctcgtggcccgctatctctgctcctgtcccccaggaacgctgggggtgctctgcgagattaatgaggatgactgcggcccaggcccaccgctggactcagggccccggtgcctacacaatggcacctgcgtggacctggtgggtggtttccgctgcacctgtcccccaggatacactggtttgcgctgcgaggcagacatcaatgagtgtcgctcaggtgcctgccacgcggcacacacccgggactgcctgcaggacccaggcggaggtttccgttgcctttgtcatgctggcttctcaggtcctcgctgtcagactgtcctgtctccctgcgagtcccagccatgccagcatggaggccagtgccgtcctagcccgggtcctgggggtgggctgaccttcacctgtcactgtgcccagccgttctggggtccgcgttgcgagcgggtggcgcgctcctgccgggagctgcagtgcccggtgggcgtcccatgccagcagacgccccgcgggccgcgctgcgcctgccccccagggttgtcgggaccctcctgccgcagcttcccggggtcgccgccgggggccagcaacgccagctgcgcggccgccccctgtctccacgggggctcctgccgccccgcgccgctcgcgcccttcttccgctgcgcttgcgcgcagggctggaccgggccgcgctgcgaggcgcccgccgcggcacccgaggtctcggaggagccgcggtgcccgcgcgccgcctgccaggccaagcgcggggaccagcgctgcgaccgcgagtgcaacagcccaggctgcggctgggacggcggcgactgctcgctgagcgtgggcgacccctggcggcaatgcgaggcgctgcagtgctggcgcctcttcaacaacagccgctgcgaccccgcctgcagctcgcccgcctgcctctacgacaacttcgactgccacgccggtggccgcgagcgcacttgcaacccggtgtacgagaagtactgcgccgaccactttgccgacggccgctgcgaccagggctgcaacacggaggagtgcggctgggatgggctggattgtgccagcgaggtgccggccctgctggcccgcggcgtgctggtgctcacagtgctgctgccgccggaggagctactgcgttccagcgccgactttctgcagcggctcagcgccatcctgcgcacctcgctgcgcttccgcctggacgcgcacggccaggccatggtcttcccttaccaccggcctagtcctggctccgaaccccgggcccgtcgggagctggcccccgaggtgatcggctcggtagtaatgctggagattgacaaccggctctgcctgcagtcgcctgagaatgatcactgcttccccgatgcccagagcgccgctgactacctgggagcgttgtcagcggtggagcgcctggacttcccgtacccactgcgggacgtgcggggggagccgctggagcctccagaacccagcgtcccgctgctgccactgctagtggcgggcgctgtcttgctgctggtcattctcgtcctgggtgtcatggtggcccggcgcaagcgcgagcacagcaccctctggttccctgagggcttctcactgcacaaggacgtggcctctggtcacaagggccggcgggaacccgtgggccaggacgcgctgggcatgaagaacatggccaagggtgagagcctgatgggggaggtggccacagactggatggacacagagtgcccagaggccaagcggctaaaggtagaggagccaggcatgggggctgaggaggctgtggattgccgtcagtggactcaacaccatctggttgctgctgacatccgcgtggcaccagccatggcactgacaccaccacagggcgacgcagatgctgatggcatggatgtcaatgtgcgtggcccagatggcttcaccccgctaatgctggcttccttctgtgggggggctctggagccaatgccaactgaagaggatgaggcagatgacacatcagctagcatcatctccgacctgatctgccagggggctcagcttggggcacggactgaccgtactggcgagactgctttgcacctggctgcccgttatgcccgtgctgatgcagccaagcggctgctggatgctggggcagacaccaatgcccaggaccactcaggccgcactcccctgcacacagctgtcacagccgatgcccagggtgtcttccagattctcatccgaaaccgctctacagacttggatgcccgcatggcagatggctcaacggcactgatcctggcggcccgcctggcagtagagggcatggtggaagagctcatcgccagccatgctgatgtcaatgctgtggatgagcttgggaaatcagccttacactgggctgcggctgtgaacaacgtggaagccactttggccctgctcaaaaatggagccaataaggacatgcaggatagcaaggaggagacccccctattcctggccgcccgcgagggcagctatgaggctgccaagctgctgttggaccactttgccaaccgtgagatcaccgaccacctggacaggctgccgcgggacgtagcccaggagagactgcaccaggacatcgtgcgcttgctggatcaacccagtgggccccgcagcccccccggtccccacggcctggggcctctgctctgtcctccaggggccttcctccctggcctcaaagcggcacagtcggggtccaagaagagcaggaggccccccgggaaggcggggctggggccgcaggggccccgggggcggggcaagaagctgacgctggcctgcccgggccccctggctgacagctcggtcacgctgtcgcccgtggactcgctggactccccgcggcctttcggtgggccccctgcttcccctggtggcttcccccttgaggggccctatgcagctgccactgccactgcagtgtctctggcacagcttggtggcccaggccgggcaggtctagggcgccagccccctggaggatgtgtactcagcctgggcctgctgaaccctgtggctgtgcccctcgattgggcccggctgcccccacctgcccctccaggcccctcgttcctgctgccactggcgccgggaccccagctgctcaacccagggacccccgtctccccgcaggagcggcccccgccttacctggcagtcccaggacatggcgaggagtacccggtggctggggcacacagcagccccccaaaggcccgcttcctgcgggttcccagtgagcacccttacctgaccccatcccccgaatcccctgagcactgggccagcccctcacctccctccctctcagactggtccgaatccacgcctagcccagccactgccactggggccatggccaccaccactggggcactgcctgcccagccacttcccttgtctgttcccagctcccttgctcaggcccagacccagctggggccccagccggaagttacccccaagaggcaagtgttggcctgagacgctcgtcagttcttagatcttgggggcctaaagagacccccgtcctgcctcctttctttctctgtctcttccttccttttagtctttttcatcctcttctctttccaccaaccctcctgcatccttgccttgcagcgtgaccgagataggtcatcagcccagggcttcagtcttcctttatttataatgggtgggggctaccacccaccctctcagtcttgtgaagagtctgggacctccttcttccccacttctctcttccctcattcctttctctctccttctggcctctcatttccttacactctgacatgaatgaattattattatttttctttttctttttttttttacattttgtatagaaacaaattcatttaaacaaacttattattattattttttacaaaatatatatatggagatgctccctccccctgtgaaccccccagtgcccccgtggggctgagtctgtgggcccattcggccaagctggattctgtgtacctagtacacaggcatgactgggatcccgtgtaccgagtacacgacccaggtatgtaccaagtaggcacccttgggcgcacccactggggccaggggtcgggggagtgttgggagcctcctccccaccccacctccctcacttcactgcattccagattggacatgttccatagccttgctggggaagggcccactgccaactccctctgccccagccccacccttggccatctccctttgggaactagggggctgctggtgggaaatgggagccagggcagatgtatgcattcctttatgtccctgtaaatgtgggactacaagaagaggagctgcctgagtggtactttctcttcctggtaatcctctggcccagccttatggcagaatagaggtatttttaggctatttttgtaatatggcttctggtcaaaatccctgtgtagctgaattcccaagccctgcattgtacagccccccactcccctcaccacctaataaaggaatagttaacactcaaaaaaaaaaaaaaaaaaa

SEQ ID NO: 13 - protein, 2003 aa, Homo sapiens
MQPPSLLLLLLLLLLLCVSVVRPRGLLCGSFPEPCANGGTCLSLSLGQGTCQCAPGFLGETCQFPDPCQNAQLCQNGGSCQALLPAPLGLPSSPSPLTPSFLCTCLPGFTGERCQAKLEDPCPPSFCSKRGRCHIQASGRPQCSCMPGWTGEQCQLRDFCSANPCVNGGVCLATYPQIQCHCPPGFEGHACERDVNECFQDPGPCPKGTSCHNTLGSFQCLCPVGQEGPRCELRAGPCPPRGCSNGGTCQLMPEKDSTFHLCLCPPGFIGPDCEVNPDNCVSHQCQNGGTCQDGLDTYTCLCPETWTGWDCSEDVDECETQGPPHCRNGGTCQNSAGSFHCVCVSGWGGTSCEENLDDCIAATCAPGSTCIDRVGSFSCLCPPGRTGLLCHLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCICLPGFSGTRCEEDIDECRSSPCANGGQCQDQPGAFHCKCLPGFEGPRCQTEVDECLSDPCPVGASCLDLPGAFFCLCPSGFTGQLCEVPLCAPNLCQPKQICKDQKDKANCLCPDGSPGCAPPEDNCTCHHGHCQRSSCVCDVGWTGPECEAELGGCISAPCAHGGTCYPQPSGYNCTCPTGYTGPTCSEEMTACHSGPCLNGGSCNPSPGGYYCTCPPSHTGPQCQTSTDYCVSAPCFNGGTCVNRPGTFSCLCAMGFQGPRCEGKLRPSCADSPCRNRATCQDSPQGPRCLCPTGYTGGSCQTLMDLCAQKPCPRNSHCLQTGPSFHCLCLQGWTGPLCNLPLSSCQKAALSQGIDVSSLCHNGGLCVDSGPSYFCHCPPGFQGSLCQDHVNPCESRPCQNGATCMAQPSGYLCQCAPGYDGQNCSKELDACQSQPCHNHGTCTPKPGGFHCACPPGFVGLRCEGDVDECLDQPCHPTGTAACHSLANAFYCQCLPGHTGQWCEVEIDPCHSQPCFHGGTCEATAGSPLGFICHCPKGFEGPTCSHRAPSCGFHHCHHGGLCLPSPKPGFPPRCACLSGYGGPDCLTPPAPKGCGPPSPCLYNGSCSETTGLGGPGFRCSCPHSSPGPRCQKPGAKGCEGRSGDGACDAGCSGPGGNWDGGDCSLGVPDPWKGCPSHSRCWLLFRDGQCHPQCDSEECLFDGYDCETPPACTPAYDQYCHDHFHNGHCEKGCNTAECGWDGGDCRPEDGDPEWGPSLALLVVLSPPALDQQLFALARVLSLTLRVGLWVRKDRDGRDMVYPYPGARAEEKLGGTRDPTYQERAAPQTQPLGKETDSLSAGFVVVMGVDLSRCGPDHPASRCPWDPGLLLRFLAAMAAVGALEPLLPGPLLAVHPHAGTAPPANQLPWPVLCSPVAGVILLALGALLVLQLIRRRRREHGALWLPPGFTRRPRTQSAPHRRRPPLGEDSIGLKALKPKAEVDEDGVVMCSGPEEGEEVGQAEETGPPSTCQLWSLSGGCGALPQAAMLTPPQESEMEAPDLDTRGPDGVTPLMSAVCCGEVQSGTFQGAWLGCPEPWEPLLDGGACPQAHTVGTGETPLHLAARFSRPTAARRLLEAGANPNQPDRAGRTPLHAAVAADAREVCQLLLRSRQTAVDARTEDGTTPLMLAARLAVEDLVEELIAAQADVGARDKWGKTALHWAAAVNNARAARSLLQAGADKDAQDNREQTPLFLAAREGAVEVAQLLLGLGAARELRDQAGLAPADVAHQRNHWDLLTLLEGAGPPEARHKATPGREAGPFPRARTVSVSVPPHGGGALPRCRTLSAGAGPRGGGACLQARTWSVDLAARGGGAYSHCRSLSGVGAGGGPTPRGRRFSAGMRGPRPNPAIMRGRYGVAAGRGGRVSTDDWPCDWVALGACGSASNIPIPPPCLTPSPERGSPQLDCGPPALQEMPINQGGEGKK

SEQ ID NO: 14 - DNA, 6122 bp, Homo sapiens
gccggccgcgtcgaccctgccccagtgagagctctgagggtccctgcctgaagagggacagggaccggggcttggagaaggggctgtggaatgcagcccccttcactgctgctgctgctgctgctgctgctgctgctatgtgtctcagtggtcagacccagagggctgctgtgtgggagtttcccagaaccctgtgccaatggaggcacctgcctgagcctgtctctgggacaagggacctgccagtgtgcccctggcttcctgggtgagacgtgccagtttcctgacccctgccagaacgcccagctctgccaaaatggaggcagctgccaagccctgcttcccgctcccctagggctccccagctctccctctccattgacacccagcttcttgtgcacttgcctccctggcttcactggtgagagatgccaggccaagcttgaagacccttgtcctccctccttctgttccaaaaggggccgctgccacatccaggcctcgggccgcccacagtgctcctgcatgcctggatggacaggtgagcagtgccagcttcgggacttctgttcagccaacccatgtgttaatggaggggtgtgtctggccacatacccccagatccagtgccactgcccaccgggcttcgagggccatgcctgtgaacgtgatgtcaacgagtgcttccaggacccaggaccctgccccaaaggcacctcctgccataacaccctgggctccttccagtgcctctgccctgtggggcaggagggtccacgttgtgagctgcgggcaggaccctgccctcctaggggctgttcgaatgggggcacctgccagctgatgccagagaaagactccacctttcacctctgcctctgtcccccaggtttcataggcccagactgtgaggtgaatccagacaactgtgtcagccaccagtgtcagaatgggggcacttgccaggatgggctggacacctacacctgcctctgcccagaaacctggacaggctgggactgctccgaagatgtggatgagtgtgagacccagggtccccctcactgcagaaacgggggcacctgccagaactctgctggtagctttcactgcgtgtgtgtgagtggctggggcggcacaagctgtgaggagaacctggatgactgtattgctgccacctgtgccccgggatccacctgcattgaccgggtgggctctttctcctgcctctgcccacctggacgcacaggactcctgtgccacttggaagacatgtgtctgagccagccgtgccatggggatgcccaatgcagcaccaaccccctcacaggctccacactctgcctgtgtcagcctggctattcggggcccacctgccaccaggacctggacgagtgtctgatggcccagcaaggcccaagtccctgtgaacatggcggttcctgcctcaacactcctggctccttcaactgcctctgtccacctggctacacaggctcccgttgtgaggctgatcacaatgagtgcctctcccagccctgccacccaggaagcacctgtctggacctacttgccaccttccactgcctctgcccgccaggcttagaagggcagctctgtgaggtggagaccaacgagtgtgcctcagctccctgcctgaaccacgcggattgccatgacctgctcaacggcttccagtgcatctgcctgcctggattctccggcacccgatgtgaggaggatatcgatgagtgcagaagctctccctgtgccaatggtgggcagtgccaggaccagcctggagccttccactgcaagtgtctcccaggctttgaagggccacgctgtcaaacagaggtggatgagtgcctgagtgacccatgtcccgttggagccagctgccttgatcttccaggagccttcttttgcctctgcccctctggtttcacaggccagctctgtgaggttcccctgtgtgctcccaacctgtgccagcccaagcagatatgtaaggaccagaaagacaaggccaactgcctctgtcctgatggaagccctggctgtgccccacctgaggacaactgcacctgccaccacgggcactgccagagatcctcatgtgtgtgtgacgtgggttggacggggccagagtgtgaggcagagctagggggctgcatctctgcaccctgtgcccatggggggacctgctacccccagccctctggctacaactgcacctgccctacaggctacacaggacccacctgtagtgaggagatgacagcttgtcactcagggccatgtctcaatggcggctcctgcaaccctagccctggaggctactactgcacctgccctccaagccacacagggccccagtgccaaaccagcactgactactgtgtgtctgccccgtgcttcaatgggggtacctgtgtgaacaggcctggcaccttctcctgcctctgtgccatgggcttccagggcccgcgctgtgagggaaagctccgccccagctgtgcagacagcccctgtaggaatagggcaacctgccaggacagccctcagggtccccgctgcctctgccccactggctacaccggaggcagctgccagactctgatggacttatgtgcccagaagccctgcccacgcaattcccactgcctccagactgggccctccttccactgcttgtgcctccagggatggaccgggcctctctgcaaccttccactgtcctcctgccagaaggctgcactgagccaaggcatagacgtctcttccctttgccacaatggaggcctctgtgtcgacagcggcccctcctatttctgccactgcccccctggattccaaggcagcctgtgccaggatcacgtgaacccatgtgagtccaggccttgccagaacggggccacctgcatggcccagcccagtgggtatctctgccagtgtgccccaggctacgatggacagaactgctcaaaggaactcgatgcttgtcagtcccaaccctgtcacaaccatggaacctgtactcccaaacctggaggattccactgtgcctgccctccaggctttgtggggctacgctgtgagggagacgtggacgagtgtctggaccagccctgccaccccacaggcactgcagcctgccactctctggccaatgccttctactgccagtgtctgcctggacacacaggccagtggtgtgaggtggagatagacccctgccacagccaaccctgctttcatggagggacctgtgaggccacagcaggatcacccctgggtttcatctgccactgccccaagggttttgaaggccccacctgcagccacagggccccttcctgcggcttccatcactgccaccacggaggcctgtgtctgccctcccctaagccaggcttcccaccacgctgtgcctgcctcagtggctatgggggtcctgactgcctgaccccaccagctcctaaaggctgtggccctccctccccatgcctatacaatggcagctgctcagagaccacgggcttggggggcccaggctttcgatgctcctgccctcacagctctccagggccccggtgtcagaaacccggagccaaggggtgtgagggcagaagtggagatggggcctgcgatgctggctgcagtggcccgggaggaaactgggatggaggggactgctctctgggagtcccagacccctggaagggctgcccctcccactctcggtgctggcttctcttccgggacgggcagtgccacccacagtgtgactctgaagagtgtctgtttgatggctacgactgtgagacccctccagcctgcactccagcctatgaccagtactgccatgatcacttccacaacgggcactgtgagaaaggctgcaacactgcagagtgtggctgggatggaggtgactgcaggcctgaagatggggacccagagtgggggccctccctggccctgctggtggtactgagccccccagccctagaccagcagctgtttgccctggcccgggtgctgtccctgactctgagggtaggactctgggtaaggaaggatcgtgatggcagggacatggtgtacccctatcctggggcccgggctgaagaaaagctaggaggaactcgggaccccacctatcaggagagagcagcccctcaaacgcagcccctgggcaaggagaccgactccctcagtgctgggttcgtggtggtcatgggtgtggatttgtcccgctgtggccctgaccacccggcatcccgctgtccctgggaccctgggcttctactccgcttccttgctgcgatggctgcagtgggagccctggagcccctgctgcctggaccactgctggctgtccaccctcatgcagggaccgcaccccctgccaaccagcttccctggcctgtgctgtgctccccagtggccggggtgattctcctggccctaggggctcttctcgtcctccagctcatccggcgtcgacgccgagagcatggagctctctggctgccccctggtttcactcgacggcctcggactcagtcagctccccaccgacgccggcccccactaggcgaggacagcattggtctcaaggcactgaagccaaaggcagaagttgatgaggatggagttgtgatgtgctcaggccctgaggagggagaggaggtgggccaggctgaagaaacaggcccaccctccacgtgccagctctggtctctgagtggtggctgtggggcgctccctcaggcagccatgctaactcctccccaggaatctgagatggaagcccctgacctggacacccgtggacctgatggggtgacacccctgatgtcagcagtttgctgtggggaagtacagtccgggaccttccaaggggcatggttgggatgtcctgagccctgggaacctctgctggatggaggggcctgtccccaggctcacaccgtgggcactggggagacccccctgcacctggctgcccgattctcccggccaaccgctgcccgccgcctccttgaggctggagccaaccccaaccagccagaccgggcagggcgcacaccccttcatgctgctgtggctgctgatgctcgggaggtctgccagcttctgctccgtagcagacaaactgcagtggacgctcgcacagaggacgggaccacacccttgatgctggctgccaggctggcggtggaagacctggttgaagaactgattgcagcccaagcagacgtgggggccagagataaatgggggaaaactgcgctgcactgggctgctgccgtgaacaacgcccgagccgcccgctcgcttctccaggccggagccgataaagatgcccaggacaacagggagcagacgccgctattcctggcggcgcgggaaggagcggtggaagtagcccagctactgctggggctgggggcagcccgagagctgcgggaccaggctgggctagcgccggcggacgtcgctcaccaacgtaaccactgggatctgctgacgctgctggaaggggctgggccaccagaggcccgtcacaaagccacgccgggccgcgaggctgggcccttcccgcgcgcacggacggtgtcagtaagcgtgcccccgcatgggggcggggctctgccgcgctgccggacgctgtcagccggagcaggccctcgtgggggcggagcttgtctgcaggctcggacttggtccgtagacttggctgcgcgggggggcggggcctattcgcattgccggagcctctcgggagtaggagcaggaggaggcccgacccctcgcggccgtaggttttctgcaggcatgcgcgggcctcggcccaaccctgcgataatgcgaggaagatacggagtggctgccgggcgcggaggcagggtctcaacggatgactggccctgtgattgggtggccctgggagcttgcggttctgcctccaacattccgatcccgcctccttgccttactccgtccccggagcggggatcacctcaacttgactgtggtcccccagccctccaagaaatgcccataaaccaaggaggagagggtaaaaaatagaagaatacatggtagggagg

SEQ ID NO: 15 - protein, 834 aa, Homo sapiens
MKYKNLMARALYDNVPECAEELAFRKGDILTVIEQNTGGLEGWWLCSLHGRQGIVPGNRVKLLIGPMQETASSHEQPASGLMQQTFGQQKLYQVPNPQAAPRDTIYQVPPSYQNQGIYQVPTGHGTQEQEVYQVPPSVQRSIGGTSGPHVGKKVITPVRTGHGYVYEYPSRYQKDVYDIPPSHTTQGVYDIPPSSAKGPVFSVPVGEIKPQGVYDIPPTKGVYAIPPSACRDEAGLREKDYDFPPPMRQAGRPDLRPEGVYDIPPTCTKPAGKDLHVKYNCDIPGAAEPVARRHQSLSPNHPPPQLGQSVGSQNDAYDVPRGVQFLEPPAETSEKANPQERDGVYDVPLHNPPDAKGSRDLVDGINRLSFSSTGSTRSNMSTSSTSSKESSLSASPAQDKRLFLDPDTAIERLQRLQQALEMGVSSLMALVTTDWRCYGYMERHINEIRTAVDKVELFLKEYLHFVKGAVANAACLPELILHNKMKRELQRVEDSHQILSQTSHDLNECSWSLNILAINKPQNKCDDLDRFVMVAKTVPDDAKQLTTTINTNAEALFRPGPGSLHLKNGPESIMNSTEYPHGGSQGQLLHPGDHKAQAHNKALPPGLSKEQAPDCSSSDGSERSWMDDYDYVHLQGKEEFERQQKELLEKENIMKQNKMQLEHHQLSQFQLLEQEITKPVENDISKWKPSQSLPTTNSGVSAQDRQLLCFYYDQCETHFISLLNAIDALFSCVSSAQPPRIFVAHSKFVILSAHKLVFIGDTLTRQVTAQDIRNKVMNSSNQLCEQLKTIVMATKMAALHYPSTTALQEMVHQVTDLSRNAQLFKRSLLEMATF

SEQ ID NO: 16 - DNA, 4532 bp, Homo sapiens
agtgacttgagggaggcgctgcgactgacaagcggctctgcccgggaccttctcgctttcatctagcgctgcactcaatggaggggcgggcaccgcagtgcttaatgctgtcttaactagtgtaggaaaacggctcaacccaccgctgccgaaatgaagtataagaatcttatggcaagggccttatatgacaatgtcccagagtgtgccgaggaactggcctttcgcaagggagacatcctgaccgtcatagagcagaacacagggggactggaaggatggtggctgtgctcgttacacggtcggcaaggcattgtcccaggcaaccgggtgaagcttctgattggtcccatgcaggagactgcctccagtcacgagcagcctgcctctggactgatgcagcagacctttggccaacagaagctctatcaagtgccaaacccacaggctgctccccgagacaccatctaccaagtgccaccttcctaccaaaatcagggaatttaccaagtccccactggccacggcacccaagaacaagaggtatatcaggtgccaccatcagtgcagagaagcattgggggaaccagtgggccccacgtgggtaaaaaggtgataacccccgtgaggacaggccatggctacgtatacgagtacccatccagataccaaaaggacgtctatgatatccctccttctcataccactcaaggggtatacgacatccctccctcatcagcaaaaggccctgtgttttcagttccagtgggagagataaaacctcaaggggtgtatgacatcccgcctacaaaaggggtatatgccattccgccctctgcttgccgggatgaagcagggcttagggaaaaagactatgacttcccccctcccatgagacaagctggaaggccggacctcagaccggagggggtttatgacattcctccaacctgcaccaagccagcagggaaggaccttcatgtaaaatacaactgtgacattccaggagctgcagaaccggtggctcgaaggcaccagagcctgtccccgaatcacccacccccgcaactcggacagtcagtgggctctcagaacgacgcatatgatgtcccccgaggcgttcagtttcttgagccaccagcagaaaccagtgagaaagcaaacccccaggaaagggatggtgtttatgatgtccctctgcataacccgccagatgctaaaggctctcgggacttggtggatgggatcaaccgattgtctttctccagtacaggcagcacccggagtaacatgtccacgtcttccacctcctccaaggagtcctcactgtcagcctccccagctcaggacaaaaggctcttcctggatccagacacagctattgagagacttcagcggctccagcaggcccttgagatgggtgtctccagcctaatggcactggtcactaccgactggcggtgttacggatatatggaaagacacatcaatgaaatacgcacagcagtggacaaggtggagctgttcctgaaggagtacctccactttgtcaagggagctgttgcaaatgctgcctgcctcccggaactcatcctccacaacaagatgaagcgggagctgcaacgagttgaagactcccaccagatcctgagtcaaaccagccatgacttaaatgagtgcagctggtccctgaatatcttggccatcaacaagccccagaacaagtgtgacgatctggaccggtttgtgatggtggcaaagacggtgcccgatgacgccaagcagctcaccacaaccatcaacaccaacgcagaggccctcttcagacccggccctggcagcttgcatctgaagaatgggccggagagcatcatgaactcaacggagtacccacacggtggctcccagggacagctgctgcatcctggtgaccacaaggcccaggcccacaacaaggcactgcccccaggcctgagcaaggagcaggcccctgactgtagcagcagtgatggttctgagaggagctggatggatgactacgattacgtccacctacagggtaaggaggagtttgagaggcaacagaaagagctattggaaaaagagaatatcatgaaacagaacaagatgcagctggaacatcatcagctgagccagttccagctgttggaacaagagattacaaagcccgtggagaatgacatctcgaagtggaagccctctcagagcctacccaccacaaacagtggcgtgagtgctcaggatcggcagttgctgtgcttctactatgaccaatgtgagacccatttcatttcccttctcaacgccattgacgcactcttcagttgtgtcagctcagcccagcccccgcgaatcttcgtggcacacagcaagtttgtcatcctcagtgcacacaaactggtgttcattggagacacgctgacacggcaggtgactgcccaggacattcgcaacaaagtcatgaactccagcaaccagctctgcgagcagctcaagaccatagtcatggcaaccaagatggccgccctccattaccccagcaccacggccctgcaggaaatggtgcaccaagtgacagacctttctagaaatgcccagctgttcaagcgctctttgctggagatggcaacgttctgagaagaaaaaaaagaggaaggggactgcgttaacggttactaaggaaaactggaaatactgtctggtttttgtaaatgttatctatttttgtagatattttatataaaaatgaaatattttaacattttatgggtcagtcaactttcagaaattcagggagctggagagggaaatctttttttttccccctgagtggttcttatgtacatagaggtatctgagacataaactgtacagaaaacttgtccacgtgcttttgtatgcccatgtattcatgtttgtttgtagatgtttgtctgatgcatttcattaaaaaaaaaaccatgaattacgaagcaccttagtaagcacctcctaatgctgcattttttttgttgttgttaaaaacataccagctggttataatattgttctccacgtccttgtgatgattctgagcctggcactcccaaatctgggaagcatagtttatttgcaagtgttcaccttccaaatcatgaggcatagcatgacttattcttgtttggaaaactcttttcaaaactgaccatcttaaacacatgatggccaagtgcccaaaagccctcttgcggagcaaatttcagaatatatatgtggatccaagctctgatagttcaggtgctggagggaagagagacctgtgtgtttagaggccaggaccacagttaggattgggttgtttcaatactgagagacagctacaataaaaggagagcaattgcctccctggggctgttcaatcttctgcatttgtgagtggttcagtcatgaggttttccaaaagatgtttttagagttgtaaaaaccatatttgcagcaaagatttacaaaggcgtatcagactatgattgttcaccaaaataggggaatggtttgatccgccagttgcaagtagaggcctttctgactcttaatattcactttggtgctactacccccattacctgagggaaactggccaggtccttgatcatggaactatagagctaccaggacatatcctgctctctaagggaatttattgctatcttgcaccttctttaaaactcacatatgcagacctgacactcaagagtggctagctacacagagtccatctaatttttgcaacttcctgtggccagtgtgtataaccccttccactatctcacagatagtcacagcgtccattccatagtctgtctcctcacatctgttagtattgacacagcacagacaccacaagccatcaggttcttcatggggcaggtgaaatacttctaccccatgggtaaatgtattcacatattaccaagagaagaagcacattatctatgatcttttggcccagttcttatttagcatttttattccagcctacttggaaacatgtttttatttgcaatatatgcctgactgaattaagcttgcttgttttaaacaaccaaatcattggaacagaaaaggatttaaaaaacaagaatgcatgatctcagagtgattaaaaaaaaatcagtggaaataaatgatcatagaaggtgcttttcaaaacaactgctattataattctcaaagtcctactctgccaaaagaagattaaaagtcatacattacattacaaggaaatgttcatgtgggaagagggttgctgaaaatcaacaacgcttgaagttaaaaagtgtgtctttgtagatttcattgtataatgtgtatttcttaggagatggctgacttgattgatctacgctaagtggagacatttcacatttttaaaaccaaatgttcaatctgtattactctttgccgtcttgtatgtagaggctatttttaaatcattaaatttttagatctctgttttcaaaaaaaaaaaaaaa

SEQ ID NO: 17 - protein, 1252 aa, Homo sapiens
MSTTVNVDSLAEYEKSQIKRALELGTVMTVFSFRKSTPERRTVQVIMETRQVAWSKTADKIEGFLDIMEIKEIRPGKNSKDFERAKAVRQKEDCCFTILYGTQFVLSTLSLAADSKEDAVNWLSGLKILHQEAMNASTPTIIESWLRKQIYSVDQTRRNSISLRELKTILPLINFKVSSAKFLKDKFVEIGAHKDELSFEQFHLFYKKLMFEQQKSILDEFKKDSSVFILGNTDRPDASAVYLHDFQRFLIHEQQEHWAQDLNKVRERMTKFIDDTMRETAEPFLFVDEFLTYLFSRENSIWDEKYDAVDMQDMNNPLSHYWISSSHNTYLTGDQLRSESSPEAYIRCLRMGCRCIELDCWDGPDGKPVIYHGWTRTTKIKFDDVVQAIKDHAFVTSSFPVILSIEEHCSVEQQRHMAKAFKEVFGDLLLTKPTEASADQLPSPSQLREKIIIKHKKLGPRGDVDVNMEDKKDEHKQQGELYMWDSIDQKWTRHYCAIADAKLSFSDDIEQTMEEEVPQDIPPTELHFGEKWFHKKVEKRTSAEKLLQEYCMETGGKDGTFLVRESETFPNDYTLSFWRSGRVQHCRIRSTMEGGTLKYYLTDNLRFRRMYALIQHYRETHLPCAEFELRLTDPVPNPNPHESKPWYYDSLSRGEAEDMLMRIPRDGAFLIRKREGSDSYAITFRARGKVKHCRINRDGRHFVLGTSAYFESLVELVSYYEKHSLYRKMRLRYPVTPELLERYNTERDINSLYDVSRMYVDPSEINPSMPQRTVKALYDYKAKRSDELSFCRGALIHNVSKEPGGWWKGDYGTRIQQYFPSNYVEDISTADFEELEKQIIEDNPLGSLCRGILDLNTYNVVKAPQGKNQKSFVFILEPKEQGDPPVEFATDRVEELFEWFQSIREITWKIDSKENNMKYWEKNQSIAIELSDLVVYCKPTSKTKDNLENPDFREIRSFVETKADSIIRQKPVDLLKYNQKGLTRVYPKGQRVDSSNYDPFRLWLCGSQMVALNFQTADKYMQMNHALFSLNGRTGYVLQPESMRTEKYDPMPPESQRKILMTLTVKVLGARHLPKLGRSIACPFVEVEICGAEYGNNKFKTTVVNDNGLSPIWAPTQEKVTFEIYDPNLAFLRFVVYEEDMFSDPNFLAHATYPIKAVKSGFRSVPLKNGYSEDIELASLLVFCEMRPVLESEEELYSSCRQLRRRQEELNNQLFLYDTHQNLRNANRDALVKEFSVNENHSSCTRRNATRG

SEQ ID NO: 18 - DNA, 8707 bp, Homo sapiens
gaggatcacgtggcgcggcgccgcggccgaagcagaagtagcgagcgccggcggcggagggcgtgagcggcgctgagtgacccgagtcgggacgcgggctgcgcgcgcgggaccccggagcccaaacccggggcaggcgggcagctgtgcccgggcggcacggccagcttcctgatttctcccgattccttccttctccctggagcggccgacaatgtccaccacggtcaatgtagattcccttgcggaatatgagaagagccagatcaagagagccctggagctggggacggtgatgactgtgttcagcttccgcaagtccacccccgagcggagaaccgtccaggtgatcatggagacgcggcaggtggcctggagcaagaccgctgacaagatcgagggcttcttggatatcatggaaataaaagaaatccgcccagggaagaactccaaagatttcgagcgagcaaaagcagttcgccagaaagaagactgctgcttcaccatcctatatggcactcagttcgtcctcagcacgctcagcttggcagctgactctaaagaggatgcagttaactggctctctggcttgaaaatcttacaccaggaagcgatgaatgcgtccacgcccaccattatcgagagttggctgagaaagcagatatattctgtggatcaaaccagaagaaacagcatcagtctccgagagttgaagaccatcttgcccctgatcaactttaaagtgagcagtgccaagttccttaaagataagtttgtggaaataggagcacacaaagatgagctcagctttgaacagttccatctcttctataaaaaacttatgtttgaacagcaaaaatcgattctcgatgaattcaaaaaggattcgtccgtgttcatcctggggaacactgacaggccggatgcctctgctgtttacctgcatgacttccagaggtttctcatacatgaacagcaggagcattgggctcaggatctgaacaaagtccgtgagcggatgacaaagttcattgatgacaccatgcgtgaaactgctgagcctttcttgtttgtggatgagttcctcacgtacctgttttcacgagaaaacagcatctgggatgagaagtatgacgcggtggacatgcaggacatgaacaaccccctgtctcattactggatctcctcgtcacataacacgtaccttacaggtgaccagctgcggagcgagtcgtccccagaagcttacatccgctgcctgcgcatgggctgtcgctgcattgaactggactgctgggacgggcccgatgggaagccggtcatctaccatggctggacgcggactaccaagatcaagtttgacgacgtcgtgcaggccatcaaagaccacgcctttgttacctcgagcttcccagtgatcctgtccatcgaggagcactgcagcgtggagcaacagcgtcacatggccaaggccttcaaggaagtatttggcgacctgctgttgacgaagcccacggaggccagtgctgaccagctgccctcgcccagccagctgcgggagaagatcatcatcaagcataagaagctgggcccccgaggcgatgtggatgtcaacatggaggacaagaaggacgaacacaagcaacagggggagctgtacatgtgggattccattgaccagaaatggactcggcactactgcgccattgccgatgccaagctgtccttcagtgatgacattgaacagactatggaggaggaagtgccccaggatataccccctacagaactacattttggggagaaatggttccacaagaaggtggagaagaggacgagtgccgagaagttgctgcaggaatactgcatggagacggggggcaaggatggcaccttcctggttcgggagagcgagaccttccccaatgactacaccctgtccttctggcggtcaggccgggtccagcactgccggatccgctccaccatggagggcgggaccctgaaatactacttgactgacaacctcaccttcagcagcatctatgccctcatccagcactaccgcgagacgcacctgcgctgcgccgagttcgagctgcggctcacggaccctgtgcccaaccccaacccccacgagtccaagccgtggtactatgacagcctgagccgcggagaggcagaggacatgctgatgaggattccccgggacggggccttcctgatccggaagcgagaggggagcgactcctatgccatcaccttcagggctaggggcaaggtaaagcattgtcgcatcaaccgggacggccggcactttgtgctggggacctccgcctattttgagagtctggtggagctcgtcagttactacgagaagcattcactctaccgaaagatgagactgcgctaccccgtgacccccgagctcctggagcgctacaatatggaaagagatataaactccctctacgacgtcagcagaatgtatgtggatcccagtgaaatcaatccgtccatgcctcagagaaccgtgaaagctctgtatgactacaaagccaagcgaagcgatgagctgagcttctgccgtggtgccctcatccacaatgtctccaaggagcccgggggctggtggaaaggagactatggaaccaggatccagcagtacttcccatccaactacgtcgaggacatctcaactgcagacttcgaggagctagaaaagcagattattgaagacaatcccttagggtctctttgcagaggaatattggacctcaatacctataacgtcgtgaaagcccctcagggaaaaaaccagaagtcctttgtcttcatcctggagcccaagcagcagggcgatcctccggtggagtttgccacagacagggtggaggagctctttgagtggtttcagagcatccgagagatcacctggaagattgacaccaaggagaacaacatgaagtactgggagaagaaccagtccatcgccatcgagctctctgacctggttgtctactgcaaaccaaccagcaaaaccaaggacaacttagaaaatcctgacttccgagaaatccgctcctttgtggagacgaaggctgacagcatcatcagacagaagcccgtcgacctcctgaagtacaatcaaaagggcctgacccgcgtctacccaaagggacaaagagttgactcttcaaactacgaccccttccgcctctggctgtgcggttctcagatggtggcactcaatttccagacggcagataagtacatgcagatgaatcacgcattgttttctctcaatgggcgcacgggctacgttctgcagcctgagagcatgaggacagagaaatatgacccgatgccacccgagtcccagaggaagatcctgatgacgctgacagtcaaggttctcggtgctcgccatctccccaaacttggacgaagtattgcctgtccctttgtagaagtggagatctgtggagccgagtatgacaacaacaagttcaagacgacggttgtgaatgataatggcctcagccctatctgggctccaacacaggagaaggtgacatttgaaatttatgacccaaacctggcatttctgcgctttgtggtttatgaagaagatatgttcagcgatcccaactttcttgctcatgccacttaccccattaaagcagtcaaatcaggattcaggtccgttcctctgaagaatgggtacagcgaggacatagagctggcttccctcctggttttctgtgagatgcggccagtcctggagagcgaagaggaactttactcctcctgtcgccagctgaggaggcggcaagaagaactgaacaaccagctctttctgtatgacacacaccagaacttgcgcaatgccaaccgggatgccctggttaaagagttcagtgttaatgagaaccagctccagctgtaccaggagaaatgcaacaagaggttaagagagaagagagtcagcaacagcaagttttactcatagaagctggggtatgtgtgtaagggtattgtgtgtgtgcgcatgtgtgtttgcatgtaggagaacgtgccctattcacactctgggaagacgctaatctgtgacatcttttcttcaagcctgccatcaaggacatttcttaagacccaactggcatgagttggggtaatttcctattattttcatcttggacaactttcttaacttatattctttatagaggattccccaaaatgtgctcctcatttttggcctctcatgttccaaacctcattgaataaaagcaatgaaaaccttgatcaattaagccttctgttgcacgacctgtgcagtgaacaggatttcttttctggccaagaagattctacctctaatgatccaggtaactgatgtccatggaggatgagctggaaatgtaagaaactattcatgagattctgaaaaggattttaactcaaaggcaaatgattccataagggcccaaagagaagccctacccacaggcagcctgctcagttcaatgtactttaactaccaccggctgcctgctgcagtccacaagaaaatggctgagtgatgggatctgttcattaagacaatttctaattaatggtgacagcttgttttgtgactagagttactgggatggagggtaggaatcttggggcctctttgttttaaaaagcccatcagagagaccagagccgtgctgcaggggcaggttctcacttgcccctggctctgccagctgctgggaggctctggccccactagtccctcatggccctactgaactggctgggaggctgctggaatggcccttggtccacagctctccacaggcaagaggtcaactgctgcttgaaagaggtagacaaaagttaggttgatggcgaaatgtctctgggttacccagtcttctggagcagcaagctgagctttaatgggctaagcattagggtgttacagaaaatttcaaatgcagccatctcccttggggcagatctacctagttcatgacagtatgtgcggctggccagggctttacacctctgcatcttaagttgttaatacataccaataatgtaatatggctttttaaaggagaggagagtgctgggttgggaagggaggtggttggtagagtcacaacttctcaatgagtgaatttacagctgatgggaaaaggagtgtaactgtgaaaaacgatggctgtggtggggaagaacaaaccagcagtaagcctgatgtttgatgtggatggaactggcccctagaaacccatctgaccctcctcttgttacccgaaatgctgggcttagtatgcatgtactgctgaaaagcagggcagaacaaatcaggctctgaccagaagatccttctggtcccttcactctacaaaaacttactgatcacctccacatgccaaatacagtgccaagatttgggggtgtggatgtttaaacaaaaagctgtgggtctcatcaatcatctccatccacaagctcctaaaagaaagccatttacctcgcttgaagccaggaacacagggaacagcagtctggccaaggaagggctgttatctggtgctatcactccagttactcctccaactgggagctgctattttatttggcagtcagcaactgaagaaagaacattcctcttagtggcagatgttcaaagcaactttcaagaaaggctaggtgagaaaggcactgggatgagtgctgcaggcactctgtagccagggccccattagcctttggccaggtagccaccagaacctatttattgcacctggcatctcccccaacccctctcagctctgttaggacttccacacagcagagctcaggtgttgctgtcattacctcctttcagctcctcacttcattctactttaaagccacagtgctaaggcctgcatcccctttctgcccaaatgggttttttgctaccatatcaaagaacctgacatatggcggcataggaagcagaagctaagcctctctccagctgctgctgtgtaaaatccatgcgtggccaaagagaagtcaggggattatgacataaatggtgctgggaagaaccctctgcctaaaactgtctccttctcctggtgctacaaccggaatccaccatgagagagtactttcttcggttctttcctcctgtccttgacagagtaacacgttaatctggttcttggtggtgttagggactgattctctcaggaaaggcacacatggtatgatggctcttcccagagtctatgtgatgctacataacttcagtatctagctgagacatgcttcctacatgactgttaaagcacagccaatccaggccaagaagactagtaacaggcacattctgaaagatggaagcagcactgatagatcaaaaccaccactgcatatgtattacactgtttttgttcaccattttcctaagtgtgttatttagaatattggttattacaaggaaaaataaagtggggaggctggttaggccttgtgagtttgggaaacttaggttataaaaactaaataaagtttttctactgtgagactagatgtgcaggagtgaaaggtgtagagggtcttgttttccaaattcgatctcagaatctttttgccagaagtgtctcatgggacttatctatagtggaacacatttgaagacctactgctctattaagaaggcagccggacaacatgttctaatacttcgtatgctttgtgacctagttaaaatctaaacttaagtcgccatggccagtggcctttagattaagctagccttacccctgggagtataccagagctttccaaggaatacacagactccagtactctcaggggagcagtgttcagagcctcatcttcctgttatattcttctctaagattcatctgcctgagaaaatgcccttttctcaccttacaaaagaaaatatggctgtctccacctctagtcttactgtagagcatgtcccaaggtgtaaaaattcaaaatgtggatatttggaaagtgaaagacttatcaacagggcacaaatctttttgcaaatggattttccaagtttttctggtggttccaaattttttgctttcaacaaagtgggaggaacagcctgtagatttctgagtctcttagcatgtaactacaaaggggttggaagaattcagtgattctgctatcataaagcttccgttcccattgatgtatctgtgtgaacaaggatcaacatctccataaatgaaattgaaaacggaaaatagaattgatgatgaactttggctcaatcttaagatgttatcaatctacatagatgaaataattgtggagaaaagccctctttatctcattaagtgatacatttccaaagaagttttactatgtttaataatttagtgaaatttgggctatgtgtttattgattcagctcaatccagaggaaaattttaaaggcttacagccttaggattataggatactatataatacttttggtacagagatagaattaaataacataaaaatcaaaaatttattaggctaaaattttgagggagaagtggtatgaaaatacaaattcaaggagtaaaaggaaaagtggggcattccttgctactaaaaattgccttgttccaggtaagactgatcataaaaaaatggccctgttcataaaatttttaaaaagatcatagtatctatcaaataacttatattaagaacctcctgggctaaatttaaaaagtaatacaacagttttatttaaacatgtagtgtctacggtatgccagcactttgcagctatttataatgagaaattttagatgtcaatatagcaatgtgcaagaagatagagattttcaaaattcacttaagagtatctgagcataaaatgttaagattgctgatcggatgtgagggcgatctggctgcgacatctgtcaccccattgatcgccagggttgattcggctgatctggctggctaggtgggtgtccccttcctacctcaccgctccatgtgcgtccctcccgaagctgcgcgctccgtcgaagaggacgaccaaccccgatagaggaggaccggtcttcggtcaagggtatacgagtagctgcgctcccctgctggaacctccaaacaagctctcaagattgctgatctagggccactaagtgatgaattgtatttggaagcaaaaaggatggctaaaaaggacctcaacccttttgactttaaaaggaaaatagcttaaccttcaacctgtgtgacatttaactttttgaacccaaccgtaaaagctatcttctaaccaacaaaaagttaataattagatttggaattatacagaattagaaaattggcatttaaaaatactcaataatttgtccctggtttttaattttcaaaatattttctttttgaagagccagattccagtgatcctgcctctcagaaatttccacatttcttatttttcattaggccttaagaagctgcatttgtaaacttgtgtttcattattaaagcttaatttattttttatataaatagtatgtgctttgtgtacatagagaattaagtgaatgagtcacacagatgttggctgttgttaatgtgaaaattaaacagctgtatcacattttgaaaaataaaagtttcatctgaatgaatatagcaa

SEQ ID NO: 19 - protein, 500 aa, Homo sapiens
MDHTEGSPAEEPPAHAPSPGKFGERPPPKRLTREAMRNYLKERGDQTVLILHAKVAQKSYGNEKRFFCPPPCVYLMGSGWKKKKEQMERDGCSEQESQPCAFIGIGNSDQEMQQLNLEGKNYCTAKTLYISDSDKRKHFMLSVKMFYGNSDDIGVFLSKRIKVISKPSKKKQSLKNADLCIASGTKVALFNRLRSQTVSTRYLHVEGGNFHASSQQWGAFFIHLLDDDESEGEEFTVRDGYIHYGQTVKLVCSVTGMALPRLIIRKVDKQTALLDADDPVSQLHKCAFYLKDTERMYLCLSQERIIQFQATPCPKEPNKEMINDGASWTIISTDKAEYTFYEGMGPVLAPVTPVPVVESLQLNGGGDVAMLELTGQNFTPNLRVWFGDVEAETMYRCGESMLCVVPDISAFREGWRWVRQPVQVPVTLVRNDGIIYSTSLTFTYTPEPGPRPHCSAAGAILRANSSQVPPNESNTNSEGSYTNASTNSTSVTSSTATVVS

SEQ ID NO: 20 - DNA, 2499 bp, Homo sapiens
gtgtgcagggttccagcgacagcagcactggactcgtccagagggcggcgggtgagcggctggggccccgtggagccaccatggaccccgcaggggcagcagacccctcagtgcctcccaatcctttgactcacctgagcctgcaggacagatcagagatgcagctgcagagcgaagccgacaggcggagcctcccgggcacttggaccaggtcatccccagagcacaccaccattctgaggggaggcgtgcgcaggtgcctgcagcaacagtgtgaacagactgtgcggatcctgcatgccaaggtggcccagaaatcatacggaaatgagaagcggttcttctgccccccgccctgtgtctacctctcggggcctggctggagggtgaagccagggcaggatcaagctcaccaggcgggggaaacggggcccacggtctgcggttacatgggactggacagcgcgtccggcagcgccactgagacgcagaagctgaatttcgagcagcagccggactccagggaattcggctgcgccaagaccctgtacatctcagatgcagacaagaggaagcactttcggctggtgctgcggctggtgctgcgcgggggccgggagctgggtaccttccacagccgccttatcaaggtcatctcgaagccctcgcagaagaagcagtcgctgaaaaacaccgatctgtgcatatcctccggctcaaaggtctccctcttcaaccgcctgcgctctcagacggtctccacacgctacctctctgtggaggatggggcctttgtggccagtgcacgacagtgggctgccttcacgctccacctggctgatgggcactctgcccaaggagacttcccaccgcgagagggctacgttcgctatggctccctggtgcagctcgtctgcacggtcaccggcatcacactacctcccatgatcatccgtaaagtagcaaaacagtgtgcgctccttgatgtggatgagcccatctcccagctgcacaagtgtgcattccagtttccaggcagtcccccaggagggggtggcacctacttatgccttgccacagagaaggtggtgcaatttcaggcctctccctgccccaaggaggcgaacagggctctgcttaacgacagctcttgctggaccatcatcggcaccgagtcggtggaattttccttcagcaccagcctggcgtgtaccctggagccggtcactccggtgcctctcatcagcaccctagagctgagcggcgggggcgacgtggccacgctggagctccacggagagaacttccacgcggggctcaaggtgtggtttggggacgtggaggcagaaaccatgtacaggagcccgcggtccctggtgtgcgtggtgccggacgtggcggccttctgcagcgactggcgctggctgcgcgctcccatcacaatccccatgagcctggtgcgcgccgacgggctcttctaccctagtgccttctccttcacctacaccccggaatacagcgtgcggccgggtcaccccggcgtccccgagcccgccaccgacgccgacgcgctcctggagagcatccatcaggagttcacgcgcaccaacttccacctcttcatccagacttaggcgcgcccggtagccccggctgcccaccctggagggctgcgcccgcgccaggcgcggggacgtgtttctgggttctaggccctgcttccttgcccctttgctgcagaagggcagctgaaggctcaccctagaaaccgggcctggtgggtcttacccggctcactccctcccttgtccttacacatacaggaagacaagacctgagtggtgctgtctttgtgtccgtcgtgtatggctctccctgtcttcatttcttctcactctgtctctaaacctctctctctctcccttccccctcagtacttagtctacagacctatgtgcgtgtccctatccttctgtccttttctctcttcagctctccctgcctctcacacacaattttacatgccccgaggagccaagtttgggacatttaccctccaggcatctgtgtcccctcttgaagagaaaacacacagcttcacacatccaggcatagggggcaagctcttggggcatcaggaccctggagcaccaggtccttcctggaatattagatccacctggagcaccgggtctctctaagtctcacctggggaattcggtcccacctggggcaccagttcccacctagagcactgtgtcctgccctagagcacaaagacctgctcctcccgagactctctctgactgcagccaggcatagtacctttgcctgtgtttgctccctggtccacagatttggtggctgggcaggtgcctggacagtgatgaggtcttgccgccttaactgtcccccccagtcacttctcccacaggcccagcaggacgcagtcctgaggatcagggattctacagctgcattaaaatcaatcctatccaa

SEQ ID NO: 21 - DNA, 61 bp, Artificial Sequence
  Description of Artificial Sequence Synthetic oligonucleotide
atagcaaatagagttttgtttccttcccacagatatcagatttcttcaaacagtacataga

SEQ ID NO: 22 - DNA, 95 bp, Artificial Sequence
  Description of Artificial Sequence Synthetic oligonucleotide
gaaggcctccacctgagaaagaggaactaggagttgggtgtaaatcagaaaccatatgaccagacacacttgagcacactctgtgagaaaaggga